(19) United States
(12) Patent Application Publication
(10) Pub. No.: US 2021/0269860 A1
(43) Pub. Date: Sep. 2, 2021

(54) PERSON-SPECIFIC ASSESSMENT OF PROBIOTICS RESPONSIVENESS

(71) Applicants: Yeda Research and Development Co. Ltd., Rehovot (IL); The Medical Research, Infrastructure and Health Services Fund of the Tel Aviv Medical Center, Tel-Aviv (IL)

(72) Inventors: Eran SEGAL, Ramat-HaSharon (IL); Eran ELINAV, Mazkeret Batya (IL); Zamir HALPERN, Tel-Aviv (IL)

(73) Assignees: Yeda Research and Development Co. Ltd., Rehovot (IL); The Medical Research, Infrastructure and Health Services Fund of the Tel Aviv Medical Center, Tel-Aviv (IL)

(21) Appl. No.: 17/258,477

(22) PCT Filed: Jul. 8, 2019
(86) PCT No.: PCT/IL2019/050760

§ 371 Date: Jan. 7, 2021

Related U.S. Application Data
(60) Provisional application No. 62/695,067, filed on Jul. 8, 2018.
(60) Provisional application No. 62/695,068, filed on Jul. 8, 2018.

(51) Int. Cl. C12Q 1/689; C12Q 1/04; A61K 45/06; A61K 35/74; A61P 1/00; A61P 31/04

(52) U.S. Cl. CPC C12Q 1/689; C12Q 1/04; A61K 45/06; C12Q 2600/106; A61P 1/00; A61P 31/04; C12Q 2600/158; A61K 35/74

(57) ABSTRACT

A method of assessing whether a candidate subject is suitable for probiotic treatment is disclosed. The method comprises determining a signature of the gut microbiome of the candidate subject, wherein when the signature of the microbiome of the candidate subject is statistically significantly similar to a signature of a gut microbiome of a control subject known to be responsive to probiotic treatment, it is indicative that the subject is suitable for probiotic treatment.

### RELATED APPLICATIONS

This application claims the benefit of priority from U.S. Provisional Patent Application Nos. 62/695,068 and 62/695,067 filed on Jul. 8 2019, the contents of which are incorporated herein by reference in their entirety.

### FIELD AND BACKGROUND OF THE INVENTION

The present invention, in some embodiments thereof, relates to methods of using probiotics in mammalian subjects. More specifically, the invention relates to personalized predictions as to whether a subject is responsiveness to a probiotic based on the gut microbiome.

Dietary supplementation with commensal microorganisms, collectively termed probiotics, is a constantly growing market, estimated to exceed 35 billion USD globally in 2015. In 2012, in the US alone, 1.6% of the adult population (3.9 million adults) consumed prebiotics or probiotics supplements, a fourfold increase in comparison to the rates in 2007, making probiotics the third most commonly consumed dietary supplement after vitamin and mineral preparations. Claimed rationales for probiotics consumption by healthy individuals vary from alleviation of gastrointestinal (GI) symptoms, ‘fortification’ of the immune system and protection against infectious diseases, prevention of weight gain, mental and behavioral augmentation and promotion of wellbeing. A recent survey demonstrated that over 60% of healthcare providers prescribed probiotics to their patients, mostly for the maintenance of ‘bowel health’, prevention of antibiotic-associated diarrhea or upon patient request.

Nevertheless, despite the popularity of probiotic products, their efficacy under homeostatic conditions remains controversial, with only a few controlled clinical studies pointing to beneficial outcomes, while others failing to establish sustained modulation of the microbiome, or objective physiological consequences.

Collectively, evidence for health-promoting activity of exogenously administered commensals remains inconclusive. As such, probiotics are often classified by regulatory authorities as dietary supplements, emphasizing their safety and lack of impact on food taste, rather than evidence-based proofs of beneficial effects. This confusing situation results in a multitude of non-evidence-based probiotics preparations introduced to the general public in their purified forms or integrated into a variety of foods, ranging from infant formulas, milk products, to pills, powders and candy-like articles, in the absence of concrete proof of efficacy. Medical authorities, such as the European Food Safety Authority or the US Food and drug administration, have therefore declined to approve probiotics formulations as medical intervention modalities.

Several major challenges limit a comprehensive assessment of probiotics effects on the mammalian host. The first stems from common utilization of 16S rDNA analysis as means of microbiome and probiotics characterization in most studies. This methodology, when utilized alone, enables to assess only taxonomic changes in relative abundance, mostly at the genus level, while being agnostic in distinguishing between similar endogenous and probiotics strains, or in quantifying impacts on microbiome function. A second limitation stems from significant inter-individual human microbiome variability, mediated by factors such as age, diet, antibiotic usage, consumption of food supplements, underlying medical conditions and disturbances to circadian activity. This variability may drive individualized probiotics-mediated colonization and host effects, as suggested by long-term stool colonization of Bifidobacterium longum AH1206 that was noted in only 30% of individuals consuming this probiotic (Maldonado-Gomez, M. X. et al. Cell Host Microbe 20, 515-526, (2016)). A third limitation stems from universal reliance on stool microbiome assessment, as a surrogate marker of GI mucosal probiotics impacts on the host and its microbiome.

Goossens, D. A., et al. Aliment Pharmacol Ther 23, 255-263, (2006) discloses a human study utilizing only culture-based techniques in individuals undergoing surveillance colonoscopy in which they failed to detect efficient probiotics gut colonization.

Clinical trial NCT03218579 examines the extent of rehabilitation of the composition and functioning of the intestinal bacteria in healthy people after the consumption of antibiotics.

### SUMMARY OF THE INVENTION

According to an aspect of some embodiments of the present invention there is provided a method of assessing whether a candidate subject is suitable for probiotic treatment comprising determining a signature of the gut microbiome of the candidate subject, wherein when the signature of the microbiome of the candidate subject is statistically significantly similar to a signature of a gut microbiome of a control subject known to be responsive to probiotic treatment, it is indicative that the subject is suitable for probiotic treatment.

According to an aspect of some embodiments of the present invention there is provided a method of treating a disease comprising administering a therapeutically effective amount of a probiotic to a subject in need thereof, the subject being deemed responsive to probiotic treatment according to the methods described herein thereby treating the disease.

According to an aspect of some embodiments of the present invention there is provided a method of maintaining the health of a subject comprising administering a probiotic to a subject who is deemed responsive to probiotic treatment according to the methods described herein, thereby maintaining the health of the subject.

According to an aspect of some embodiments of the present invention there is provided a method of treating a disease of a subject for which an antibiotic is therapeutic comprising:

(a) assessing whether the subject is suitable for probiotic treatment according to the methods described herein;

(b) administering to the subject an antibiotic which is suitable for treating the disease; and subsequently

(c) administering to the subject a probiotic if the subject is deemed suitable for probiotic treatment; or administering to the subject a fecal transplant if the subject is deemed not suitable for probiotic treatment, thereby treating the disease.

According to an aspect of the present invention there is provided a method of predicting a signature of a microbiome of a GI location of a subject, the method comprising determining an amount and/or activity of at least one genus or order of bacteria of a fecal sample of the subject, the genus or order being set forth in Table N, wherein the amount and/or activity is predicative of the signature of the microbiome of a GI location of the subject.

According to an aspect of the present invention there is provided a method of predicting a signature of a microbiome of a GI location of a subject, the method comprising determining an amount and/or activity of at least one species of bacteria of a fecal sample of the subject, the species being set forth in Table O, wherein the amount and/or activity is predicative of the signature of the microbiome of a GI location of the subject.

According to an aspect of the present invention there is provided a predicting a signature of a microbiome of a GI location of a subject, the method comprising determining an amount and/or activity of at least one KO annotation of bacteria of a fecal sample of the subject, the KO annotation being set forth in Table P, wherein the amount and/or activity is predicative of the signature of the microbiome of a GI location of the subject.

According to an aspect of the present invention there is provided a method of predicting a signature of a microbiome of a GI location of a subject, the method comprising determining an amount and/or activity of bacteria utilizing at least one KEGG pathway of a fecal sample of the subject, the KEGG pathway being set forth in Table Q, wherein the amount and/or activity is predicative of the signature of the microbiome of a GI location of the subject.

According to further features in the described preferred embodiments, the determining the signature is effected by analyzing feces of the subject.

According to further features in the described preferred embodiments, the gut microbiome comprises a mucosal gut microbiome or a lumen gut microbiome.

According to further features in the described preferred embodiments, the probiotic comprises at least one of the bacterial species selected from the group consisting of B. bifidum, L. rhamnosus, L. lactis, L. casei, B. breve, S. thermophilus, B. longum, L. paracasei, L. plantarum and B. infantis.

According to further features in the described preferred embodiments, the candidate subject does not have a chronic disease.

According to further features in the described preferred embodiments, the signature of the gut microbiome is a presence or level of microbes of the microbiome.

According to further features in the described preferred embodiments, the signature of the gut microbiome is a presence or level of genes of microbes of the microbiome.

According to further features in the described preferred embodiments, the signature of the gut microbiome is a presence or level of a product generated by microbes of the microbiome.

According to further features in the described preferred embodiments, the signature of the gut microbiome is an alpha diversity.

According to further features in the described preferred embodiments, the product is selected from the group consisting of a mRNA, a polypeptide, a carbohydrate and a metabolite.

According to further features in the described preferred embodiments, the microbes of the microbiome are of an identical species to the microbes of the probiotic.

According to further features in the described preferred embodiments, the determining the signature is effected by analyzing feces of the subject.

According to further features in the described preferred embodiments, the microbes of the microbiome are of the species selected from the group consisting of those set forth in Table A and/or are of the genus Bifidobacterium or Dialister.

According to further features in the described preferred embodiments, the microbes of the microbiome utilize at least one pathway set forth in Table B.

According to further features in the described preferred embodiments, the determining the signature is effected by analyzing the lower gastrointestinal tract (LGI) mucosal microbiome of the subject.

According to further features in the described preferred embodiments, the microbes of the LGI mucosal microbiome are selected from the group consisting of bacteria of the genus Odoribacter, bacteria of the genus Bacteroides, bacteria of the genus Bifidobacterium, bacteria of the family Rikenellaceae and a species set forth in Table C.

According to further features in the described preferred embodiments, the microbes of the LGI mucosal microbiome utilize at least one pathway set forth in Table D.

According to further features in the described preferred embodiments, the determining the signature is effected by analyzing the rectal microbiome of the subject.

According to further features in the described preferred embodiments, the microbes of the rectal microbiome are selected from the group consisting of bacteria of the genus Streptococcus, bacteria of the genus Odoribacter, bacteria of the genus Bifidobacterium, bacteria of the genus Bacteroides, bacteria of the family Rikenellaceae and bacteria of the species Barnesiella_intestinihominis.

According to further features in the described preferred embodiments, the microbes of the rectal microbiome utilize at least one pathway listed in Table E.

According to further features in the described preferred embodiments, the determining the signature is effected by analyzing the sigmoid colon (SM) microbiome of the subject.

According to further features in the described preferred embodiments, the SM microbiome are selected from the group consisting of bacteria of the family Rikenellaceae and bacteria of the species listed in Table F.

According to further features in the described preferred embodiments, the microbes of the SM microbiome utilize at least one pathway listed in Table G.

According to further features in the described preferred embodiments, the determining the signature is effected by analyzing the descending colon (DC) microbiome of the subject.

According to further features in the described preferred embodiments, the microbes of the DC microbiome are selected from the group consisting of bacteria of the genus Bacteroides, bacteria of the genus Odoribacter, bacteria of the family Rikenellaceae and bacteria of the species set forth in Table H.

According to further features in the described preferred embodiments, the microbes of the DC microbiome utilize at least one pathway listed in Table I.

According to further features in the described preferred embodiments, the determining the signature is effected by analyzing the transverse colon (TC) microbiome of the subject.

According to further features in the described preferred embodiments, the microbes of the TC microbiome are selected from the group consisting of Bacteria of the genus Odoribacter, bacteria of the genus Dorea, bacteria of the family Rikenellaceae and bacteria of the species set forth in Table J.

According to further features in the described preferred embodiments, the microbes of the TC microbiome utilize at least one pathway listed in Table K.

According to further features in the described preferred embodiments, the determining the signature is effected by analyzing the ascending colon (AC) microbiome of the subject.

According to further features in the described preferred embodiments, the microbes of the AC microbiome are selected from the group consisting of Bacteria of the genus Odoribacter, bacteria of the family Rikenellaceae and bacteria of the species set forth in Table L.

According to further features in the described preferred embodiments, the microbes of the AC microbiome utilize a fatty acid degradation pathway.

According to further features in the described preferred embodiments, the determining the signature is effected by analyzing the cecum (Ce) microbiome of the subject.

According to further features in the described preferred embodiments, the microbes of the Ce microbiome are selected from the group consisting of Bacteria of the genus Odoribacter, bacteria of the family Rikenellaceae and bacteria of the species Barnesiella_intestinihominis.

According to further features in the described preferred embodiments, the microbes of the Ce microbiome utilize a propanoate metabolism Kegg pathway or the primary bile acid biosynthesis Kegg pathway.

According to further features in the described preferred embodiments, the determining the signature is effected by analyzing the ileum (Ti) microbiome of the subject.

According to further features in the described preferred embodiments, the microbes of the Ti microbiome are selected from the group consisting of bacteria of the genus Faecalibacterium, bacteria of the family Rikenellaceae, bacteria of the genus Bifidobacterium, bacteria of the family Ruminococcaceae.

According to further features in the described preferred embodiments, the microbes of the Ti microbiome utilize a limonene and pinene degradation Kegg pathway or the valine, leucine and isoleucine degradation Kegg pathway.

According to further features in the described preferred embodiments, the determining the signature is effected by analyzing the fundus (GO microbiome of the subject.

According to further features in the described preferred embodiments, the microbes of the Gf microbiome are of the genus Actinobacillus.

According to further features in the described preferred embodiments, the microbes of the Gf microbiome utilize a Kegg pathway set forth in Table M.

According to further features in the described preferred embodiments, the fecal transplant is an autologous fecal transplant.

According to further features in the described preferred embodiments, the predicting is based on the level and/or activity of no more than 10 bacterial genii or orders in the fecal sample.

According to further features in the described preferred embodiments, the predicting is based on the level and/or activity of no more than 10 bacterial species in the fecal sample.

According to further features in the described preferred embodiments, the predicting is based on the level and/or activity of no more than KO annotations in the fecal sample.

According to further features in the described preferred embodiments, the predicting is based on the level and/or activity of no more than 10 KEGG pathways in the fecal sample.

According to further features in the described preferred embodiments, the GI location is selected from the group consisting of the mucosa of the lower gastrointestinal tract, the rectum; the sigmoid colon; the distal colon; the transverse colon; the ascending colon; the cecum; the ileum; the jejunum; the duodenum; the antrum; and the fundus.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-J. Human fecal microbiome is a limited indicator of gut mucosal-associated microbiome composition and metagenomic function. (A) Anatomical regions sampled during endoscopy procedures. (B) Bacterial load in mucosal samples as quantified by qPCR of the 16S rDNA global primer, normalized to a detection threshold of 40. (C-D) 16S rDNA sequencing-based Unweighted UniFrac distances between stool and the gut microbiome in the upper gastrointestinal tract (UGI), terminal ileum (TI) and lower gastrointestinal (LGI) tract, portrayed in (C) principal coordinate analysis (PCoA) and (D) quantification of distances to stool. (E) Relative abundances of the ten most common genera in each anatomical region and stool. (F) Species significantly variable between the LGI mucosa and stool samples in red. (G-H) Shotgun metagenomic sequencing-based analysis of bacterial KEGG orthologous (KO) genes, (G) Principal component analysis (PCA) of KO relative abundances; (H) Spearman's rank correlation matrices of KOs in stool versus endoscopic samples of luminal and mucosal microbiome; (I) Groups of KEGG pathways significantly different between stool samples, the LGI lumen or mucosa, or the TI. (J) Specific pathways significantly variable between stool and the LGI lumen in red. St, stomach; GF, gastric fundus; GA, gastric antrum; Je, jejunum; Du, duodenum; TI, terminal ileum; Ce, cecum; AC, ascending colon; TC, transverse colon; DC, descending colon; SC, sigmoid colon; Re, rectum. Symbols or horizontal lines represent the mean, error bars SEM or 10-90 percentiles. *, P<0.05; **, P<0.01; ***, P<0.001; ****, P<0.0001. Kruskal-Wallis & Dunn's (panels B, D & H); Wilcoxon rank sum with FDR correction (panels F, J).

FIGS. 2A-G. Colonization resistance to probiotics by the murine gut microbiome. SPF mice were gavaged daily with probiotics (Prob) or remained untreated (Ctrl) for 28 days. Relative or absolute abundance of probiotics strains was determined by qPCR in stool samples at the indicated time points or in GI tract tissues on day 28. (A) Experimental design in SPF mice. (B) Quantification of specific probiotics species in stool by qPCR. Significant differences from the baseline are denoted. (C) Aggregated qPCR-based quantification of all probiotics targets in stool samples, normalized to baseline. Inset: area under incremental bacterial load curve. (D) Species-specific qPCR quantification of probiotics in mucosal and luminal samples throughout the murine GI tract. Significant differences from control are denoted. (E) Experimental outline in GF (G) mice. (F) Same as D but in GF mice. (G) qPCR-based enumeration of pooled probiotics targets in luminal and mucosal subregions of SPF and GF GI tracts, normalized to a detection threshold CT of 40. BBI, Bifidobacterium bifidum; BBR, Bifidobacterium breve; BIN, Bifidobacterium infantis; BLO, Bifidobacterium longum; LAC, Lactobacillus acidophilus; LCA, Lactobacillus casei; LLA, Lactococcus lactis; LPA, Lactobacillus paracasei; LPL, Lactobacillus plantarum; LRH, Lactobacillus rhamnosus; STH, Streptococcus thermophilus. ST, stomach; DU, duodenum; PJ, proximal jejunum; DJ, distal jejunum; IL, ileum; CE, cecum; PC, proximal colon; DC, distal colon. UGI, upper GI; LGI, lower GI. Symbols and horizontal lines represent the mean, error bars SEM. *, P<0.05; **, P<0.01; ***, P<0.001; ****, P<0.0001. Two-Way ANOVA and Dunnett (B) or Sidak (C, E), Mann-Whitney (Inset, C), or Kruskal-Wallis test and Dunn's (panel G). In B & D, *, any P<0.05-0.0001 for clarity. The experiment was repeated 3 times.

FIGS. 3A-F. Probiotics alter the murine gastrointestinal microbiome. Microbiota alterations were assessed following probiotics administration in GI mucosal and luminal samples. (A-C) PCoA of weighted UniFrac distances between probiotics-administered mice or controls in GI tract tissues and quantification in the (B) UGI or (C) LGI. (D-E) Observed species in the (D) LGI or the (E) UGI. (F) Taxa significantly different between control and probiotics in the LGI mucosa in red. Horizontal lines represent the mean, error bars 10-90 percentiles. **, P<0.01; ***, P<0.001; ****, P<0.0001. Kruskal-Wallis and Dunn's (panel B-C), Mann-Whitney (D). Lum, lumen; Muc, mucosa; Ctrl, control; Prob, probiotics; UGI, upper gastrointestinal tract; LGI, lower gastrointestinal tract.

FIGS. 4A-K. Global and individualized probiotics colonization patterns in the human GI tract. Human participants were treated with probiotics pills or placebo bidaily for a period of 28 days. (A) Experimental outline in humans. (B) qPCR-based quantification of probiotics species fecal shedding in supplemented individuals or placebo on day 19 of consumption and one month after probiotics cessation, normalized to baseline. *, any P<0.05-0.0001 for clarity, two-way ANOVA & Dunnett. (C) Aggregated probiotics load in feces. (D) Same as B but in the LGI and UGI mucosa at day 28 normalized to baseline. Two-way ANOVA for species, with Dunnett per species per region. (E) Aggregated probiotics load in the LGI mucosa normalized to baseline. (F-G) qPCR-based quantification of mucosal colonization with the 11 probiotics strains pooled for each participant in (F) each anatomical region or (G) aggregated, values during probiotics/placebo normalized to each individual baseline in each region. (H) Same as B but per participant. (I) Same as C but aggregated per group of individuals. (J-K) Probiotics strain quantification based on mapping of metagenomic sequences to unique genes, which correspond to the strains found in the probiotics pill in (J) the GI tract or (K) stool samples. Dark gray marks the presence of the probiotics species and red marks the presence of the probiotics strains. GA, gastric antrum; Je, jejunum; Du, duodenum; TI, terminal ileum; Ce, cecum; AC, ascending colon; TC, transverse colon; DC, descending colon; SC, sigmoid colon; Re, rectum. BBI, Bifidobacterium bifidum; BBR, Bifidobacterium breve; BIN, Bifidobacterium infantis; BLO, Bifidobacterium longum; LAC, Lactobacillus acidophilus; LCA, Lactobacillus casei; LLA, Lactococcus lactis; LPA, Lactobacillus paracasei; LPL, Lactobacillus plantarum; LRH, Lactobacillus rhamnosus; STH, Streptococcus thermophilus. P, permissive; R, resistant. N.S., non-significant. LGI, lower gastrointestinal tract. Prob, probiotics. Horizontal lines represent the mean, error bars SEM or 10-90 percentiles. *, P<0.05; **, P<0.01; ****, P<0.0001. Two-Way ANOVA and Dunnett's (panels B & D), Kruskal-Wallis & Dunn's (C), Mann-Whitney and permutation tests (panels E & G).

FIGS. 5A-I. Microbiome and host factors determine colonization by probiotics. (A) Aggregated probiotics load and specific species significantly distinct at baseline between permissive (P) and resistant (R) individuals. (B) 16S-based unweighted UniFrac distance separating stool microbiome composition of permissive from resistant individuals prior to probiotics supplementation. (C) MetaPhlAn2-based PCA separating permissive and resistant individuals in the LGI mucosa at baseline. (D) Experimental validation of a causative role for the microbiome in resistance; pre-supplementation fecal samples from a permissive or resistant individual were used to conventionalize (CONV) GF mice, followed by daily gavage with probiotics and GI dissection after 28 days. (E-F) Probiotics load normalized to a detection threshold of 40 (E) per species per anatomical region or (F) aggregated in the GI of conventionalized germ-free mice. (G) Heatmap representing genes that differ in abundance between permissive and resistant individuals in the stomach prior to probiotics supplementation. (H) Pathways that are significantly enriched after FDR-correction in resistant stomachs at baseline. No pathways were significantly enriched in permissive. (I) Same as H but in the terminal ileum with both groups showing discriminating pathways. Horizontal lines represent the mean, error bars SEM. *, P<0.05; **, P<0.01; ****, P<0.0001. Mann-Whitney (panels A, B & F). In E, * represent s any P<0.05-0.0001 for clarity, two-way ANOVA & Sidak.

FIGS. 6A-H. Global effects of probiotics on the human GI microbiome and host transcriptome. (A) Unweighted UniFrac distances between 16S rDNA sequencing-based taxa abundances of stool samples collected throughout the study and their respective baseline samples. Asterisks on horizontal lines compare periods according to a paired Friedman's test & Dunn's, excluding days 1-3. Asterisks on symbols according to two-way ANOVA & Dunnett to baseline. (B) Taxa that significantly differ in stool before and on the last day of probiotics supplementation in red. (C-E) 16S-based weighted UniFrac distance between probiotics and placebo consuming individuals after 21 days in the (C) UGI or the (D-E) LGI. (F) KEGG pathways-based 1-Spearman's correlation to baseline in probiotics and placebo LGI mucosa. Significance according to Mann-Whitney test. (G) PCA based on KEGG pathways in the LGI mucosa of probiotics and placebo on day 21. (H) Genes significantly altered in expression levels in the ileum of probiotics consuming individuals between day 21 and baseline in red. Horizontal lines represent the mean, error bars SEM. *, P<0.05; **, P<0.01.

FIGS. 7A-K. Probiotics differentially affect the stool and LGI mucosal microbiome in permissive and resistant individuals. (A) 16S-based distances to baseline in stools of permissive (P) and resistant (R) individuals. Inset: area under the distance to baseline curve. (B) Species that changed in relative abundance in permissive individuals before (B) and during (D) probiotics consumption but not in resistant. (C-D) Same as A-B but with KEGG pathways and 1-Spearman's correlation. (E-F) MetaPhlAn2-based (E) PCA and (F) Bray-Curtis dissimilarity indices separating permissive and resistant individuals in the LGI after 21 days of probiotics consumption. (G) Same as B but in the LGI mucosa and also compared to no change in placebo. (H) Alpha diversity in fecal microbiome before and during probiotics supplementation in the both groups; (I-J) Bacterial load as quantified by qPCR of the 16S rDNA global primer and normalized to baseline in (I) stool samples or (J) the LGI mucosa. (K) Host pathways that distinguish significantly between permissive and resistant individuals in the cecum following probiotics supplementation, FDR corrected. Horizontal lines or symbols represent the mean, error bars SEM or 10-90 percentiles. *, P<0.05; **, P<0.01; ****, P<0.0001. Mann-Whitney.

FIGS. 8A-L. Murine stool microbiome configuration only partially correlates with the gut mucosa microbiome. (A) Scheme of the gastrointestinal regions sampled from 14 weeks old male C57Bl/6 mice housed at the Weizmann institute SPF animal facility for six weeks without intervention (N=10). (B-C) Unweighted UniFrac distances between upper gastrointestinal (UGI), lower gastrointestinal (LGI) and stool samples in a (B) Principal coordinate analysis (PCoA) and (C) quantification of distances to stool; (D) Global taxonomic differences; (E-G) FDR-corrected significant differences in composition between (E) UGI and LGI (F) LGI mucosa and stool (G) LGI lumen and stool. (H-I) Taxa significantly different between lumen and mucosa in the (H) UGI and (I) LGI. (J) Per anatomical region abundance of taxa significantly different from stool. (K) alpha diversity. (L) qPCR based quantification of bacterial load normalized to a detection threshold of 40. ST, stomach; DU, duodenum; PJ, proximal jejunum; DJ, distal jejunum; IL, ileum; CE, cecum; PC, proximal colon; distal colon. UGI, upper gastrointestinal tract; TI, terminal ileum; LGI, lower gastrointestinal tract. Muc, mucosa; Lum, Lumen. Symbols represent the mean, error bars SEM. *, P<0.05; **, P<0.01; ***, P<0.001; **** P<0.0001. One-Way ANOVA & Tukey (C), Mann-Whitney (H, I, K, L) and two-way ANOVA & Dunnett (J).

FIGS. 9A-J. Bowel preparation alters the human gut microbiome composition and function. (A) Experimental outline in humans. (B) 16S rDNA sequencing-based unweighted UniFrac distances between the gut microbiome in prepped and non-prepped samples, paired by anatomical region (n=2). (C) Principal coordinate analysis (PCoA) separating prepped and non-prepped LGI endoscopic samples. (D-F) Same as C for (D) MetaPhlAn2-, (E) KEGG orthologous (KO) genes and (E) functional pathways-based PCAs. (G) Features that differed in prepped and non-prepped LGI mucosa, based on 16S and shotgun metagenomic sequencing. (H) 16S-based alpha diversity and (I) bacterial load as determined by qPCR of the 16S rDNA global primer in the UGI, TI and LGI. (J) Species-specific qPCR quantification of probiotics in mucosal samples throughout the human GI tract. BBI, Bifidobacterium bifidum; BBR, Bifidobacterium breve; BIN, Bifidobacterium infantis; BLO, Bifidobacterium longum; LAC, Lactobacillus acidophilus; LCA, Lactobacillus casei; LLA, Lactococcus lactis; LPA, Lactobacillus paracasei; LPL, Lactobacillus plantarum; LRH, Lactobacillus rhamnosus; STH, Streptococcus thermophilus. GF, gastric fundus; GA, gastric antrum; Du, duodenum; Je, jejunum; TI, terminal ileum; Ce, cecum; AC, ascending colon; TC, transverse colon; DC, descending colon; SC, sigmoid colon; Re, rectum. UGI, upper gastrointestinal tract; LGI, lower gastrointestinal tract.

FIGS. 10A-L. Human fecal microbiome is a limited indicator of gut mucosal-associated microbiome composition. (A) 16S rDNA sequencing-based unweighted UniFrac distance matrix stool, lumen and mucosa samples. (B) Shotgun sequencing-based Bray-Curtis dissimilarity between stool, lumen and mucosa samples (MetaPhlAn2). Quantification of distances to stool (Kruskal-Wallis & Dunn's). (C) Significant differences in composition between UGI mucosa and LGI mucosa by 16S rDNA sequencing. (D) Top 24 species with the greatest (absolute) fold differences in abundance between UGI mucosa and LGI mucosa by MetaPhlAn2, paired by participant. (E) Significant differences in composition between UGI lumen and UGI mucosa by 16S rDNA sequencing. (F-G) Significant differences in composition between LGI lumen and LGI mucosa by (F) 16S rDNA sequencing and (G) shotgun metagenomic sequencing. (H-J) Significant differences in composition between (H) UGI mucosa and stool, (I) UGI lumen and stool and (J) LGI mucosa and stool by 16S rDNA sequencing. (K-L) Significant differences in composition between LGI lumen and stool by (K) 16S rDNA sequencing and (L) shotgun metagenomic sequencing. UGI, upper gastrointestinal tract; TI, terminal ileum; LGI, lower gastrointestinal tract. Muc, mucosa; Lum, Lumen. Symbols represent the mean, error bars SEM. **, P<0.01; ***, P<0.001; ****, P<0.0001. Kruskal-Wallis & Dunn's (panel B); Wilcoxon rank sum with FDR correction (panels C, E-L).

FIGS. 11A-H. Human fecal microbiome is a limited indicator of gut mucosal-associated microbiome function. (A-H) Shotgun metagenomic sequencing-based analysis of bacterial KEGG orthologous (KO) genes and functional pathways for fecal and gut microbiome. (A) Spearman's rank correlation matrix between stool, lumen and mucosa samples. (B) Quantification of 1-Spearman's rank correlations between KEGG pathway abundance in endoscopic samples to stool (Kruskal-Wallis & Dunn's) and (C) distance matrix. (D) Relative abundances of the ten most common KEGG pathways in each anatomical region and stool. (E-H) Significant differences in bacterial functional pathways between (E) UGI and LGI mucosa, (F) LGI lumen and LGI mucosa, (G) LGI mucosa and stool and (H) LGI lumen and stool. UGI, upper gastrointestinal tract; TI, terminal ileum; LGI, lower gastrointestinal tract. Muc, mucosa; Lum, Lumen. Symbols represent the mean, error bars SEM. *, P<0.05, **, P<0.01; ***, P<0.001; ****, P<0.0001. Kruskal-Wallis & Dunn's (panel B); Wilcoxon rank sum with FDR correction (panels E-H).

FIGS. 12A-C. Human transcriptome in homeostasis. (A) Principal component analysis (PCA) plot depicting clustering of the human transcriptome by various anatomical regions along the gastrointestinal tract. (B) Heat map of the 100 most variable genes between anatomical regions, (C) Distances between terminal ileum to duodenal and jejunal samples and to colonic samples: bacterial taxonomical similarity assessed by unweighted UniFrac distances (left) versus host transcriptional similarity assessed by Euclidean distances. St, stomach; Du, duodenum; Je, jejunum; TI, terminal ileum; Ce, cecum; DC, descending colon. Symbols represent the mean, error bars SEM. ****, P<0.0001. Mann-Whitney U test (panel C).

FIGS. 13A-G. Probiotic strains present in probiotic pill are identifiable and culturable. (A) Probiotic pill composition by 16S rDNA sequencing (genera level). (B) Quantification of live bacteria genera cultured from probiotic pill on selective and non-selective media by 16S rDNA sequencing. (C) Probiotic pill composition by shotgun sequencing. (D) qPCR amplification of probiotics strains target in templates obtained from pure cultures. (E) Receiver-operator curve of the CT values obtained from true and mismatched pairs of D. (F-G) qPCR-based enumeration of bacteria derived from probiotics pill (F) and stool samples (G) either with or without culturing. St, stomach; GF, gastric fundus; GA, gastric antrum; Je, jejunum; Du, duodenum; TI, terminal ileum; Ce, cecum; AC, ascending colon; TC, transverse colon; DC, descending colon; SC, sigmoid colon; Re, rectum. Symbols represent the mean, error bars SEM.

FIG. 14. Quantification of probiotics genera in the murine GI tract. SPF mice were gavaged daily with probiotics (green) or remained untreated (gray) for 28 days. Relative abundance of probiotics genera was determined by 16S rDNA sequencing in GI tract tissues during the last day. ST, stomach; DU, duodenum; PJ, proximal jejunum; DJ, distal jejunum; IL, ileum; CE, cecum; PC, proximal colon; DC, distal colon. Symbols represent the mean, error bars SEM. The experiment was repeated 3 times.

FIGS. 15A-C. Characterization of fecal microbiome in probiotics consuming mice and controls. (A) Unweighted UniFrac distance of fecal microbiome composition to baseline in both groups. (B) Fecal observed species. (C) Genera significantly (FDR-corrected Mann-Whitney P<0.05) variable in stools from the last day of exposure to probiotics between treatment and controls in red. Symbols represent the mean, error bars SEM **, P<0.01; ****, P<0.0001, two-Way ANOVA & Tukey.

FIGS. 16A-D. Probiotics alter the murine gastrointestinal microbiome, which is not explained by presence of probiotics genera. The following metrics were recalculated after omitting the 4 probiotics genera (Lactobacillus, Bifidobacterium, Lactococcus, Streptococcus) from the analysis, renormalizing relative abundances to one and rarefying to 10000 (stool) or 5000 (tissues). (A) Unweighted UniFrac distances in stool samples (B) Alpha diversity in the LGI. (C-D) Weighted UniFrac distances in tissues of the (C) UGI or (D) LGI. Symbols and horizontal lines represent the mean, error bars SEM or 10-90 percentile. *, P<0.05; **, P<0.01; ***, P<0.001; ****, P<0.0001. N.S., non-significant. Two-Way ANOVA & Tukey (A), Mann-Whitney (B), Kruskal-Wallis & Dunn's (C-D).

FIGS. 17A-J. Probiotic genera are not enriched during exogenous supplementation. (A-D) 16S rDNA sequencing-based detection of probiotic genera in stool before, during and after supplementation: (A) Lactobacillus, (B) Bifidobacterium, (C) Streptococcus and (D) Lactococcus. (E-F) 16S rDNA sequencing-based detection of probiotic genera in the gastrointestinal (E) lumen and (F) mucosa for the probiotics and placebo arms. Probiotic species are sparsely identifiable in LGI mucosa samples, while increase in abundance in stool during supplementation period. (G-J) qPCR-based quantification of probiotic species (G) in stool, (H) in LGI lumen and (I) mucosa normalized to baseline abundances for the probiotics and placebo arms. (J) Aggregated probiotics load in the LGI mucosa normalized to baseline in both groups. St, stomach; GF, gastric fundus; GA, gastric antrum; Je, jejunum; Du, duodenum; TI, terminal ileum; Ce, cecum; AC, ascending colon; TC, transverse colon; DC, descending colon; SC, sigmoid colon; Re, rectum. BBI, Bifidobacterium bifidum; BBR, Bifidobacterium breve; BIN, Bifidobacterium infantis; BLO, Bifidobacterium longum; LAC, Lactobacillus acidophilus; LCA, Lactobacillus casei; LLA, Lactococcus lactis; LPA, Lactobacillus paracasei; LPL, Lactobacillus plantarum; LRH, Lactobacillus rhamnosus; STH, Streptococcus thermophilus. Asterisks within a cell denote significant enrichment of a strain compared to baseline. *, P<0.05; **, P<0.01. Two-way ANOVA & Dunn's (panels E-I).

FIGS. 18A-D. Humans feature varying degrees of probiotics association with the lower gastrointestinal mucosa, which is not reflected in stool. (A-B) Quantification of probiotics species in LGI mucosa by (A) qPCR and (B) MetaPhlAn2 three weeks through supplementation, normalized to baseline. (C) qPCR quantification of probiotics species fecal shedding in supplemented individuals on day 19 of consumption and one month after probiotics cessation, normalized to baseline. (D) Same as C but with MetaPhlAn2 on days 4-28 of consumption and days 2-4 weeks following probiotics cessation. GF, gastric fundus; GA, gastric antrum; Je, jejunum; Du, duodenum; TI, terminal ileum; Ce, cecum; AC, ascending colon; TC, transverse colon; DC, descending colon; SC, sigmoid colon; Re, rectum. Asterisks above a participant number denote a significant enrichment in overall probiotic strain abundance compared to baseline. *, P<0.05; **, P<0.01, ***, P<0.001. Mann-Whitney test (panels A-B). Two-way ANOVA & Dunn's (panels C-D).

FIGS. 19A-L. Baseline personalized host and mucosal microbiome features are associated with probiotics colonization efficacy. (A) Spearman's correlation between the initial bacterial load of a probiotic target in a specific mucosal niche and its fold change after probiotics supplementation, as determined by qPCR. (B-C) 16S-based PCoA of (B) unweighted and (C) weighted UniFrac distances separating stool microbiome composition of probiotics-permissive (P) from resistant (R) individuals prior to probiotics supplementation. (D) Same as B-C for MetaPhlAn2 PCA. (E) Bray-Curtis dissimilarity indices separating permissive and resistant individuals in stool prior to probiotics consumption. Significance according to Mann-Whitney test. (F-G) PCA based on bacterial KOs separating stool of probiotics-permissive (P) from resistant individuals prior to probiotics consumption, with (G) Euclidean distances enumerated and compared according to Mann-Whitney test. (H-I) Same as F-G for KEGG pathways. (J) 16S-based PCoA of unweighted UniFrac distances separating LGI mucosa and lumen composition of probiotics-permissive (P) from resistant (R) individuals prior to probiotics supplementation. (K) Unweighted UniFrac distances and (L) Bray-Curtis dissimilarity indices separating permissive and resistant individuals in LGI prior to probiotics consumption. Significance according to Mann-Whitney tests. **, P<0.01; ***, P<0.001, ****, P<0.0001. Mann-Whitney test (panels E, G, I, K, L).

FIGS. 20A-H. Global effects of probiotics on the human GI microbiome. (A) Bray-Curtis dissimilarity indices between shotgun sequencing-based taxa abundances of stool samples collected throughout the study and their respective baseline samples (MetaPhlAn2). Asterisks on horizontal lines compare periods according to a paired Friedman's test & Dunn's, excluding days 1-3. Asterisks on symbols according to two-way ANOVA & Dunnett to baseline. (B) Species that significantly differ in stool at baseline and one month following probiotics cessation (MetaPhlAn2). (C-D) Same as A, but with 1-Spearman's correlation to baseline for (C) bacterial KOs and (D) KEGG pathways. (E) Same as A, but with alpha diversity, normalized to baseline stool samples. (F) PCA based on MetaPhlAn2 in the LGI mucosa of probiotics and placebo on day 21. (G) Shotgun sequencing-based Bray-Curtis dissimilarity to baseline in probiotics and placebo LGI mucosa (MetaPhlAn2). (H) Same as F, but for bacterial KOs. Horizontal lines represent the mean, error bars SEM. *, P<0.05; **, P<0.01, ***, P<0.001. Friedman's test & Dunn's and two-way ANOVA & Dunnett (panels A, C, D).

FIGS. 21A-D. Probiotics differentially affect the stool and LGI mucosal microbiome in permissive and resistant individuals. (A) Shotgun sequencing-based Bray-Curtis dissimilarity indices to baseline in stools of permissive (P) and resistant (R) individuals. Inset: area under the distance to baseline curve. (B) Genera that changed in relative abundance in permissive individuals before (B) and during (D) probiotics consumption but not in resistant. (C) Same as A with bacterial KOs and 1-Spearman's correlation. (D) Host pathways that distinguish significantly between permissive and resistant individuals in the distal colon following probiotics supplementation, FDR corrected. Horizontal lines or symbols represent the mean, error bars SEM or 10-90 percentiles.

FIGS. 22A-F. Antibiotics do not alleviate mucosal colonization resistance to probiotics in mice. Four groups of WT mice (N=10) were treated for 14 days with cipro-flagyl in drinking water, after which one group was immediately dissected, and three others were followed by either daily probiotics administration, a single auto-FMT with a pre-antibiotics fecal sample, or no intervention (spontaneous recovery). A fifth group (N=10) remained untreated throughout. Absolute abundances of probiotics species were determined by qPCR in fecal samples collected at the various experimental stages or in GI tract tissues 28 days post-antibiotics. (A) Experimental design. (B) qPCR-based fold change of pooled probiotics targets in fecal samples, normalized to baseline (before antibiotics). ****, P<0.0001, Two-Way ANOVA & Tukey. Inset: incremental area under the ddCT curve, calculated from day zero post-antibiotics. ****, P<0.0001, Kruskal-Wallis & Dunn's. (C) Same as B but for each probiotics species separately without normalization. * denotes any P-value <0.05-0.0001 for clarity, two-way ANOVA & Dunnett. (D-E) qPCR based enumeration of pooled probiotics targets in tissues of the (D) LGI or (E) UGI. **, P<0.01, ****, P<0.0001, Kruskal-wallis & Dunn's. (F) Same as D-E but for each probiotics species separately. Symbols represent the mean, error bars SEM. ST, stomach; DU, duodenum; PJ, proximal jejunum; DJ, distal jejunum; IL, ileum; CE, cecum; PC, proximal colon; DC, distal colon; Ctrl, control; Abx, antibiotics; Sp, spontaneous recovery; Prob, probiotics; BBI, Bifidobacterium bifidum; BBR, Bifidobacterium breve; BIN, Bifidobacterium longum subsp. infantis; BLO, Bifidobacterium longum; LAC, Lactobacillus acidophilus; LCA, Lactobacillus casei; LLA, Lactococcus lactis; LPA, Lactobacillus casei subsp. paracasei; LPL, Lactobacillus plantarum; LRH, Lactobacillus rhamnosus; STH, Streptococcus thermophilus. The experiment was repeated three times.

FIGS. 23A-K. Probiotics maintain dysbiosys and delay return to homeostasis of the post-antibiotics treated murine GI tract. 16S rDNA based comparison of post cipro-flagyl reconstitution in probiotics treated mice (N=10) compared to mice treated with aFMT (N=10), and mice that did not receive post-antibiotics treatment and were followed up for 28 days (N=10) or sacrificed immediately after antibiotics (N=10), and no antibiotics controls (N=10). (A) Alpha diversity quantified as observed species in fecal samples. *, P<0.05, ****, P<0.0001 between probiotics and spontaneous recovery, Two-Way ANOVA and Dunnett. (B) Unweighted UniFrac distances to baseline in feces. ****, P<0.0001 between probiotics and spontaneous recovery, Two-Way ANOVA and Dunnett. (C) Genera significantly reduced by antibiotics in feces, which returned to baseline levels in FMT and spontaneous recovery but not in probiotics. In square brackets, the lowest taxonomic rank for which information was available; 0, order, F, family, G, genus. Significance according to Mann-Whitney. (D) Relative abundance of Blautia in fecal samples. (E-F) Alpha diversity in tissues of the (E) LGI or (F) UGI. *, P<0.05, **, P<0.01, ***, P<0.001, ****, P<0.0001, Kruskal-Wallis & Dunn's. (G) qPCR based quantification of probiotics load according to 16S, values are normalized to a detection threshold of 40. (H) Weighted UniFrac PCoA of all tissues. (I) Weighted UniFrac distances to control. ****, P<0.0001, Kruskal-Wallis & Dunn's. (J) Same as C but in tissues of the LGI mucosa. (K) Top taxa significantly anti-correlated with alpha diversity in the LGI mucosa. Samples are colored according to group. Significance and r-values according to Spearman. Symbols and horizontal lines represent the mean, error bars SEM or 10-90 percentile. Abx, antibiotics; LGI, lower gastrointestinal tissues; UGI, upper gastrointestinal tissues; Ctrl, control; Sp, spontaneous recovery; Prob, probiotics.

FIGS. 24A-G. Antibiotics subvert colonization resistance to probiotics in the human LGI. Three groups of humans were treated for 7 days with cipro-flagyl, followed by either bi-daily probiotics pill administration (N=8), a single autologous FMT of stool obtained before the antibiotics intervention (N=6), or no intervention (spontaneous recovery, N=7). (A) Outline of the three arms of intervention in humans. (B) Probiotics strain quantification in stool based on mapping of metagenomic sequences to unique genes, which correspond to the strains found in the probiotics pill. Dark gray marks the presence of the probiotics species and red marks the presence of the probiotics strains. (C) qPCR quantification of probiotics species in stools from last day of antibiotics, day 19 of probiotics supplementation, day 56 of the experiment (one month after cessation), and then two, three and four months after cessation, normalized to samples from the last baseline day before antibiotics. * denotes any P-value <0.05-0.0001 for clarity, two-way ANOVA & Dunnett. (D) Aggregated Probiotics load in stool in the three groups from the last day of antibiotics till 4 months of follow-up. S, probiotics significantly higher compared to spontaneous recovery; F, probiotics significantly higher than FMT. Number of letters represents the magnitude of p-value. *, P<0.05; **, P<0.01; ***, P<0.001; ****, P<0.0001, Two-Way ANOVA & Tukey. Inset, incremental area under the curve from each group's baseline, Kruskal-Wallis & Dunn's. (E) MetaPhlAn2-based aggregated quantification of probiotics species in tissues of individuals pre-treated with antibiotics or antibiotics naive (see Example 1), day 21 of probiotics normalized to baseline. ****, P<0.0001, Mann-Whitney. (F) qPCR-based fold changes of probiotics species in each mucosal tissue of each group. * denotes any P-value <0.05-0.0001 for clarity, two-way ANOVA for tissues and Dunnett per-species per-tissue, relative to baseline. (G) q-PCR based Aggregated fold change in probiotics species. ***, P<0.001, ****, P<0.0001, Kruskal-Wallis & Dunn's. Symbols represent the mean, error bars SEM. GF, gastric fundus; GA, gastric antrum; J, jejunum; D, duodenum; TI, terminal ileum; Ce, cecum; AC, ascending colon; TC, transverse colon; DC, descending colon; SC, sigmoid colon; R, rectum. BBI, Bifidobacterium bifidum; BBR, Bifidobacterium breve; BIN, Bifidobacterium infantis; BLO, Bifidobacterium longum; LAC, Lactobacillus acidophilus; LCA, Lactobacillus casei; LLA, Lactococcus lactis; LPA, Lactobacillus paracasei; LPL, Lactobacillus plantarum; LRH, Lactobacillus rhamnosus; STH, Streptococcus thermophilus. Sp, spontaneous recovery; Prob, probiotics. Abx, antibiotics, Intervent, intervention, F.U., follow up.

FIGS. 25A-K. Probiotics delay fecal microbiome reconstitution to baseline following antibiotics treatment. Stool samples collected during reconstitution from all treatment arms (starting from day 4 post-abx) were compared between them and to their own baseline during (abx) and before antibiotics (naive). (A) PCoA plot of unweighted UniFrac distances between stool samples collected during reconstitution in each of the treatment arms and during or before antibiotics. (B) Distance to baseline of each participant (mean of a group is plotted) throughout the experiment. Colored asterisks indicate any P-value <0.05-0.0001 vs. baseline for clarity, two-way ANOVA & Dunnett. Inset, area under the post-abx reconstitution curve for each group, *, P<0.05, Kruskal-Wallis & Dunn's. (C) Same as A but based on species (MetaPhlAn2). (D) Same as B but with Bray-Curtis dissimilarity indices according to MetaPhlAn2. (E) Same as B but with observed species. (F) 16S qPCR-based quantification of bacterial load, normalized to baseline before antibiotics, **, P<0.01 probiotics vs. spontaneous, Two-Way ANOVA & Tukey. (G) Intersection analysis of species significantly reduced or increased compared to baseline by antibiotics, and reverted by FMT and spontaneous recovery but not probiotics. Listed are species with minimal coefficient of variation between FMT and spontaneous recovery and maximal between probiotics and the other two arms. (H) Fold change (FC) between the last day of probiotics and baseline in humans and mice of genera detected in both organisms. (I) Top species significantly anti-correlated with alpha diversity in feces. Samples are colored according to group. Significance and r values according to Spearman. (J) Same as E but for KEGG pathways. (K) Same as I but for KEGG pathways. Symbols represent the mean, error bars SEM.

FIGS. 26A-J. Probiotics delay the microbiome reconstitution in the antibiotics-perturbed human LGI. Lumen and mucosa samples collected 3 weeks post antibiotics in each of the study arms were compared to samples collected on the last day of antibiotics (abx) and samples from naive non-antibiotics treated individuals. (A-C) PCoA and PCA plots demonstrate different reconstitution patterns 3 weeks after antibiotics treatment in subjects receiving probiotics after antibiotics therapy in terms of (A) 16S rDNA sequencing, (B) MetaPhlAn2 and (C) KO abundances. (D-F) Distance from antibiotics-naive mucosal samples in terms of (D) unweighted UniFrac distance (E) Bray-Curtis dissimilarity based on species and (F) KO abundances. Significance according to Kruskal-Wallis & Dunn's. (G) Observed species in the LGI lumen and mucosa on day 21 post antibiotics. Significance according to Kruskal-Wallis & Dunn's. (H) Bacterial load in the LGI mucosa as determined by 16S qPCR. CT values are normalized to a detection threshold of 40. Significance according to Kruskal-Wallis & Dunn's. (I) Intersection analysis of species significantly reduced or increased compared to baseline by antibiotics, and reverted by FMT and spontaneous recovery but not probiotics. Listed are species with minimal coefficient of variation between FMT and spontaneous recovery and maximal between probiotics and the other two arms. (J) Same as I but for KEGG pathways. Symbol and horizontal bar represent the mean; error bars represent SEM; *, P<0.05; **, P<0.01; ***, P<0.001; ****, P<0.0001. N.S., non-significant.

FIGS. 27A-K. Reconstitution of antibiotics-naive human GI transcriptional landscape is delayed by probiotics. (A) Pathways that are significantly affected by antibiotics in the descending colon, FDR-corrected P<0.05. (B) Genes that are significantly altered by antibiotics compared to the naive state and reverted by FMT and spontaneous recovery but not by probiotics in every region. (C-E) Quantification of genes in the duodenum distinct between the naive state and (C) post spontaneous-recovery, (D) post-FMT or (E) post probiotics. (F-H) same as C-E but comparing to the post-antibiotics transcriptome in the jejunum. (I) Genes significantly different after 3 weeks of post antibiotics spontaneous reconstitution or probiotics in the duodenum. (J) Normalized number of transcripts for IL1B in the descending colon after 3 weeks reconstitution. (K) Same as J but for REG3G in the ileum. St, stomach; Du, duodenum; Je, jejunum; IL, ileum; Ce, Cecum; DC, descending colon. *, P<0.05; **, P<0.01, Kruskal-Wallis & Dunn's. Prob, probiotics, Spont, spontaneous recovery. Horizontal lines represent the mean, error bars S.E.M.

FIGS. 28A-H. Probiotics-associated soluble factors inhibit the human fecal microbiome. The content of a probiotics pill was cultured in various media to enhance differential growth. The supernatant was filtered using a 0.22 uM filter and added to a lag-phase human fecal microbiome culture in BHI, and growth was quantified by optical density. (A) Experimental design. (B) OD measured after 8 hours of fecal culture with filtrates from the various probiotics cultures. *, P<0.05, One-Way ANOVA and Dunnett. -, fecal culture with PBS (no filtrate). (C-D) OD-based growth curves of fecal microbiome cultured with probiotics-MRS filtrate or a sterile acidified MRS. In (C) also compared to non-acidified sterile MRS. In (D) also with a filtrate mixed from pure cultures of each of the 5 Lactobacillus species present in the pill. *, P<0.05; **, P<0.01; ***, P<0.001; ****, P<0.0001, two-way ANOVA & Tukey. (E) Alpha diversity based on 16S rDNA of cultures from D harvested after 11 hours. **, P<0.01, two-sided t-test. (F-G) Weighted UniFrac distances of samples from the three conditions in D harvested after 11 hours. ****, P<0.0001, Kruskal-Wallis & Dunn's. (H) Taxa under or over-represented in the culture with probiotics filtrate compared to acidified MRS. In red, Mann-Whitney P<0.05. Each condition was represented by 3-5 tubes. The experiment was repeated three times. Symbol and horizontal bars represent the mean; error bars represent SEM or 10-90 percentile.

FIGS. 29A-H. Transient enrichment of specific probiotics-associated genera in mice stools during supplementation. 16S rDNA-based quantification of probiotics genera in (A-D) stool or (E-H) lumen and mucosa GI samples of mice treated with cipro-flagyl followed by no intervention (N=10, spontaneous recovery, S), auto-FMT of a pre-antibiotics fecal sample (F, N=10) or daily administration of probiotics (P, N=10). A fourth control group was antibiotics naive (C, N=10). In E-H, a fifth group (N=10) dissected immediately after antibiotics is included in black. Portrayed are the relative abundances (RA) of (A,E) Lactobacillus (B,F) Bifidobacterium (C-G) Streptococcus (D-H) Lactococcus. Letters above symbols denote probiotics higher and significant versus control (C), aFMT (F) or spontaneous recovery (S), repeated letters correspond to magnitude of p-value according to two-way ANOVA & Dunnett, *, P<0.05; **, P<0.01; ***, P<0.001, ****, P<0.0001. Symbols represent the mean; error bars represent SEM. N.S., non-significant.

FIGS. 30A-J. Probiotics delay post-antibiotics fecal and GI murine microbiome reconstitution. (A) qPCR-based aggregated probiotics load in UGI and LGI tissues of antibiotics-treated (+) or naive mice (-, independent cohort described elsewhere story 1 ref). *, P<0.05; **, P<0.01; ****, P<0.0001, Mann-Whitney. (B) UniFrac distances in fecal samples were recalculated after omitting the 4 probiotics genera (Lactobacillus, Bifidobacterium, Lactococcus, and Streptococcus) from the OTU table, followed by rarefaction to 10000 reads and renormalizing to 1. *, P<0.05, **, P<0.01, ****, P<0.0001, two-way ANOVA and Dunnett between probiotics and spontaneous recovery. (C-E) Significant differences (FDR corrected Wilcoxon rank sum test P<0.05) in fecal microbiome following the various post-antibiotics treatments highlighted in red. (C) 28-days probiotics (D) no post-antibiotics treatment, spontaneous recovery (E) auto-FMT. (F-G) Macroscopic differences in mice ceca between post-antibiotics probiotics and spontaneous recovery. Ceca were harvested 28 days post-antibiotics and probiotics supplementation or no treatment. (F) Larger ceca are observed in probiotics mice, some with a black spot. (G) Probiotics mice have heavier ceca, Mann-Whitney P<0.0001. (H) Weighted UniFrac distances to control. ****, P<0.0001, Kruskal-Wallis & Dunn's. (I-J) Same as B but in tissues, re-rarefied to 5000 reads. Symbols and horizontal lines represent the mean, error bars SEM or 10-90 percentile. Ctrl, control; Sp, spontaneous recovery; Prob, probiotics. L, lumen; M, mucosa. N.S., non-significant.

FIGS. 31A-K. Probiotics delay return to homeostasis of the post-antibiotics treated murine GI tract in a non-vivarium dependent manner. Experimental conditions detailed in FIGS. 23A-K were repeated in an independent group of mice in a different vivarium. 16S rDNA based comparison of post cipro-flagyl reconstitution in probiotics treated mice (N=10) compared to mice treated with aFMT (N=10), mice that did not receive post-antibiotics treatment (N=10), and a fourth antibiotics-naive control group (N=10). (A) Taxa significantly different between the vivaria represented in stool samples, red circles denote a Mann-Whitney P<0.05. (B) Stool alpha diversity. *, P<0.05; ***, P<0.001, two-way ANOVA & Tukey between spontaneous recovery and probiotics. (C) Post-antibiotics incremental area under the alpha diversity reconstitution curve from day 14 (iAUC). *, P<0.05; ****, P<0.0001, Kruskal-Wallis & Dunn's. (D) Unweighted UniFrac distances to baseline in feces, asterisks denote significance between probiotics and spontaneous recovery, Two-Way ANOVA & Tukey. (E) Taxa significantly over represented in stool samples after 28 days of probiotics compared to no treatment. (F-G) Alpha diversity in tissues of the (F) LGI and (G) UGI, significance according to Kruskal-Wallis & Dunn's. (H-I) Weighted UniFrac distances to control in tissues. Significance is according to Kruskal-Wallis & Dunn's. (J-K) Taxa significantly enriched or decreased in probiotics compared to spontaneous recovery and aFMT together in the (J) LGI or (K) UGI. Symbols and horizontal lines represent the mean, error bars SEM or 10-90 percentile. *, P<0.05; **, P<0.01; ***, P<0.001; ****, P<0.00001. Abx, antibiotics; LGI, lower gastrointestinal tissues; UGI, upper gastrointestinal tissues; L, lumen; M, mucosa. Ctrl, control; Sp, spontaneous recovery; Prob, probiotics.

FIGS. 32A-K. Antibiotics administration triggers profound changes in gut bacterial composition and function. (A) Reduction in shotgun sequencing reads from stool mapped to bacteria by Bowtie2 during antibiotics. (B) PCoA based on 16S rDNA composition post-antibiotics or in an antibiotics-naive cohort (story 1 ref). (C-D) Genera (C) or species (D) significantly altered by antibiotics in stool samples, red circles have a Mann-Whitney P<0.05. All pre-antibiotics stool samples from all participants compared to 7 days of antibiotics. (E-F) Same as C-D but in the LGI mucosa. (G) Same as E but in the UGI mucosa. (H) Unweighted UniFrac distances of various GI regions to the corresponding region in a separate, non-antibiotics treated cohort (N=19, STORY 1 REF). Significance according to Kruskal-Wallis & Dunn's. (I) Same as B but PCA based on KEGG pathways. (J) Same as C but with KEGG pathways. (K) Same as J but in the LGI mucosa. Horizontal lines represent the mean; error bars represent SEM or 10-90 percentile. *, P<0.05; **, P<0.01; ***, P<0.001; ****, P<0.00001. Abx, antibiotics, UGI, upper gastrointestinal, LGI, lower gastrointestinal, TI, terminal ileum.

FIGS. 33A-F. Quantification of probiotics in stools of supplemented individuals and controls. (A-D) 16S rDNA-based quantification probiotics-associated genera in stools of the probiotics consuming individuals, namely (A) Lactobacillus (B) Bifidobacterium (C) Lactococcus (D) Streptococcus. Significance according to Kruskal-Wallis & Dunn's. (E) MetaPhlAn2-based quantification of probiotics species relative abundance in stools. *, any P<0.05-0.0001, Two-Way ANOVA & Dunnett compared to baseline. (F) Probiotics species abundances as determined by qPCR in all participants from last day antibiotics till four months of follow up, normalized to baseline pre-antibiotics. Symbols represent the mean; error bars represent SEM. RA, relative abundance, Abx, antibiotics, Spont, spontaneous recovery.

FIGS. 34A-D. Quantification of probiotics in GI samples of supplemented individuals and controls. (A-B) 16S rDNA-based quantification probiotics-associated genera in the (A) GI lumen or (B) mucosa of the probiotics-consuming individuals. (C-D) Same as A-B but based on MetaPhlAn2. *, any P<0.05-0.0001, two-way ANOVA for tissues and Sidak per-species per-tis sue.

FIGS. 35A-B. Inter-individual differences in probiotics colonization in the antibiotics perturbed gut. (A) Average fold differences calculated between the last antibiotics and last probiotics supplementation day for each participant for each probiotics species in each region. *, P<0.05, **, P<0.01, ****, P<0.0001, Wilcoxon signed-rank test. (B) Probiotics strain quantification in the GI mucosa based on mapping of metagenomic sequences to unique genes, which correspond to the strains found in the probiotics pill. Dark gray marks the presence of the probiotics species and red marks the presence of the probiotics strains.

FIGS. 36A-D. Greater distance to stool baseline in probiotics consuming individuals is not due to presence of probiotics genera or species. (A-B) UniFrac distances in fecal samples were recalculated after omitting the 4 probiotics genera (Lactobacillus, Bifidobacterium, Lactococcus, and Streptococcus) from the OTU table, followed by rarefaction to 10000 reads and renormalizing to 1. Inset, area under the curve for each group, significance according to two-sided t-test. (C-D) Bray-Curtis dissimilarity indices were recalculated after omitting the 10 probiotics species from the MetaPhlAn2 output table and renormalizing to 1. Colored asterisks indicate significant difference of a time-point to baseline (Two-Way ANOVA & Dunnett P<0.05-0.0001). Inset, area under the curve for each group.

FIGS. 37A-F. The effect of each treatment arm on reconstitution of species and KOs in stool. (A-C) Relative abundance of species before antibiotics and after (A) aFMT, (B) Probiotics or (C) spontaneous recovery (spont). (D-F) same as A-C but with KOs. Colored species or KOs remained more than 2-fold differential in their abundance before and after the treatment.

FIGS. 38A-D. Greater distance to antibiotics-naive LGI configuration in probiotics consuming individuals is not due to presence of probiotics genera or species. (A-B) UniFrac distances in LGI samples were recalculated after omitting the 4 probiotics genera (Lactobacillus, Bifidobacterium, Lactococcus, and Streptococcus) from the OTU table, followed by rarefaction to 10000 reads and renormalizing to 1. (C-D) Bray-Curtis dissimilarity indices were recalculated after omitting the 10 probiotics species from the MetaPhlAn2 output table and renormalizing to 1. **, P<0.01; ***, P<0.001; ****, P<0.0001, Kruskal-Wallis & Dunn's. Abx, antibiotics, Spont, spontaneous recovery, Prob, probiotics.

FIGS. 39A-B. LGI reconstitution based on KEGG pathways. (A) PCA demonstrating reconstitution patterns 3 weeks after antibiotics treatment in each of the arms and antibiotics-naive individuals based on KEGG pathways. (B) 1-Spearman correlation to the antibiotics-naive cohort based on KEGG pathways. **, P<0.01; ***, P<0.001; ****, P<0.0001, Kruskal-Wallis & Dunn's. Abx, antibiotics, Spont, spontaneous recovery, Prob, probiotics.

### DESCRIPTION OF SPECIFIC EMBODIMENTS OF THE INVENTION

The present invention, in some embodiments thereof, relates to methods of using probiotics in mammalian subjects. More specifically, the invention relates to personalized predictions based on the gut microbiome as to whether a subject is responsiveness to a probiotic based on the gut microbiome.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Probiotics supplements are commonly consumed as means of life quality improvement and disease prevention. However, evidence of probiotics colonization efficacy, upon encountering the adult well-entrenched mucosal-associated gut microbiome, remains sparse and controversial.

In Example 1, the present inventors profiled the homeostatic mucosal, luminal and fecal microbiome along the entirety of the gastrointestinal tract of mice and humans. They demonstrate that solely relying on stool sampling as a proxy of mucosal GI composition and function yields inherently limited conclusions. Whilst the abundance of particular bacterial species in the stool mirror their abundance along other locations in the GI tract, many do not.

In contrast, direct gastrointestinal sampling in mice and humans, before and during an 11-strain probiotic consumption showed that probiotics readily pass through the gastrointestinal tract into stool, but encounter along the way a substantial microbiome-mediated mucosal colonization resistance, the level of which significantly impacted probiotics effects on the indigenous mucosal microbiome composition, function, and host gene expression profile. In humans, a person-, strain- and region-specific variability in gut mucosal colonization resistance significantly correlated with baseline host transcriptional and microbiome characteristics, but not with stool levels of probiotics during consumption.

Identification of such baseline microbial and host factors potentially enables prediction of a probiotics responsiveness or resistant state. The results obtained call for consideration of a transition from an empiric ‘one size fits all’ probiotics regiment design, to one which is based on the individual. Such a measurement-based approach would enable integration of person-specific features in tailoring particular probiotics interventions for a particular person at a given clinical context. Thus, the present invention can be used to devise more effective means of colonizing and impacting the host gut mucosa.

In Example 2, the present inventors addressed the issue as to whether probiotics efficiently reconstitute the indigenous human gut mucosal microbiome. They compared the effects of the probiotic cocktail described above with autologous fecal microbiome transplantation (aFMT) on post-antibiotic reconstitution of the mucosal gut microbiome, via a sequential invasive multi-omics assessment of the human gut before and during probiotics supplementation. In the antibiotics-perturbed gut, these probiotics feature enhanced colonization in humans and to a lesser degree in mice. Importantly, probiotics in this setting induce a markedly delayed mucosal microbiome reconstitution compared to spontaneous recovery or aFMT. As such, post-antibiotic probiotics-induced benefits may be offset by a delayed indigenous microbiome recovery.

These results highlight a need for development of personalized, targeted and aFMT-based approaches achieving post-antibiotic mucosal protection, without compromising microbiome recolonization in the perturbed host.

Thus, according to a first aspect of the present invention, there is provided a method of assessing whether a candidate subject is suitable for probiotic treatment comprising determining a signature of the gut microbiome of the candidate subject, wherein when the signature of the microbiome of the candidate subject is statistically significantly similar to a signature of a gut microbiome of a control subject known to be responsive to probiotic treatment, it is indicative that the subject is suitable for probiotic treatment.

As used herein the term “subject” refers to a mammalian subject (e.g. mouse, cow, dog, cat, horse, monkey, human), preferably human.

In one embodiment, the candidate subject is a healthy subject.

In another embodiment, the candidate subject has an infection. In still another embodiment, the candidate subject has recovered from an infection following antibiotic treatment.

In another embodiment, the candidate subject does not have a chronic disease.

The term “probiotic” as used herein, refers to one or more microorganisms which, when administered appropriately, can confer a health benefit on the host or subject and/or reduction of risk and/or symptoms of a disease, disorder, condition, or event in a host organism.

In some embodiments, probiotics comprise bacteria. Some non-limiting examples of known probiotics include: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, and any combination thereof.

The probiotic may comprise one, at least two, at least three, at least four, at least five, at least six, at least seven, at least eight, at least nine, at least ten or more bacterial species.

According to a particular embodiment, the probiotic comprises at least one of the following species of bacteria: B. bifidum, L. rhamnosus, L. lactis, L. casei, B. breve, S. thermophilus, B. longum, L. paracasei, L. plantarum and B. infantis.

A control subject may be classified as being a “responder” to a probiotic if there is a statistically significant elevation in the absolute abundance of that probiotic strain in his GI mucosa (e.g. as determined by Mann-Whitney test).

A control subject may be classified as being a “non-responder” to a probiotic if there is no statistically significant elevation in the absolute abundance of that probiotic strain in his GI mucosa (e.g. as determined by Mann-Whitney test).

As used herein, the term “microbiome” refers to the totality of microbes (bacteria, fungae, protists), their genetic elements (genomes) in a defined environment.

According to a particular embodiment, the microbiome is a gut microbiome (i.e. microbiota of the digestive track). In one embodiment, the environment is the small intestine. In another embodiment, the environment is the large intestine. The microbiome may be of the lumen or the mucosa of the small intestine or large intestine. In still another embodiment, the gut microbiome is a fecal microbiome.

In some embodiments, a microbiota sample is collected by any means that allows recovery of the microbes and without disturbing the relative amounts of microbes or components or products thereof of a microbiome. In some embodiments, the microbiota sample is a fecal sample. In other embodiments, the microbiota sample is retrieved directly from the gut—e.g. by endoscopy from the lower gastrointestinal (GI) tract or from the upper GI tract. The microbiota sample may be of the lumen of the GI tract or the mucosa of the GI tract.

According to one embodiment, the microbiome sample (e.g. fecal sample) is frozen and/or lyophilized prior to analysis. According to another embodiment, the sample may be subjected to solid phase extraction methods.

In some embodiments, the presence, level, and/or activity of between 5 and 10 species of microbes are measured. In some embodiments, the presence, level, and/or activity of between 5 and 20 species of microbes are measured. In some embodiments, the presence, level, and/or activity of between 5 and 50 species of microbes are measured. In some embodiments, the presence, level, and/or activity of between 5 and 100 species of microbes are measured. In some embodiments, the presence, level, and/or activity of between 5 and 500 species of microbes are measured. In some embodiments, the presence, level, and/or activity of between 5 and 1000 species of microbes are measured. In some embodiments, the presence, level, and/or activity of between 50 and 500 species of microbes (e.g. bacteria) are measured. In some embodiments, the presence, level, and/or activity of substantially all species/classes/families of bacteria within the microbiome are measured. In still more embodiments, the presence, level, and/or activity of substantially all the bacteria within the microbiome are measured.

Measuring a level or presence of a microbe may be effected by analyzing for the presence of microbial component or a microbial by-product. Thus, for example the level or presence of a microbe may be effected by measuring the level of a DNA sequence. In some embodiments, the level or presence of a microbe may be effected by measuring 16S rRNA gene sequences or 18S rRNA gene sequences. In other embodiments, the level or presence of a microbe may be effected by measuring RNA transcripts. In still other embodiments, the level or presence of a microbe may be effected by measuring proteins. In still other embodiments, the level or presence of a microbe may be effected by measuring metabolites.

Quantifying Microbial Levels:

It will be appreciated that determining the abundance of microbes may be affected by taking into account any feature of the microbiome. Thus, the abundance of microbes may be affected by taking into account the abundance at different phylogenetic levels; at the level of gene abundance; gene metabolic pathway abundances; sub-species strain identification; SNPs and insertions and deletions in specific bacterial regions; growth rates of bacteria, the diversity of the microbes of the microbiome, as further described herein below.

In some embodiments, determining a level or set of levels of one or more types of microbes or components or products thereof comprises determining a level or set of levels of one or more DNA sequences. In some embodiments, one or more DNA sequences comprises any DNA sequence that can be used to differentiate between different microbial types. In certain embodiments, one or more DNA sequences comprises 16S rRNA gene sequences. In certain embodiments, one or more DNA sequences comprises 18S rRNA gene sequences. In some embodiments, 1, 2, 3, 4, 5, 10, 15, 20, 25, 50, 100, 1,000, 5,000 or more sequences are amplified.

16S and 18S rRNA gene sequences encode small subunit components of prokaryotic and eukaryotic ribosomes respectively. rRNA genes are particularly useful in distinguishing between types of microbes because, although sequences of these genes differs between microbial species, the genes have highly conserved regions for primer binding. This specificity between conserved primer binding regions allows the rRNA genes of many different types of microbes to be amplified with a single set of primers and then to be distinguished by amplified sequences.

In some embodiments, a microbiota sample (e.g. fecal sample) is directly assayed for a level or set of levels of one or more DNA sequences. In some embodiments, DNA is isolated from a microbiota sample and isolated DNA is assayed for a level or set of levels of one or more DNA sequences. Methods of isolating microbial DNA are well known in the art. Examples include but are not limited to phenol-chloroform extraction and a wide variety of commercially available kits, including QIAamp DNA Stool Mini Kit (Qiagen, Valencia, Calif.).

In some embodiments, a level or set of levels of one or more DNA sequences is determined by amplifying DNA sequences using PCR (e.g., standard PCR, semi-quantitative, or quantitative PCR) and then sequencing. In some embodiments, a level or set of levels of one or more DNA sequences is determined by amplifying DNA sequences using quantitative PCR. These and other basic DNA amplification procedures are well known to practitioners in the art and are described in Ausebel et al. (Ausubel F M, Brent R, Kingston R E, Moore D, Seidman J G, Smith J A, Struhl K (eds). 1998. Current Protocols in Molecular Biology. Wiley: New York).

In some embodiments, DNA sequences are amplified using primers specific for one or more sequence that differentiate(s) individual microbial types from other, different microbial types. In some embodiments, 16S rRNA gene sequences or fragments thereof are amplified using primers specific for 16S rRNA gene sequences. In some embodiments, 18S DNA sequences are amplified using primers specific for 18S DNA sequences.

In some embodiments, a level or set of levels of one or more 16S rRNA gene sequences is determined using phylochip technology. Use of phylochips is well known in the art and is described in Hazen et al. (“Deep-sea oil plume enriches indigenous oil-degrading bacteria.” Science, 330, 204-208, 2010), the entirety of which is incorporated by reference. Briefly, 16S rRNA genes sequences are amplified and labeled from DNA extracted from a microbiota sample. Amplified DNA is then hybridized to an array containing probes for microbial 16S rRNA genes. Level of binding to each probe is then quantified providing a sample level of microbial type corresponding to 16S rRNA gene sequence probed. In some embodiments, phylochip analysis is performed by a commercial vendor. Examples include but are not limited to Second Genome Inc. (San Francisco, Calif.).

In some embodiments, determining a level or set of levels of one or more types of microbes comprises determining a level or set of levels of one or more microbial RNA molecules (e.g., transcripts). Methods of quantifying levels of RNA transcripts are well known in the art and include but are not limited to northern analysis, semi-quantitative reverse transcriptase PCR, quantitative reverse transcriptase PCR, and microarray analysis.

Methods for sequence determination are generally known to the person skilled in the art. Preferred sequencing methods are next generation sequencing methods or parallel high throughput sequencing methods. For example, a bacterial genomic sequence may be obtained by using Massively Parallel Signature Sequencing (MPSS). An example of an envisaged sequence method is pyrosequencing, in particular 454 pyrosequencing, e.g. based on the Roche 454 Genome Sequencer. This method amplifies DNA inside water droplets in an oil solution with each droplet containing a single DNA template attached to a single primer-coated bead that then forms a clonal colony. Pyrosequencing uses luciferase to generate light for detection of the individual nucleotides added to the nascent DNA, and the combined data are used to generate sequence read-outs. Yet another envisaged example is Illumina or Solexa sequencing, e.g. by using the Illumina Genome Analyzer technology, which is based on reversible dye-terminators. DNA molecules are typically attached to primers on a slide and amplified so that local clonal colonies are formed. Subsequently one type of nucleotide at a time may be added, and non-incorporated nucleotides are washed away. Subsequently, images of the fluorescently labeled nucleotides may be taken and the dye is chemically removed from the DNA, allowing a next cycle. Yet another example is the use of Applied Biosystems' SOLiD technology, which employs sequencing by ligation. This method is based on the use of a pool of all possible oligonucleotides of a fixed length, which are labeled according to the sequenced position. Such oligonucleotides are annealed and ligated. Subsequently, the preferential ligation by DNA ligase for matching sequences typically results in a signal informative of the nucleotide at that position. Since the DNA is typically amplified by emulsion PCR, the resulting bead, each containing only copies of the same DNA molecule, can be deposited on a glass slide resulting in sequences of quantities and lengths comparable to Illumina sequencing. A further method is based on Helicos' Heliscope technology, wherein fragments are captured by polyT oligomers tethered to an array. At each sequencing cycle, polymerase and single fluorescently labeled nucleotides are added and the array is imaged. The fluorescent tag is subsequently removed and the cycle is repeated. Further examples of sequencing techniques encompassed within the methods of the present invention are sequencing by hybridization, sequencing by use of nanopores, microscopy-based sequencing techniques, microfluidic Sanger sequencing, or microchip-based sequencing methods.

According to one embodiment, the sequencing method allows for quantitating the amount of microbe—e.g. by deep sequencing such as Illumina deep sequencing.

As used herein, the term “deep sequencing” refers to a sequencing method wherein the target sequence is read multiple times in the single test. A single deep sequencing run is composed of a multitude of sequencing reactions run on the same target sequence and each, generating independent sequence readout.

In some embodiments, determining a level or set of levels of one or more types of microbes comprises determining a level or set of levels of one or more microbial polypeptides. Methods of quantifying polypeptide levels are well known in the art and include but are not limited to Western analysis and mass spectrometry.

As mentioned herein above, as well as (or instead of) analyzing the level of microbes, the present invention also contemplates analyzing the level of microbial products.

Examples of microbial products include, but are not limited to mRNAs, polypeptides, carbohydrates and metabolites.

In some embodiments, the presence, level, and/or activity of metabolites of at least ten species of microbes are measured. In other embodiments, the presence, level, and/or activity of metabolites of between 5 and 50 species of microbes are measured. In other embodiments, the presence, level, and/or activity of metabolites of between 5 and 20 species of microbes are measured. In other embodiments, the presence, level, and/or activity of metabolites of between 5 and 100 species of microbes are measured. In some embodiments, the presence, level, and/or activity of metabolites of between 100 and 1000 or more species of microbes are measured. In other embodiments, the presence, level, and/or activity of metabolites of all bacteria within the microbiome are analyzed. In other embodiments, the presence, level, and/or activity of metabolites of all microbes within the microbiome are measured.

As used herein, a “metabolite” is an intermediate or product of metabolism. The term metabolite is generally restricted to small molecules and does not include polymeric compounds such as DNA or proteins. A metabolite may serve as a substrate for an enzyme of a metabolic pathway, an intermediate of such a pathway or the product obtained by the metabolic pathway.

According to a particular embodiment, the metabolite is one that alters the composition or function of the microbiome.

In preferred embodiments, metabolites include but are not limited to sugars, organic acids, amino acids, fatty acids, hormones, vitamins, oligopeptides (less than about 100 amino acids in length), as well as ionic fragments thereof. Cells can also be lysed in order to measure cellular products present within the cell. In particular, the metabolites are less than about 3000 Daltons in molecular weight, and more particularly from about 50 to about 3000 Daltons.

The metabolite of this aspect of the present invention may be a primary metabolite (i.e. essential to the microbe for growth) or a secondary metabolite (one that does not play a role in growth, development or reproduction, and is formed during the end or near the stationary phase of growth.

Representative examples of metabolic pathways in which the metabolites of the present invention are involved include, without limitation, citric acid cycle, respiratory chain, photosynthesis, photorespiration, glycolysis, gluconeogenesis, hexose monophosphate pathway, oxidative pentose phosphate pathway, production and β-oxidation of fatty acids, urea cycle, amino acid biosynthesis pathways, protein degradation pathways such as proteasomal degradation, amino acid degrading pathways, biosynthesis or degradation of: lipids, polyketides (including, e.g., flavonoids and isoflavonoids), isoprenoids (including, e.g., terpenes, sterols, steroids, carotenoids, xanthophylls), carbohydrates, phenylpropanoids and derivatives, alkaloids, benzenoids, indoles, indole-sulfur compounds, porphyrines, anthocyans, hormones, vitamins, cofactors such as prosthetic groups or electron carriers, lignin, glucosinolates, purines, pyrimidines, nucleosides, nucleotides and related molecules such as tRNAs, microRNAs (miRNA) or mRNAs.

Representative examples of metabolites that may be analyzed according to this aspect of the present invention include, but are not limited to bile acid components such as ursodeoxycholate, glycocholate, phenylacetate and heptanoate and flavonoids such as apigenin and naringenin.

In some embodiments, levels of metabolites are determined by mass spectrometry. In some embodiments, levels of metabolites are determined by nuclear magnetic resonance spectroscopy, as further described herein below. In some embodiments, levels of metabolites are determined by enzyme-linked immunosorbent assay (ELISA). In some embodiments, levels of metabolites are determined by colorimetry. In some embodiments, levels of metabolites are determined by spectrophotometry, as further described herein below.

According to one embodiment of this aspect of the present invention two microbiomes can be statistically significantly similar when they comprise at least 50% of the same microbial species, at least 60% of the same microbial species, at least 70% of the same microbial species, at least 80% of the same microbial species, at least 90% of the same microbial species, at least 91% of the same microbial species, at least 92% of the same microbial species, at least 93% of the same microbial species, at least 94% of the same microbial species, at least 95% of the same microbial species, at least 96% of the same microbial species, at least 97% of the same microbial species, at least 98% of the same microbial species, at least 99% of the same microbial species or 100% of the same microbial species.

According to one embodiment of this aspect of the present invention two microbiomes can be statistically significantly similar when they comprise at least 50% of the same microbial genus, at least 60% of the same microbial genus, at least 70% of the same microbial genus, at least 80% of the same microbial genus, at least 90% of the same microbial genus, at least 91% of the same microbial genus, at least 92% of the same microbial genus, at least 93% of the same microbial genus, at least 94% of the same microbial genus, at least 95% of the same microbial genus, at least 96% of the same microbial genus, at least 97% of the same microbial genus, at least 98% of the same microbial genus, at least 99% of the same microbial genus or 100% of the same microbial genus.

Additionally, or alternatively, microbiomes may be statistically similar when the relative quantity (e.g. occurrence) of at least five microbes of interest is identical. According to another embodiment, microbiomes may be statistically significantly similar when the relative amount of at least 10% of microbial bacterial species is identical. According to another embodiment, microbiomes may be statistically significantly similar when the relative amount of at least 20% of microbial bacterial species is identical. According to another embodiment, microbiomes may be statistically significantly similar when the relative amount of at least 30% of microbial bacterial species is identical. According to another embodiment, microbiomes may be statistically significantly similar when the relative amount of at least 40% of microbial bacterial species is identical. According to another embodiment, microbiomes may be statistically significantly similar when the relative amount of at least 50% of microbial bacterial species is identical. According to another embodiment, microbiomes may be statistically significantly similar when the relative amount of at least 60% of microbial bacterial species is identical. According to another embodiment, microbiomes may be statistically significantly similar when the relative amount of at least 70% of microbial bacterial species is identical. According to another embodiment, microbiomes may be statistically significantly similar when the relative amount of at least 80% of microbial bacterial species is identical. According to another embodiment, microbiomes may be statistically significantly similar when the relative amount of at least 90% of microbial bacterial species is identical.

Additionally, or alternatively, microbiomes may be statistically significant similar when the quantity (e.g. occurrence) in the microbiome of at least five microbe of interest is identical. According to another embodiment, microbiomes may be statistically significantly similar when the absolute amount of at least 10% of their species is identical. According to another embodiment, microbiomes may be statistically significantly similar when the absolute amount of at least 20% of their species is identical. According to another embodiment, microbiomes may be statistically significantly similar when the absolute amount of at least 30% of their species is identical. According to another embodiment, microbiomes may be statistically significantly similar when the absolute amount of at least 40% of their species is identical. According to another embodiment, microbiomes may be statistically significantly similar when the absolute amount of at least 50% of their species is identical. According to another embodiment, microbiomes may be statistically significantly similar when the absolute amount of at least 60% of their species is identical. According to another embodiment, microbiomes may be statistically significantly similar when the absolute amount of at least 70% of their species are identical. According to another embodiment, microbiomes may be statistically significantly similar when the absolute amount of at least 80% of their species is identical. According to another embodiment, microbiomes may be statistically significantly similar when the absolute amount of at least 90% of their species is identical.

According to another embodiment, microbiomes may be statistically significantly similar when the absolute amount of at least 10% of their genus is identical. According to another embodiment, microbiomes may be statistically significantly similar when the absolute amount of at least 20% of their genus is identical. According to another embodiment, microbiomes may be statistically significantly similar when the absolute amount of at least 30% of their genus is identical. According to another embodiment, microbiomes may be statistically significantly similar when the absolute amount of at least 40% of their genus is identical. According to another embodiment, microbiomes may be statistically significantly similar when the absolute amount of at least 50% of their genus is identical. According to another embodiment, microbiomes may be statistically significantly similar when the absolute amount of at least 60% of their genus is identical. According to another embodiment, microbiomes may be statistically significantly similar when the absolute amount of at least 70% of their genus is identical. According to another embodiment, microbiomes may be statistically significantly similar when the absolute amount of at least 80% of their genus is identical. According to another embodiment, microbiomes may be statistically significantly similar when the absolute amount of at least 90% of their genus is identical.

Thus, the fractional percentage of microbes (e.g. relative amount, ratio, distribution, frequency, percentage, etc.) of the total may be statistically similar.

According to another embodiment, in order to classify a microbe as belonging to a particular genus, family, order, class or phylum, it must comprise at least 90% sequence homology, at least 91% sequence homology, at least 92% sequence homology, at least 93% sequence homology, at least 94% sequence homology, at least 95% sequence homology, at least 96% sequence homology, at least 97% sequence homology, at least 98% sequence homology, at least 99% sequence homology to a reference microbe known to belong to the particular genus. According to a particular embodiment, the sequence homology is at least 95%.

According to another embodiment, in order to classify a microbe as belonging to a particular species, it must comprise at least 90% sequence homology, at least 91% sequence homology, at least 92% sequence homology, at least 93% sequence homology, at least 94% sequence homology, at least 95% sequence homology, at least 96% sequence homology, at least 97% sequence homology, at least 98% sequence homology, at least 99% sequence homology to a reference microbe known to belong to the particular species. According to a particular embodiment, the sequence homology is at least 97%.

In determining whether a nucleic acid or protein is substantially homologous or shares a certain percentage of sequence identity with a sequence of the invention, sequence similarity may be defined by conventional algorithms, which typically allow introduction of a small number of gaps in order to achieve the best fit. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule of the invention. Equally, BLAST protein searches may be performed with the BLASTX program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed. See www(dot)ncbi(dot)nlm(dot)nih(dot)gov for more details.

The present embodiments encompass the recognition that microbial signatures can be relied upon as proxy for microbiome composition and/or activity. Microbial signatures comprise data points that are indicators of microbiome composition and/or activity. Thus, according to the present invention, changes in microbiomes can be detected and/or analyzed through detection of one or more features of microbial signatures.

Thus, in some embodiments only the microbes (or activity thereof) of a microbial signature are measured. In other embodiments, additional microbes are measured (e.g. all the bacteria of the microbiome are sequenced), but the analysis for the prediction relies on those microbes of the microbial signature.

In some embodiments, a microbial signature includes information relating to absolute amount of five or more types of microbes, and/or products thereof. In some embodiments, a microbial signature includes information relating to relative amounts of five, ten, twenty, fifty, one hundred or more species of microbes and/or products thereof. In some embodiments, a microbial signature includes information relating to relative amounts of two, three, four, five, ten, twenty, fifty, one hundred or more genus of microbes and/or products thereof.

In the fecal microbiome, the present inventors have found that levels of the following genii of microbes are indicative as to whether a subject is a responder or not.

1. Bacteria of the genus Bifidobacterium

2. Bacteria of the genus Dialister

More specifically, the present inventors showed that lower abundance (i.e. levels below a predetermined level) of Bifidobacterium in the feces signifies a responder (i.e. permissive), whereas a higher abundance (i.e. above a predetermined level) of Dialister in the feces is indicative of a responder.

Furthermore, in the fecal microbiome, the present inventors have found that the species of microbes listed in Table A are indicative as to whether a subject is a responder or not.

TABLE A

s_Lachnospiraceae_bacterium_5_1_63FAA
s_Bacteroides_vulgatus
s_Bacteroides_caccae
s_Alistipes_onderdonkii
s_Lachnospiraceae_bacterium_1_1_57FAA
s_Parabacteroides_unclassified
s_Parabacteroides_johnsonii
s_Bifidobacterium_pseudocatenulatum
s_Megasphaera_unclassified

More specifically, the present inventors showed that lower abundance (i.e. levels below a predetermined level) of the species listed in Table A in the feces signifies a responder (i.e. permissive).

Furthermore, in the fecal microbiome, the present inventors have found that the level of microbes utilizing a Kegg pathway listed in Table B are indicative as to whether a subject is a responder or not.

TABLE B

| | |
|---|---|
| ko00670 | One carbon pool by folate |
| ko00360 | Phenylalanine metabolism |
| ko00030 | Pentose phosphate pathway |
| ko00052 | Galactose metabolism |
| ko00010 | Glycolysis/Gluconeogenesis |
| ko00040 | Pentose and glucuronate interconversions |
| ko00960 | Tropane, piperidine and pyridine alkaloid |
| | biosynthesis |
| ko00363 | Bisphenol degradation |
| ko00260 | Glycine, serine and threonine metabolism |
| ko00190 | Oxidative phosphorylation |
| ko00340 | Histidine metabolism |
| ko00330 | Arginine and proline metabolism |
| ko00983 | Drug metabolism - other enzymes * |
| ko00770 | Pantothenate and CoA biosynthesis |
| ko00562 | Inositol phosphate metabolism |
| ko00521 | Streptomycin biosynthesis * |
| ko00523 | Polyketide sugar unit biosynthesis * |
| ko00910 | Nitrogen metabolism |
| ko00633 | Nitrotoluene degradation |
| ko00440 | Phosphonate and phosphinate metabolism |
| ko00750 | Vitamin B6 metabolism |

More specifically, the present inventors showed that increase abundance in the feces (i.e. levels above a predetermined level) of bacteria utilizing a Kegg pathway listed in Table B in which no * appear signifies resistance to probiotic (i.e. non-permissive), whereas lower abundance in the feces (i.e. levels below a predetermined level) of the species listed in Table B in which an * appear signifies a resistance to probiotic (i.e. non-permissive).

In the microbiome of the mucosa of the lower gastrointestinal tract (LGIM), the present inventors have found that levels of the following genii of microbes are indicative as to whether a subject is a responder or not.

1. Bacteria of the genus Odoribacter

2. Bacteria of the genus Bacteroides

3. Bacteria of the genus Bifidobacterium

4. Bacteria of the family Rikenellaceae

More specifically, the present inventors showed that lower abundance (i.e. levels below a predetermined level) of these genii in the LGIM microbiome signifies a responder (i.e. permissive)

Furthermore, in the LGIM microbiome, the present inventors have found that the species of microbes listed in Table C are indicative as to whether a subject is a responder or not.

TABLE C

s_Barnesiella_intestinihominis
s_Bacteroides_caccae
s_Coprobacter_fastidiosus
s_Bacteroides_coprophilus

More specifically, the present inventors showed that lower abundance (i.e. levels below a predetermined level) of the species listed in Table C in the LGIM microbiome signifies a responder (i.e. permissive).

Furthermore, in the LGIM microbiome, the present inventors have found that the level of microbes utilizing a Kegg pathway listed in Table D are indicative as to whether a subject is a responder or not.

TABLE D

| | |
|---|---|
| ko00071 | Fatty acid degradation |
| ko00311 | Penicillin and cephalosporin biosynthesis |
| ko00531 | Glycosaminoglycan degradation |
| ko05111 | Biofilm formation - Vibrio cholera * |
| ko00640 | Propanoate metabolism * |
| ko00440 | Phosphonate and phosphinate metabolism |
| ko00120 | Primary bile acid biosynthesis |
| Ko03018 | RNA degradation |

More specifically, the present inventors showed that increase abundance in the LGIM microbiome (i.e. levels above a predetermined level) of bacteria utilizing a Kegg pathway listed in Table D in which no * appear signifies resistance to probiotic (i.e. non-permissive), whereas lower abundance in the LGIM microbiome (i.e. levels below a predetermined level) of bacteria utilizing a Kegg pathway listed in Table D in which an * appear signifies a resistance to probiotic (i.e. non-permissive).

In the microbiome of the rectum, the present inventors have found that levels of the following genii of microbes are indicative as to whether a subject is a responder or not.

1. Bacteria of the genus Streptococcus

2. Bacteria of the genus Odoribacter

3. Bacteria of the genus Bifidobacterium

4. Bacteria of the genus Bacteroides

5. Bacteria of the family Rikenellaceae

More specifically, the present inventors showed that lower abundance (i.e. levels below a predetermined level) of all these genii except Streptococcus in the rectal microbiome signifies a responder (i.e. permissive). Lower abundance (i.e. levels below a predetermined level) of Streptococcus in the rectal microbiome signifies resistance (i.e. non-permissive).

Furthermore, in the rectal microbiome, the present inventors have found that the level of the species Barnesiella intestinihominis is indicative as to whether a subject is a responder or not.

More specifically, the present inventors showed that lower abundance (i.e. levels below a predetermined level) of Barnesiella intestinihominis in the rectal microbiome signifies a responder (i.e. permissive).

Furthermore, in the rectal microbiome, the present inventors have found that the level of microbes utilizing a Kegg pathway listed in Table E are indicative as to whether a subject is a responder or not.

TABLE E

| | |
|---|---|
| ko00640 | Propanoate metabolism |
| ko00660 | C5-Branched dibasic acid metabolism |

More specifically, the present inventors showed that lower abundance in the rectal microbiome (i.e. levels below a predetermined level) of bacteria utilizing the pathways listed in Table E signifies a resistance to probiotic (i.e. non-permissive).

In the sigmoid colon (SC) microbiome, the present inventors have found that levels of the Rikenellaceae family of microbes are indicative as to whether a subject is a responder or not.

More specifically, the present inventors showed that lower abundance (i.e. levels below a predetermined level) of Rikenellaceae in the SC signifies a responder (i.e. permissive).

Furthermore, in the SC microbiome, the present inventors have found that the level of species of microbes listed in Table F are indicative as to whether a subject is a responder or not.

TABLE F

s_Barnesiella_intestinihominis
s_Bacteroides_caccae

More specifically, the present inventors showed that lower abundance (i.e. levels below a predetermined level) of the species listed in Table F in the SC microbiome signifies a responder (i.e. permissive).

Furthermore, in the SC microbiome, the present inventors have found that the level of microbes utilizing a Kegg pathway listed in Table G are indicative as to whether a subject is a responder or not.

TABLE G

| | |
|---|---|
| ko00040 | Pentose and glucuronate interconversions |
| ko01040 | Biosynthesis of unsaturated fatty acids |
| ko00281 | Geraniol degradation |
| ko00071 | Fatty acid degradation |
| ko00960 | Tropane, piperidine and pyridine alkaloid |
| | biosynthesis |
| ko00120 | Primary bile acid biosynthesis |
| ko00440 | Phosphonate and phosphinate metabolism |
| ko00473 | D-Alanine metabolism |
| ko00380 | Tryptophan metabolism |
| ko00740 | Riboflavin metabolism |
| ko00311 | Penicillin and cephalosporin biosynthesis |
| ko03410 | Base excision repair |
| ko03060 | Protein export |
| ko02020 | Two-component system |
| ko00785 | Lipoic acid metabolism |
| ko00500 | Starch and sucrose metabolism |
| ko00330 | Arginine and proline metabolism |
| ko00730 | Thiamine metabolism |
| ko03440 | Homologous recombination |
| ko00230 | Purine metabolism |
| ko00790 | Folate biosynthesis |
| ko00360 | Phenylalanine metabolism |
| ko03018 | RNA degradation |
| ko00630 | Glyoxylate and dicarboxylate metabolism |
| ko00620 | Pyruvate metabolism |
| ko00052 | Galactose metabolism |
| ko03430 | Mismatch repair |
| ko00061 | Fatty acid biosynthesis |
| ko00511 | Other glycan degradation |
| ko00290 | Valine, leucine and isoleucine biosynthesis |
| ko00531 | Glycosaminoglycan degradation |
| ko00750 | Vitamin B6 metabolism |
| ko00908 | Zeatin biosynthesis |

More specifically, the present inventors showed that increase abundance in the SC microbiome (i.e. levels above a predetermined level) of bacteria utilizing a Kegg pathway listed in Table G signifies resistance to probiotic (i.e. non-permissive).

In the descending colon (DC) microbiome, the present inventors have found that levels of the following genii of microbes are indicative as to whether a subject is a responder or not.

1. Bacteria of the genus Bacteroides

2. Bacteria of the genus Odoribacter

3. Bacteria of the family Rikenellaceae

More specifically, the present inventors showed that lower abundance (i.e. levels below a predetermined level) of these genii/family in the DC signifies a responder (i.e. permissive).

Furthermore, in the DC microbiome, the present inventors have found that the levels of species of microbes listed in Table H are indicative as to whether a subject is a responder or not.

TABLE H

s_Barnesiella_intestinihominis
s_Escherichia_coli

More specifically, the present inventors showed that lower abundance (i.e. levels below a predetermined level) of Barnesiella intestinihominis in the DC signifies a responder (i.e. permissive), whereas lower abundance (i.e. levels below a predetermined level) of Escherichia_coli signifies a non-responder (i.e. resistant).

Furthermore, in the DC microbiome, the present inventors have found that the levels of microbes utilizing a Kegg pathway listed in Table I are indicative as to whether a subject is a responder or not.

TABLE I

| | |
|---|---|
| ko00311 | Penicillin and cephalosporin biosynthesis |
| ko00740 | Riboflavin metabolism |
| ko00562 | Inositol phosphate metabolism |
| ko00650 | Butanoate metabolism |
| ko00531 | Glycosaminoglycan degradation |
| ko00480 | Glutathione metabolism |
| ko00071 | Fatty acid degradation |
| ko00040 | Pentose and glucuronate interconversions |
| ko00640 | Propanoate metabolism * |
| ko00790 | Folate biosynthesis |
| ko00053 | Ascorbate and aldarate metabolism |

More specifically, the present inventors showed that increase abundance in the DC microbiome (i.e. levels above a predetermined level) of bacteria utilizing a Kegg pathway listed in Table I in which no * appear signifies resistance to probiotic (i.e. non-permissive), whereas lower abundance in the DI (i.e. levels below a predetermined level) of the species listed in Table I in which an * appear signifies a resistance to probiotic (i.e. non-permissive).

In the transverse colon (TC) microbiome, the present inventors have found that levels of the following genii of microbes are indicative as to whether a subject is a responder or not.

1. Bacteria of the genus Odoribacter

2. Bacteria of the genus Dorea

3. Bacteria of the family Rikenellaceae

More specifically, the present inventors showed that lower abundance (i.e. levels below a predetermined level) of these genii/family in the TC microbiome signifies a responder (i.e. permissive).

Furthermore, in the TC microbiome, the present inventors have found that the levels of species of microbes listed in Table J are indicative as to whether a subject is a responder or not.

TABLE J

s_Bacteroides_massiliensis
s_Bacteroides_cellulosilyticus
s_Dorea_unclassified

More specifically, the present inventors showed that lower abundance (i.e. levels below a predetermined level) of S. Dorea in the TC microbiome signifies a responder (i.e. permissive), whereas lower abundance (i.e. levels below a predetermined level) of Bacteroides_cellulosilyticus or s_Bacteroides_massiliensis in the TC microbiome signifies resistance (i.e. non-permissive).

Furthermore, in the TC microbiome, the present inventors have found that the level of microbes utilizing a Kegg pathway listed in Table K are indicative as to whether a subject is a responder or not.

TABLE K

| | |
|---|---|
| ko00640 | Propanoate metabolism |
| ko02060 | Phosphotransferase system (PTS) |
| ko05111 | Biofilm formation - Vibrio cholerae |
| ko00363 | Bisphenol degradation |

More specifically, the present inventors showed that lower abundance in the TC microbiome (i.e. levels below a predetermined level) of the species utilizing the Kegg pathway listed in Table K signifies a resistance to probiotic (i.e. non-permissive).

In the ascending colon (AC) microbiome, the present inventors have found that levels of the following genii/family of microbes are indicative as to whether a subject is a responder or not.

1. Bacteria of the genus Odoribacter

2. Bacteria of the family Rikenellaceae

More specifically, the present inventors showed that lower abundance (i.e. levels below a predetermined level) of these genii/family in the AC microbiome signifies a responder (i.e. permissive).

Furthermore, in the AC microbiome, the present inventors have found that the levels of species of microbes listed in Table L are indicative as to whether a subject is a responder or not.

TABLE L

s_Alistipes_onderdonkii
s_Odoribacter_unclassified
s_Roseburia_intestinalis
s_Bacteroides_caccae
s_Bacteroides_salyersiae
s_Eubacterium_ramulus

More specifically, the present inventors showed that lower abundance (i.e. levels below a predetermined level) of the above species in the AC microbiome signifies a responder (i.e. permissive).

Furthermore, in the AC microbiome, the present inventors have found that the levels of microbes utilizing fatty acid degradation Kegg pathway are indicative as to whether a subject is a responder or not.

More specifically, the present inventors showed that lower abundance in the AC microbiome (i.e. levels below a predetermined level) of microbes utilizing the fatty acid degradation Kegg pathway signifies a responder to probiotic (i.e. permissive).

In the cecum (Ce) microbiome, the present inventors have found that levels of the following genii/family of microbes are indicative as to whether a subject is a responder or not.

1. Bacteria of the genus Odoribacter

2. Bacteria of the family Rikenellaceae

More specifically, the present inventors showed that lower abundance (i.e. levels below a predetermined level) of these genii/family in the Ce microbiome signifies a responder (i.e. permissive).

Furthermore, in the Ce microbiome, the present inventors have found that the levels of species of Barnesiella_intestinihominis are indicative as to whether a subject is a responder or not.

More specifically, the present inventors showed that lower abundance (i.e. levels below a predetermined level) of the above species in the Ce microbiome signifies a responder (i.e. permissive).

Furthermore, in the Ce microbiome, the present inventors have found that the microbes utilizing propanoate metabolism Kegg pathway or the primary bile acid biosynthesis Kegg pathway are indicative as to whether a subject is a responder or not.

More specifically, the present inventors showed that lower abundance in the Ce microbiome (i.e. levels below a predetermined level) of microbes utilizing the primary bile acid biosynthesis pathway signifies a responder to probiotic (i.e. permissive), whereas lower abundance in the Ce microbiome (i.e. levels below a predetermined level) of microbes utilizing the propanoate metabolism Kegg pathway signifies a resistance to probiotic (i.e. non-permissive).

In the ileum (Ti) microbiome, the present inventors have found that levels of the following genii/family of microbes are indicative as to whether a subject is a responder or not.

1. Bacteria of the genus Faecalibacterium

2. Bacteria of the family Rikenellaceae

3. Bacteria of the genus Bifidobacterium

4. Bacteria of the family Ruminococcaceae

More specifically, the present inventors showed that lower abundance (i.e. levels below a predetermined level) of these genii/family in the Ti microbiome signifies a responder (i.e. permissive).

Furthermore, in the Ti microbiome, the present inventors have found that the levels of microbes utilizing limonene and pinene degradation Kegg pathway or the valine, leucine and isoleucine degradation Kegg pathway are indicative as to whether a subject is a responder or not.

More specifically, the present inventors showed that lower abundance in the Ti microbiome (i.e. levels below a predetermined level) of microbes utilizing these pathways signifies a responder to probiotic (i.e. permissive).

In the fundus (GF) microbiome, the present inventors have found that levels of the genus Actinobacillus are indicative as to whether a subject is a responder or not.

More specifically, the present inventors showed that lower abundance (i.e. levels below a predetermined level) of this genus in the GF microbiome signifies resistance (i.e. non-permissive).

Furthermore, in the GF microbiome, the present inventors have found that the level of microbes utilizing a Kegg pathway listed in Table M are indicative as to whether a subject is a responder or not.

TABLE M

| | |
|---|---|
| ko00710 | Carbon fixation in photosynthetic organisms |
| ko00910 | Nitrogen metabolism * |
| ko00051 | Fructose and mannose metabolism * |

More specifically, the present inventors showed that increase abundance in the GF microbiome (i.e. levels above a predetermined level) of bacteria utilizing a Kegg pathway listed in Table M in which no * appear signifies resistance to probiotic (i.e. non-permissive), whereas lower abundance in the GF (i.e. levels below a predetermined level) of the species listed in Table M in which an * appear signifies a resistance to probiotic (i.e. non-permissive).

Thus, according to a particular embodiment, the microbial signature comprises the absolute or relative amount of at least one, two, three, four, five, six, seven, eight, nine or ten or more of any of the bacterial species/genus/family/pathway listed in Tables A-M.

In one embodiment, the bacterial signature comprises the relative or absolute amount of the bacterial species that are provided as the probiotic. The present inventors have shown that a relatively low level of such species in a subject indicates that the subject is more likely to be a responder to such species in a probiotic.

In other embodiments, the microbial signature of the gut microbiome comprises a microbe diversity—for example alpha diversity. The present inventors have shown that the alpha diversity of responders was higher than that of non-responders at baseline.

In other embodiments, the microbial signature of the gut microbiome comprises a metabolite signature.

In other embodiments, the microbial signature of the gut microbiome comprises a bacterial signature.

In still other embodiments, the microbial signature refers to the relative abundance of genes or metabolites belonging to a particular pathway.

Preferably, the signature relates to at least 1, 2, 3, 4, 5, 6, 7, 8, 9, 10, 15, 20, 25, 30, 40, 50, 60, 70, 80, 90, 100, 150, 200, 250, 300 (e.g. 1-10, 1-20, 1-30, 1-40, 50, 10-100, 10-50, 20-50, 20-100) microbial species or product thereof.

It will be appreciated that the signature may comprise additional taxa of microbes other than species, including families, strains, genus, order etc.

As mentioned, the method is carried out by analyzing the microbes of a microbiome signature of the subject and comparing its microbial composition to the microbial composition of a microbiome of control subject known to be responsive to a probiotic. Additionally, the microbiome of the subject may be compared with a control subject known to be non-responsive to a probiotic. Measuring the microbial composition of the control subject may be carried out prior to, at the same time as, or following measuring the microbial composition of the test subject. Preferably, the microbiome (or signature thereof) of a plurality of control subject is measured. The data from such measurements may be stored in a database, as further described herein below.

When the test microbiome and the control microbiome from a subject known to be responsive have a statistically significant similar signature, then the likelihood of being responsive to the probiotic is increased as compared to a subject having a microbiome which is not statistically significantly similar to that of the responsive subject. Alternatively, a comparison can be made with a control subject known not to be response to a probiotic. When the two microbiomes have a statistically significant similar signature, then the likelihood of being responsive to the probiotic is decreased as compared to a subject having a microbiome which is statistically significantly similar to that of the non-responsive subject.

In another embodiment, the method is carried out by analyzing the metabolites of the metabolome of the subject and comparing its metabolite composition to the metabolite composition of a metabolome of a probiotic-responsive subject. When the two metabolomes have a statistically significant similar signature, then the likelihood of being responsive to a probiotic is increased as compared to a subject having a metabolome, which is not statistically significantly similar to that of the responsive subject.

According to still another embodiment, two microbiome signatures can be classified as being similar, if the number of genes belonging to a particular pathway expressed by both microbes is similar.

According to still another embodiment, two microbiome signatures can be classified as being similar, if the expression level of genes belonging to a particular pathway in both microbes is similar.

According to still another embodiment, two microbiome signatures can be classified as being similar, if the amount of a product generated by both microbes is similar.

The prediction of this aspect of the present invention may be made using an algorithm (e.g. a machine learning algorithm) which takes into account the relevance (i.e. weight) of particular microbes and/or products thereof in the composition. The algorithm may be built using gut microbiome data of a population of subjects classified according to their responsiveness to a probiotic.

The database may include other parameters relating to the subjects, for example the weight of the subject, the health of the subject, the blood chemistry of the subject, the genetic profile of the subject, the BMI of the subject, the eating habits of the subject and/or the health of the subject (e.g. diabetic, pre-diabetic, other metabolic disorder, hypertension, cardiac disorder etc.).

As used, herein the term “machine learning” refers to a procedure embodied as a computer program configured to induce patterns, regularities, or rules from previously collected data to develop an appropriate response to future data, or describe the data in some meaningful way.

Use of machine learning is particularly, but not exclusively, advantageous when the database includes multidimensional entries.

The database can be used as a training set from which the machine learning procedure can extract parameters that best describe the dataset. Once the parameters are extracted, they can be used to predict the likelihood of a subject responding to a probiotic treatment.

In machine learning, information can be acquired via supervised learning or unsupervised learning. In some embodiments of the invention the machine learning procedure comprises, or is, a supervised learning procedure. In supervised learning, global or local goal functions are used to optimize the structure of the learning system. In other words, in supervised learning there is a desired response, which is used by the system to guide the learning.

In some embodiments of the invention the machine learning procedure comprises, or is, an unsupervised learning procedure. In unsupervised learning there are typically no goal functions. In particular, the learning system is not provided with a set of rules. One form of unsupervised learning according to some embodiments of the present invention is unsupervised clustering in which the data objects are not class labeled, a priori.

Representative examples of “machine learning” procedures suitable for the present embodiments, including, without limitation, clustering, association rule algorithms, feature evaluation algorithms, subset selection algorithms, support vector machines, classification rules, cost-sensitive classifiers, vote algorithms, stacking algorithms, Bayesian networks, decision trees, neural networks, instance-based algorithms, linear modeling algorithms, k-nearest neighbors analysis, ensemble learning algorithms, probabilistic models, graphical models, regression methods, gradient ascent methods, singular value decomposition methods and principle component analysis. Among neural network models, the self-organizing map and adaptive resonance theory are commonly used unsupervised learning algorithms. The adaptive resonance theory model allows the number of clusters to vary with problem size and lets the user control the degree of similarity between members of the same clusters by means of a user-defined constant called the vigilance parameter.

Following is an overview of some machine learning procedures suitable for the present embodiments.

Association rule algorithm is a technique for extracting meaningful association patterns among features.

The term “association”, in the context of machine learning, refers to any interrelation among features, not just ones that predict a particular class or numeric value. Association includes, but it is not limited to, finding association rules, finding patterns, performing feature evaluation, performing feature subset selection, developing predictive models, and understanding interactions between features.

The term “association rules” refers to elements that co-occur frequently within the databases. It includes, but is not limited to association patterns, discriminative patterns, frequent patterns, closed patterns, and colossal patterns.

A usual primary step of association rule algorithm is to find a set of items or features that are most frequent among all the observations. Once the list is obtained, rules can be extracted from them.

The aforementioned self-organizing map is an unsupervised learning technique often used for visualization and analysis of high-dimensional data. Typical applications are focused on the visualization of the central dependencies within the data on the map. The map generated by the algorithm can be used to speed up the identification of association rules by other algorithms. The algorithm typically includes a grid of processing units, referred to as “neurons”. Each neuron is associated with a feature vector referred to as observation. The map attempts to represent all the available observations with optimal accuracy using a restricted set of models. At the same time, the models become ordered on the grid so that similar models are close to each other and dissimilar models far from each other. This procedure enables the identification as well as the visualization of dependencies or associations between the features in the data.

Feature evaluation algorithms are directed to the ranking of features or to the ranking followed by the selection of features based on their impact on the likelihood of the subject to respond to probiotic administration.

The term “feature” in the context of machine learning refers to one or more raw input variables, to one or more processed variables, or to one or more mathematical combinations of other variables, including raw variables and processed variables. Features may be continuous or discrete.

Information gain is one of the machine learning methods suitable for feature evaluation. The definition of information gain requires the definition of entropy, which is a measure of impurity in a collection of training instances. The reduction in entropy of the target feature that occurs by knowing the values of a certain feature is called information gain. Information gain may be used as a parameter to determine the effectiveness of a feature in explaining the likelihood of the subject under analysis to respond to a probiotic. Symmetrical uncertainty is an algorithm that can be used by a feature selection algorithm, according to some embodiments of the present invention. Symmetrical uncertainty compensates for information gain's bias towards features with more values by normalizing features to a [0,1] range.

Subset selection algorithms rely on a combination of an evaluation algorithm and a search algorithm. Similarly to feature evaluation algorithms, subset selection algorithms rank subsets of features. Unlike feature evaluation algorithms, however, a subset selection algorithm suitable for the present embodiments aims at selecting the subset of features with the highest impact on the likelihood of the subject under analysis to respond to an antibiotic, while accounting for the degree of redundancy between the features included in the subset. The benefits from feature subset selection include facilitating data visualization and understanding, reducing measurement and storage requirements, reducing training and utilization times, and eliminating distracting features to improve classification.

Two basic approaches to subset selection algorithms are the process of adding features to a working subset (forward selection) and deleting from the current subset of features (backward elimination). In machine learning, forward selection is done differently than the statistical procedure with the same name. The feature to be added to the current subset in machine learning is found by evaluating the performance of the current subset augmented by one new feature using cross-validation. In forward selection, subsets are built up by adding each remaining feature in turn to the current subset while evaluating the expected performance of each new subset using cross-validation. The feature that leads to the best performance when added to the current subset is retained and the process continues. The search ends when none of the remaining available features improves the predictive ability of the current subset. This process finds a local optimum set of features.

Backward elimination is implemented in a similar fashion. With backward elimination, the search ends when further reduction in the feature set does not improve the predictive ability of the subset. The present embodiments contemplate search algorithms that search forward, backward or in both directions. Representative examples of search algorithms suitable for the present embodiments include, without limitation, exhaustive search, greedy hill-climbing, random perturbations of subsets, wrapper algorithms, probabilistic race search, schemata search, rank race search, and Bayesian classifier.

A decision tree is a decision support algorithm that forms a logical pathway of steps involved in considering the input to make a decision.

The term “decision tree” refers to any type of tree-based learning algorithms, including, but not limited to, model trees, classification trees, and regression trees.

A decision tree can be used to classify the databases or their relation hierarchically. The decision tree has tree structure that includes branch nodes and leaf nodes. Each branch node specifies an attribute (splitting attribute) and a test (splitting test) to be carried out on the value of the splitting attribute, and branches out to other nodes for all possible outcomes of the splitting test. The branch node that is the root of the decision tree is called the root node. Each leaf node can represent a classification (e.g., whether a particular portion of the group database matches a particular portion of the subject-specific database) or a value (e.g., a predicted the likelihood of the subject to respond to a probiotic). The leaf nodes can also contain additional information about the represented classification such as a confidence score that measures a confidence in the represented classification (i.e., the likelihood of the classification being accurate). For example, the confidence score can be a continuous value ranging from 0 to 1, which a score of 0 indicating a very low confidence (e.g., the indication value of the represented classification is very low) and a score of 1 indicating a very high confidence (e.g., the represented classification is almost certainly accurate).

Support vector machines are algorithms that are based on statistical learning theory. A support vector machine (SVM) according to some embodiments of the present invention can be used for classification purposes and/or for numeric prediction. A support vector machine for classification is referred to herein as “support vector classifier,” support vector machine for numeric prediction is referred to herein as “support vector regression”.

An SVM is typically characterized by a kernel function, the selection of which determines whether the resulting SVM provides classification, regression or other functions. Through application of the kernel function, the SVM maps input vectors into high dimensional feature space, in which a decision hyper-surface (also known as a separator) can be constructed to provide classification, regression or other decision functions. In the simplest case, the surface is a hyper-plane (also known as linear separator), but more complex separators are also contemplated and can be applied using kernel functions. The data points that define the hyper-surface are referred to as support vectors.

The support vector classifier selects a separator where the distance of the separator from the closest data points is as large as possible, thereby separating feature vector points associated with objects in a given class from feature vector points associated with objects outside the class. For support vector regression, a high-dimensional tube with a radius of acceptable error is constructed which minimizes the error of the data set while also maximizing the flatness of the associated curve or function. In other words, the tube is an envelope around the fit curve, defined by a collection of data points nearest the curve or surface.

An advantage of a support vector machine is that once the support vectors have been identified, the remaining observations can be removed from the calculations, thus greatly reducing the computational complexity of the problem. An SVM typically operates in two phases: a training phase and a testing phase. During the training phase, a set of support vectors is generated for use in executing the decision rule. During the testing phase, decisions are made using the decision rule. A support vector algorithm is a method for training an SVM. By execution of the algorithm, a training set of parameters is generated, including the support vectors that characterize the SVM. A representative example of a support vector algorithm suitable for the present embodiments includes, without limitation, sequential minimal optimization.

The Least Absolute Shrinkage and Selection Operator (LASSO) algorithm is a shrinkage and/or selection algorithm for linear regression. The LASSO algorithm may minimize the usual sum of squared errors, with a regularization, that can be an L1 norm regularization (a bound on the sum of the absolute values of the coefficients), an L2 norm regularization (a bound on the sum of squares of the coefficients), and the like. The LASSO algorithm may be associated with soft-thresholding of wavelet coefficients, forward stagewise regression, and boosting methods. The LASSO algorithm is described in the paper: Tibshirani, R, Regression Shrinkage and Selection via the Lasso, J. Royal. Statist. Soc B., Vol. 58, No. 1, 1996, pages 267-288, the disclosure of which is incorporated herein by reference.

A Bayesian network is a model that represents variables and conditional interdependencies between variables. In a Bayesian network, variables are represented as nodes, and nodes may be connected to one another by one or more links. A link indicates a relationship between two nodes. Nodes typically have corresponding conditional probability tables that are used to determine the probability of a state of a node given the state of other nodes to which the node is connected. In some embodiments, a Bayes optimal classifier algorithm is employed to apply the maximum a posteriori hypothesis to a new record in order to predict the probability of its classification, as well as to calculate the probabilities from each of the other hypotheses obtained from a training set and to use these probabilities as weighting factors for future predictions about the likelihood of a subject to respond to a probiotic. An algorithm suitable for a search for the best Bayesian network, includes, without limitation, global score metric-based algorithm. In an alternative approach to building the network, Markov blanket can be employed. The Markov blanket isolates a node from being affected by any node outside its boundary, which is composed of the node's parents, its children, and the parents of its children.

Instance-based algorithms generate a new model for each instance, instead of basing predictions on trees or networks generated (once) from a training set.

The term “instance”, in the context of machine learning, refers to an example from a database.

Instance-based algorithms typically store the entire database in memory and build a model from a set of records similar to those being tested. This similarity can be evaluated, for example, through nearest-neighbor or locally weighted methods, e.g., using Euclidian distances. Once a set of records is selected, the final model may be built using several different algorithms, such as the naive Bayes.

Once a subject has been determined to be “responsive to a probiotic”, the present invention further contemplates treating the subject with a probiotic.

Thus, according to another aspect of the present invention, there is provided a method of treating a disease comprising administering a therapeutically effective amount of a probiotic to a subject in need thereof, the subject being deemed responsive to probiotic treatment according to the methods described herein thereby treating the disease.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Diseases, which may be treated with probiotics, include, but are not limited to allergic diseases (atopic dermatitis, possibly allergic rhinitis), gastrointestinal diseases such as colitis, inflammatory bowel disease and Diarrheal diseases, bacterial vaginosis, urinary tract infections, prevention of dental caries or respiratory infections.

In one embodiment, the disease is a chronic disease. In another embodiment, the disease is an acute disease.

The probiotic microorganism may be in any suitable form, for example in a powdered dry form. In addition, the probiotic microorganism may have undergone processing in order for it to increase its survival. For example, the microorganism may be coated or encapsulated in a polysaccharide, fat, starch, protein or in a sugar matrix. Standard encapsulation techniques known in the art can be used. For example, techniques discussed in U.S. Pat. No. 6,190,591, which is hereby incorporated by reference in its entirety, may be used.

According to a particular embodiment, the probiotic composition is formulated in a food product, functional food or nutraceutical.

In some embodiments, a food product, functional food or nutraceutical is or comprises a dairy product. In some embodiments, a dairy product is or comprises a yogurt product. In some embodiments, a dairy product is or comprises a milk product.

In some embodiments, a dairy product is or comprises a cheese product. In some embodiments, a food product, functional food or nutraceutical is or comprises a juice or other product derived from fruit. In some embodiments, a food product, functional food or nutraceutical is or comprises a product derived from vegetables. In some embodiments, a food product, functional food or nutraceutical is or comprises a grain product, including but not limited to cereal, crackers, bread, and/or oatmeal. In some embodiments, a food product, functional food or nutraceutical is or comprises a rice product. In some embodiments, a food product, functional food or nutraceutical is or comprises a meat product.

Prior to administration, the subject may be pretreated with an agent which reduces the number of naturally occurring microbes in the microbiome (e.g. by antibiotic treatment). According to a particular embodiment, the treatment significantly eliminates the naturally occurring gut microflora by at least 20%, 30% 40%, 50%, 60%, 70%, 80% or even 90%.

In some embodiments, administering comprises any means of administering an effective (e.g., therapeutically effective) or otherwise desirable amount of a composition to an individual. In some embodiments, administering a composition comprises administration by any route, including for example parenteral and non-parenteral routes of administration. Parenteral routes include, e.g., intraarterial, intracerebroventricular, intracranial, intramuscular, intraperitoneal, intrapleural, intraportal, intraspinal, intrathecal, intravenous, subcutaneous, or other routes of injection. Non-parenteral routes include, e.g., buccal, nasal, ocular, oral, pulmonary, rectal, transdermal, or vaginal. Administration may also be by continuous infusion, local administration, sustained release from implants (gels, membranes or the like), and/or intravenous injection.

In some embodiments, a composition is administered in an amount and/or according to a dosing regimen that is correlated with a particular desired outcome (e.g., with a particular change in microbiome composition and/or signature that correlates with an outcome of interest).

Particular doses or amounts to be administered in accordance with the present invention may vary, for example, depending on the nature and/or extent of the desired outcome, on particulars of route and/or timing of administration, and/or on one or more characteristics (e.g., weight, age, personal history, genetic characteristic, lifestyle parameter, etc., or combinations thereof). Such doses or amounts can be determined by those of ordinary skill. In some embodiments, an appropriate dose or amount is determined in accordance with standard clinical techniques. Alternatively or additionally, in some embodiments, an appropriate dose or amount is determined through use of one or more in vitro or in vivo assays to help identify desirable or optimal dosage ranges or amounts to be administered.

In some particular embodiments, appropriate doses or amounts to be administered may be extrapolated from dose-response curves derived from in vitro or animal model test systems. The effective dose or amount to be administered for a particular individual can be varied (e.g., increased or decreased) over time, depending on the needs of the individual. In some embodiments, where bacteria are administered, an appropriate dosage comprises at least about 100, 200, 300, 400, 500, 600, 700, 800, 900, 1000 or more bacterial cells. In some embodiments, the present invention encompasses the recognition that greater benefit may be achieved by providing numbers of bacterial cells greater than about 1000 or more (e.g., than about 1500, 2000, 2500, 3000, 35000, 4000, 4500, 5000, 5500, 6000, 7000, 8000, 9000, 10,000, 15,000, 20,000, 25,000, 30,000, 40,000, 50,000, 75,000, 100,000, 200,000, 300,000, 400,000, 500,000, 600,000, 700,000, 800,000, 900,000, 1×10⁶, 2×10⁶, 3×10⁶, 4×10⁶, 5×10⁶, 6×10⁶, 7×10⁶, 8×10⁶, 9×10⁶, 1×10⁷, 1×10⁸, 1×10⁹, 1×10¹⁰, 1×10¹¹, 1×10¹², 1×10¹³ or more bacteria.

Since probiotics are contemplated for health maintenance, and not necessarily for treatment of a disease, once a subject has been determined to be “responsive to a probiotic”, the present invention further contemplates providing the subject with the probiotic for health-promoting benefits.

Knowledge as to whether a subject is responsive to a probiotic is also useful to determine whether it is advantageous to treat that subject with a probiotic following antibiotic administration.

Thus, according to another aspect of the present invention, there is provided a method of treating a disease of a subject for which an antibiotic is therapeutic comprising:

(a) assessing whether the subject is suitable for probiotic treatment according to the method described herein;

(b) administering to the subject an antibiotic which is suitable for treating the disease; and subsequently

(c) administering to the subject a probiotic if the subject is deemed suitable for probiotic treatment; or administering to the subject an autologous fecal transplant if the subject is deemed not suitable for probiotic treatment, thereby treating the disease.

In one embodiment, the disease is a bacterial disease. In another embodiment, the disease is not a bacterial disease. In one embodiment, the disease is chronic. In another embodiment, the disease is acute.

Examples of diseases which may be treated using antibiotics include but are not limited to acne, appendicitis, atrial septal defect, bacterial arthritis, bacterial vaginosis, balance disorder, Bartholin's cyst, bursitis, pressure ulcer, bronchitis, conductive hearing loss, croup, cystic fibrosis, Granuloma inguinale, duodenitis, dermatitis, emphysema, endocarditis, enteritis, gastritis, Glomerulonephritis, Gonorrhea, cardiovascular disease, Hidradenitis suppurativa, laryngitis, Livedo reticularis, Lymphogranuloma venereum, marasmus, mastoiditis, meningitis, myocarditis, nephrotic syndrome, Neurogenic bladder dysfunction, Non-gonococcal urethritis, noonan syndrome, osteomyelitis, Onychocryptosis, otitis externa, otitis media, Patent ductus arteriosus, pelvic inflammatory disease, perforated eardrum, pericarditis, peritonitis, pharyngitis, pilonidal cyst, pleurisy, Prepatellar bursitis, Pyelonephritis, sepsis, Stevens-Johnson syndrome, Streptococcal pharyngitis, syphilis, tonsillitis, Trichomoniasis, tuberculosis, Ureterocele, urethral syndrome, urethritis, urinary tract infection and vertigo.

Examples of antibiotics contemplated by the present invention include, but are not limited to Daptomycin; Gemifloxacin; Telavancin; Ceftaroline; Fidaxomicin; Amoxicillin; Ampicillin; Bacampicillin; Carbenicillin; Cloxacillin; Dicloxacillin; Flucloxacillin; Mezlocillin; Nafcillin; Oxacillin; Penicillin G; Penicillin V; Piperacillin; Pivampicillin; Pivmecillinam; Ticarcillin; Aztreonam; Imipenem; Doripenem; Meropenem; Ertapenem; Clindamycin; Lincomycin; Pristinamycin; Quinupristin; Cefacetrile (cephacetrile); Cefadroxil (cefadroxyl); Cefalexin (cephalexin); Cefaloglycin (cephaloglycin); Cefalonium (cephalonium); Cefaloridine (cephaloridine); Cefalotin (cephalothin); Cefapirin (cephapirin); Cefatrizine; Cefazaflur; Cefazedone; Cefazolin (cephazolin); Cefradine (cephradine); Cefroxadine; Ceftezole; Cefaclor; Cefamandole; Cefmetazole; Cefonicid; Cefotetan; Cefoxitin; Cefprozil (cefproxil); Cefuroxime; Cefuzonam; Cefcapene; Cefdaloxime; Cefdinir; Cefditoren; Cefetamet; Cefixime; Cefmenoxime; Cefodizime; Cefotaxime; Cefpimizole; Cefpodoxime; Cefteram; Ceftibuten; Ceftiofur; Ceftiolene; Ceftizoxime; Ceftriaxone; Cefoperazone; Ceftazidime; Cefclidine; Cefepime; Cefluprenam; Cefoselis; Cefozopran; Cefpirome; Cefquinome; Fifth Generation; Ceftobiprole; Ceftaroline; Not Classified; Cefaclomezine; Cefaloram; Cefaparole; Cefcanel; Cefedrolor; Cefempidone; Cefetrizole; Cefivitril; Cefmatilen; Cefmepidium; Cefovecin; Cefoxazole; Cefrotil; Cefsumide; Cefuracetime; Ceftioxide; Azithromycin; Erythromycin; Clarithromycin; Dirithromycin; Roxithromycin; Telithromycin; Amikacin; Gentamicin; Kanamycin; Neomycin; Netilmicin; Paromomycin; Streptomycin; Tobramycin; Flumequine; Nalidixic acid; Oxolinic acid; Piromidic acid; Pipemidic acid; Rosoxacin; Ciprofloxacin; Enoxacin; Lomefloxacin; Nadifloxacin; Norfloxacin; Ofloxacin; Pefloxacin; Rufloxacin; Balofloxacin; Gatifloxacin; Grepafloxacin; Levofloxacin; Moxifloxacin; Pazufloxacin; Sparfloxacin; Temafloxacin; Tosufloxacin; Besifloxacin; Clinafloxacin; Gemifloxacin; Sitafloxacin; Trovafloxacin; Prulifloxacin; Sulfamethizole; Sulfamethoxazole; Sulfisoxazole; Trimethoprim-Sulfamethoxazole; Demeclocycline; Doxycycline; Minocycline; Oxytetracycline; Tetracycline; Tigecycline; Chloramphenicol; Metronidazole; Tinidazole; Nitrofurantoin; Vancomycin; Teicoplanin; Telavancin; Linezolid; Cycloserine 2; Rifampin; Rifabutin; Rifapentine; B acitracin; Polymyxin B; Viomycin; Capreomycin.

As used herein, the term “fecal transplant” refers to fecal bacteria isolated from a subject and thereby processed by the hand of man, which is transplanted into a recipient. In a particular embodiment, the fecal transplant is manmade processed fecal material (fecal filtrate) having reduced volume and/or fecal aroma relative to unprocessed fecal material. In a more particular embodiment, the fecal transplant is a fecal bacterial sample. The term fecal transplant may also be used to refer to the process of transplantation of fecal bacteria isolated from a healthy individual into a recipient. It is also referred to as fecal microbiota transplantation (FMT), stool transplant or bacteriotherapy.

Preferably, the fecal transplant is derived from a healthy subject. In a particular embodiment, the fecal transplant is an autologous fecal transplant.

An autologous fecal transplant is derived from the subject being treated prior to antibiotic administration and preferably prior to disease onset.

Methods of determining the amount of particular bacteria are provided herein above.

The present inventors have also found that the human fecal microbiome is a limited indicator of gut mucosal-associated microbiome composition and metagenomic function and particular taxa are more indicative than others.

Thus, for example Table N provides a list of bacterial genii or orders whose abundance in the stool is indicative of the abundance at particular locations along the GI tract.

TABLE N
orders of bacteria whose abundance in the stool
mirror the abundance of that bacteria in microbiomes
of different locations of the GI tract

| location | Genus/order |
|---|---|
| LGIM | g_Akkermansia |
| LGIM | g_Ruminococcus |
| LGIM | g_Faecalibacterium |
| LGIM | g_Prevotella |
| LGIM | o_Clostridiales |
| UGIM | g_Akkermansia |
| Re | g_Sutterella |
| Re | o_Clostridiales |
| Re | g_Faecalibacterium |
| Re | g_Prevotella |
| SC | g_Ruminococcus |
| SC | g_Faecalibacterium |
| SC | o_Clostridiales |
| SC | g_Prevotella |
| DC | g_Sutterella |
| DC | g_Ruminococcus |
| DC | g_Faecalibacterium |
| DC | g_Prevotella |
| DC | o_Clostridiales |
| TC | o_Clostridiales |
| TC | g_Prevotella |
| AC | g_Sutterella |
| AC | g_Faecalibacterium |
| AC | g_Prevotella |
| AC | o_Clostridiales |
| Ce | g_Sutterella |
| Ce | g_Faecalibacterium |
| Ce | g_[Ruminococcus] |
| Ce | o_Clostridiales |
| Ce | g_Prevotella |
| TI | g_Faecalibacterium |
| TI | g_Prevotella |
| TI | o_Clostridiales |
| TI | g_Streptococcus |
| Je | g_Bacteroides |
| Je | g_Akkermansia |
| Du | g_Bacteroides |
| Du | g_Akkermansia |
| GA | g_Akkermansia |
| GF | g_Akkermansia |

LGIM—mucosa of the lower GI;
Re—rectum;
SC—sigmoid colon;
DC—distal colon;
TC—transverse colon;
AC—ascending colon;
Ce—cecum;
TI—ileum;
Je—jejunum;
Du—duodenum;
GA—antrum;
GF—fundus;
g—genus;
o—order

In addition, Table O provides a list of bacterial species whose abundance in the stool is indicative of the abundance at particular locations along the GI tract.

TABLE O
Species of bacteria whose abundance in the stool
mirror the abundance of that bacteria in microbiomes
of different locations of the GI tract

| location | species |
|---|---|
| LGIM | s_Subdoligranulum_unclassified |
| LGIM | s_Bacteroides_dorei |
| LGIM | s_Bamesiella_intestinihominis |
| LGIM | s_Ruminococcus_torques |
| LGIM | s_Bacteroides_coprocola |
| LGIM | s_Bacteroides_caccae |
| LGIM | s_Bacteroides_uniformis |
| LGIM | s_Faecalibacterium_prausnitzii |
| UGIM | s_Bacteroides_dorei |
| UGIM | s_Bacteroides_vulgatus |
| Re | s_Bamesiella_intestinihominis |
| Re | s_Bacteroides_dorei |
| Re | s_Bacteroides_coprocola |
| Re | s_Bacteroides_uniformis |
| Re | s_Bacteroides_caccae |
| Re | s_Ruminococcus_torques |
| Re | s_Faecalibacterium_prausnitzii |
| SC | s_Bacteroides_dorei |
| SC | s_Bacteroides_coprocola |
| SC | s_Bacteroides_caccae |
| SC | s_Bamesiella_intestinihominis |
| SC | s_Bacteroides_uniformis |
| SC | s_Ruminococcus_torques |
| SC | s_Faecalibacterium_prausnitzii |
| DC | s_Bacteroides_caccae |
| DC | s_Bacteroides_coprocola |
| DC | s_Prevotella_copri |
| DC | s_Barnesiella_intestinihominis |
| DC | s_Ruminococcus_torques |
| DC | s_Bacteroides_uniformis |
| DC | s_Faecalibacterium_prausnitzii |
| DC | s_Coprococcus_comes |
| TC | s_Bacteroides_coprocola |
| TC | s_Bacteroides_caccae |
| TC | s_Barnesiella_intestinihominis |
| TC | s_Bacteroides_uniformis |
| TC | s_Faecalibacterium_prausnitzii |
| TC | s_Alistipes_putredinis |
| TC | s_Ruminococcus_torques |
| AC | s_Bacteroides_dorei |
| AC | s_Subdoligranulum_unclassified |
| AC | s_Bacteroides_coprocola |
| AC | s_Bacteroides_caccae |
| AC | s_Faecalibacterium_prausnitzii |
| AC | s_Barnesiella_intestinihominis |
| AC | s_Coprococcus_comes |
| Ce | s_Bacteroides_dorei |
| Ce | s_Bacteroides_vulgatus |
| Ce | s_Bacteroides_coprocola |
| Ce | s_Ruminococcus_torques |
| Ce | s_Bacteroides_caccae |
| Ce | s_Alistipes_putredinis |
| Ce | s_Barnesiella_intestinihominis |
| Ce | s_Faecalibacterium_prausnitzii |
| TI | s_Bacteroides_vulgatus |
| TI | s_Bacteroides_uniformis |
| TI | s_Bacteroides_dorei |
| TI | s_Bacteroides_caccae |
| TI | s_Alistipes_putredinis |
| TI | s_Barnesiella_intestinihominis |
| TI | s_Ruminococcus_torques |
| TI | s_Faecalibacterium_prausnitzii |
| GA | s_Bacteroides_dorei |
| GA | s_Bacteroides_vulgatus |
| GA | s_Prevotella_copri |
| GF | s_Bacteroides_vulgatus |

LGIM—mucosa of the lower GI;
Re—rectum;
SC—sigmoid colon;
DC—distal colon;
TC—transverse colon;
AC—ascending colon;
Ce—cecum;
TI—ileum;
Je—jejunum;
Du—duodenum;
GA—antrum;
GF—fundus

In addition, Table P provides a list of KO annotations whose abundance in the stool is indicative of the abundance at particular locations along the GI tract.

TABLE P
KO annotations whose abundance in the stool mirror the abundance of
that bacteria in microbiomes of different locations of the GI tract

| Organ | feature |
|---|---|
| LGIM | K01190 |
| LGIM | K03088 |
| LGIM | K07495 |
| LGIM | K07165 |
| LGIM | K07114 |
| LGIM | K03296 |
| LGIM | K02014 |
| Re | K07114 |
| SC | K01238 |
| SC | K07165 |
| SC | K03088 |
| SC | K01190 |
| SC | K07114 |
| SC | K02014 |
| SC | K03296 |
| DC | K07165 |
| DC | K01238 |
| DC | K07114 |
| DC | K03296 |
| DC | K02014 |
| DC | K03088 |
| DC | K01190 |
| TC | K07484 |
| TC | K00754 |
| TC | K00936 |
| TC | K01190 |
| TC | K03088 |
| TC | K04763 |
| TC | K00540 |
| TC | K07495 |
| TC | K01238 |
| AC | K07495 |
| AC | K03088 |
| AC | K01190 |
| AC | K02014 |
| AC | K03296 |
| AC | K07114 |
| Ce | K07484 |
| Ce | K07165 |
| Ce | K01238 |
| Ce | K07495 |
| Ce | K02014 |
| Ce | K07114 |
| Ce | K03296 |
| Ce | K01190 |
| Ce | K03088 |

LGIM—mucosa of the lower GI;
Re—rectum;
SC—sigmoid colon;
DC—distal colon;
TC—transverse colon;
AC—ascending colon;
Ce—cecum;

In addition, Table Q provides a list of KEGG pathways whose abundance in the stool is indicative of the abundance at particular locations along the GI tract.

TABLE Q
KEGG pathways whose abundance in the stool mirror
the abundance of that bacteria in microbiomes
of different locations of the GI tract

| Organ | feature |
|---|---|
| LGIM | ko01053 |
| LGIM | ko00480 |
| LGIM | ko00281 |
| LGIM | ko00363 |
| LGIM | ko00350 |
| LGIM | ko00785 |
| LGIM | ko00380 |
| LGIM | ko04146 |
| LGIM | ko00310 |
| LGIM | ko05111 |
| LGIM | ko00511 |
| LGIM | ko00121 |
| LGIM | ko00540 |
| LGIM | ko00280 |
| LGIM | ko00053 |
| LGIM | ko00311 |
| LGIM | ko00984 |
| Re | ko00280 |
| Re | ko00785 |
| Re | ko05111 |
| Re | ko00531 |
| SC | ko00071 |
| SC | ko00020 |
| SC | ko00650 |
| SC | ko00360 |
| SC | ko00531 |
| SC | ko04146 |
| SC | ko00281 |
| SC | ko00440 |
| SC | ko00052 |
| SC | ko00480 |
| SC | ko00130 |
| SC | ko01040 |
| SC | ko00350 |
| SC | ko00363 |
| SC | ko00380 |
| SC | ko00121 |
| SC | ko00785 |
| SC | ko00310 |
| SC | ko00053 |
| SC | ko00540 |
| SC | ko00280 |
| SC | ko00984 |
| SC | ko00311 |
| SC | ko00511 |
| SC | ko05111 |
| DC | ko00650 |
| DC | ko01040 |
| DC | ko00052 |
| DC | ko00440 |
| DC | ko00480 |
| DC | ko00350 |
| DC | ko00280 |
| DC | ko00281 |
| DC | ko00790 |
| DC | ko00130 |
| DC | ko01053 |
| DC | ko00380 |
| DC | ko00020 |
| DC | ko00785 |
| DC | ko00984 |
| DC | ko04146 |
| DC | ko00511 |
| DC | ko00121 |
| DC | ko00053 |
| DC | ko00540 |
| DC | ko00311 |
| DC | ko05111 |
| TC | ko00071 |
| TC | ko00311 |
| TC | ko04614 |
| TC | ko00061 |
| TC | ko00908 |
| TC | ko00540 |
| TC | ko00633 |
| TC | ko00130 |
| TC | ko00020 |
| TC | ko00310 |
| TC | ko03018 |
| TC | ko00281 |
| TC | ko00740 |
| TC | ko00053 |
| TC | ko00350 |
| TC | ko00040 |
| TC | ko00360 |
| TC | ko01040 |
| TC | ko00780 |
| TC | ko00480 |
| TC | ko00984 |
| TC | ko00440 |
| TC | ko00790 |
| TC | ko00650 |
| TC | ko00280 |
| TC | ko00562 |
| TC | ko05111 |
| TC | ko04146 |
| TC | ko00363 |
| TC | ko00121 |
| TC | ko00052 |
| TC | ko00380 |
| AC | ko00380 |
| AC | ko01040 |
| AC | ko00480 |
| AC | ko00640 |
| AC | ko00650 |
| AC | ko00020 |
| AC | ko00281 |
| AC | ko00130 |
| AC | ko00633 |
| AC | ko00984 |
| AC | ko00531 |
| AC | ko01053 |
| AC | ko04146 |
| AC | ko00311 |
| AC | ko00363 |
| AC | ko00052 |
| AC | ko00540 |
| AC | ko00121 |
| AC | ko00511 |
| AC | ko05111 |
| AC | ko00053 |
| AC | ko00785 |
| AC | ko00280 |
| Ce | ko00363 |
| Ce | ko00910 |
| Ce | ko00780 |
| Ce | ko00061 |
| Ce | ko00360 |
| Ce | ko00531 |
| Ce | ko00633 |
| Ce | ko00350 |
| Ce | ko00785 |
| Ce | ko00020 |
| Ce | ko00562 |
| Ce | ko01040 |
| Ce | ko00790 |
| Ce | ko01053 |
| Ce | ko00480 |
| Ce | ko00121 |
| Ce | ko00380 |
| Ce | ko00130 |
| Ce | ko00281 |
| Ce | ko00511 |
| Ce | ko05111 |
| Ce | ko00650 |
| Ce | ko00052 |
| Ce | ko00440 |
| Ce | ko00310 |
| Ce | ko00311 |
| Ce | ko00053 |
| Ce | ko04146 |
| Ce | ko00540 |
| Ce | ko00280 |
| Ce | ko00984 |
| TI | ko00785 |
| TI | ko00531 |

LGIM—mucosa of the lower GI;
Re—rectum;
SC—sigmoid colon;
DC—distal colon;
TC—transverse colon;
AC—ascending colon;
Ce—cecum;
TI—ileum;

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion. Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Person-Specific Microbiome-Mediated Gut Mucosal Colonization Resistance to Empiric Probiotics in the Naive Host

### Materials and Methods

TABLE 1
Reagents and Resources

| | | |
|---|---|---|
| REAGENT or RESOURCE | SOURCE | IDENTIFIER |

Bacterial and Virus Strains

| | | |
|---|---|---|
| Lactobacillus acidophilus | | ATCC 4356 |
| Lactobacillus rhamnosus | Clinical isolate | |
| Lactobacillus casei | | ATCC 393 |
| Lactobacillus casei subsp. paracasei | | ATCC BAA- |
| | | 52 |
| Lactobacillus plantarum | | ATCC 8014 |
| Bifidobacterium longum subsp. infantis | | ATCC 15697 |
| Bifidobacterium bifidum | | ATCC 29521 |
| Bifidobacterium breve | | ATCC 15700 |
| Bifidobacterium longum subsp. longum | | ATCC 15707 |
| Lactococcus lactis | Isolated from | |
| | Bio 25 Supherb | |
| Streotococcus thermophilus | | ATCC BAA- |
| | | 491 |
| Biological Samples | | |

Chemicals, Peptides, and Recombinant Proteins

| | |
|---|---|
| Bio 25 Supherb | Supherb Ltd, |
| | Nazareth Ilit, |
| | Israel |

Critical Commercial Assays

| | | |
|---|---|---|
| NextSeq 500/550 High Output v2 kit (150 cycles) | illumina | FC-404-2002 |
| Was used for Metagenome shotgun sequencing | | |
| NextSeq 500/550 High Output v2 kit (75 cycles) | illumina | FC-404-2005 |
| Was used for RNA-Seq | | |
| MiSeq Reagent Kit v2 (500-cycles) | illumina | MS-102-2003 |
| RNeasy mini kit | Qiagen | RNAeasy |
| | | 74104 |
| PowerSoil DNA Isolation Kit (MOBIO Laboratories) | Qiagen | DNeasy Power |
| | | Lyzer |
| | | PowerSoil, |
| | | 12855-100 |
| NEBNext Ultra Directional RNA Library Prep Kit for | New England | E7420S |
| Illumina | Biolabs | |
| NEB Next Multiplex Oligos for Illumina | New England | E7600S |
| | Biolabs | |

Experimental Models: Organisms/Strains

| | |
|---|---|
| C57BL/6J01aHsd males 8-9 weeks of age | Envigo, Israel |
| Germ-free Swiss-Webster males 8-9 weeks of age | Weizmann |
| | institute of |
| | Science |

Sequence-Based Reagents

| | | |
|---|---|---|
| Miseq Illumina sequencing primers | | |
| Read 1- | | |
| TATGGTAATTGTGTGCCAGCMGCCGCGGTAA | | |
| (SEQ ID NO: 1) | | |
| Read 2- | | |
| AGTCAGTCAGCCGGACTACHVGGGTWTCTAA | | |
| T (SEQ ID NO: 2) | | |
| Index primer - | | |
| ATTAGAWACCCBDGTAGTCCGGCTGACTGAC | | |
| T (SEQ ID NO: 3) | | |
| qPCR primers | | |
| LAC- | L. acidophilus | ⁸⁸ |
| F:CTTTGACTCAGGCAATTGCTCGTGAAGGTAT | qPCR | |
| G (SEQ ID NO: 4) | | |
| LAC- | L. acidophilus | ⁸⁸ |
| R:CAACTTCTTTAGATGCTGAAGAAACAGCAG | qPCR | |
| CTACG (SEQ ID NO: 5) | | |
| LRH-F:GTGCTTGCATCTTGATTTAATTTT | L. rhamnosus | ⁸⁹ |
| (SEQ ID NO: 6) | qPCR | |
| LRH-R:TGCGGTTCTTGGATCTATGCG | L. rhamnosus | ⁸⁹ |
| (SEQ ID NO: 7) | qPCR | |
| LCA-F:GTGCTTGCACTGAGATTCGACTTA | L. casei qPCR | ⁸⁹ |
| (SEQ ID NO: 8) | | |
| LCA-R:TGCGGTTCTTGGATCTATGCG | L. casei qPCR | ⁸⁹ |
| (SEQ ID NO: 9) | | |
| LPA-F:GTGCTTGCACCGAGATTCAACATG | L. paracasei | ⁸⁹ |
| (SEQ ID NO: 10) | qPCR | |
| LPA-R:TGCGGTTCTTGGATCTATGCG | L. paracasei | ⁸⁹ |
| (SEQ ID NO: 11) | qPCR | |
| LPL-F:TTACATTTGAGTGAGTGGCGAACT | L. plantarum | ⁹⁰ |
| (SEQ ID NO: 12) | qPCR | |
| LPL-R:AGGTGTTATCCCCCGCTTCT | L. plantarum | ⁹⁰ |
| (SEQ ID NO: 13) | qPCR | |
| BIN-F:CGC GAG CAA AAC AAT GGT T | B. infantis qPCR | ⁹¹ |
| (SEQ ID NO: 14) | | |
| BIN-R:AAC GAT CGA AAC GAA CAA TAG AGT | B. infantis qPCR | ⁹¹ |
| T | | |
| (SEQ ID NO: 15) | | |
| BBI-F:GTT GAT TTC GCC GGA CTC TTC | B. bifidum qPCR | ⁹¹ |
| (SEQ ID NO: 16) | | |
| BBI-R:GCA AGC CTA TCG CGC AAA | B. bifidum qPCR | ⁹¹ |
| (SEQ ID NO: 17) | | |
| BBR-F:GTG GTG GCT TGA GAA CTG GAT AG | B. breve qPCR | ⁹¹ |
| (SEQ ID NO: 18) | | |
| BBR-R:CAA AAC GAT CGA AAC AAA CAC TAA | B. breve qPCR | ⁹¹ |
| A | | |
| (SEQ ID NO: 19) | | |
| BLO-F:TGG AAG ACG TCG TTG GCT TT | B. longum qPCR | ⁹¹ |
| (SEQ ID NO: 20) | | |
| BLO-R:ATC GCG CCA GGC AAA A | B. longum qPCR | ⁹¹ |
| (SEQ ID NO: 21) | | |
| LLA-F:TGA ACC ACA ATG GGT TGC TA | L. lactis qPCR | ⁹² |
| (SEQ ID NO: 22) | | |
| LLA-R:TCG ACT GGA AGA AGG AGT GG | L. lactis qPCR | ⁹² |
| (SEQ ID NO: 23) | | |
| STH-F:TTATTTGAAAGGGGCAATTGCT | S. thermophilus | ⁸⁹ |
| (SEQ ID NO: 24) | qPCR | |
| STH-R:GTGAACTTTCCACTCTCACAC | S. thermophilus | ⁸⁹ |
| (SEQ ID NO: 25) | qPCR | |
| qPCR primers for 16S gene | | |
| 111-967F-PP:CNACGCGAAGAACCTTANC | Total 16S qPCR | ⁹³ |
| (SEQ ID NO: 26) | | |
| 112-967F-UC3:ATACGCGARGAACCTTACC | Total 16S qPCR | ⁹³ |
| (SEQ ID NO: 27) | | |
| 113-967F-AQ:CTAACCGANGAACCTYACC | Total 16S qPCR | ⁹³ |
| (SEQ ID NO: 28) | | |
| 114-967F-S :CAACGCGMARAACCTTACC | Total 16S qPCR | ⁹³ |
| (SEQ ID NO: 29) | | |
| 115- 1046R-S :CGACRRCCATGCANCACCT | Total 16S qPCR | ⁹³ |
| (SEQ ID NO: 30) | | |

Software and Algorithms

| | | |
|---|---|---|
| QIIME | | ⁹⁴ |
| Trimmomatic | | ⁹⁵ |
| MetaPhlAn2 | | ⁹⁶ |
| Bowtie2 | | ⁹⁷ |
| EMPANADA | | ⁹⁸ |
| RNASeq analysis software | GOrilla (Gene | ⁹⁹ |
| | Ontology | |
| | enRIchment | |
| | anaLysis and | |
| | visuaLizAtion | |
| | tool) | |

### Experimental Model and Subject Details

Clinical trial: The human trial was approved by the Tel Aviv Sourasky Medical Center Institutional Review Board (IRB approval numbers TLV-0553-12, TLV-0658-12 and 0196-13-TLV) and Weizmann Institute of Science Bioethics and Embryonic Stem Cell Research oversight committee (IRB approval numbers 421-1, 430-1 and 444-1), and was reported to clinical trials (Identifier: NCT03218579). Written informed consent was obtained from all subjects. No changes were done to the study protocol and methods after the trial commenced.

Exclusion and inclusion criteria (human cohorts): All subjects fulfilled the following inclusion criteria: males and females, aged 18-70, who are currently not following any diet regime or dietitian consultation and are able to provide informed consent. Exclusion criteria included: (i) pregnancy or fertility treatments; (ii) usage of antibiotics or antifungals within three months prior to participation; (iii) consumption of probiotics in any form within one month prior to participation, (iv) chronically active inflammatory or neoplastic disease in the three years prior to enrollment; (v) chronic gastrointestinal disorder, including inflammatory bowel disease and celiac disease; (vi) active neuropsychiatric disorder; (vii) myocardial infarction or cerebrovascular accident in the 6 months prior to participation; (viii) coagulation disorders; (ix) chronic immunosuppressive medication usage; (x) pre-diagnosed type I or type II diabetes mellitus or treatment with anti-diabetic medication. Adherence to inclusion and exclusion criteria was validated by medical doctors.

TABLE 2
Participants details

| | | | Age | Weight | Height | BMI | | |
|---|---|---|---|---|---|---|---|---|
| # | Sex | Group | (years) | (Kg) | (cm) | (kg/m2) | Smoking | Diet |
| 1 | F | No intervention | 40 | 50 | 158 | 20.03 | Never | Omnivore |
| 2 | M | No intervention | 46 | 100 | 191 | 27.41 | Never | Vegetarian |
| 3 | M | No intervention | 32 | 63 | 178 | 19.88 | Never | Omnivore |
| 4 | F | No intervention | 45 | 59 | 159 | 23.34 | Never | Omnivore |
| 5 | M | No intervention | 58 | 76 | 175 | 24.82 | Never | Omnivore |
| 6 | M | No intervention | 58 | 100 | 184 | 29.54 | Never | Omnivore |
| 7 | F | No intervention | 40 | 65 | 160 | 25.39 | Never | Omnivore |
| 8 | F | No intervention | 66 | 64 | 164 | 23.8 | Never | Omnivore |
| 9 | F | No intervention | 25 | 60 | 172 | 20.28 | Past | Omnivore |
| 10 | F | No intervention | 27 | 66 | 170 | 22.84 | Never | Omnivore |
| 11 | M | Probiotics | 19 | 80 | 186 | 23.12 | Past | Omnivore |
| 12 | F | Probiotics | 35 | 50 | 168 | 17.72 | Never | Vegetarian |
| 13 | M | Probiotics | 47 | 84 | 187 | 24.02 | Never | Vegetarian |
| 14 | F | Probiotics | 23 | 60 | 170 | 20.76 | Never | Vegan |
| 15 | F | Probiotics | 25 | 37 | 149 | 16.67 | Never | Vegan |
| 16 | M | Probiotics | 35 | 77 | 172 | 26.03 | Present | Vegetarian |
| 17 | M | Probiotics | 65 | 80 | 176 | 25.83 | Never | Omnivore |
| 18 | F | Probiotics | 64 | 67 | 164 | 24.91 | Past | Omnivore |
| 19 | M | Probiotics | 43 | 69 | 176 | 22.28 | Past | Omnivore |
| 20 | M | Probiotics | 39 | 62 | 180 | 19.14 | Never | Omnivore |
| 21 | M | Placebo | 29 | 67 | 190 | 18.56 | Never | Omnivore |
| 22 | F | Placebo | 32 | 70 | 162 | 26.67 | Never | Vegetarian |
| 23 | M | Placebo | 35 | 78 | 175 | 25.47 | Never | Omnivore |
| 24 | F | Placebo | 65 | 82 | 167 | 29.40 | Never | Omnivore |
| 25 | F | Placebo | 40 | 50 | 158 | 20.03 | Never | Omnivore |
| 26 | F | Validation | 51 | 68 | 168 | 24.09 | Never | Omnivore |
| 27 | F | Validation | 52 | 51 | 167 | 18.29 | Past | Omnivore |
| 28 | M | Validation | 50 | 70 | 172 | 23.66 | Present | Omnivore |
| 29 | M | Validation | 48 | 85 | 187 | 24.31 | Past | Omnivore |

Human Study Design: Twenty-nine healthy volunteers were recruited for this study between the years 2016 and 2018. Upon enrollment, participants were required to fill up medical, lifestyle and food frequency questionnaires, which were reviewed by medical doctors before the acceptance to participate in the study. Two cohorts were recruited, a naive cohort (n=10) and a case-control cohort (n=19), subdivided into 2 interventions of probiotics (n=14) and placebo pills (n=5). For the latter cohort, the study design consisted of four phases, baseline (7 days), intervention (28 days) and follow-up (28 days). During the 4-week intervention phase (days 1 thru 28), participants from the probiotics arm were instructed to consume a commercial probiotic supplement (Bio-25) bidaily; participants from the placebo arm were instructed to consume a similar-looking pill bidaily (see “Drugs and biological preparations”). In the case-control cohort stool samples were collected daily during the baseline phase and during the first week of intervention, and then weekly throughout the rest of the intervention and follow-up phases. Ten participants in the probiotics arm and the entire placebo arm underwent two endoscopic examinations, one immediately before the intervention, at the end of the baseline phase (day 0), and another three weeks through the intervention phase (day 21). Participants in the naive cohort underwent a single endoscopic examination; and four participants in the probiotics arm (“validation arm”) underwent only a single colonoscopy three weeks through the intervention phase (day 21).

The trial was completed as planned. All 29 subjects completed the trial and there were no dropouts or withdrawals. Adverse effects were mild and did not tamper with the study protocol. They included minor bleeding following endoscopic mucosal sampling and throat pain and hoarseness following the endoscopic examination.

All participants received payment for their participation in the study upon discharge from their last endoscopic session.

Drugs and Biological Preparations

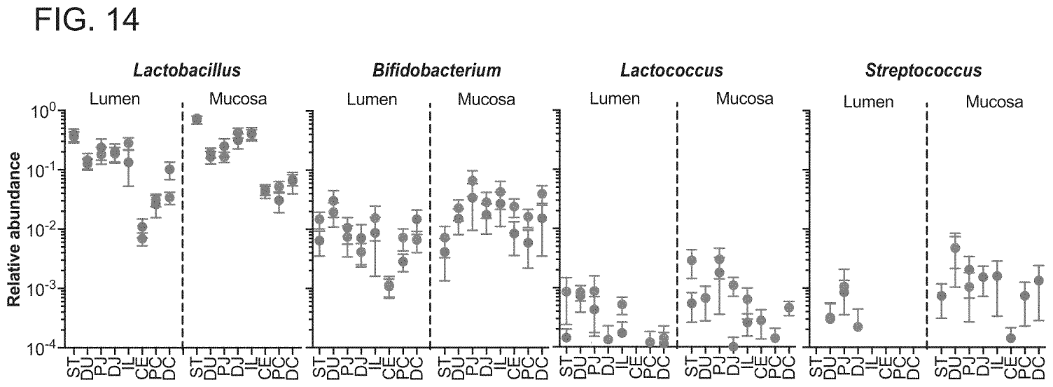

Probiotics: During the probiotics phase participants were treated by oral Supherb Bio-25 twice daily, which is described by the manufacturer to contain at least 25 billion active bacteria of the following species: B. bifidum, L. rhamnosus, L. lactis, L. casei, B. breve, S. thermophilus, B. longum, L. paracasei, L. plantarum and B. infantis. According to the manufacturer, the pills underwent double coating to ensure their survival under stomach acidity condition and their proliferation in the intestines. Validation of the aforementioned strains quantity and viability was performed as part of the study, see FIG. 14.

Placebo pills: Placebo pills (Trialog, Inc.) were composed of a hydroxypropylmethyl cellulose (HPMC) capsule, filled with 600 mg microcrystalline cellulose PH.EU (MCC). Placebo pill manufacturing process was approved for pharmaceutical use by the Israeli Ministry of Health, and underwent a microbial burden examination prior to administration. Placebo and probiotic pills were labeled identically to maintain blinding.

Gut Microbiome Sampling

Stool sampling: Participants were requested to self-sample their stool on pre-determined intervals using a swab following detailed printed instructions. Collected samples were immediately stored in a home freezer (−20° C.) for no more than 7 days and transferred in a provided cooler to our facilities, where they were stored at −80° C.

Endoscopic examination: Forty-eight hours prior to the endoscopic examination, participants were asked to follow a pre-endoscopy diet. 20 hours prior to the examination diet was restricted to clear liquids. All participants underwent a sodium picosulfate (Pico Salax)-based bowel preparation. Participants were equipped with two fleet enemas, which they were advised to use in case of unclear stools. The examination was performed using a Pentax 90i endoscope (Pentax Medical) under light sedation with propofol-midazolam.

Luminal content was aspirated from the stomach, duodenum, jejunum, terminal ileum, cecum and descending colon into 15 ml tubes by the endoscope suction apparatus and placed immediately liquid nitrogen. Brush cytology (US Endoscopy) was used to scrape the gut lining to obtain mucosal content from the gastric fundus, gastric antrum, duodenal bulb, jejunum, terminal ileum, cecum, ascending colon, transverse colon, descending colon, sigmoid colon and rectum. Brushes were placed in a screw cap micro tube and were immediately stored in liquid nitrogen. Biopsies from the gut epithelium were obtained from the stomach, duodenum, jejunum, terminal ileum, cecum and descending colon and were immediately stored in liquid nitrogen. By the end of each session, all samples were transferred to Weizmann Institute of Science and stored in −80° C. In the two endoscopic examinations arm the endoscopies were scheduled in sessions 3 weeks apart

Mouse study design; C57BL/6 male mice were purchased from Harlan Envigo and allowed to acclimatize to the animal facility environment for 2 weeks before used for experimentation. Germ-free Swiss-Webster mice were born in the Weizmann Institute germ-free facility, kept in gnotobiotic isolators and routinely monitored for sterility. In all experiments, age- and gender-matched mice were used. Mice were 8-9 weeks of age and weighed 20 gr at average at the beginning of experiments. All mice were kept at a strict 24 hr light-dark cycle, with lights being turned on from 6 am to 6 pm. Each experimental group consisted of two cages to control for cage effect. For probiotics consumption, a single pill (Supherb Bio-25) was dissolved in 10 mL of sterile PBS and immediately fed to mice by oral gavage during the dark phase. For FMT experiments, 200 mg of stored human stool samples were resuspended in sterile PBS under anaerobic conditions (Coy Laboratory Products, 75% N2, 20% CO2, 5% H2), vortexed for 3 minutes and allowed to settle by gravity for 2 min. Samples were immediately transferred to the animal facility in Hungate anaerobic culture tubes and the supernatant was administered to germ-free mice by oral gavage. Mice were allowed to conventionalize for three days prior to probiotics treatment, as previously described. Stool was collected on pre-determined days at the beginning of the dark phase, and immediately snap-frozen and transferred for storage at −80° C. until further processing. Upon the termination of experiments, mice were sacrificed by CO2 asphyxiation, and laparotomy was performed by employing a vertical midline incision. After the exposure and removal of the digestive tract, it was dissected into eight parts: the stomach; beginning at the pylorus, the proximal 4 cm of the small intestine was collected as the duodenum; the following third of the small intestine was collected as the proximal and distal jejunum; the ileum was harvested as the distal third of the small intestine; the cecum; lastly, the colon was divided into its proximal and distal parts. For each section, the content within the cavity was extracted and collected for luminal microbiome isolation, and the remaining tissue was rinsed three times with sterile PBS and collected for mucosal microbiome isolation. During each time point, each group was handled by a different researcher in one biological hood to minimize cross-contamination. All animal studies were approved by the Weizmann Institute of Science Institutional Animal Care and Use committee (IACUC), application number 29530816-2.

Bacterial cultures: Bacterial strains used in this study are listed in Key Resource Table. Lactobacillus strains were grown in De Man, Rogosa and Sharpe (MRS) broth or agar, Bifidobacterium strains in modified Bifidobacterium agar or modified reinforced clostridial broth, Lactococcus and Streptococcus were grown in liquid or solid M17 medium. Liquid or solid Brain-Heart Infusion (BHI) was used for non-selective growth of probiotic bacteria. Cultures were grown under anaerobic conditions (Coy Laboratory Products, 75% N2, 20% CO2, 5% H2) in 37° C. without shaking. All growth media were purchased from BD. For enumeration of viable bacteria from the probiotics pill, a single pill (Supherb Bio-25) was dissolved in 10 mL of sterile PBS and serially diluted on all growth media.

Nucleic Acid Extraction

DNA purification: DNA was isolated from endoscopic samples, both luminal content and mucosal brushes, using PowerSoil DNA Isolation Kit (MOBIO Laboratories). DNA was isolated from stool swabs using PowerSoil DNA Isolation Kit (MOBIO Laboratories) optimized for an automated platform.

RNA Purification: Gastrointestinal biopsies obtained from the participants were purified using RNAeasy kit (Qiagen, 74104) according to the manufacturer's instructions. Most of the biopsies were kept in RNAlater solution (ThermoFisher, AM7020) and were immediately frozen at liquid nitrogen.

Nucleic Acid Processing and Library Preparation

16S qPCR Protocol for Quantification of Bacterial DNA: DNA templates were diluted to 1 ng/ul before amplifications with the primer sets (indicated in Table 3) using the Fast Sybr™ Green Master Mix (ThermoFisher) in duplicates. Amplification conditions were: Denaturation 95° C. for 3 minutes, followed by 40 cycles of Denaturation 95° C. for 3 seconds; annealing 64° C. for 30 seconds followed by meting curve. Duplicates with >2 cycle difference were excluded from analysis. The CT value for any sample not amplified after 40 cycles was defined as 40 (threshold of detection).

TABLE 3
Primers used in qPCR analysis.

| Sequence | Target & reference |
|---|---|
| LAC-F:CTTTGACTCAGGCAATTGCTCGTGAAGGTATG | L. acidophilus |
| (SEQ ID NO: 31) | qPCR⁸⁸ |
| LAC-R:CAACTTCTTTAGATGCTGAAGAAACAGCAGCTACG | L. acidophilus |
| (SEQ ID NO: 32) | qPCR⁸⁸ |
| LRH-F:GTGCTTGCATCTTGATTTAATTTT (SEQ ID NO: 33) | L. rhamnosus |
| | qPCR⁸⁹ |
| LRH-R:TGCGGTTCTTGGATCTATGCG (SEQ ID NO: 34) | L. rhamnosus |
| | qPCR⁸⁹ |
| LCA-F:GTGCTTGCACTGAGATTCGACTTA (SEQ ID NO: 35) | L. casei qPCR⁸⁹ |
| LCA-R:TGCGGTTCTTGGATCTATGCG (SEQ ID NO: 36) | L. casei qPCR⁸⁹ |
| LPA-F:GTGCTTGCACCGAGATTCAACATG (SEQ ID NO: 37) | L. paracasei qPCR⁸⁹ |
| LPA-R:TGCGGTTCTTGGATCTATGCG (SEQ ID NO: 38) | L. paracasei qPCR⁸⁹ |
| LPL-F:TTACATTTGAGTGAGTGGCGAACT (SEQ ID NO: 39) | L. plantarum qPCR⁹⁰ |
| LPL-R:AGGTGTTATCCCCCGCTTCT (SEQ ID NO: 40) | L. plantarum qPCR⁹⁰ |
| BIN-F:CGC GAG CAA AAC AAT GGT T (SEQ ID NO: 41) | B. infantis qPCR⁹¹ |
| BIN-R:AAC GAT CGA AAC GAA CAA TAG AGT T (SEQ ID NO: | B. infantis qPCR⁹¹ |
| 42) | |
| BBI-F:GTT GAT TTC GCC GGA CTC TTC (SEQ ID NO: 43) | B. bifidum qPCR⁹¹ |
| BBI-R:GCA AGC CTA TCG CGC AAA (SEQ ID NO: 44) | B. bifidum qPCR⁹¹ |
| BBR-F:GTG GTG GCT TGA GAA CTG GAT AG (SEQ ID NO: 45) | B. breve qPCR⁹¹ |
| BBR-R:CAA AAC GAT CGA AAC AAA CAC TAA A (SEQ ID NO: | B. breve qPCR⁹¹ |
| 46) | |
| BLO-F:TGG AAG ACG TCG TTG GCT TT (SEQ ID NO: 47) | B. longum qPCR⁹¹ |
| BLO-R:ATC GCG CCA GGC AAA A (SEQ ID NO: 48) | B. longum qPCR⁹¹ |
| LLA-F:TGA ACC ACA ATG GGT TGC TA (SEQ ID NO: 49) | L. lactis qPCR⁹² |
| LLA-R:TCG ACT GGA AGA AGG AGT GG (SEQ ID NO: 50) | L. lactis qPCR⁹² |
| STH-F:TTATTTGAAAGGGGCAATTGCT (SEQ ID NO: 51) | S. thermophilus |
| | qPCR⁸⁹ |
| STH-R:GTGAACTTTCCACTCTCACAC (SEQ ID NO: 52) | S. thermophilus |
| | qPCR⁸⁹ |
| qPCR primers for 16S gene⁹³ | |
| 111-967F-PP:CNACGCGAAGAACCTTANC (SEQ ID NO: 53) | Total 16S qPCR |
| 112-967F-UC3:ATACGCGARGAACCTTACC (SEQ ID NO: 54) | Total 16S qPCR |
| 113-967F-AQ:CTAACCGANGAACCTYACC (SEQ ID NO: 55) | Total 16S qPCR |
| 114-967F-S:CAACGCGMARAACCTTACC (SEQ ID NO: 56) | Total 16S qPCR |
| 115-1046R-S:CGACRRCCATGCANCACCT (SEQ ID NO: 57) | Total 16S qPCR |

16S rDNA Sequencing: For 16S amplicon pyrosequencing, PCR amplification was performed spanning the V4 region using the primers 515F/806R of the 16S rRNA gene and subsequently sequenced using 2×250 bp paired-end sequencing (Illumina MiSeq). Custom primers were added to Illumina MiSeq kit resulting in 253 bp fragment sequenced following paired end joining to a depth of 110,998±66,946 reads (mean±SD).

Read1:
(SEQ ID NO: 58)
TATGGTAATTGTGTGCCAGCMGCCGCGGTAA
Read2:
(SEQ ID NO: 59)
AGTCAGTCAGCCGGACTACHVGGGTWTCTAAT
Index sequence primer:
(SEQ ID NO: 60)
ATTAGAWACCCBDGTAGTCCGGCTGACTGACTATTAGAA

Whole genome shotgun sequencing: 100 ng of purified DNA was sheared with a Covaris E220X sonicator. Illumina compatible libraries were prepared as described⁶⁰, and sequenced on the Illumina NextSeq platform with a read length of 80 bp to a depth of 5,041,171±3,707,376 (mean±SD) reads for stool samples and 2,000,661±4,196,093 (mean±SD) for endoscopic samples.

RNA-Seq: Ribosomal RNA was selectively depleted by RnaseH (New England Biolabs, M0297) according to a modified version of a published method (pubmed ID:23685885). Specifically, a pool of 50 bp DNA oligos (25 nM, IDT, indicated in Table 4) that is complementary to murine rRNA18S and 28S, was resuspended in 75 μl of 10 mM Tris pH 8.0. Total RNA (100-1000 ng in 10 μl H₂O) were mixed with an equal amount of rRNA oligo pool, diluted to 2 μl and 3 μl 5×rRNA hybridization buffer (0.5 M Tris-HCl, 1 M NaCl, titrated with HCl to pH 7.4) was added. Samples were incubated at 95° C. for 2 minutes, then the temperature was slowly decreased (−0.1° C./s) to 37° C. RNAseH enzyme mix (2 μl of 10 U RNAseH, 2 μl 10×RNAseH buffer, 1 μl H₂O, total 5 μl mix) was prepared 5 minutes before the end of the hybridization and preheated to 37° C. The enzyme mix was added to the samples when they reached 37° C. and they were incubated at this temperature for 30 minutes. Samples were purified with 2.2×SPRI beads (Ampure XP, Beckmann Coulter) according to the manufacturers' instructions. Residual oligos were removed with DNAse treatment (ThermoFisher Scientific, AM2238) by incubation with 5 μl DNAse reaction mix (1 μl Trubo DNAse, 2.5 μl Turbo DNAse 10× buffer, 1.5 μl H₂O) that was incubated at 37° C. for 30 minutes. Samples were again purified with 2.2×SPRI beads and suspended in 3.6 μl priming mix (0.3 μl random primers of New England Biolab, E7420, 3.3 μl H₂O). Samples were subsequently primed at 65° C. for 5 minutes. Samples were then transferred to ice and 2 μl of the first strand mix was added (1 μl 5× first strand buffer, NEB E7420; 0.125 μl RNAse inhibitor, NEB E7420; 0.25 μl ProtoScript II reverse transcriptase, NEB E7420; and 0.625 μl of 0.2 μl/ml Actinomycin D, Sigma, A1410). The first strand synthesis and all subsequent library preparation steps were performed using NEBNext Ultra Directional RNA Library Prep Kit for Illumina (NEB, E7420) according to the manufacturers' instructions (all reaction volumes reduced to a quarter).

TABLE 4
DNA oligos used for rRNA depletion

| Oligo name | Sequence |
|---|---|
| AG9327_18_1 | TAATGATCCTTCCGCAGGTTCACCTACGGAAACCTTGTTA |
| | CGACTTTTAC (SEQ ID NO: 61) |
| AG9328_18_2 | TTCCTCTAGATAGTCAAGTTCGACCGTCTTCTCAGCGCTC |
| | CGCCAGGGCC (SEQ ID NO: 62) |
| AG9329_18_3 | GTGGGCCGACCCCGGCGGGGCCGATCCGAGGGCCTCACT |
| | AAACCATCCAA (SEQ ID NO: 63) |
| AG9330_18_4 | TCGGTAGTAGCGACGGGCGGTGTGTACAAAGGGCAGGG |
| | ACTTAATCAACG (SEQ ID NO: 64) |
| AG9331_18_5 | CAAGCTTATGACCCGCACTTACTCGGGAATTCCCTCGTTC |
| | ATGGGGAATA (SEQ ID NO: 65) |
| AG9332_18_6 | ATTGCAATCCCCGATCCCCATCACGAATGGGGTTCAACG |
| | GGTTACCCGCG (SEQ ID NO: 66) |
| AG9333_18_7 | CCTGCCGGCGTAGGGTAGGCACACGCTGAGCCAGTCAGT |
| | GTAGCGCGCGT (SEQ ID NO: 67) |
| AG9334_18_8 | GCAGCCCCGGACATCTAAGGGCATCACAGACCTGTTATT |
| | GCTCAATCTCG (SEQ ID NO: 68) |
| AG9335_18_9 | GGTGGCTGAACGCCACTTGTCCCTCTAAGAAGTTGGGGG |
| | ACGCCGACCGC (SEQ ID NO: 69) |
| AG9336_18_10 | TCGGGGGTCGCGTAACTAGTTAGCATGCCAGAGTCTCGT |
| | TCGTTATCGGA (SEQ ID NO: 70) |
| AG9337_18_11 | ATTAACCAGACAAATCGCTCCACCAACTAAGAACGGCCA |
| | TGCACCACCAC (SEQ ID NO: 71) |
| AG9338_18_12 | CCACGGAATCGAGAAAGAGCTATCAATCTGTCAATCCTG |
| | TCCGTGTCCGG (SEQ ID NO: 72) |
| AG9339_18_13 | GCCGGGTGAGGTTTCCCGTGTTGAGTCAAATTAAGCCGC |
| | AGGCTCCACTC (SEQ ID NO: 73) |
| AG9340_18_14 | CTGGTGGTGCCCTTCCGTCAATTCCTTTAAGTTTCAGCTT |
| | TGCAACCATA (SEQ ID NO: 74) |
| AG9341_18_15 | CTCCCCCCGGAACCCAAAGACTTTGGTTTCCCGGAAGCT |
| | GCCCGGCGGGT (SEQ ID NO: 75) |
| AG9342_18_16 | CATGGGAATAACGCCGCCGCATCGCCGGTCGGCATCGTT |
| | TATGGTCGGAA (SEQ ID NO: 76) |
| AG9343_18_17 | CTACGACGGTATCTGATCGTCTTCGAACCTCCGACTTTCG |
| | TTCTTGATTA (SEQ ID NO: 77) |
| AG9344_18_18 | ATGAAAACATTCTTGGCAAATGCTTTCGCTCTGGTCCGTC |
| | TTGCGCCGGT (SEQ ID NO: 78) |
| AG9345_18_19 | CCAAGAATTTCACCTCTAGCGGCGCAATACGAATGCCCC |
| | CGGCCGTCCCT (SEQ ID NO: 79) |
| AG9346_18_20 | CTTAATCATGGCCTCAGTTCCGAAAACCAACAAAATAGA |
| | ACCGCGGTCCT (SEQ ID NO: 80) |
| AG9347_18_21 | ATTCCATTATTCCTAGCTGCGGTATCCAGGCGGCTCGGGC |
| | CTGCTTTGAA (SEQ ID NO: 81) |
| AG9348_18_22 | CACTCTAATTTTTTCAAAGTAAACGCTTCGGGCCCCGCGG |
| | GACACTCAGC (SEQ ID NO: 82) |
| AG9349_18_23 | TAAGAGCATCGAGGGGGCGCCGAGAGGCAAGGGGCGGG |
| | GACGGGCGGTGG (SEQ ID NO: 83) |
| AG9350_18_24 | CTCGCCTCGCGGCGGACCGCCCGCCCGCTCCCAAGATCC |
| | AACTACGAGCT (SEQ ID NO: 84) |
| AG9351_18_25 | TTTTAACTGCAGCAACTTTAATATACGCTATTGGAGCTGG |
| | AATTACCGCG (SEQ ID NO: 85) |
| AG9352_18_26 | GCTGCTGGCACCAGACTTGCCCTCCAATGGATCCTCGTTA |
| | AAGGATTTAA (SEQ ID NO: 86) |
| AG9353_18_27 | AGTGGACTCATTCCAATTACAGGGCCTCGAAAGAGTCCT |
| | GTATTGTTATT (SEQ ID NO: 87) |
| AG9354_18_28 | TTTCGTCACTACCTCCCCGGGTCGGGAGTGGGTAATTTGC |
| | GCGCCTGCTG (SEQ ID NO: 88) |
| AG9355_18_29 | CCTTCCTTGGATGTGGTAGCCGTTTCTCAGGCTCCCTCTC |
| | CGGAATCGAA (SEQ ID NO: 89) |
| AG9356_18_30 | CCCTGATTCCCCGTCACCCGTGGTCACCATGGTAGGCAC |
| | GGCGACTACCA (SEQ ID NO: 90) |
| AG9357_18_31 | TCGAAAGTTGATAGGGCAGACGTTCGAATGGGTCGTCGC |
| | CGCCACGGG (SEQ ID NO: 91) |
| AG9358_18 32 | GCGTGCGATCGGCCCGAGGTTATCTAGAGTCACCAAAGC |
| | CGCCGGCGCCC (SEQ ID NO: 92) |
| AG9359_18_33 | GCCCCCCGGCCGGGGCCGGAGAGGGGCTGACCGGGTTG |
| | GTTTTGATCTGA (SEQ ID NO: 93) |
| AG9360_18_34 | TAAATGCACGCATCCCCCCCGCGAAGGGGGTCAGCGCCC |
| | GTCGGCATGTA (SEQ ID NO: 94) |
| AG9361_18_35 | TTAGCTCTAGAATTACCACAGTTATCCAAGTAGGAGAGG |
| | AGCGAGCGACC (SEQ ID NO: 95) |
| AG9362_18_36 | AAAGGAACCATAACTGATTTAATGAGCCATTCGCAGTTT |
| | CACTGTACCGG (SEQ ID NO: 96) |
| AG9363_18_37 | CCGTGCGTACTTAGACATGCATGGCTTAATCTTTGAGACA |
| | AGCATATGCT (SEQ ID NO: 97) |
| AG9364_18_38 | TGGCTTAATCTTTGAGACAAGCATATGCTACTGGCAGGA |
| | TCAACCAGGTA (SEQ ID NO: 98) |
| AG9466_5.8_1 | AAGCGACGCTCAGACAGGCGTAGCCCCGGGAGGAACCC |
| | GGGGCCGCAAGT (SEQ ID NO: 99) |
| AG9467_5.8_2 | GCGTTCGAAGTGTCGATGATCAATGTGTCCTGCAATTCAC |
| | ATTAATTCTC (SEQ ID NO: 100) |
| AG9468_5.8_3 | GCAGCTAGCTGCGTTCTTCATCGACGCACGAGCCGAGTG |
| | ATCCACCGCTA (SEQ ID NO: 101) |
| AG9469_16_1 | AAACCCTGTTCTTGGGTGGGTGTGGGTATAATACTAAGTT |
| | GAGATGATAT (SEQ ID NO: 102) |
| AG9470_16_2 | CATTTACGGGGGAAGGCGCTTTGTGAAGTAGGCCTTATT |
| | TCTCTTGTCCT (SEQ ID NO: 103) |
| AG9471_16_3 | TTCGTACAGGGAGGAATTTGAANGTAGATAGAAACCGAC |
| | CTGGATTACTC (SEQ ID NO: 104) |
| AG9472_16_4 | CGGTCTGAACTCAGATCACGTAGGACTTTAATCGTTGAA |
| | CAAACGAACCT (SEQ ID NO: 105) |
| AG9473_16_5 | TTAATAGCGGCTGCACCATCGGGATGTCCTGATCCAACA |
| | TCGAGGTCGTA (SEQ ID NO: 106) |
| AG9474_16_6 | AACCCTATTGTTGATATGGACTCTAGAATAGGATTGCGCT |
| | GTTATCCCTA (SEQ ID NO: 107) |
| AG9475_16_7 | GGGTAACTTGTTCCGTTGGTCAAGTTATTGGATCAATTGA |
| | GTATAGTAGT (SEQ ID NO: 108) |
| AG9476_16_8 | TCGCTTTGACTGGTGAAGTCTTAGCATGTACTGCTCGGAG |
| | GTTGGGTTCT (SEQ ID NO: 109) |
| AG9477_16_9 | GCTCCGAGGTCGCCCCAACCGAAATTTTTAATGCAGGTTT |
| | GGTAGTTTAG (SEQ ID NO: 110) |
| AG9478_16_10 | GACCTGTGGGTTTGTTAGGTACTGTTTGCATTAATAAATT |
| | AAAGCTCCAT (SEQ ID NO: 111) |
| AG9479_16_11 | AGGGTCTTCTCGTCTTGCTGTGTTATGCCCGCCTCTTCAC |
| | GGGCAGGTCA (SEQ ID NO: 112) |
| AG9480_16_12 | ATTTCACTGGTTAAAAGTAAGAGACAGCTGAACCCTCGT |
| | GGAGCCATTCA (SEQ ID NO: 113) |
| AG9481_16_13 | TACAGGTCCCTATTTAAGGAACAAGTGATTATGCTACCTT |
| | TGCACGGTTA (SEQ ID NO: 114) |
| AG9482_16_14 | GGGTACCGCGGCCGTTAAACATGTGTCACTGGGCAGGCG |
| | GTGCCTCTAAT (SEQ ID NO: 115) |
| AG9483_16_15 | ACTGGTGATGCTAGAGGTGATGTTTTTGGTAAACAGGCG |
| | GGGTAAGATTT (SEQ ID NO: 116) |
| AG9484_16_16 | GCCGAGTTCCTTTTACTTTTTTTAACCTTTCCTTATGAGCA |
| | TGCCTGTGT (SEQ ID NO: 117) |
| AG9485_16_17 | TGGGTTGACAGTGAGGGTAATAATGACTTGTTGGTTGATT |
| | GTAGATATTG (SEQ ID NO: 118) |
| AG9486_16_18 | GGCTGTTAATTGTCAGTTCAGTGTTTTAATCTGACGCAGG |
| | CTTATGCGGA (SEQ ID NO: 119) |
| AG9487_16_19 | GGAGAATGTTTTCATGTTACTTATACTAACATTAGTTCTT |
| | CTATAGGGTG (SEQ ID NO: 120) |
| AG9488_16_20 | ATAGATTGGTCCAATTGGGTGTGAGGAGTTCAGTTATAT |
| | GTTTGGGATTT (SEQ ID NO: 121) |
| AG9489_16_21 | TTTAGGTAGTGGGTGTTGAGCTTGAACGCTTTCTTAATTG |
| | GTGGCTGCTT (SEQ ID NO: 122) |
| AG9490_16_22 | TTAGGCCTACTATGGGTGTTAAATTTTTTACTCTCTCTAC |
| | AAGGTTTTTT (SEQ ID NO: 123) |
| AG9491_16_23 | CCTAGTGTCCAAAGAGCTGTTCCTCTTTGGACTAACAGTT |
| | AAATTTACAA (SEQ ID NO: 124) |
| AG9492_16_24 | GGGATTTAGAGGGTTCTGTGGGCAAATTTAAAGTTGAAC |
| | TAAGATTCTA (SEQ ID NO: 125) |
| AG9493_16_25 | TCTTGGACAACCAGCTATCACCAGGCTCGGTAGGTTTGTC |
| | GCCTCTACCT (SEQ ID NO: 126) |
| AG9494_16_26 | ATAAATCTTCCCACTATTTTGCTACATAGACGGGTGTGCT |
| | CTTTTAGCTG (SEQ ID NO: 127) |
| AG9495_ 16_27 | TTCTTAGGTAGCTCGTCTGGTTTCGGGGGTCTTAGCTTTG |
| | GCTCTCCTTG (SEQ ID NO: 128) |
| AG9496_16_28 | CAAAGTTATTTCTAGTTAATTCATTATGCAGAAGGTATAG |
| | GGGTTAGTCC (SEQ ID NO: 129) |
| AG9497_16_29 | TTGCTATATTATGCTTGGTTATAATTTTTCATCTTTCCCTT |
| | GCGGTACTA (SEQ ID NO: 130) |
| AG9498_16_30 | TATCTATTGCGCCAGGTTTCAATTTCTATCGCCTATACTTT |
| | ATTTGGGTA (SEQ ID NO: 1301) |
| AG9499_16_31 | AATGGTTTGGCTAAGGTTGTCTGGTAGTAAGGTGGAGTG |
| | GGTTTGGGGCT (SEQ ID NO: 132) |
| AG9500_12_1 | GTTCGTCCAAGTGCACTTTCCAGTACACTTACCATGTTAC |
| | GACTTGTCTC (SEQ ID NO: 133) |
| AG9501_12_2 | CTCTATATAAATGCGTAGGGGTTTTAGTTAAATGTCCTTT |
| | GAAGTATACT (SEQ ID NO: 134) |
| AG9502_12_3 | TGAGGAGGGTGACGGGCGGTGTGTACGCGCTTCAGGGCC |
| | CTGTTCAACTA (SEQ ID NO: 135) |
| AG9503_12_4 | AGCACTCTACTCTTAGTTTACTGCTAAATCCACCTTCGAC |
| | CCTTAAGTTT (SEQ ID NO: 136) |
| AG9504_12_5 | CATAAGGGCTATCGTAGTTTTCTGGGGTAGAAAATGTAG |
| | CCCATTTCTTG (SEQ ID NO: 137) |
| AG9505_12_6 | CCACCTCATGGGCTACACCTTGACCTAACGTCTTTACGTG |
| | GGTACTTGCG (SEQ ID NO: 138) |
| AG9506_12_7 | CTTACTTTGTAGCCTTCATCAGGGTTTGCTGAAGATGGCG |
| | GTATATAGGC (SEQ ID NO: 139) |
| AG9507_12_8 | TGAGCAAGAGGTGGTGAGGTTGATCGGGGTTTATCGATT |
| | ACAGAACAGGC (SEQ ID NO: 140) |
| AG9508_12_9 | TCCTCTAGAGGGATATGAAGCACCGCCAGGTCCTTTGAG |
| | TTTTAAGCTGT (SEQ ID NO: 141) |
| AG9509_12_10 | GGCTCGTAGTGTTCTGGCGAGCAGTTTTGTTGATTTAACT |
| | GTTGAGGTTT (SEQ ID NO: 142) |
| AG9510_12_11 | AGGGCTAAGCATAGTGGGGTATCTAATCCCAGTTTGGGT |
| | CTTAGCTATTG (SEQ ID NO: 143) |
| AG9511_12_12 | TGTGTTCAGATATGTTAAAGCCACTTTCGTAGTCTATTTT |
| | GTGTCAACTG (SEQ ID NO: 144) |
| AG9512_12_13 | GAGTTTTTTACAACTCAGGTGAGTTTTAGCTTTATTGGGG |
| | AGGGGGTGAT (SEQ ID NO: 145) |
| AG9513_12_14 | CTAAAACACTCTTTACGCCGGCTTCTATTGACTTGGGTTA |
| | ATCGTGTGAC (SEQ ID NO: 146) |
| AG9514_12_15 | CGCGGTGGCTGGCACGAAATTGACCAACCCTGGGGTTAG |
| | TATAGCTTAGT (SEQ ID NO: 147) |
| AG9515_12_16 | TAAACTTTCGTTTATTGCTAAAGGTTAATCACTGCTGTTT |
| | CCCGTGGG (SEQ ID NO: 148) |
| AG9516_12_17 | TGTGGCTAGGCTAAGCGTTTTGAGCTGCATTGCTGCGTGC |
| | TTGATGCTTG (SEQ ID NO: 149) |
| AG9517_12_18 | TTCCTTTTGATCGTGGTGATTTAGAGGGTGAACTCACTGG |
| | AACGGGGATG (SEQ ID NO: 150) |
| AG9518_12_ 19 | CTTGCATGTGTAATCTTACTAAGAGCTAATAGAAAGGCT |
| | AGGACCAAACC (SEQ ID NO: 151) |
| AG9519_5_1 | AAAGCCTACAGCACCCGGTATTCCCAGGCGGTCTCCCAT |
| | CCAAGTACTAA (SEQ ID NO: 152) |
| AG9520_5_2 | CCAGGCCCGACCCTGCTTAGCTTCCGAGATCAGACGAGA |
| | TCGGGCGCGTT (SEQ ID NO: 153) |
| AG9521_5_3 | TTCCGAGATCAGACGAGATCGGGCGCGTTCAGGGTGGTA |
| | TGGCCGTAGAC (SEQ ID NO: 154) |

Analyses

16S rDNA analysis: The 2×250 bp reads were processed using the QIIME (Quantitative Insights Into Microbial Ecology, www(dot)qiime(dot)org) analysis pipeline⁹⁴. In brief, fasta quality files and a mapping file indicating the barcode sequence corresponding to each sample were used as inputs, paired reads were first assembled into longer reads based on sequence similarity, the assembled reads were then split to samples according to the barcodes, Sequences sharing 97% nucleotide sequence identity in the 16S rRNA region were binned into operational taxonomic units (97% ID OTUs). Each OTU was assigned a taxonomical classification by applying the Uclust algorithm against the Greengenes database, and an OTU table was created.

Metagenomic analysis: Data from the sequencer was converted to fastq files with bcl2fastq. Reads were then QC trimmed using Trimmomatic⁹⁵ with parameters PE -threads 10 -phred33 -validatePairs ILLUMINACLIP:TruSeq3-PE.fa:2:30:10 LEADING:3 TRAILING:3 MINLEN:50. We used MetaPhlAn2⁹⁶ for taxonomic analysis with parameters: —ignore_viruses —ignore_archaea —ignore_eukaryotes.

Host sequences were removed by aligning the reads against human genome reference hg19 using bowtie2⁹⁷ with parameters: -D 5 -R 1 -N 0 -L 22 -i 5,0,2.50. The resulting non-host reads were then mapped to the integrated gene catalogue¹⁰⁰ using bowtie2 with parameters: —local -D 25 -R 3 -N 1 -L 19 -i 5,1,0.25 -k 5 allowing to a single read to match up to five different entries.

Further filtering of the bacterial reads consisted of retaining only records with minimal base quality of 26. The bacterial quality filtered resulting bam files were then subsampled to 100,000 bacterial hits. An entry's score was defined by its length, divided by the gene length. Entries scores were summarized according to KO annotations¹⁰¹. Each sample was scaled to 1M. KEGG Pathway analysis was conducted using EMPANADA⁹⁸.

Probiotics strain identification by unique genomic sequences: Recovery of genomes for probiotic strains from pill metagenomics samples: Genomes for 9 of the 11 probiotic strains were recovered at >93% completeness and <4% contamination from metagenomics samples of the probiotics pill (Table 5). For one of the species (B. longum) only part of the genome was recovered due to strain heterogeneity. The samples were assembled in multiple cycles using IDBA-UD¹⁰². Assemblies were manually improved using a mini-assembly approach⁸². Genomes were recovered based on similarity to reference genomes and connectivity between scaffolds as deduced from the mini-assembly analysis.

TABLE 5
statics for genomes recovered from metagenomics samples
of probiotics pill used in the study. Completeness and contamination
were evaluated using CheckM¹⁰³.

| | | #Scaf- | Complete- | Contam- |
|---|---|---|---|---|
| Species | Size | folds | ness | ination |
| Bifidobacterium breve | 2,051,417 | 128 | 93.66 | 0.69 |
| Bifidobacterium bifidum | 2,196,275 | 11 | 99.54 | 0.12 |
| Bifidobacterium longum | 1,200,324 | 180 | 46.03 | 0.96 |
| Lactococcus lactis | 2,472,057 | 36 | 100 | 0 |
| Lactobacillus acidophilus | 1,963,581 | 22 | 98.94 | 0 |
| Lactobacillus casei | 2,968,946 | 33 | 94.64 | 1.72 |
| Lactobacillus paracasei | 3,038,895 | 92 | 98.79 | 3.56 |
| Lactobacillus plantarum | 3,299,766 | 31 | 99.38 | 2.79 |
| Lactobacillus rhamnosus | 2,921,071 | 29 | 99.02 | 0 |
| Streptococcus thermophilus | 1,789,952 | 74 | 99.89 | 0.29 |

Strain-Level Analysis Probiotic Strains in Human Samples.

Identifying reads that belong to the probiotic strains in each sample: All human reads were first removed from all samples by mapping against the human genome (assembly GRCh38.p7) using bowtie2⁹⁷ with the -very_sensitive flag. Next, the non-human reads were mapped against all probiotics genomes recovered from the pill using bowtie2 to identify reads that potentially belong to these strains. Finally, the reads were mapped against a database of genomes for all species in the orders Lactobacillales and Bifidobacteriales to which the probiotic strains belong, including the probiotic genomes. Only reads that received their best hit from one of the probiotics strains were further analyzed.

Determining presence of probiotic species: we counted the number of genes in each probiotic genome whose coverage is greater than 0. A probiotic species was determined to be present in a sample if at least 400 of its genes were detected, with the threshold being set based on comparison to MetaPhlAn2 results and an analysis of gene number distribution across the different samples.

Determining strain-specific genes: we clustered each probiotic genome's proteins with other genomes available for the its species using USEARCH¹⁰⁴ with 90% identity threshold. All genes in clusters whose size was <10% of the number of genomes analyzed were determined to be strain specific. The analysis could be applied to the genomes of B. bifidum, B. breve, B. longum, L. acidophilus, L. casei, L. lactis, L. paracasei, L. plantarum and S. thermophilus. For B. longum, it is not possible to determine which of the probiotic strains is present.

Determining samples with probiotic strains: For each strain that passed the 400-genes threshold from step 3 we compared the fraction of strain-specific genes detected with the fraction of all genes on the genome that were detected. The probiotic strain was determined to be present if at least 65% of the total number of genes were detected and the difference between the fraction of the total and strain-specific genes that were found was 20% or less.

RNAseq Analysis

Data normalization: Initially, we normalized the sequenced data as previously described¹⁰⁵. Briefly, genes with mean TPM<1 across all samples were filtered out from the analysis, and a value of 0.001 was added to remaining TPM values to avoid zero-values in downstream calculations. Then, sample median normalization was performed based on all constitutive gene reads with positive counts for all samples. Thus, all TPM values in each sample were scaled by the median TPM of constitutive reads in that sample, divided by the median TPM across all samples. We then performed a per-gene normalization by dividing each expression value by the median value of that gene across all samples. Finally, expression data was log-transformed (base 2). The above normalization steps were performed separately to data acquired from each of the different experimental batches, determined by the presence or absence of RNAlater solution for sample preservation.

Comparison of expression levels before and after treatment with probiotics: To account for inter-personal differences and reduce noise, we compared the effects of probiotics treatment on host expression patterns using a repeated-measures design. Thus, for each individual, in each biopsy region, the relative fold-changes (log, base 2) in expression levels of each gene were calculated between samples taken at baseline and after treatment with probiotics. Then, for each individual, genes were ranked from low to high, and sorted by their median rank across all available samples. These sorted lists were subsequently used for gene ontology (GO) enrichment analysis using GOrilla⁹⁹ with a p-value threshold of 10⁻³ and a false-discovery rate (FDR) threshold of q<0.05.

Comparison of expression levels between probiotics persistent and resistant individuals: For each gene, median relative expression was calculated in probiotics-persistent and resistant individuals within each biopsy region and experimental batch. Then, genes were sorted by the ratio (log, base 2) between median relative expression levels across probiotics-persistent compared to resistant individuals. Finally, to combine findings from both experimental batches, we intersected the top and the bottom 10% of the genes across the two batches. Intersected lists were used as target sets for GOrilla GO enrichment analysis as described above, with the entire set of genes that passed the initial filtering as a background set.

Quantification and statistical analysis: The following statistical analyses were applied unless specifically stated otherwise: For 16S data, rare OTUs (<0.1% in relative abundance) were filtered out, and samples were then rarefied to a depth of 10,000 reads (5000 in mouse tissues). For metagenomic data, samples with <10⁵ assigned bacterial reads (after host removal) were excluded from further analysis. In the remaining samples, rare KEGG orthologous (KO) genes (<10-5) were removed. Beta diversity was calculated on OTUs (16S) or species (metagenomics) relative abundances using UniFrac distances or Bray-Curtis dissimilarity (R Vegan package, www(dot)CRAN.R-project(dot)org/package=vegan) respectively. PCA for KOs and functional bacterial pathways were calculated using Spearman's rank correlation coefficient. Alpha diversity was calculated on OTUs (16S) using the observed species index. For 16S data, measurements of alpha and beta diversity were calculated using QIIME tools v 1.9.1. In order to determine the effect of treatment on microbiota taxonomic composition and functional capacity repeated-measures Kruskal Wallis with Dunn's test was used. In order to compare the effect of treatment over time between two groups or more two-way ANOVA with Dunnet's test, or permutation tests performed by switching labels between participants, including all their assigned samples, were used. Mann-Whitney and Wilcoxon tests were used to conduct pairwise comparisons between two treatment arms or two groups of participants. Permutational multivariate ANOVA (Adonis PERMANOVA with 10,000 permutations) based on sample distances was used to test for changes in the community composition and function. To analyze qPCR data, two way ANOVA with Sidak or Dunnett test was used. The threshold of significance was determined to be 0.05 both for p and q-values. Statistically significant findings were marked according to the following cutoffs: *, P<0.05; **, P<0.01; ***, P<0.001; ****, P<0.0001. Data were plotted with GraphPad Prism version 7.0c. Statistical details for all experiments, including sample size, the statistical test used dispersion and precision measures and statistical significance, are specified in the result section and denoted in figure legends.

### Results

### Murine Stool Microbiome Configuration Only Partially Correlates with the Gut Mucosa Microbiome

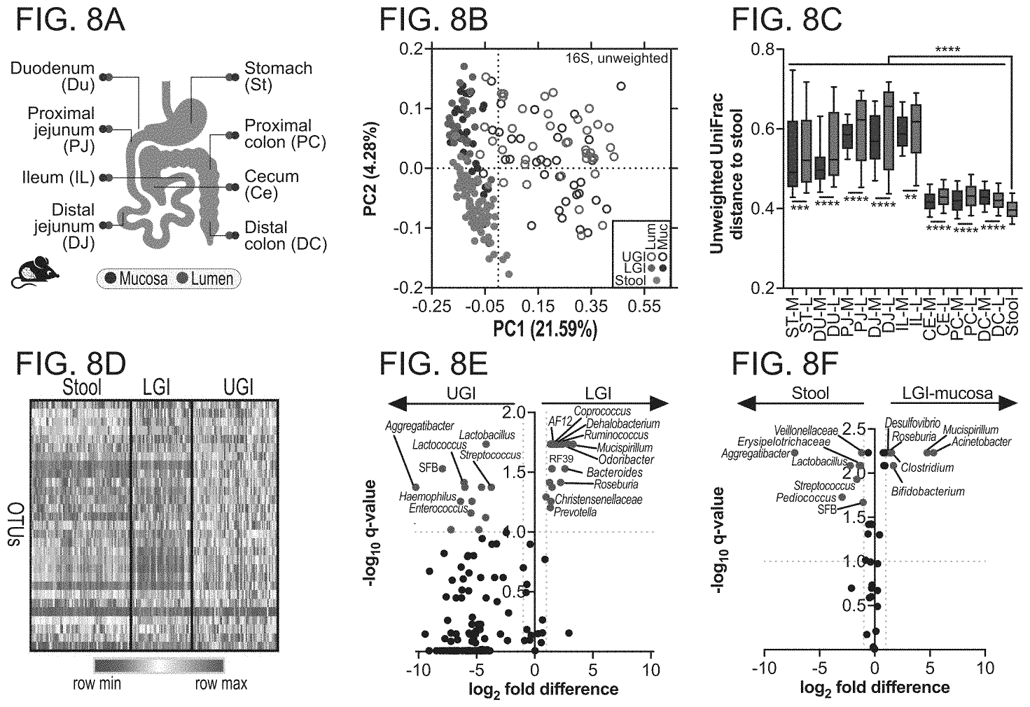

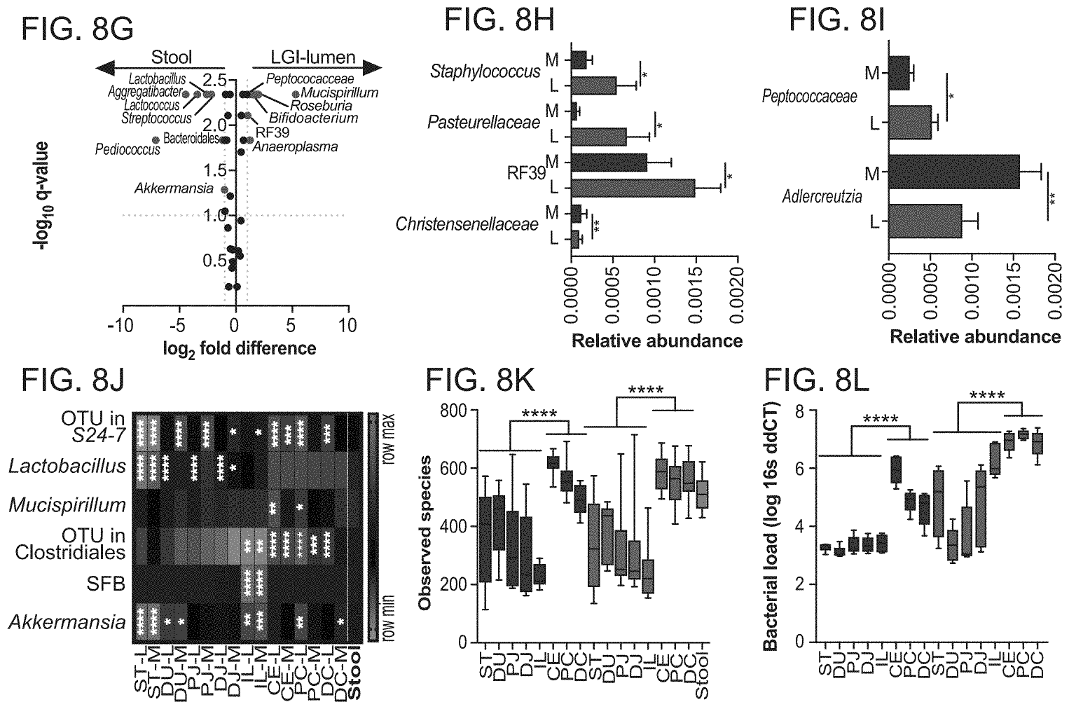

Most evidence supporting beneficial effects of probiotic microorganisms stem from animal and human studies extrapolating from stool microbiome analysis to potential probiotics impacts on host physiology^{32,35,39,49-52,77,78}. To assess whether stool microbiome represents an accurate marker of upper and lower GI mucosal microbiome architecture, we began our investigation by performing a comparative analysis of lumen and mucosa-associated microbiome samples collected from multiple regions of the upper gastrointestinal (UGI) and lower gastrointestinal (LGI) tract of 10 naïve 10-week-old male wild type (WT) C57Bl/6 mice (FIG. 8A, see methods for collection technique). After rarefying to 10000 reads, 39 UGI lumen, 35 UGI mucosa, 28 LGI lumen, 27 LGI mucosa and 87 fecal samples were collected from the same mice.

Unweighted UniFrac distances based on 16S rDNA sequencing separated both luminal and mucosal GI samples from stool samples collected from the same mice during the 4 weeks prior to dissection (One-way ANOVA and Tukey post-hoc P<0.0001, FIGS. 8B-D). Samples from the LGI were more similar to stool than UGI (FIGS. 8B-D), with the distance to stool being significant for both UGI and LGI (FIG. 8D, One-way ANOVA and Tukey post-hoc P<0.0001). 100/324 taxa were significantly variable between stool, UGI and LGI (FIG. 8E, One-Way ANOVA P<0.05). Among the taxa significantly enriched in the UGI over the LGI of naive mice (FIG. 8E, FDR-corrected Mann-Whitney P<0.05) were all common probiotics genera, namely Lactobacillus, Bifidobacterium, Lactococcus and Streptococcus, as well as Haemophilus and Enterobacteriaceae, whereas the LGI was enriched with Prevotella, Bacteroides, Ruminococcus and Mucispirillum. 17 OTUs were significantly variably represented between the mucosa of the LGI and stool samples (FIG. 8F), and even the LGI lumen was distinct from stool, with 19 significantly variably represented OTUs (FIG. 8G). Significant differences in the relative abundances of several taxa were also noted between the lumen and the mucosa of the UGI (FIG. 8H) and the LGI (FIG. 8I). We then identified several taxa that are significantly over- or under-represented throughout the GI tract compared to stool (FIG. 8J). Both the mucosal and luminal samples of the LGI were richer in the number of species (FIG. 8K) and total bacterial load as determined by qPCR of the 16S gene (FIG. 8L) compared to the UGI (Mann-Whitney P<0.0001). To conclude, the murine gastrointestinal tract displays a gradient of bacterial richness and a shifting compositional landscape, in which even the most distal lumen samples are significantly different than stool samples, limiting the applicability of stool in assessing mucosal GI colonization.

### Human Fecal Microbiome is a Limited Indicator of Gut Mucosal-Associated Microbiome Composition and Metagenomic Function

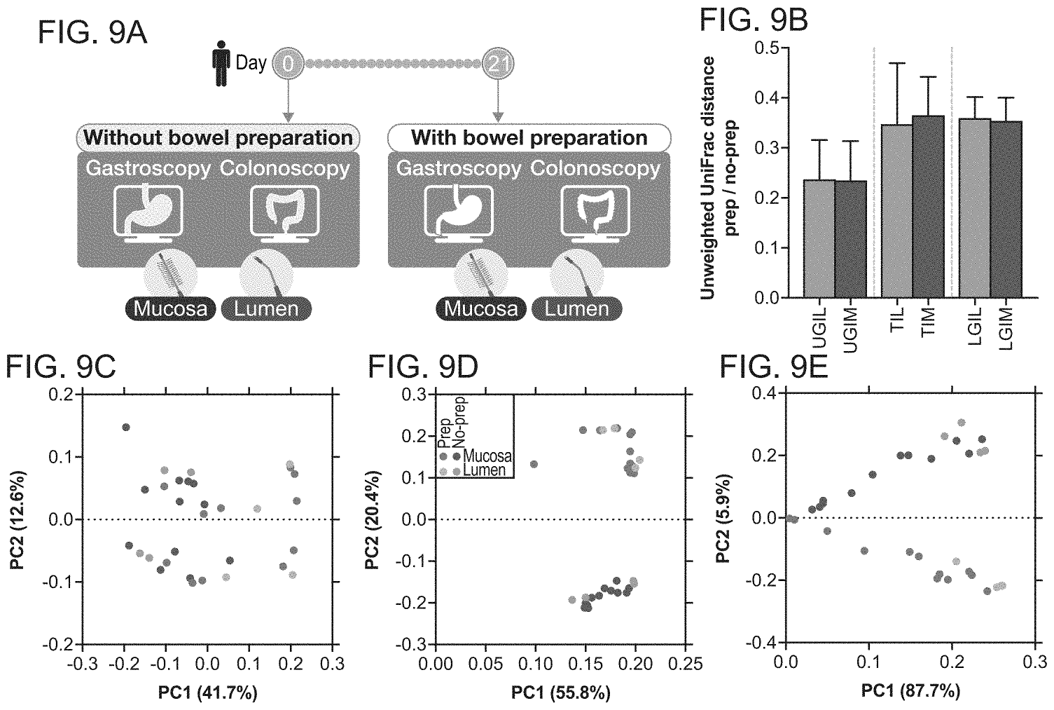

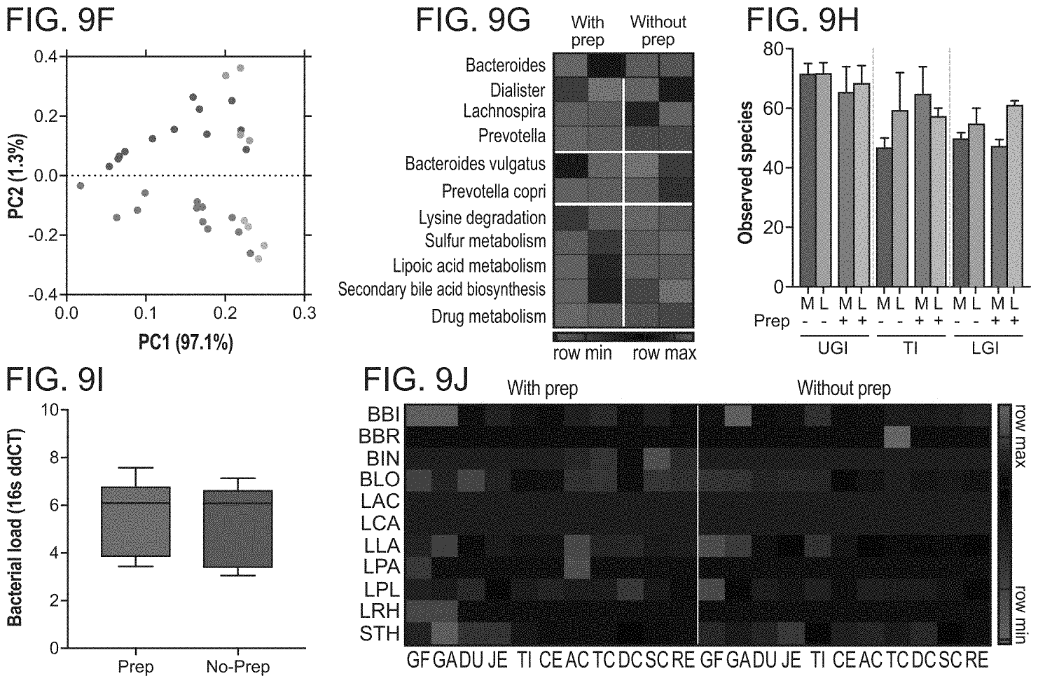

Similar to mice, studies on the human GI microbiome rely almost exclusively on stool sampling, despite insufficient evidence that these samples accurately reflect the microbial gut mucosal composition or function. We therefore sought to investigate the potential of stool samples as markers for the mucosal GI microbial community by directly sampling throughout the GI tract. To account for mucosal microbiome-altering impacts of bowel preparation^{79,80}, we sampled along the UGI and LGI tract 2 healthy female adult participants (aged 25 and 27, BMI 20.3 and 22.8) undergoing two consecutive colonoscopies, the first performed in the absence of any form of bowel preparation, followed by a second procedure three weeks later performed using a routine Picolax bowel preparation protocol (FIG. 9A, methods). The TI and LGI were more affected by bowel preparation than the UGI (FIG. 9B), resulting in separation of the prepped and the non-prepped samples according to 16S (Unweighted UniFrac, FIG. 9C), MetaPhLan2 (FIG. 9D), KOs (FIG. 9E), and pathways (FIGS. 9F-G), but no significant differences in observed species (FIG. 9H) or bacterial load (FIG. 9I). These limitations notwithstanding, bowel preparation, greatly facilitating direct gut mucosal sampling at the entirety of the human GI tract, was uniformly applied to all intervention and control cases thereafter.

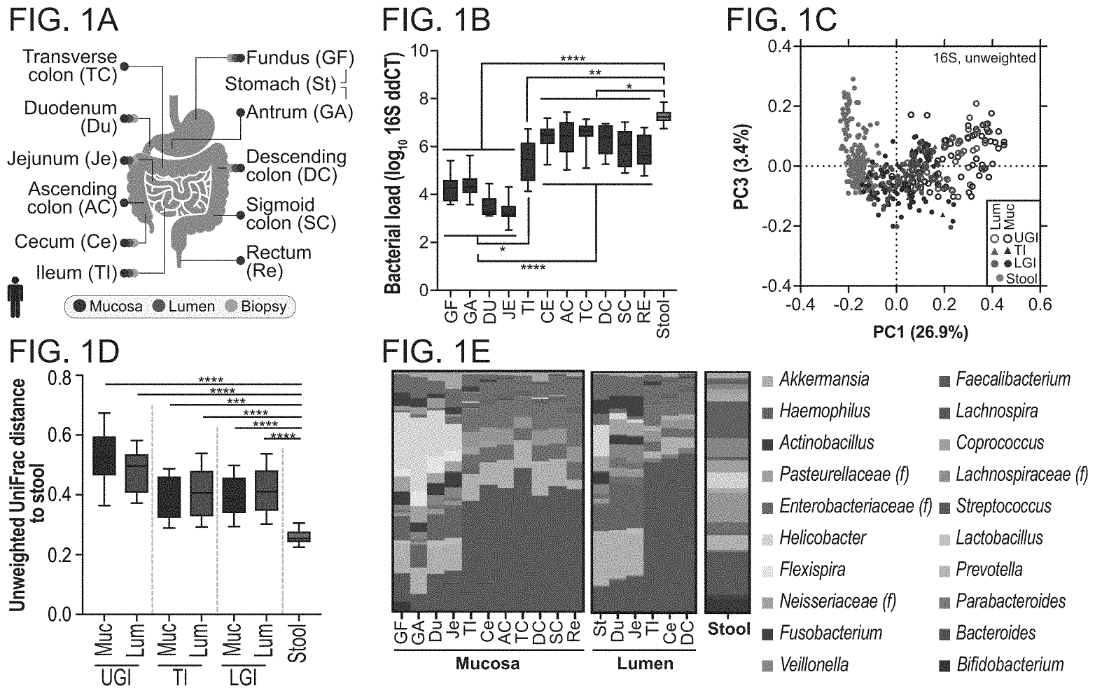

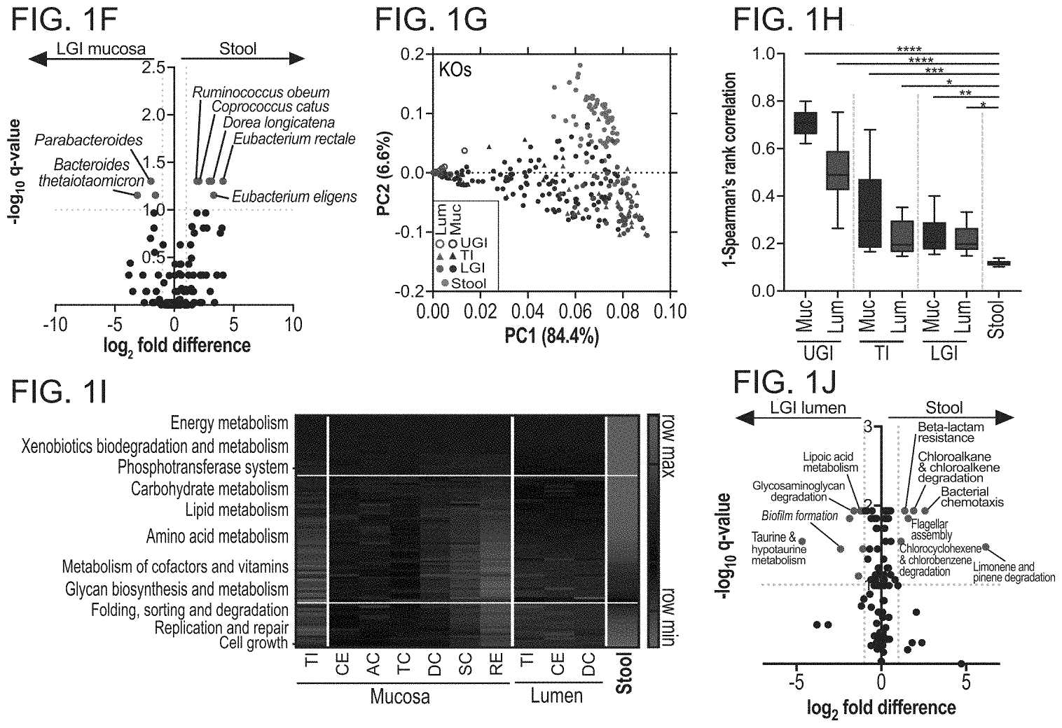

We began by characterizing the gut microbiome of a cohort in healthy human adults at different bio-geographical regions and directly compared these to stool microbiome configuration of the same individuals. To this aim, 25 healthy participants aged 20-66 (mean age 41.32±14.28, 13 females, mean BMI 23.1±3.5) underwent a multi-omic microbiome characterization at multiple gut mucosal and luminal regions spanning the LGI and UGI (FIG. 1A) via sampling through deep enteroscopy and colonoscopy coupled with stool collection. Luminal endoscopic samples were collected via suctioning of lumen fluid, mucosal microbiome samples were collected using cytology brushes, and host GI biopsies were collected using endoscopic forceps. All the interventional procedures were performed using an identical protocol (methods) by one of three highly experienced board-certified gastroenterologists in a single tertiary medical center. Collectively, 598 homeostatic samples, of which 61 fecal samples, 262 mucosal microbiome samples, 134 luminal microbiome samples, and 141 regional GI biopsies were collected, processed and analyzed using both 16S rDNA and shotgun metagenomic sequencing. Host GI biopsies were processed and analyzed for their transcriptional profile using RNA sequencing.

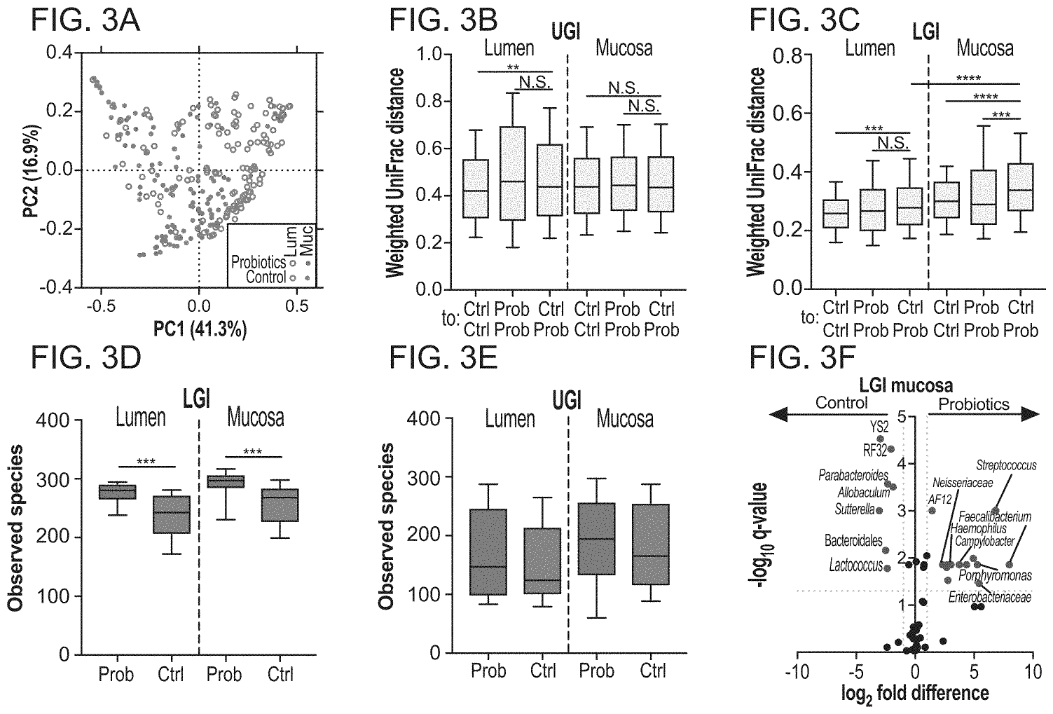

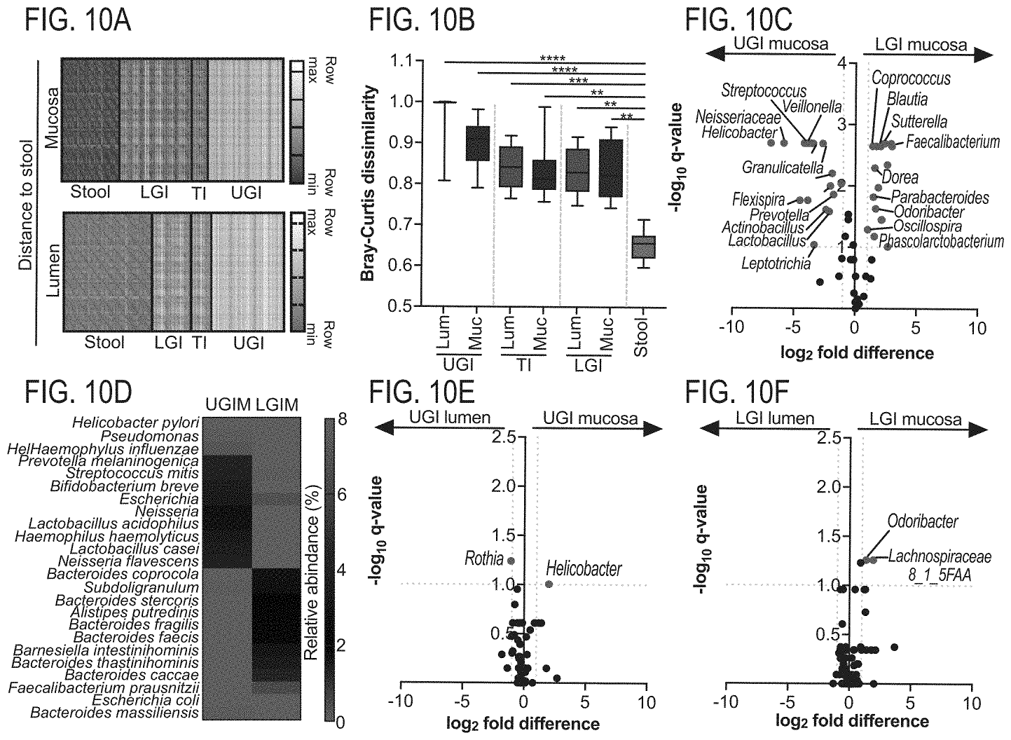

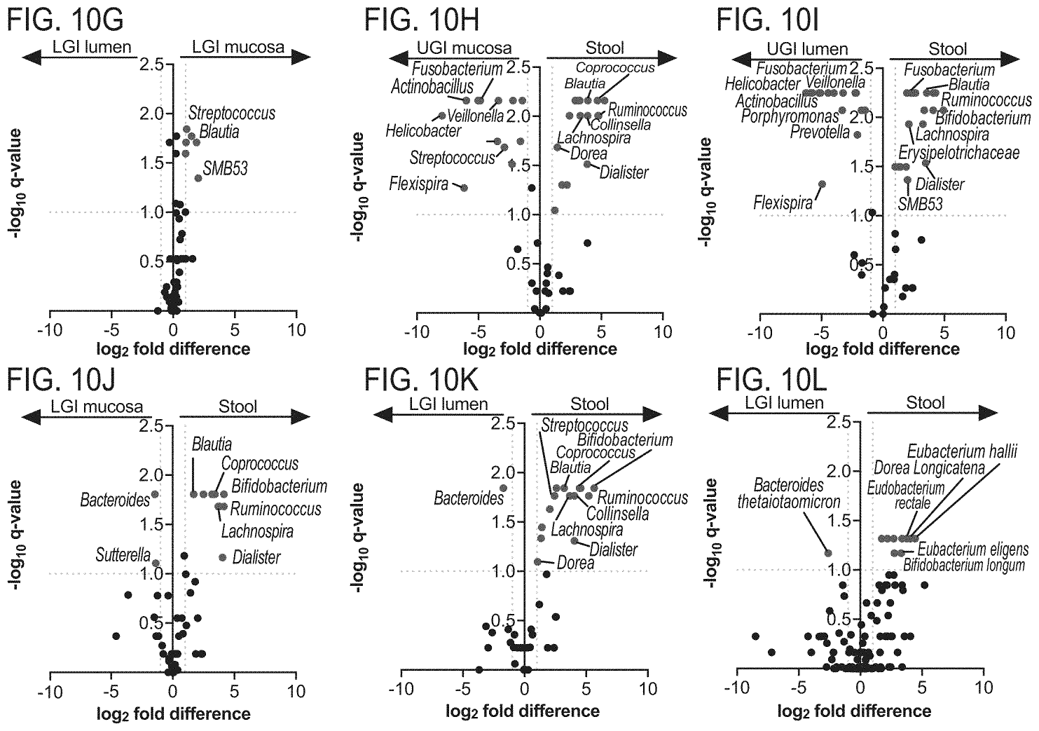

Expectedly, microbiome load varied throughout the GI tract. qPCR-based amplification of the 16S gene demonstrated stool samples to harbor the highest bacterial load compared to more proximal GI regions, with a gradient starting from the sparsely populated UGI regions, which were significantly less colonized than their most distal region (TI) and the LGI (FIG. 1B, Kruskal-Wallis & Dunn's). To assess the similarity between stool and GI samples, we rarefied all 16s rDNA samples to 10000 reads and calculated unweighted UniFrac distances (FIGS. 1C-D), which demonstrated a significant compositional gradient in which LGI samples were distinct from stool, but more similar to stool than UGI samples. The terminal ileum (TI) was more similar to stool than more proximal regions of the UGI. A compositional dissimilarity gradient was also observed in shotgun metagenomic sequencing, using MetaPhlAn2 species-based Bray-Curtis dissimilarity indices (FIGS. 10A-B). This was reflected by the differences in proportions between the most common genera in each region (FIG. 1E). More than 35 taxa were significantly variable between the UGI and LGI (FDR-corrected Mann-Whitney P<0.05, FIGS. 3C-D), including Helicobacter pylori, Prevotella melaninogenica, Hemophilus, Fusobacterium, Neisseria, Porphyromonas, Lactobacillus, Bifidobacterium and Streptococcus that were higher in the UGI, and Bacteroides thetaiotaomicron, B. vulgatus, B. uniformis, Parabacteroides distasonis, Faecalibacterium prausnitzii, Lachnospiraceae and Ruminococcaceae which were more abundant in the LGI. Several differences between the lumen and the mucosa were observed in both the UGI (FIG. 10E) and LGI (FIGS. 10F-G). Multiple OTUs were significantly over or under-represented in stool compared to the UGI mucosa (31 genera, FIG. 10H), UGI lumen (34 genera, FIG. 10I), LGI mucosa (11 genera, FIG. 10J; and 10 species, FIG. 1F), and LGI lumen (15 genera, FIG. 10K; and 10 species, FIG. 10L).

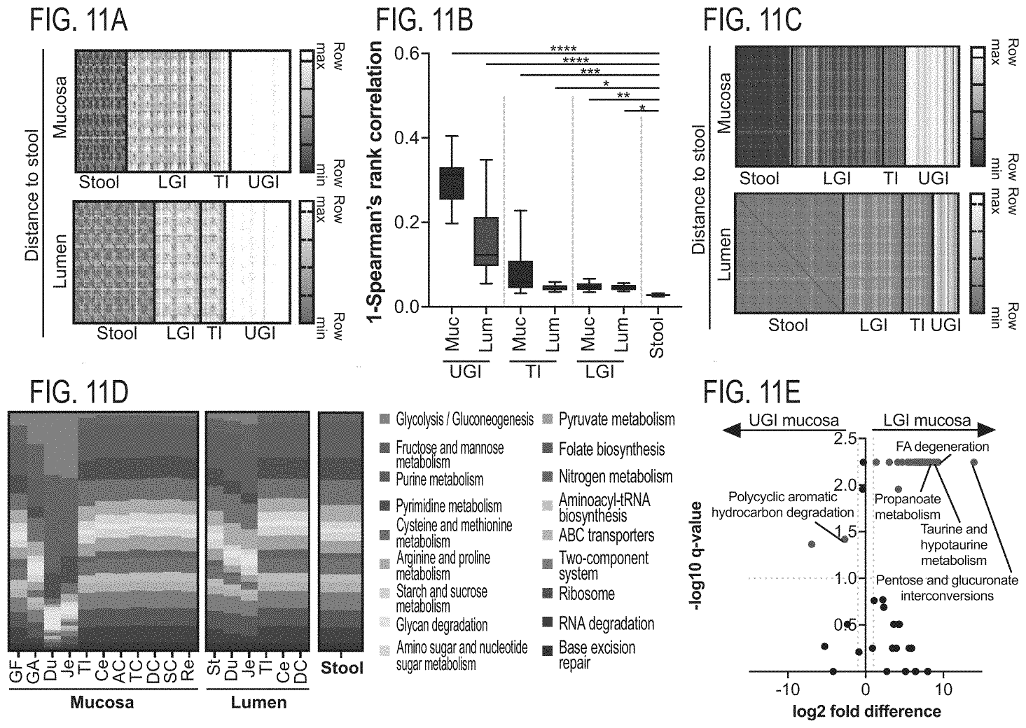

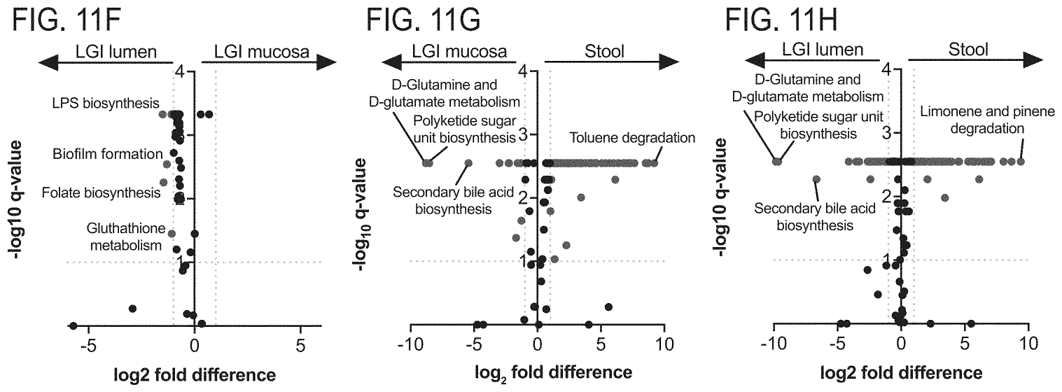

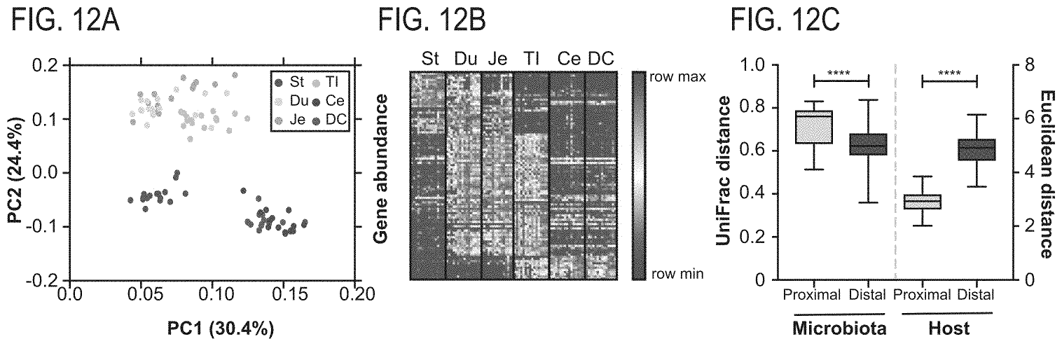

Given the redundancy in microbial genes and pathways encoded by different microbiome members⁸¹, and at different bio-geographical locations along the GI tract⁷⁵, we next set out to determine whether the different regions of the human GI tract display variation in microbial-encoded functions, and whether such variation is reflected in stool. Mapping whole DNA shotgun metagenomic sequencing reads to KEGG orthologous genes (KOs) revealed that, like microbial composition, microbial functions display a dissimilarity gradient throughout the GI tract, starting from stool, to LGI, TI and UGI samples, with all regions significantly different from stool (Kruskal-Wallis P<0.0001, FIGS. 1G-H, FIG. 11A). Mapping KOs to pathways resulted in a similar gradient and significant separation (P<0.0001, FIGS. 11B-C), and thus we compared the abundance of the most common pathways in each region to all other regions (FIG. 11D). Indeed, the UGI was clearly separated from the LGI, with 73 pathways significantly differentially represented (FIG. 11E). We then specifically assessed the degree of functional agreement between stool samples and the LGI, resulting in 100 pathways that were significantly differentially represented between stool samples and either the lumen or the mucosa of the LGI (FIG. 11, FIGS. 11F-H). Some pathways that were highly abundant in stool samples were very low in either luminal or mucosal samples, while others, mostly relating to macronutrients metabolism, were high both in stool samples and in luminal samples but low in mucosal samples. Interestingly, a group of pathways, mostly relating to genetic information processing, was high in luminal samples, intermediate in mucosal samples, and very low in stool samples (FIG. 11). Thus, even the LGI lumen was functionally distinct than stool (FIG. 1J). Likewise, host transcriptome obtained from 6 anatomical locations along the human GI tract (stomach, duodenum, jejunum, terminal ileum, cecum and descending colon (FIG. 1A), featured a region-specific clustering (FIGS. 12A-B). Interestingly, in contrast to the TI microbial configuration, which mainly resembled that of the LGI, its host transcriptional profile featured a ‘watershed’ profile clustering in between the small intestinal and colonic transcriptomes (FIG. 12C, average Euclidean distances 4.92 versus 2.90 respectively, Mann-Whitney P<0.0001). In all, our multi-omics approach demonstrated differential microbiome signatures across GI tract regions and sub-regions in both mice and humans, with even the most distal luminal samples significantly distinct in composition and function from stool. These findings point out the limitations of solely relying on stool as a correlate for intestinal probiotics colonization and impact on the indigenous GI microbiome.

### Probiotics Strains are Present and Viable in the Administered Supplement

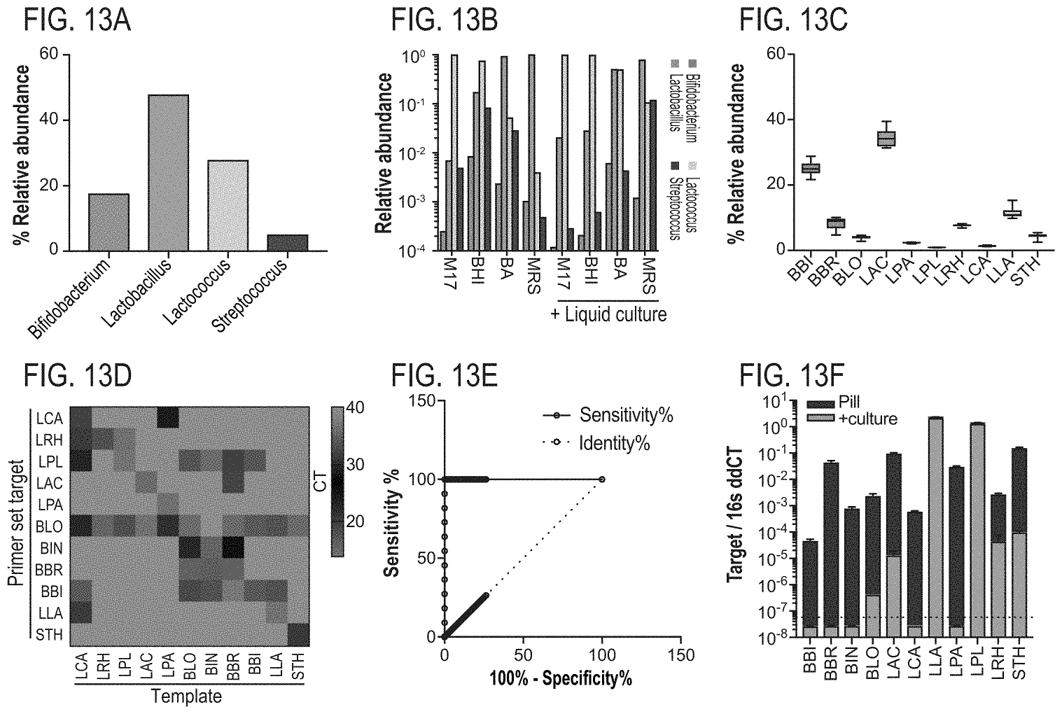

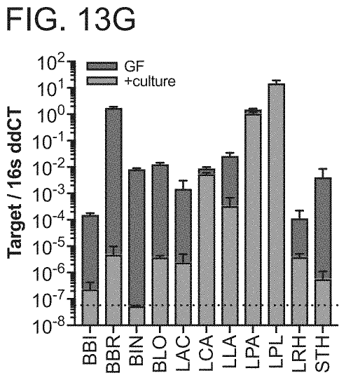

To study the effects of commonly consumed probiotics on the mammalian gut, we focused on a commercial probiotics preparation that includes 11 strains belonging to the four major Gram-positive bacterial genera used for this purpose: Lactobacillus, Bifidobacterium, Lactococcus and Streptococcus. Specifically, the preparation contained the following 11 strains: Lactobacillus acidophilus (abbreviated henceforth as LAC), Lactobacillus casei (LCA), Lactobacillus casei sbsp. paracasei (LPA), Lactobacillus plantarum (LPL), Lactobacillus rhamnosus (LRH), Bifidobacterium longum (BLO), Bifidobacterium bifidum (BBI), Bifidobacterium breve (BBR), Bifidobacterium longum sbsp. infantis (BIN), Lactococcus lactis (LLA) and Streptococcus thermophilus (STH). In order to determine the presence and viability of these 11 strains in the supplement, we first analyzed 16S rDNA amplicons obtained from the supplement pill with and without culturing. All four genera (and no others), but only 4/11 species (BBI, BLO, LAC, LCA) were identified by 16s rDNA analysis in the pill (FIG. 13A), and in colonies resulting from plating of the pill on different solid media with or without prior overnight culture in liquid medium (FIG. 13B). As this result might stem from insufficient sensitivity of 16S DNA sequencing rather than the actual absence of the strains, we employed shotgun metagenomic sequencing-based MetaPhlAn2 analysis that indeed identified 10/11 species, excluding BIN (FIG. 13C). MetaPhlAn2 analysis of a pure culture of BIN indicated that it is identified at the species level as B. longum. As an additional validation of probiotics strains presence, genomes for 9 of the 11 probiotic strains were recovered at >93% completeness and <4% contamination from metagenomics samples of the probiotics pill using reference-based and mini-assembly approaches⁸² (Table 5); For one of the species (B. longum) only part of the genome was recovered due to strain heterogeneity between BLO and BIN. As the abundance of several strains noted using MetaPhlAn2 was close to the detection threshold, we utilized species-specific qPCR primers and validated them on DNA obtained from pure cultures. Indeed, all targets were identified in their corresponding templates at CT (cycle threshold) values significantly lower than those observed in mismatched target-template pairs, which did not pass the detection threshold (40) for most mismatched pairs (FIG. 13D), resulting in a near-perfect area under the receiver-operator curve (ROC AUC) of 1 and P<0.0001 (FIG. 13E). qPCR amplification identified all 11 species in DNA purified independently from six different batches of pills with high reproducibility, though only 6/11 (BLO, LAC, LLA, LPL, LRH and STH) were found above the detection threshold after two subsequent cultures in liquid and solid BHI (FIG. 13F). To assess viability in the in vivo setting, we inoculated germ-free (GF) mice with the probiotics pill content and quantified the 11 species in stool samples collected five days post-inoculation, with all strains but BIN being cultivable (FIG. 13G). Live count of colonies grown from the pill on BHI resulted in 5*10⁹ CFU, in line with the manufacturer's statement. The effects of bowel preparation on probiotics species abundance were unchanged for most specifies (with the exception of BIN, BLO and STH), resulting in a significant positive correlation between per-tissue and individual levels of the probiotics species with or without bowel preparation (Spearman r=0.77, P<0.0001, FIG. 9J). Together, a combined culture-dependent and -independent approach utilizing 16S rDNA and shotgun metagenomic sequencing and qPCR validation readily identified all probiotics strains with high specificity, and all but BIN were proven to be viable under the aforementioned experimental conditions.

### Murine Microbiome-Driven Colonization Resistance Limits Probiotics Mucosal Colonization and Impact on the Indigenous Microbiome

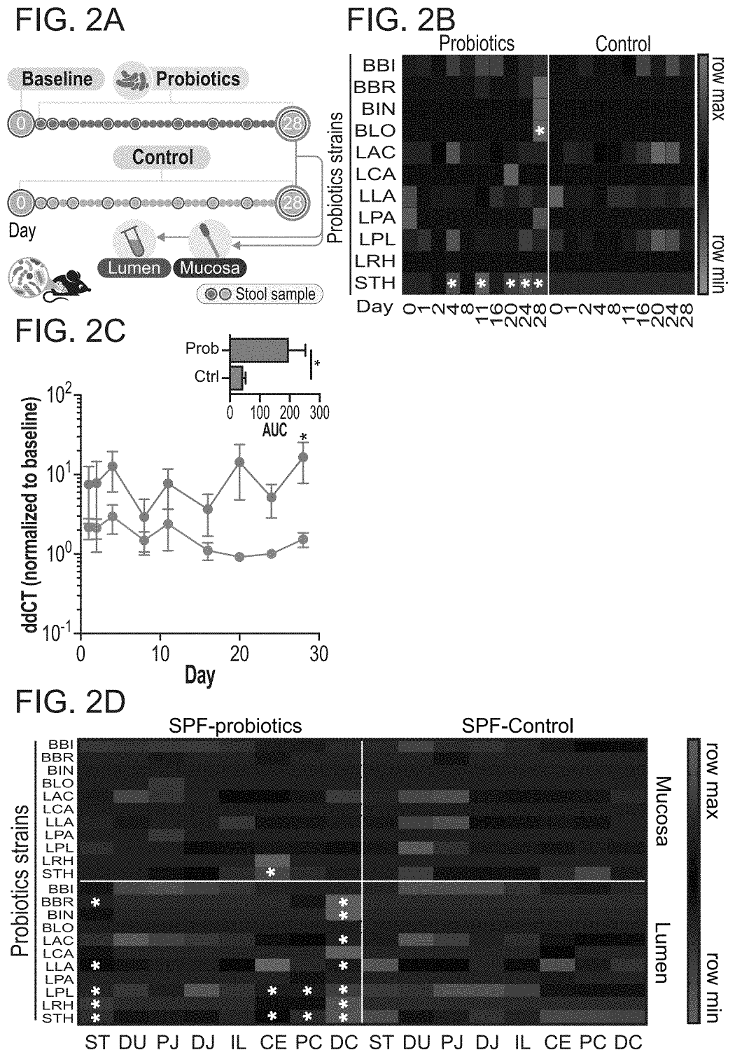

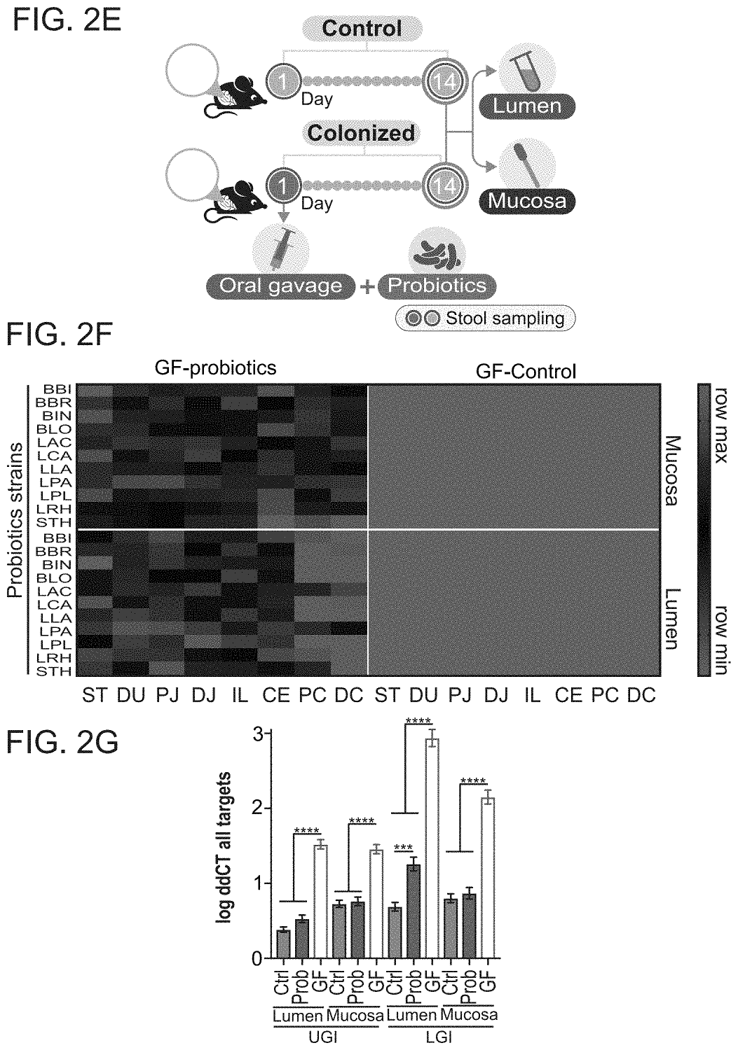

To assess the degree of murine GI colonization by the probiotics, we administered the contents of one pill daily by oral gavage (4*10⁹ CFU kg⁻¹ day⁻¹) to male 10-week-old SPF WT mice (N=10), with an additional group of untreated mice (N=10) serving as controls (FIG. 2A). Stool samples were analyzed at the indicated time-points, followed by a dissection of the gastrointestinal tract (FIG. 8A) on day 28 of supplementation. Highly specific qPCR amplification demonstrated significant stool shedding of BLO and STH in the probiotics group relative to baseline (STH 8-18.8-fold increase, Two-Way ANOVA & Dunnett P<0.03; BLO 22.4-fold increase P=0.0004, FIG. 2B) and no significant shedding in the control. When all the probiotic targets were considered together, an average 8.6-fold enrichment compared to baseline was observed in the treated group, resulting in significant differences between probiotics and control after 28 days of supplementation (15-fold, Two-Way ANOVA & Sidak P=0.03, FIG. 2C) and significant higher area under the probiotics daily fold increase curve (4.5-fold difference, Mann-Whitney P=0.01, FIG. 2C).

16S rDNA-based compositional analysis of luminal and mucosal samples collected throughout the GI tract did not indicate any significant differences between the probiotics and control groups in any region for any of the four probiotics genera (FIG. 14). Species-specific qPCR also demonstrated minimal differences between the probiotics and the control groups. The only significant difference in the mucosa was in cecal levels of STH (11.6-fold, Two-Way ANOVA & Sidak P=0.001, FIG. 2D). Significant differences in the lumen were restricted to the stomach and the LGI (average 4-fold difference to control, P<0.05), and were most pronounced in the stomach (average 5-fold, P<0.05) and distal colon (average 8.7-fold, P<0.02, FIG. 2D).

We hypothesized that this limited colonization of probiotics at the mucosal regions may result from colonization resistance of the murine microbiome to the supplemented strains. To address this possibility, we inoculated GF mice with an identical probiotics preparation by oral gavage and housed them in sterile isocages for 14 days before dissecting their GI tract (FIG. 2E) and utilized qPCR to directly compare the GF-probiotics, SPF-probiotics, and SPF-control mouse groups. No amplification was detected by any of the primer sets in GI tissues from control GF mice (FIG. 2F). In contrast, significant colonization of the probiotic strains was observed in GF-probiotics mice compared to both SPF groups (P<0.0001, Kruskal-Wallis & Dunn's, FIGS. 2F-G), with an average fold difference of 10 in UGI-lumen and 5 in UGI-mucosa compared to SPF-probiotics, 20 in the LGI mucosa and 50 in the LGI lumen. In comparison to this striking colonization in GF mice, aggregated fold increase of probiotics was only significant in the LGI lumen of SPF mice (3.7-fold difference, P=0.005, FIG. 2G).

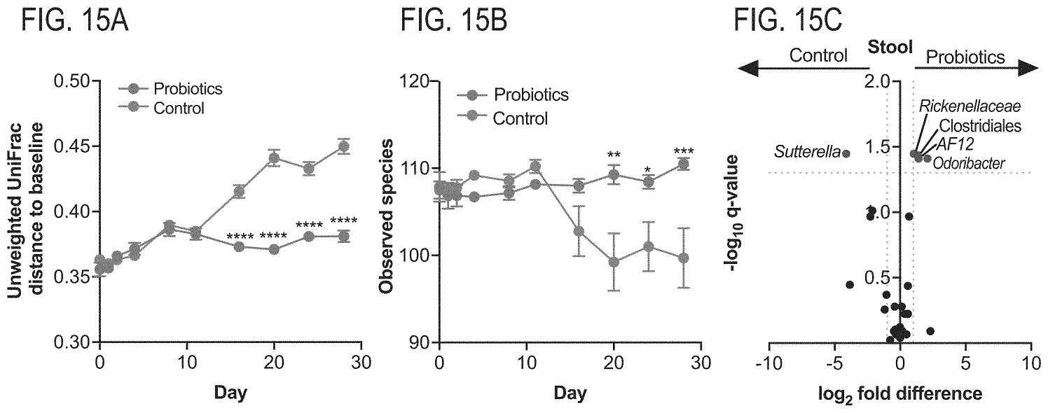

We next assessed the impact of the above low level probiotic colonization in the murine indigenous microbiome configuration. Both unweighted and weighted UniFrac distances of fecal samples (rarefied to 20000 reads) to baseline indicated no differences between the probiotics and control groups (Unweighted PERMANOVA P=0.35, weighted P=0.75) at early time points, with several later time-points becoming significantly different between the groups due to a drift observed only in the control group (FIGS. 15A-B), collectively resulting in 5 taxa that were significantly different between probiotics and control mice on the last day of supplementation (FDR-corrected Mann-Whitney P<0.05, FIG. 15C).

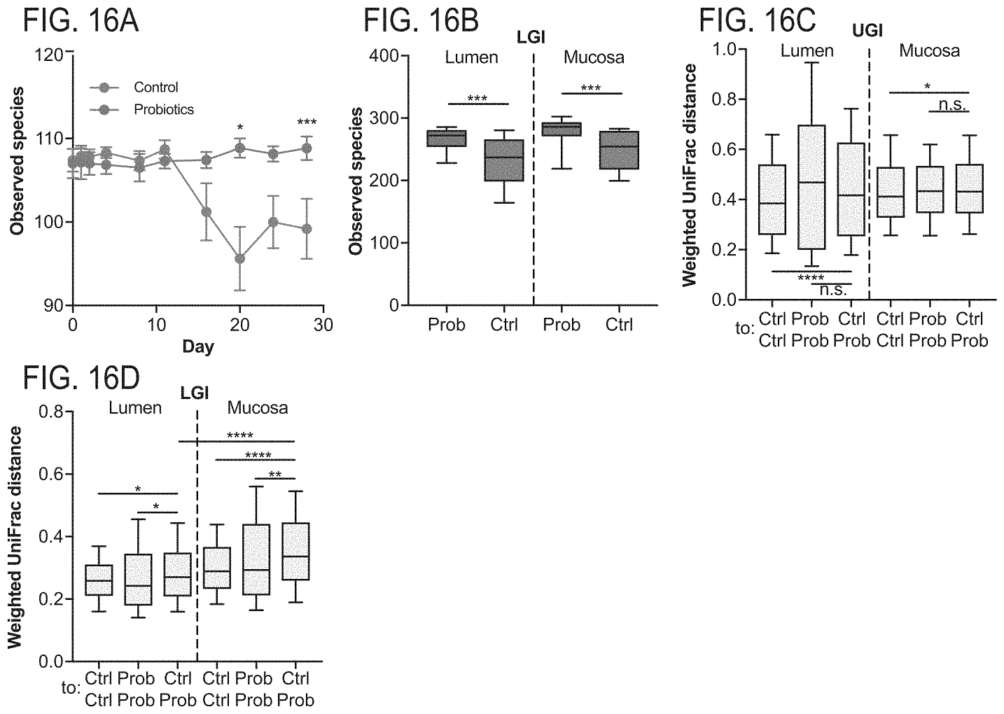

While no consistent probiotics-induced alterations of the UGI luminal (PERMANOVA P=0.2) and mucosal (PERMANOVA P=0.59) microbiome were observed (FIG. 3A-B), a significant shift was noted in the LGI microbiome, which was more pronounced in the mucosa compared to the lumen (mucosa PERMANOVA P=0.002, lumen P=0.02, FIG. 3C). These changes were accompanied with an increase in observed species both in LGI lumen (Mann-Whitney P=0.0001, FIG. 3D) and mucosa (P=0.0003, FIG. 3D) of probiotic-administered mice, but not the UGI (P>0.45, FIG. 3E). None of the aforementioned significant differences was merely due to the presence of the probiotics genera, as removal of the relevant genera and reanalysis after rarefaction to 20000 reads (stool) or 5000 reads (lumen and mucosa) did not affect the significantly higher alpha diversity in stool (FIG. 16A) or the LGI (FIG. 16B), as well as the weighted UniFrac distances in the UGI (lumen PERMANOVA P=0.1, mucosa P=0.29, FIG. 16C) or the LGI (lumen P=0.02, mucosa P=0.001, FIG. 16D). Collectively, 21 OTUS were differentially represented between probiotics and control in the LGI mucosa (FDR-corrected Mann-Whitney P<0.05, FIG. 3F). Interestingly, 10/14 OTUs that bloomed in the LGI mucosa of the probiotics group are characteristic of the oral cavity, the stomach, or both, as reported both by our mouse and human homeostatic analysis (FIGS. 8E, 10C-D) and by others^{81,83}.

Taken together, these findings suggest that despite daily administration, human-targeted probiotics feature low-level murine mucosal colonization, mediated by resistance exerted by the indigenous murine gut microbiome. Even at these low colonization levels, probiotics induced significant modulation of the LGI mucosal microbiome, which was not observed in stool samples.

### Inter-Individual Differences in Probiotics Colonization of the Human GI Tract

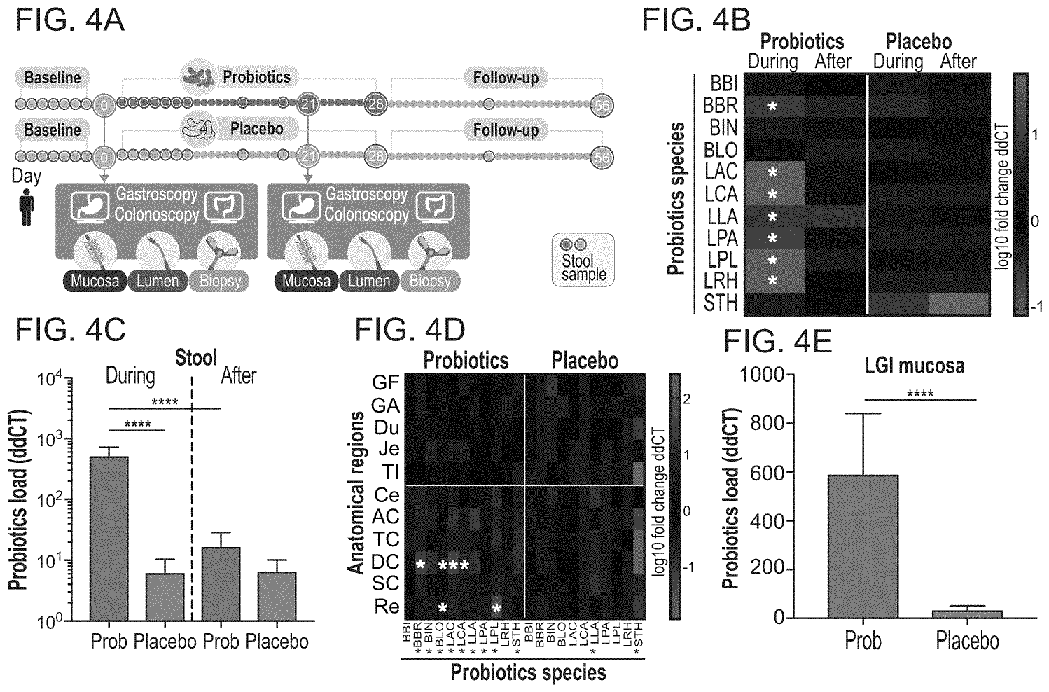

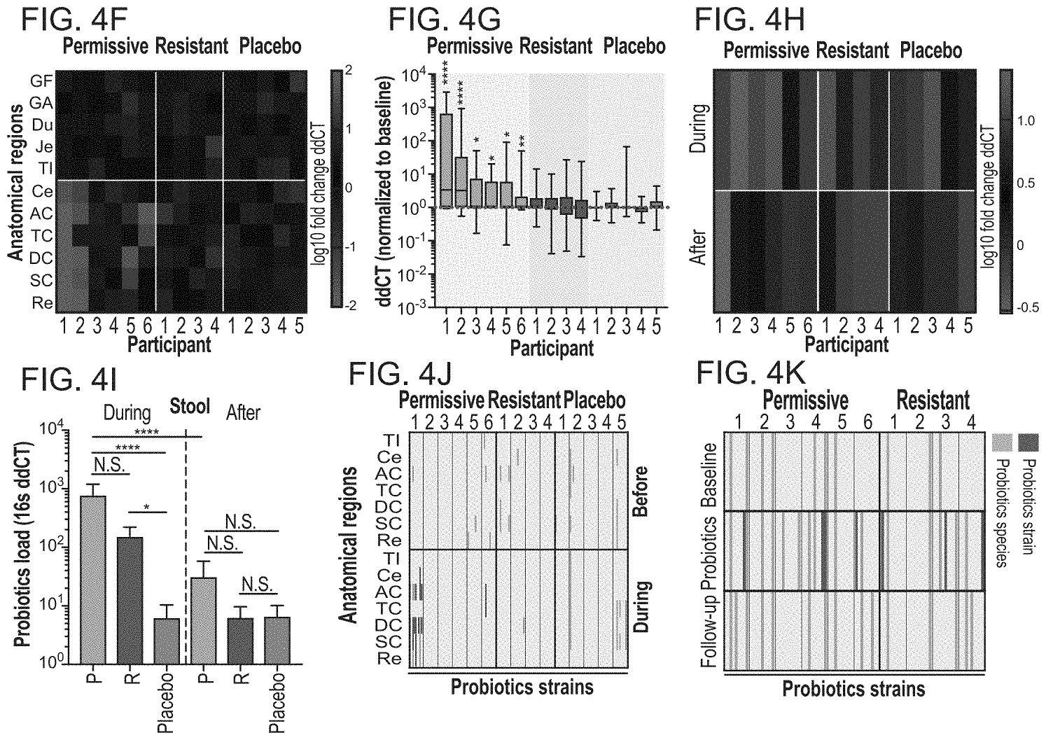

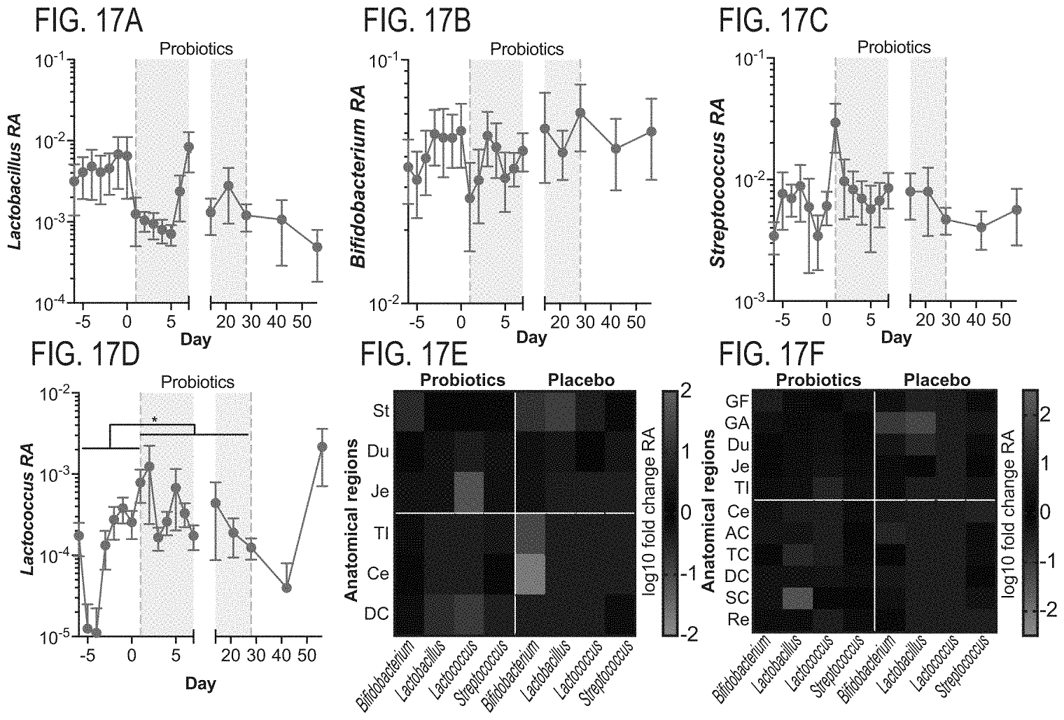

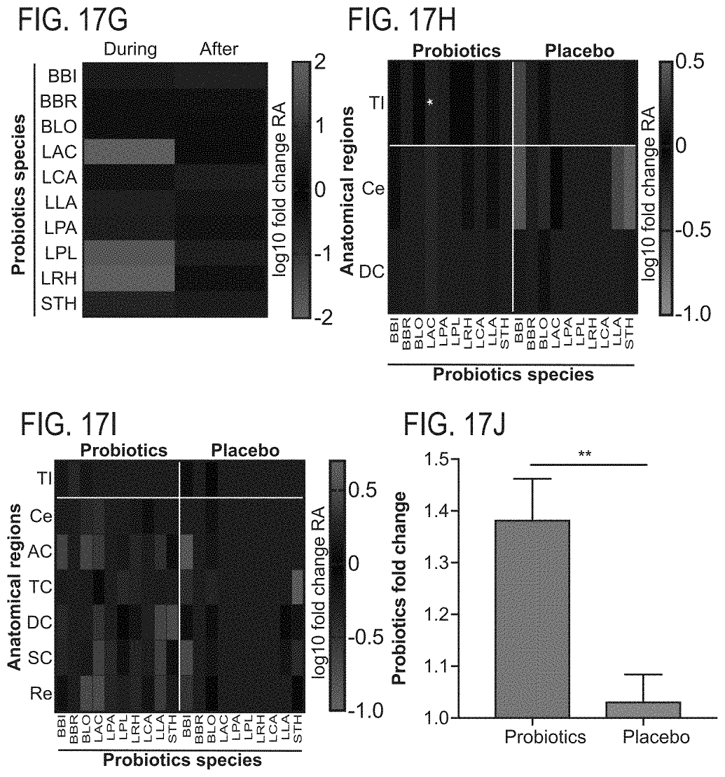

In contrast to inbred mice, humans display considerable person-to-person variation in gut microbiome composition, which may be more permissive to colonization with exogenous probiotics bacteria. To test this notion, we conducted a placebo-controlled trial, in which 15 healthy volunteers (see inclusion and exclusion criteria in methods) received either an identical 11-strain probiotics preparation or a cellulose placebo bi-daily for a 4-week period. Stool was sampled at multiple time points before, during, and after the administration of probiotics or placebo; colonoscopy and deep enteroscopy were performed prior to intervention and three weeks after the initiation of probiotics or placebo consumption in all participants (FIG. 4A). Probiotics colonization in humans was cross-validated by four different methods, including genus-level determination by 16S rDNA analysis; phylogenetic analysis of shotgun metagenomic sequences based on bacterial marker genes (MetaPhlAn2); amplification of the probiotics targets with qPCR; and strain-level analysis on shotgun metagenomic sequences based on unique genomic sequences⁸⁴. 16S rDNA analysis could not detect significant enrichment of Lactobacillus (Kruskal-Wallis & Dunn's P>0.28, FIG. 17A), Bifidobacterium (P>0.999, FIG. 17B) or Streptococcus (P>0.68, FIG. 17C) in stool samples during or after the supplementation period compared to baseline, whereas a 2.4-fold increase was observed for Lactococcus (P=0.02, FIG. 17D) Likewise, no significant differences were found when comparing the relative abundances of the probiotics genera in the luminal and mucosal samples of the supplemented group either to their own baseline or to the placebo group (FIGS. 17E-F). The more sensitive species-specific qPCR demonstrated significant shedding of 7/11 strains during consumption (FIG. 4B), namely BBR (8.6-fold expansion compared to baseline, Two-Way ANOVA & Dunnett P=0.025), LAC (137.3-fold, P=0.0001), LCA (75.3-fold, P=0.0001), LLA (7.8-fold, P=0.03), LPA (10.6-fold, P=0.015), LPL (70.7-fold, P=0.0001), and LRH (79.9-fold, P=0.0001). Aggregated probiotics fold difference significantly dropped to baseline after probiotics cessation (Kruskal-Wallis & Dunn's P<0.0001, FIG. 4B-C). There were no significant differences in the placebo group compared to baseline for any of the strains (FIG. 4B-C). Species-based MetaPhlAn2 analysis mirrored the qPCR findings with an average aggregated 86.8-fold increase in RA during consumption, though none of the species reached statistical significance (FIG. 17G). With the exception of a significant increase of LAC in the TI lumen (Two-Way ANOVA & Sidak P=0.01, FIG. 17H), none of the probiotics species were significantly increased in any of the luminal samples compared to either baseline or placebo (FIG. 17H). In contrast, qPCR demonstrated that 9/11 probiotics species were significantly enriched in the mucosa of the supplemented group compared to baseline, which was more pronounced in the LGI, especially the AC and DC (BBR 2.9-fold, Two-Way ANOVA P<0.0001; BIN 2-fold, P=0.016; BLO 2.28-fold, P<0.0001; LAC 4.2-fold, P<0.0001; LCA 2-fold, P<0.0001; LLA 2.5-fold, P=0.013; LPA 1.8-fold, P=0.0024; LPL 2.7-fold, P=0.0043; STH 3.54-fold, P=0.0022, FIG. 4D). LRH was significant when only the LGI was analyzed (2.9-fold, P=0.02, FIG. 4D). Compared to the 9 species that bloomed in the treated group, 2 species also significantly bloomed in the placebo group compared to baseline: LLA (3.39-fold, P=0.0008) and STH (5.7-fold, P=0.02). nonetheless the aggregated probiotics fold change was significantly higher in the treated group (Mann-Whitney P<0.0001, FIG. 4E). MetaPhlAn2 validated this observation (Mann-Whitney P=0.0022, FIGS. 17I-J).

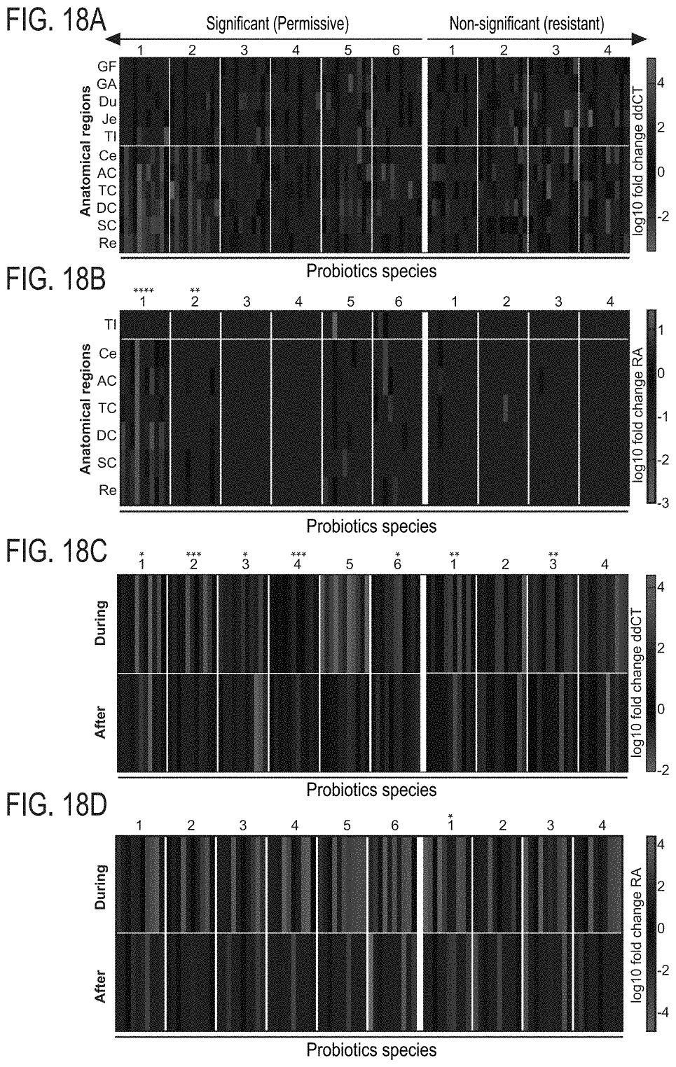

Surprisingly, when each participant was analyzed independently compared to its own baseline, the gastrointestinal mucosal load of probiotics strains considerably varied, with both qPCR (FIG. 4F, FIG. 18A) and MetaPhlAn2 (FIG. 18B) analyses pointing out to some individuals as featuring significant mucosal association of gut probiotics, while others do not. Both MetaPhlAn2 and qPCR identified two participants (Permissive 1 & 2, 10,000 permutations P=0.003 and P<0.0001 respectively) as significantly colonizing (FIGS. 4F-G, FIGS. 18A-B), and qPCR also identified 4 more participants (Permissive 3-6 P=0.034, p=0.026, P=0.03 and P=0.002, FIG. 4G) as probiotics colonizers. We defined individuals with a significant elevation in the absolute abundance of probiotic strains in their GI mucosa (as determined by Mann-Whitney test and validated by 10,000 permutations) as ‘permissive’ (FIGS. 4F-G, FIG. 18A). Of note, even among the permissive some (1 and 2) were more colonized than others (3-6), with participant 1 featuring the highest probiotics colonization, following by participant 2, then by the other four permissive.

Importantly, both the relative (FIG. 18C) and absolute (FIGS. 4H-I, FIG. 18D) abundance of probiotics strains in stools did not reflect this personalized mucosal colonization trait, with both permissive and resistant individuals featuring significant stool shedding during consumption (Permissive 1-4 and 6, and resistant 1 and 3, Two-Way ANOVA & Dunnett P<0.05, FIGS. 4H-I, FIGS. 18C-D), and even resistant individuals shedding significantly more probiotics in stool as compared to the placebo group (Mann-Whitney P=0.0066, FIG. 41). Once probiotic supplementation ceased, neither permissive nor resistant individuals featured a persistently significant stool shedding compared to placebo (P>0.3, FIG. 41). Moreover, strain-level analysis indicated that probiotic species found in stool and mucosal samples during the intervention period indeed were identical to the strains present in the administered pill, but were distinct from the ones excreted in stool at baseline (FIGS. 4J-K), or the follow-up period (gray, FIGS. 4J-K). Thus, and in contrast to a previous stool-focused study^{39,85}, we found shedding of probiotics species in stool to be non-indicative of person-specific gut mucosal colonization. Taken together, these findings point out that human consumption of 11 probiotic strains results in universal shedding in stools but with a highly individualized LGI mucosa colonization patterns.

### Baseline Personalized Host and Mucosal Microbiome Features are Associated with Probiotics Persistence

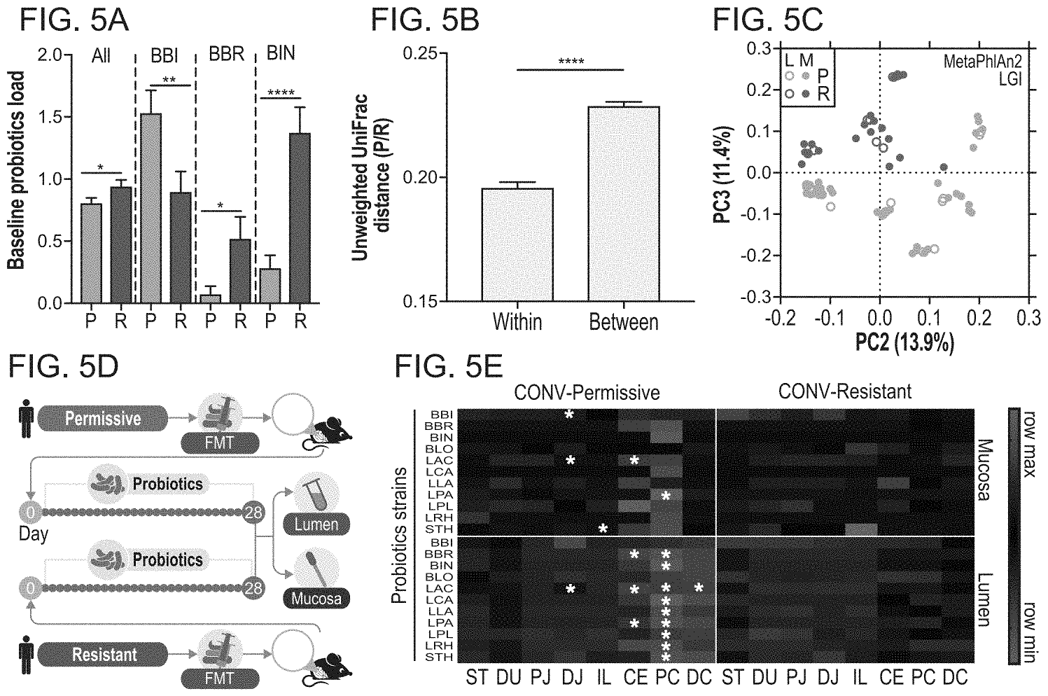

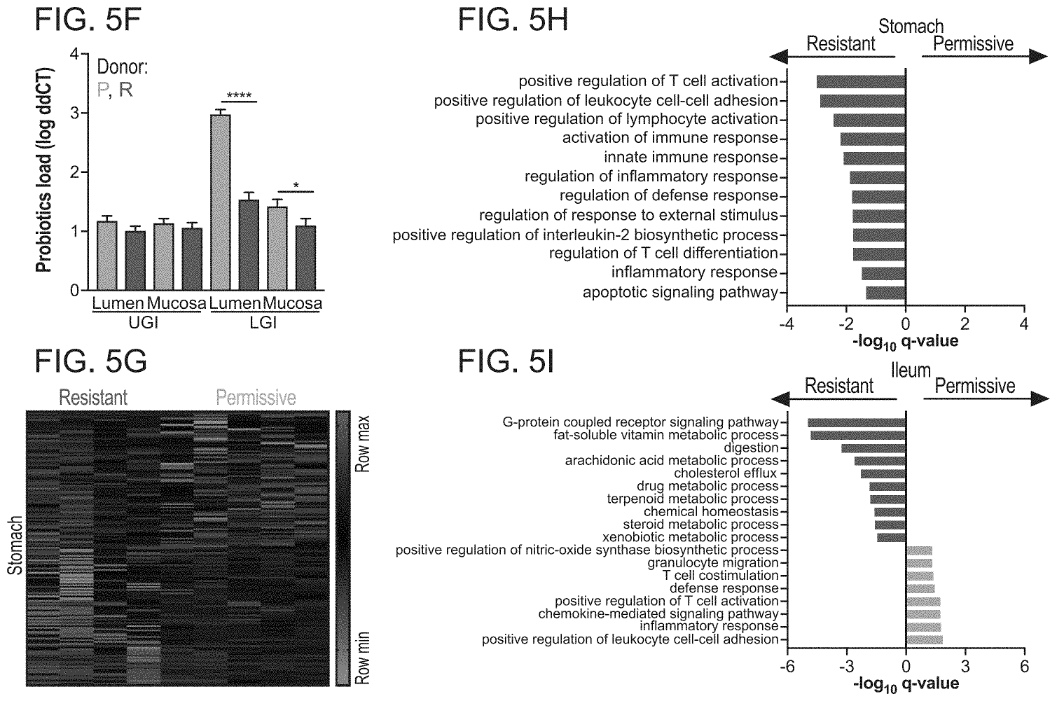

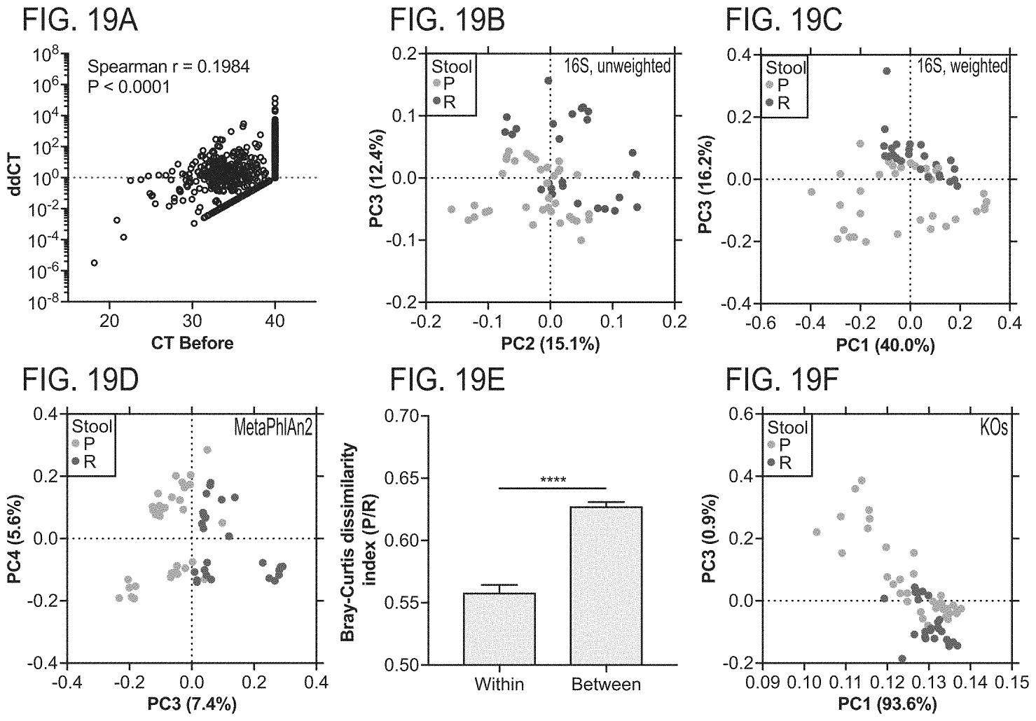

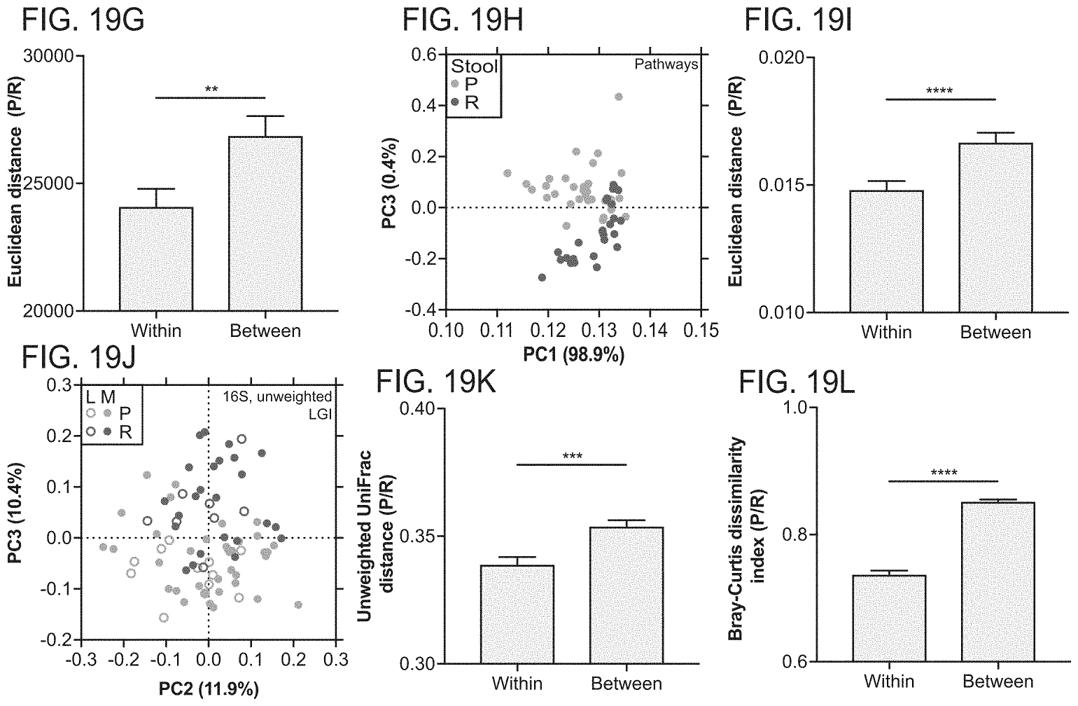

We next set out to identify factors that may dictate or mark the extent to which probiotics colonize the human GI mucosa. Interestingly, we observed a significant inverse correlation between initial levels of a given probiotics strain in a given GI region and its fold change, i.e. low abundant strains were more likely to expand than those already present in high loads (FIG. 19A, Spearman correlation P<0.0001). When taken together, permissive individuals had significantly lower baseline levels of the probiotics strains in the LGI mucosa (Mann-Whitney P=0.019, FIG. 5A), but not in stools. When each strain was compared individually between the two groups baselines, both BBR and BIN were significantly lower in permissive (BBR P=0.01, BIN P<0.0001, FIG. 5A), while LAC was marginally but non-significantly lower (P=0.056). In contrast, BBI, the only strain that did not significantly colonize the LGI mucosa (FIG. 4D), was higher in permissive at baseline (P=0.0095, FIG. 5A). In addition, permissive and resistant individuals clustered separately at baseline according to stool microbiome composition (16S-based unweighted UniFrac distances Mann-Whitney P<0.0001, FIG. 5B, FIG. 19B; weighted UniFrac distances, FIG. 19C; MetaPhLan2 P<0.0001, FIGS. 19D-E) and function (KOs P=0.0043, FIGS. 19F-G; Pathways P<0.0001, FIGS. 19H-I), as well as LGI composition (Unweighted UniFrac distances P=0.0002, FIGS. 19J-K; MetaPhlAn2 P<0.0001, FIG. 5C & FIG. 19L).

To determine whether these compositional and functional microbiome differences between permissive and resistant individuals impact colonization capacity of probiotics, we conventionalized two groups of GF mice with stool samples from either a permissive or a resistant participant. Probiotics were administered to the conventionalized mice daily by oral gavage for 4 weeks, after which the load of probiotics in the GI tract lumen and mucosa was quantified by qPCR (FIG. 5D). Both the LGI lumen and mucosa of mice conventionalized with ‘permissive’ microbiome were significantly more colonized compared to those of mice conventionalized with non-responder' microbiome (Lumen P<0.0001, Mucosa P=0.04, FIGS. 5E-F). Thus, and as observed in mice (FIG. 2A-G), the resident microbiome may play a role in permissiveness or resistance exerted on exogenous probiotics.

In order to identify host factors that may affect permissiveness or resistance to probiotics colonization, we performed a global gene expression analysis through RNA sequencing of transcripts collected from stomach, duodenum, jejunum, terminal ileum and descending colon biopsies before probiotics supplementation. Two clusters of genes that were higher in permissive vs resistant and vice versa were visible in the stomach (FIG. 5G). Interestingly, host pathways significantly enriched in resistant as compared to permissive were related to both adaptive and innate immune responses, inflammation and T cells activation and differentiation (FDR-corrected P<0.05, FIG. 5H). In contrast to the stomach, immune-related pathways were significantly enriched in the ilea of permissive vs. resistant, whereas pathways enriched in resistant included were related to digestion, metabolism, and xenobiotics metabolic processes (FIG. 5I). To conclude, both indigenous microbiome and host factors are differentially expressed in probiotics permissive and resistant individuals, even prior to exposure to probiotics. These host and microbiome factors may contribute to a differential colonization susceptibility to probiotics, potentially through competitive exclusion of related species and site-specific immune responses.

### Probiotics Differentially Affect Human Responders and Non-Responders

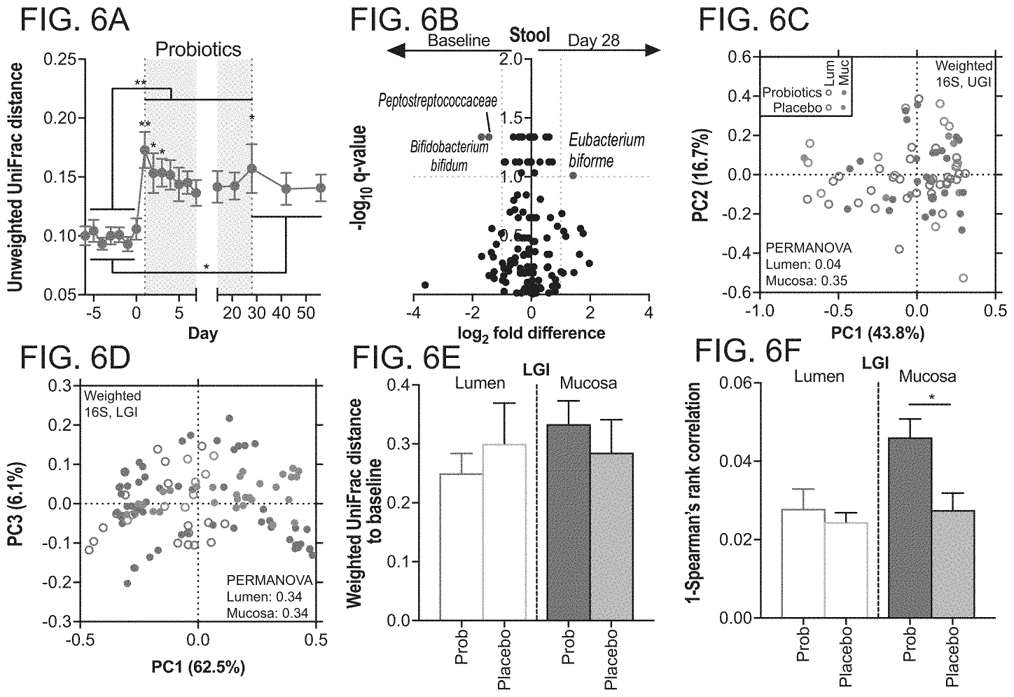

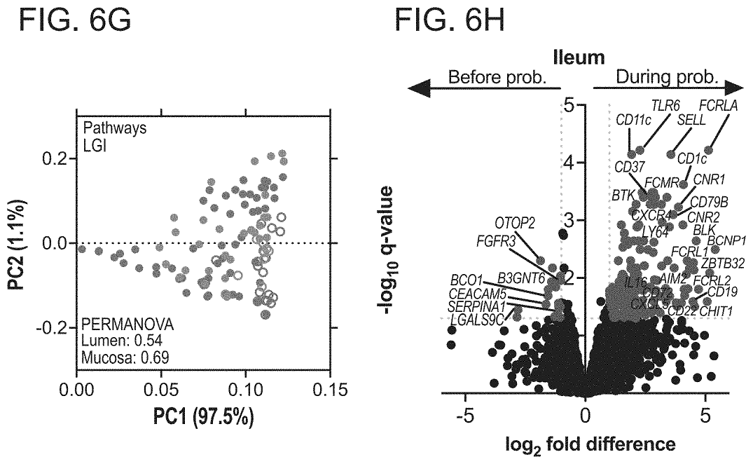

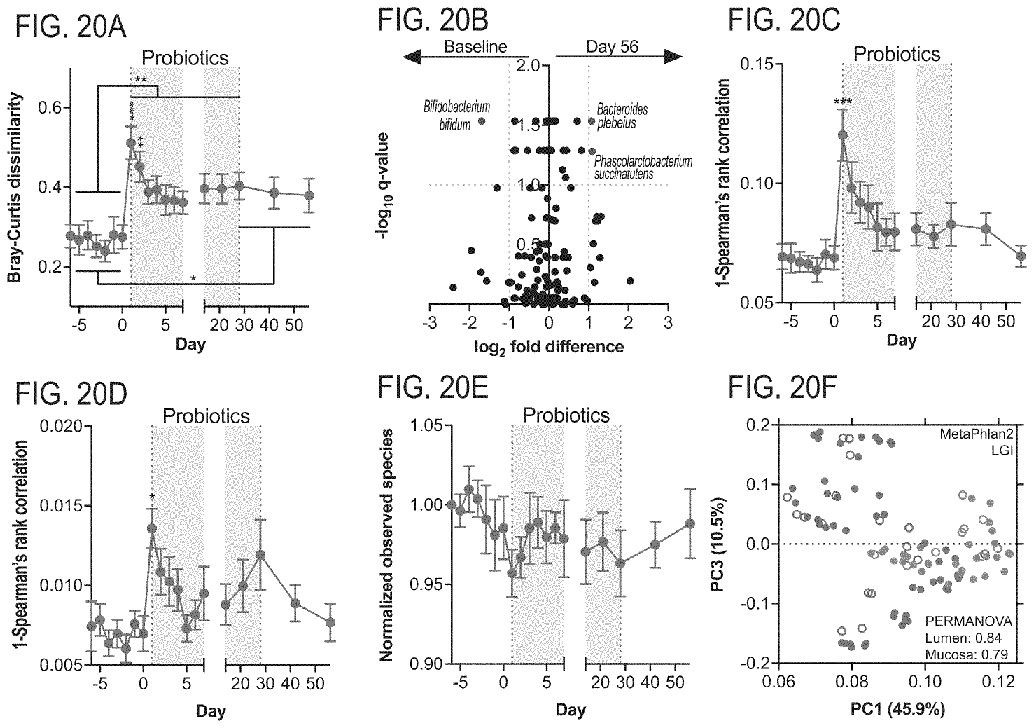

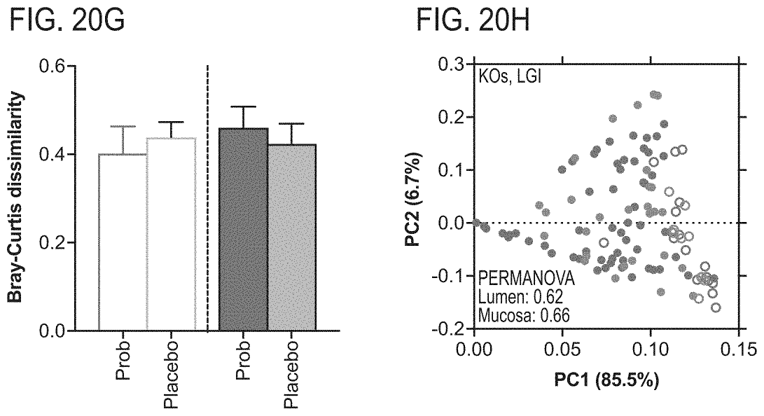

Finally, as the effect of probiotics on the human GI microbiome remains inconclusive⁴⁷, we sought to determine probiotics impact on microbiome composition and function and the host transcriptome, and whether these follow personalized patterns. We compared stool samples collected during and after probiotics supplementation to each participant's baseline, using 16S rDNA and MetaPhlAn2 compositional analysis, and shotgun metagenomic functional mapping to KOs and KEGG pathways. Stool microbiome composition was distinct from baseline during the probiotic exposure period (days 4-28 to baseline, Friedman & Dunn's P=0.0044 for 16s rDNA and MetaPhlAn2 analyses, FIG. 6A, FIG. 20A). Nonetheless, only a few species were significantly different (Mann-Whitney FDR-corrected P<0.1) between baseline and the last day of supplementation (FIG. 6B) or one month following probiotics cessation (FIG. 20B). These compositional changes were not reflected by significant alterations to microbiome function, according to KOs (FIG. 20C) or pathways (FIG. 20D), or by the number of observed species (FIG. 20E). We then compared compositional and functional differences between probiotics and placebo consuming individuals in luminal and mucosal samples from each anatomical region. In all four modalities (16S, MetaPhLan2, KOs, and pathways) we could not detect a single feature that was significantly different between the groups (FDR-corrected Mann-Whitney P<0.1). We therefore clustered luminal or mucosal samples to UGI and LGI and utilized a permutations-based test for all the modalities. In the UGI, weighted UniFrac separated the lumen of probiotics from that of placebo (99999 permutations P=0.04, FIG. 6C), although significance of this separation was lost when the probiotics genera were omitted from the analysis (99999 permutations P=0.071). The UGI mucosa did not differ between the probiotics to placebo groups according to weighted UniFrac (P=0.35, FIG. 6C), and no other significant differences were detected in the UGI by MetaPhlan2 (Lumen P=0.75, Mucosa P=0.11), KOs (Lumen P=0.6, Mucosa P=0.5) or pathways (Lumen P=0.6, Mucosa P=0.37). Likewise, weighted UniFrac did not distinguish between the groups after one month of consumption in the LGI lumen (99999 permutation P=0.34, FIG. 6D) or mucosa (P=0.34, FIG. 6D), and both groups changed to the same extent compared to baseline (Lumen Mann-Whitney P=0.68, Mucosa P=0.44, FIG. 6E). MetaPhlan2 reflected the absence of compositional differences (FIGS. 20F-G). Compared to baseline, more microbiome pathways were altered in the LGI mucosa of probiotics than in placebo (P=0.019, FIG. 6F). Nonetheless, neither KOs (Lumen 99999 permutations P=0.62, mucosa P=0.66, FIG. 20H) nor pathways (Lumen P=0.54, mucosa P=0.69, FIG. 20G) separated between the groups after one month of consumption. To conclude, when all probiotics consumers are compared either to placebo or to their own baseline, significant but minimal compositional changes are observed in stool samples. This is not reflected in the GI tract, where no probiotic effect is noted on the UGI and LGI microbiome. Nonetheless, probiotics consumption led to transcriptional changes in the ileum, with 19 down-regulated genes and 194 up-regulated, many of which related to the immune system and specifically to B cells (FIG. 6H).

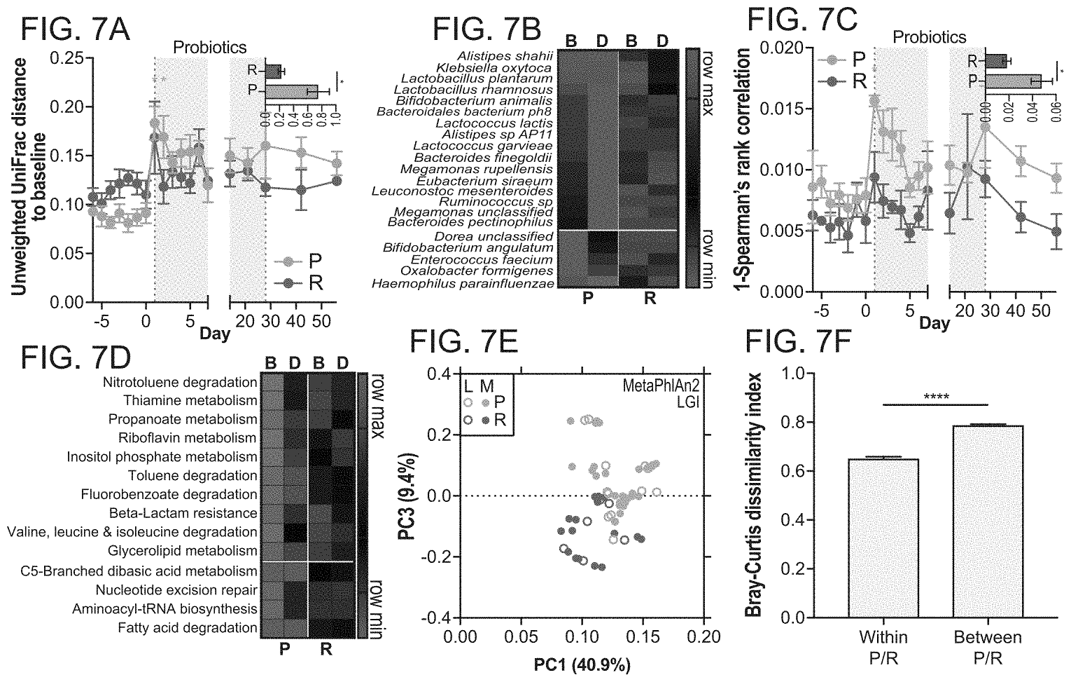

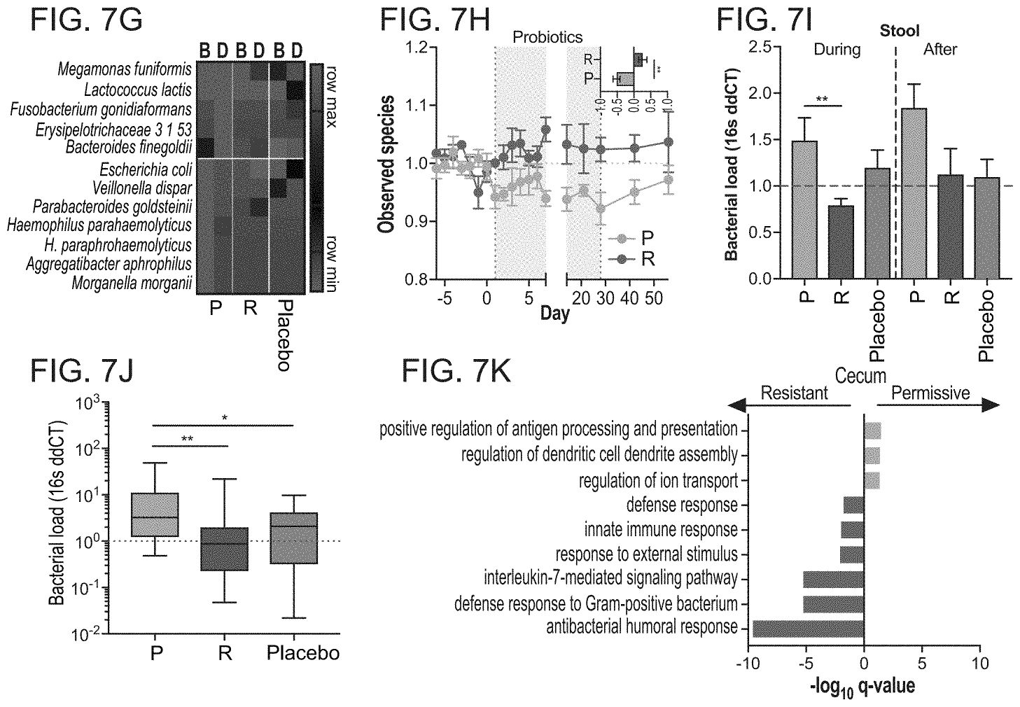

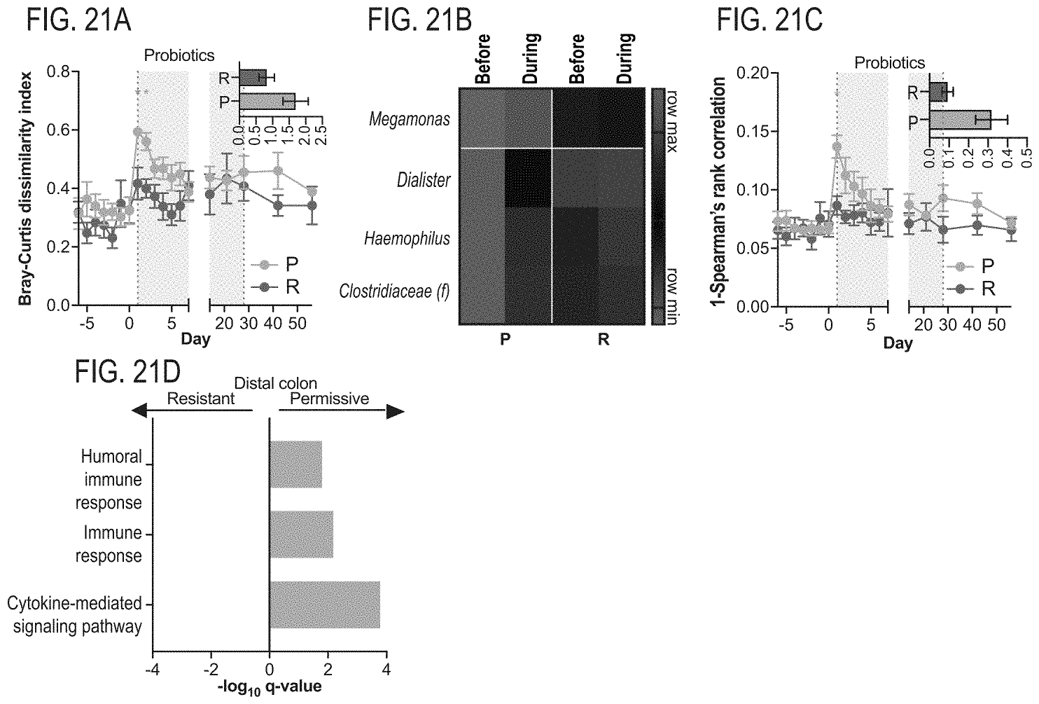

We next hypothesized that differential probiotics colonization between participants may result in differential effects on the microbiome, which can be obscured when all individuals are considered together. Indeed, during probiotic supplementation, compositional changes were pronounced in stools of permissive than in resistant participants, as evident by higher distances to baseline (unweighted UniFrac incremental AUC Mann-Whitney P=0.038, FIG. 7A; Bray-Curtis dissimilarity P=0.1, FIG. 21A). Some taxa, mostly characteristic of the UGI, were higher in permissive at baseline and were reduced to levels comparable to resistant following probiotics (e.g. Dialister, Haemophilus parainfulenzae, Enterococcus faecium), while others bloomed only in permissive participants (e.g. Megamonas and Bacteroides, FIG. 7B, FIG. 21B). Stool also recapitulated functional differences between the two groups, with changes from baseline more evident in permissive participants (KOs iAUC P=0.06, FIG. 21C; Pathways P=0.034, FIG. 7C) and generally reflecting, to some extent, probiotics-associated convergence with resistant (FIG. 7D). The initial microbiome composition difference in the LGI mucosa between permissive and resistant participants was maintained with probiotics supplementation (P<0.0001, FIGS. 7E-F), with a reduction of UGI-characteristic species coupled to multiple blooming taxa noted in permissive participants (FIG. 7G). Interestingly, probiotic supplementation was associated with a decrease in observed species (Mann-Whitney P=0.0095, FIG. 7H) but an increased total bacterial load in stool (1.49-fold compared to baseline, P=0.0095, FIG. 7I) in permissive individuals. Total bacterial load remained higher (1.84-fold) in permissive participants even one month following probiotics cessation, while it returned to baseline in resistant participants (1.12-fold, FIG. 7I), and remained stable throughout in placebo controls. Like in stool, bacterial load was significantly elevated in the LGI mucosa of permissive participants, compared to either resistant participants (3.7-fold difference, P=0.0019, FIG. 7J) or placebo controls (5.3-fold, P=0.038, FIG. 7J).

Probiotics also differentially affected the host GI transcriptome. Following initiation of probiotic consumption, all significant baseline ileum host pathways that distinguished permissive from resistant individuals (FIG. 7I) were ablated. Instead, following probiotics exposure the cecum emerged as a distinguishing region between the permissive and resistant groups (FIG. 7K), with the former enriched for pathways related to dendritic cells, antigen presentation and ion transport, while the later featuring multiple pathways associated with responses to exogenous stimuli, innate immune activation, anti-bacterial defense and specifically against Gram-positive bacteria (potentially related to all probiotics species assessed in this study being Gram-positive.) The distal colon of permissive individuals was enriched with three pathways associated with humoral immune response and cytokine-mediated signaling, but no pathways were enriched in the colon of resistant individuals following probiotics (FIG. 21D). Taken together, probiotics had a person-specific differential effect on GI microbiome composition and function and the host GI transcriptome, whose potential mechanisms of health impacts on the responding host merit further studies.

### REFERENCES FOR EXAMPLE 1

1 Clarke, T. C., Black, L. I., Stussman, B. J., Barnes, P. M. & Nahin, R. L. Trends in the use of complementary health approaches among adults: United States, 2002-2012. Natl Health Stat Report, 1-16 (2015).

2 Pinto-Sanchez, M. I. et al. Probiotic Bifidobacterium longum NCC3001 Reduces Depression Scores and Alters Brain Activity: A Pilot Study in Patients With Irritable Bowel Syndrome. Gastroenterology 153, 448-459 e448, doi:10.1053/j.gastro.2017.05.003 (2017).
3 Rondanelli, M. et al. Using probiotics in clinical practice: Where are we now? A review of existing meta-analyses. Gut Microbes, 1-23, doi:10.1080/19490976.2017.1345414 (2017).
4 Rijkers, G. T. et al. Health benefits and health claims of probiotics: bridging science and marketing. Br J Nutr 106, 1291-1296, doi:10.1017/5000711451100287X (2011).
5 Shen, N. T. et al. Timely Use of Probiotics in Hospitalized Adults Prevents Clostridium difficile Infection: A Systematic Review With Meta-Regression Analysis. Gastroenterology 152, 1889-1900 e1889, doi:10.1053/j.gastro.2017.02.003 (2017).
6 D'Souza, A. L., Rajkumar, C., Cooke, J. & Bulpitt, C. J. Probiotics in prevention of antibiotic associated diarrhoea: meta-analysis. BMJ 324, 1361 (2002).
7 Sanders, M. E. et al. An update on the use and investigation of probiotics in health and disease. Gut 62, 787-796, doi:10.1136/gutjnl-2012-302504 (2013).
8 Aoki, R. et al. A proliferative probiotic Bifidobacterium strain in the gut ameliorates progression of metabolic disorders via microbiota modulation and acetate elevation. Sci Rep 7, 43522, doi:10.1038/srep43522 (2017).
9 Chen, M. et al. Dairy consumption and risk of type 2 diabetes: 3 cohorts of US adults and an updated meta-analysis. BMC Med 12, 215, doi:10.1186/s12916-014-0215-1 (2014).
10 Xue, L. et al. Probiotics may delay the progression of nonalcoholic fatty liver disease by restoring the gut microbiota structure and improving intestinal endotoxemia. Sci Rep 7, 45176, doi:10.1038/srep45176 (2017).
11 Crovesy, L., Ostrowski, M., Ferreira, D., Rosado, E. L. & Soares-Mota, M. Effect of Lactobacillus on body weight and body fat in overweight subjects: a systematic review of randomized controlled clinical trials. Int J Obes (Lond) 41, 1607-1614, doi:10.1038/ijo.2017.161 (2017).
12 Hsiao, E. Y. et al. Microbiota modulate behavioral and physiological abnormalities associated with neurodevelopmental disorders. Cell 155, 1451-1463, doi:10.1016/j.cell.2013.11.024 (2013).
13 Bruce-Keller, A. J., Salbaum, J. M. & Berthoud, H. R. Harnessing Gut Microbes for Mental Health: Getting From Here to There. Biol Psychiatry, doi:10.1016/j.biopsych.2017.08.014 (2017).
14 Motevaseli, E., Dianatpour, A. & Ghafouri-Fard, S. The Role of Probiotics in Cancer Treatment: Emphasis on their In Vivo and In Vitro Anti-metastatic Effects. Int J Mol Cell Med 6, 66-76, doi:10.22088/acadpub.BUMS.6.2.1 (2017).
15 Draper, K., Ley, C. & Parsonnet, J. Probiotic guidelines and physician practice: a cross-sectional survey and overview of the literature. Benef Microbes 8, 507-519, doi:10.3920/BM2016.0146 (2017).
16 Panigrahi, P. et al. A randomized synbiotic trial to prevent sepsis among infants in rural India. Nature 548, 407-412, doi:10.1038/nature23480 (2017).
17 Bibiloni, R. et al. VSL #3 probiotic-mixture induces remission in patients with active ulcerative colitis. The American journal of gastroenterology 100, 1539 (2005).
18 Kruis, W. et al. Maintaining remission of ulcerative colitis with the probiotic Escherichia coli Nissle 1917 is as effective as with standard mesalazine. Gut 53, 1617-1623 (2004).
19 Furrie, E. et al. Synbiotic therapy (Bifidobacterium longum/Synergy 1) initiates resolution of inflammation in patients with active ulcerative colitis: a randomised controlled pilot trial. Gut 54, 242-249 (2005).
20 Mimura, T. et al. Once daily high dose probiotic therapy (VSL #3) for maintaining remission in recurrent or refractory pouchitis. Gut 53, 108-114 (2004).
21 Gionchetti, P. et al. Prophylaxis of pouchitis onset with probiotic therapy: a double-blind, placebo-controlled trial. Gastroenterology 124, 1202-1209 (2003).
22 Guerin-Danan, C. et al. Milk fermented with yogurt cultures and Lactobacillus casei compared with yogurt and gelled milk: influence on intestinal microflora in healthy infants. Am J Clin Nutr 67, 111-117 (1998).
23 Karlsson, C. et al. Probiotic therapy to men with incipient arteriosclerosis initiates increased bacterial diversity in colon: a randomized controlled trial. Atherosclerosis 208, 228-233, doi:10.1016/j.atherosclerosis.2009.06.019 (2010).
24 Allen, S. J. et al. Lactobacilli and bifidobacteria in the prevention of antibiotic-associated diarrhoea and Clostridium difficile diarrhoea in older inpatients (PLACIDE): a randomised, double-blind, placebo-controlled, multicentre trial. Lancet 382, 1249-1257, doi:10.1016/S0140-6736(13)61218-0 (2013).
25 Besselink, M. G. et al. Probiotic prophylaxis in predicted severe acute pancreatitis: a randomised, double-blind, placebo-controlled trial. The Lancet 371, 651-659 (2008).
26 Honeycutt, T. C. et al. Probiotic administration and the incidence of nosocomial infection in pediatric intensive care: a randomized placebo-controlled trial. Pediatric Critical Care Medicine 8, 452-458 (2007).
27 Vogel, G. (American Association for the Advancement of Science, 2008).
28 Saldanha, L. G. US Food and Drug Administration regulations governing label claims for food products, including probiotics. Clin Infect Dis 46 Suppl 2, S119-121; discussion S144-151, doi:10.1086/523328 (2008).
29 Tuomola, E., Crittenden, R., Playne, M., Isolauri, E. & Salminen, S. Quality assurance criteria for probiotic bacteria—. The American journal of clinical nutrition 73, 393s-398s (2001).
30 Degnan, F. H. Clinical studies involving probiotics: when FDA's investigational new drug rubric applies—and when it may not. Gut Microbes 3, 485-489, doi:10.4161/gmic.22158 (2012).
31 Sierra, S. et al. Intestinal and immunological effects of daily oral administration of Lactobacillus salivarius CECT5713 to healthy adults. Anaerobe 16, 195-200, doi:10.1016/j.anaerobe.2010.02.001 (2010).
32 Wang, C. et al. Intestinal Microbiota Profiles of Healthy Pre-School and School-Age Children and Effects of Probiotic Supplementation. Ann Nutr Metab 67, 257-266, doi:10.1159/000441066 (2015).
33 Rochet, V. et al. Effects of orally administered Lactobacillus casei DN-114 001 on the composition or activities of the dominant faecal microbiota in healthy humans. Br J Nutr 95, 421-429 (2006).
34 Goossens, D. et al. The effect of Lactobacillus plantarum 299v on the bacterial composition and metabolic activity in faeces of healthy volunteers: a placebo-controlled study on the onset and duration of effects. Aliment Pharmacol Ther 18, 495-505 (2003).
35 Lahti, L. et al. Associations between the human intestinal microbiota, Lactobacillus rhamnosus GG and serum lipids indicated by integrated analysis of high-throughput profiling data. PeerJ 1, e32, doi:10.7717/peerj.32 (2013).
36 Jacobsen, C. N. et al. Screening of probiotic activities of forty-seven strains of Lactobacillus spp. by in vitro techniques and evaluation of the colonization ability of five selected strains in humans. Applied and environmental microbiology 65, 4949-4956 (1999).
37 Tannock, G. et al. Analysis of the fecal microflora of human subjects consuming a probiotic product containing Lactobacillus rhamnosus DR20. Applied and environmental microbiology 66, 2578-2588 (2000).
38 Goldin, B. R. et al. Survival of Lactobacillus species (strain GG) in human gastrointestinal tract. Digestive diseases and sciences 37, 121-128 (1992).
39 Maldonado-Gomez, M. X. et al. Stable Engraftment of Bifidobacterium longum AH1206 in the Human Gut Depends on Individualized Features of the Resident Microbiome. Cell Host Microbe 20, 515-526, doi:10.1016/j.chom.2016.09.001 (2016).
40 Turroni, F. et al. Role of sortase-dependent pili of Bifidobacterium bifidum PRL2010 in modulating bacterium-host interactions. Proc Natl Acad Sci USA 110, 11151-11156, doi:10.1073/pnas.1303897110 (2013).
41 Kaushik, J. K. et al. Functional and probiotic attributes of an indigenous isolate of Lactobacillus plantarum. PLoS One 4, e8099, doi:10.1371/journal.pone.0008099 (2009).
42 Van Tassell, M. L. & Miller, M. J. Lactobacillus adhesion to mucus. Nutrients 3, 613-636 (2011).
43 Lee, Y. et al. Quantitative approach in the study of adhesion of lactic acid bacteria to intestinal cells and their competition with enterobacteria. Applied and environmental microbiology 66, 3692-3697 (2000).
44 Bernet, M.-F., Brassart, D., Neeser, J.-R. & Servin, A. Lactobacillus acidophilus LA 1 binds to cultured human intestinal cell lines and inhibits cell attachment and cell invasion by enterovirulent bacteria. Gut 35, 483-489 (1994).
45 He, F. et al. Differences in composition and mucosal adhesion of bifidobacteria isolated from healthy adults and healthy seniors. Current microbiology 43, 351-354 (2001).
46 Kankaanpää, P. E., Salminen, S. J., Isolauri, E. & Lee, Y. K. The influence of polyunsaturated fatty acids on probiotic growth and adhesion. FEMS microbiology letters 194, 149-153 (2001).
47 Kristensen, N. B. et al. Alterations in fecal microbiota composition by probiotic supplementation in healthy adults: a systematic review of randomized controlled trials. Genome Med 8, 52, doi:10.1186/s13073-016-0300-5 (2016).
48 Laursen, M. F. et al. Administration of two probiotic strains during early childhood does not affect the endogenous gut microbiota composition despite probiotic proliferation. BMC Microbiol 17, 175, doi:10.1186/s12866-017-1090-7 (2017).
49 Bjerg, A. T. et al. The effect of Lactobacillus paracasei subsp. paracasei L. casei W8(R) on blood levels of triacylglycerol is independent of colonisation. Benef Microbes 6, 263-269, doi:10.3920/BM2014.0033 (2015).
50 Hanifi, A. et al. Evaluation of Bacillus subtilis R0179 on gastrointestinal viability and general wellness: a randomised, double-blind, placebo-controlled trial in healthy adults. Benef Microbes 6, 19-27, doi:10.3920/BM2014.0031 (2015).
51 Eloe-Fadrosh, E. A. et al. Functional dynamics of the gut microbiome in elderly people during probiotic consumption. MBio 6, doi:10.1128/mBio.00231-15 (2015).
52 Ferrario, C. et al. Modulation of fecal Clostridiales bacteria and butyrate by probiotic intervention with Lactobacillus paracasei DG varies among healthy adults. J Nutr 144, 1787-1796, doi:10.3945/jn.114.197723 (2014).
53 Goossens, D. A., Jonkers, D. M., Russel, M. G., Stobberingh, E. E. & Stockbrugger, R. W. The effect of a probiotic drink with Lactobacillus plantarum 299v on the bacterial composition in faeces and mucosal biopsies of rectum and ascending colon. Aliment Pharmacol Ther 23, 255-263, doi:10.1111/j.1365-2036.2006.02749.x (2006).
54 Martin, F. P. et al. Probiotic modulation of symbiotic gut microbial-host metabolic interactions in a humanized microbiome mouse model. Mol Syst Biol 4, 157, doi:10.1038/msb4100190 (2008).
55 Yuan, F. et al. Efficacy of Bifidobacterium infantis 35624 in patients with irritable bowel syndrome: a meta-analysis. Curr Med Res Opin 33, 1191-1197, doi:10.1080/03007995.2017.1292230 (2017).
56 Hegarty, J., Guinane, C., Ross, R., Hill, C. & Cotter, P. Lack of Heterogeneity in Bacteriocin Production Across a Selection of Commercial Probiotic Products. Probiotics and antimicrobial proteins 9, 459-465 (2017).
57 Yatsunenko, T. et al. Human gut microbiome viewed across age and geography. Nature 486, 222-227, doi:10.1038/nature11053 (2012).
58 David, L. A. et al. Diet rapidly and reproducibly alters the human gut microbiome. Nature 505, 559 (2014).
59 Claesson, M. J. et al. Gut microbiota composition correlates with diet and health in the elderly. Nature 488, 178 (2012).
60 Suez, J. et al. Artificial sweeteners induce glucose intolerance by altering the gut microbiota. Nature 514, 181 (2014).
61 Chassaing, B. et al. Dietary emulsifiers impact the mouse gut microbiota promoting colitis and metabolic syndrome. Nature 519, 92 (2015).
62 Forslund, K. et al. Disentangling type 2 diabetes and metformin treatment signatures in the human gut microbiota. Nature 528, 262 (2015).
63 Thaiss, C. A. et al. Transkingdom control of microbiota diurnal oscillations promotes metabolic homeostasis. Cell 159, 514-529 (2014).
64 Fuller, R. Probiotics in human medicine. Gut 32, 439-442 (1991).
65 Ouwehand, A. C., Salminen, S. & Isolauri, E. Probiotics: an overview of beneficial effects. Antonie Van Leeuwenhoek 82, 279-289 (2002).
66 Eckburg, P. B. et al. Diversity of the human intestinal microbial flora. science 308, 1635-1638 (2005).
67 De Cárcer, D. A. et al. Numerical ecology validates a biogeographical distribution and gender-based effect on mucosa-associated bacteria along the human colon. The ISME journal 5, 801 (2011).
68 Stearns, J. C. et al. Bacterial biogeography of the human digestive tract. Sci Rep 1, 170, doi:10.1038/srep00170 (2011).
69 Booijink, C. C. et al. High temporal and inter-individual variation detected in the human ileal microbiota. Environmental microbiology 12, 3213-3227, doi:10.1111/j.1462-2920.2010.02294.x (2010).
70 Wang, M., Ahrne, S., Jeppsson, B. & Molin, G. Comparison of bacterial diversity along the human intestinal tract by direct cloning and sequencing of 16S rRNA genes. FEMS Microbiol Ecol 54, 219-231, doi:10.1016/j.femsec.2005.03.012 (2005).
71 Yasuda, K. et al. Biogeography of the intestinal mucosal and lumenal microbiome in the rhesus macaque. Cell Host Microbe 17, 385-391, doi:10.1016/j.chom.2015.01.015 (2015).
72 Donaldson, G. P., Lee, S. M. & Mazmanian, S. K. Gut biogeography of the bacterial microbiota. Nature reviews. Microbiology 14, 20-32, doi:10.1038/nrmicro3552 (2016).
73 Suzuki, T. A. & Nachman, M. W. Spatial Heterogeneity of Gut Microbial Composition along the Gastrointestinal Tract in Natural Populations of House Mice. PLoS One 11, e0163720, doi:10.1371/journal.pone.0163720 (2016).
74 Mowat, A. M. & Agace, W. W. Regional specialization within the intestinal immune system. Nature reviews. Immunology 14, 667-685, doi:10.1038/nri3738 (2014).
75 Yang, H. et al. Uncovering the composition of microbial community structure and metagenomics among three gut locations in pigs with distinct fatness. Scientific reports 6, 27427 (2016).
76 Albenberg, L. et al. Correlation between intraluminal oxygen gradient and radial partitioning of intestinal microbiota. Gastroenterology 147, 1055-1063.e1058, doi: 10.1053/j.gastro.2014.07.020 (2014).
77 McNulty, N. P. et al. The impact of a consortium of fermented milk strains on the gut microbiome of gnotobiotic mice and monozygotic twins. Sci Transl Med 3, 106ra106, doi:10.1126/scitranslmed.3002701 (2011).
78 Charbonneau, D., Gibb, R. D. & Quigley, E. M. Fecal excretion of Bifidobacterium infantis 35624 and changes in fecal microbiota after eight weeks of oral supplementation with encapsulated probiotic. Gut Microbes 4, 201-211, doi:10.4161/gmic.24196 (2013).
79 Drago, L., Toscano, M., De Grandi, R., Casini, V. & Pace, F. Persisting changes of intestinal microbiota after bowel lavage and colonoscopy. European journal of gastroenterology & hepatology 28, 532-537 (2016).
80 O'Brien, C. L., Allison, G. E., Grimpen, F. & Pavli, P. Impact of colonoscopy bowel preparation on intestinal microbiota. PloS one 8, e62815 (2013).
81 Human Microbiome Project, C. Structure, function and diversity of the healthy human microbiome. Nature 486, 207-214, doi:10.1038/nature11234 (2012).
82 Sharon, I. et al. Time series community genomics analysis reveals rapid shifts in bacterial species, strains, and phage during infant gut colonization. Genome research 23, 111-120 (2013).
83 Bik, E. M. et al. Molecular analysis of the bacterial microbiota in the human stomach. Proceedings of the National Academy of Sciences of the United States of America 103, 732-737 (2006).
84 Sharon, I. et al. Accurate, multi-kb reads resolve complex populations and detect rare microorganisms. Genome Res 25, 534-543, doi:10.1101/gr.183012.114 (2015).
85 Frese, S. A., Hutkins, R. W. & Walter, J. Comparison of the colonization ability of autochthonous and allochthonous strains of lactobacilli in the human gastrointestinal tract. Advances in Microbiology 2, 399 (2012).
86 Del Piano, M. et al. Evaluation of the intestinal colonization by microencapsulated probiotic bacteria in comparison with the same uncoated strains. Journal of clinical gastroenterology 44, S42-S46 (2010).
87 Suez, J., Korem, T., Zilberman-Schapira, G., Segal, E. & Elinav, E. Non-caloric artificial sweeteners and the microbiome: findings and challenges. Gut Microbes 6, 149-155, doi:10.1080/19490976.2015.1017700 (2015).
88 Herbel, S. et al. Species—specific quantification of probiotic lactobacilli in yoghurt by quantitative real-time PCR. Journal of applied microbiology 115, 1402-1410 (2013).
89 Furet, J.-P., Quénée, P. & Tailliez, P. Molecular quantification of lactic acid bacteria in fermented milk products using real-time quantitative PCR. International journal of food microbiology 97, 197-207 (2004).
90 Schwendimann, L., Kauf, P., Fieseler, L., Gantenbein-Demarchi, C. & Schwenninger, S. M. Development of a quantitative PCR assay for rapid detection of Lactobacillus plantarum and Lactobacillus fermentum in cocoa bean fermentation. Journal of microbiological methods 115, 94-99 (2015).
91 Haarman, M. & Knol, J. Quantitative real-time PCR assays to identify and quantify fecal Bifidobacterium species in infants receiving a prebiotic infant formula. Applied and environmental microbiology 71, 2318-2324 (2005).
92 Ruggirello, M., Dolci, P. & Cocolin, L. Detection and viability of Lactococcus lactis throughout cheese ripening. PloS one 9, e114280 (2014).
93 Huber, J. A. et al. Microbial population structures in the deep marine biosphere. science 318, 97-100 (2007).
94 Caporaso, J. G. et al. QIIME allows analysis of high-throughput community sequencing data. Nature methods 7, 335 (2010).
95 Bolger, A. M., Lohse, M. & Usadel, B. Trimmomatic: a flexible trimmer for Illumina sequence data. Bioinformatics 30, 2114-2120 (2014).
96 Truong, D. T. et al. MetaPhlAn2 for enhanced metagenomic taxonomic profiling. Nature methods 12, 902 (2015).
97 Langmead, B. & Salzberg, S. L. Fast gapped-read alignment with Bowtie 2. Nature methods 9, 357 (2012).
98 Manor, O. & Borenstein, E. Revised computational metagenomic processing uncovers hidden and biologically meaningful functional variation in the human microbiome. Microbiome 5, 19 (2017).
99 Eden, E., Navon, R., Steinfeld, I., Lipson, D. & Yakhini, Z. GOrilla: a tool for discovery and visualization of enriched GO terms in ranked gene lists. BMC bioinformatics 10, 48 (2009).
100 Qin, J. et al. A human gut microbial gene catalogue established by metagenomic sequencing. nature 464, 59 (2010).
101 Kanehisa, M. & Goto, S. KEGG: kyoto encyclopedia of genes and genomes. Nucleic acids research 28, 27-30 (2000).
102 Peng, Y., Leung, H. C., Yiu, S.-M. & Chin, F. Y. IDBA-UD: a de novo assembler for single-cell and metagenomic sequencing data with highly uneven depth. Bioinformatics 28, 1420-1428 (2012).
103 Parks, D. H., Imelfort, M., Skennerton, C. T., Hugenholtz, P. & Tyson, G. W. CheckM: assessing the quality of microbial genomes recovered from isolates, single cells, and metagenomes. Genome research 25, 1043-1055 (2015).
104 Edgar, R. C. Search and clustering orders of magnitude faster than BLAST. Bioinformatics 26, 2460-2461 (2010).
105 Li, X. et al. A comparison of per sample global scaling and per gene normalization methods for differential expression analysis of RNA-seq data. PloS one 12, e0176185 (2017).

### Example 2

### Post-Antibiotic Gut Mucosal Microbiome Reconstitution is Impaired by Probiotics and Improved by Autologous FMT

Reagents and resources: see Table 1 of Example 1

Clinical trial: The human trial was approved by the Tel Aviv Sourasky Medical Center Institutional Review Board (IRB approval numbers TLV-0553-12 and TLV-0658-12) and Weizmann Institute of Science Bioethics and Embryonic Stem Cell Research oversight committee (IRB approval numbers 421-1 and 430-1), and was reported to clinical trials (Identifier: NCT03218579). Written informed consent was obtained from all subjects. No changes were done to the study protocol and methods after the trial commenced.

Exclusion and inclusion criteria (human cohorts): All subjects fulfilled the following inclusion criteria: males and females, aged 18-70, who are currently not following any diet regime or dietitian consultation and are able to provide informed consent. Exclusion criteria included: (i) pregnancy or fertility treatments; (ii) usage of antibiotics or antifungals within three months prior to participation; (iii) consumption of probiotics in any form within one month prior to participation, (iv) chronically active inflammatory or neoplastic disease in the three years prior to enrollment; (v) chronic gastrointestinal disorder, including inflammatory bowel disease and celiac disease; (vi) active neuropsychiatric disorder; (vii) myocardial infarction or cerebrovascular accident in the 6 months prior to participation; (viii) coagulation disorders; (ix) chronic immunosuppressive medication usage; (x) pre-diagnosed type I or type II diabetes mellitus or treatment with anti-diabetic medication. Adherence to inclusion and exclusion criteria was validated by medical doctors.

TABLE 6
Participants details

| | | | Age | Weight | Height | BMI | | |
|---|---|---|---|---|---|---|---|---|
| #Participant | Sex | Group | (years) | (Kg) | (cm) | (kg/m2) | Smoking | Diet |
| 1 | M | No intervention | 46 | 100 | 191 | 27.41 | Never | Vegetarian |
| 2 | M | No intervention | 32 | 63 | 178 | 19.88 | Never | Omnivore |
| 3 | F | No intervention | 45 | 59 | 159 | 23.34 | Never | Omnivore |
| 4 | M | No intervention | 58 | 76 | 175 | 24.82 | Never | Omnivore |
| 5 | M | No intervention | 58 | 100 | 184 | 29.54 | Never | Omnivore |
| 6 | F | No intervention | 40 | 65 | 160 | 25.39 | Never | Omnivore |
| 7 | F | No intervention | 66 | 64 | 164 | 23.8 | Never | Omnivore |
| 8 | F | No intervention | 25 | 60 | 172 | 20.28 | Past | Omnivore |
| 9 | F | No intervention | 27 | 66 | 170 | 22.84 | Never | Omnivore |
| 10 | M | No intervention | 19 | 80 | 186 | 23.12 | Past | Omnivore |
| 11 | F | No intervention | 35 | 50 | 168 | 17.72 | Never | Vegetarian |
| 12 | M | No intervention | 47 | 84 | 187 | 24.02 | Never | Vegetarian |
| 13 | F | No intervention | 23 | 60 | 170 | 20.76 | Never | Vegan |
| 14 | F | No intervention | 25 | 37 | 149 | 16.67 | Never | Vegan |
| 15 | M | No intervention | 35 | 77 | 172 | 26.03 | Present | Vegetarian |
| 16 | M | No intervention | 65 | 80 | 176 | 25.83 | Never | Omnivore |
| 17 | F | No intervention | 64 | 67 | 164 | 24.91 | Past | Omnivore |
| 18 | M | No intervention | 43 | 69 | 176 | 22.28 | Past | Omnivore |
| 19 | M | No intervention | 39 | 62 | 180 | 19.14 | Never | Omnivore |
| 20 | M | No intervention | 29 | 67 | 190 | 18.56 | Never | Omnivore |
| 21 | F | No intervention | 40 | 49.5 | 158 | 19.83 | Never | Omnivore |
| 22 | | No intervention | 32 | 70 | 162 | 26.67 | Never | Vegetarian |
| 23 | | No intervention | 35 | 78 | 175 | 25.47 | Never | Omnivore |
| 24 | | No intervention | 65 | 82 | 167 | 29.40 | Never | Omnivore |
| 25 | | No intervention | 40 | 49.5 | 158 | 19.83 | Never | Omnivore |
| 26 | M | Probiotics | 29 | 55 | 168 | 19.49 | Never | Omnivore |
| 27 | M | Probiotics | 27 | 71 | 179 | 22.16 | Past | Omnivore |
| 28 | F | Probiotics | 32 | 70 | 177 | 22.34 | Never | Vegan |
| 29 | M | Probiotics | 28 | 71 | 174 | 23.45 | Present | Omnivore |
| 30 | F | Probiotics | 25 | 59 | 170 | 20.42 | Never | Vegan |
| 31 | F | Probiotics | 27 | 58 | 170 | 20.07 | Never | Omnivore |
| 32 | | Probiotics | 26 | 80 | 183 | 23.89 | Present | Omnivore |
| 33 | | Probiotics | | 60 | 173 | 20.05 | Never | Omnivore |
| 34 | M | aFMT | 28 | 63 | 175 | 20.57 | Past | Omnivore |
| 35 | F | aFMT | 46 | 78 | 158 | 31.24 | Past | Omnivore |
| 36 | F | aFMT | 46 | 59 | 159 | 23.34 | Never | Omnivore |
| 37 | F | aFMT | 32 | 85 | 175 | 27.76 | Present | Omnivore |
| 38 | M | aFMT | 31 | 62 | 172 | 20.96 | Never | Omnivore |
| 39 | M | aFMT | 30 | 73 | 169 | 25.56 | Past | Omnivore |
| 40 | M | Spontaneous | 41 | 74 | 175 | 24.16 | Never | Vegetarian |
| | | recovery | | | | | | |
| 41 | M | Spontaneous | 45 | 80 | 180 | 24.69 | Past | Omnivore |
| | | recovery | | | | | | |
| 42 | M | Spontaneous | 40 | 82 | 183 | 24.49 | Past | Omnivore |
| | | recovery | | | | | | |
| 43 | M | Spontaneous | 30 | 66 | 170 | 22.84 | Past | Omnivore |
| | | recovery | | | | | | |
| 44 | M | Spontaneous | 36 | 73 | 167 | 26.18 | Never | Omnivore |
| | | recovery | | | | | | |
| 45 | F | Spontaneous | 25 | 53 | 163 | 19.95 | Never | Omnivore |
| | | recovery | | | | | | |
| 46 | M | Spontaneous | 35 | 78 | 180 | 24.07 | Never | Omnivore |
| | | recovery | | | | | | |

Human Study Design: Forty-six healthy volunteers were recruited for this study between the years 2014 and 2018. Upon enrollment, participants were required to fill up medical, lifestyle and food frequency questionnaires, which were reviewed by medical doctors before the acceptance to participate in the study. Two cohorts were recruited, a naive cohort (n=25) and an antibiotics-treated cohort (n=21), subdivided into 3 interventions of probiotics (n=8), autologous fecal microbiome transplantation (aFMT, n=6) and spontaneous reconstitution (n=7). For the latter, the study design consisted of four phases, baseline (7 days), antibiotics (7 days), intervention (28 days) and follow-up (28 days). During the 4-week intervention phase (days 1 thru 28), participants from the probiotics arm were instructed to consume a commercial probiotic supplement (Bio-25) bidaily; participants from the aFMT arm received an intraduodenal infusion of processed microbiome (on day 0), which had been obtained prior to the antibiotic therapy; and participants from the spontaneous reconstitution group did not undergo any treatment. Stool samples were collected daily during the baseline and antibiotics phases, daily during the first week of intervention and then weekly throughout the rest of the intervention and follow-up phases. Participants in the antibiotics cohort underwent two endoscopic examinations, one at the end of the antibiotics phase (day 0) and another three weeks through the intervention phase (day 21). Participants in the naive cohort underwent a single endoscopic examination, and ten of which collected daily stool samples on the seven days prior to the endoscopy.

The trial was completed as planned. All 46 subjects completed the trial and there were no dropouts or withdrawals. Adverse effects were mild and did not tamper with the study protocol. They included weakness, headaches, abdominal discomfort, anorexia, regurgitation, nausea and oral thrush during the administration of antibiotics and a minor corneal laceration during the endoscopic procedure.

All participants received payment for their participation in the study upon discharge from their last endoscopic session.

Drugs and Biological Preparations

Antibiotics: During the antibiotics phase participants were required to consume oral ciprofloxacin 500 mg bidaily and oral metronidazole 500 mg tridaily for a period of 7 days. This is a broad-spectrum antibiotic regimen is commonly prescribed for treatment of gastrointestinal infections and inflammatory bowel disease exacerbation.

Probiotics: During the probiotics phase participants were treated by oral Supherb Bio-25 twice daily, which is described by the manufacturer to contain at least 25 billion active bacteria of the following species: B. bifidum, L. rhamnosus, L. lactis, L. casei subsp. casei, B. breve, S. thermophilus, B. longum subsp. longum, L. casei subsp. paracasei, L. plantarum and B. longum subsp. infantis. According to the manufacturer, the pills underwent double coating to ensure their survival under stomach acidity condition and their proliferation in the intestines. Validation of the aforementioned species quantity and viability was performed as part of the study (story 1 ref).

Autologous fecal microbiome transplantation (FMT): Participants assigned to the FMT study arm were requested to attend the bacteriotherapy unit of TASMC and deposit a fresh stool sample of at least 350 g. Sample promptly underwent embedding in glycerol, homogenization, filtering and was transferred to storage at −80° C. Sample was thawed 30 minutes prior to the endoscopic procedure and placed in syringes. A volume of 150 ml of the preparation was given as an intraduodenal infusion at the end of the first (post-antibiotics) endoscopic examination. The average fecal content was 70.02±22.28 gr per 150 ml suspension.

Gut Microbiome Sampling

Stool sampling: Participants were requested to self-sample their stool on pre-determined intervals (as previously described) using a swab following detailed printed instructions. Collected samples were immediately stored in a home freezer (−20° C.) for no more than 7 days and transferred in a provided cooler to our facilities, where they were stored at −80° C.

Endoscopic examination: Forty-eight hours prior to the endoscopic examination, participants were asked to follow a pre-endoscopy diet. 20 hours prior to the examination diet was restricted to clear liquids. All participants underwent a sodium picosulfate (Pico Salax)-based bowel preparation. Participants were equipped with two fleet enemas, which they were advised to use in case of unclear stools. The examination was performed using a Pentax 90i endoscope (Pentax Medical) under light sedation with propofol-midazolam.

Luminal content was aspirated from the stomach, duodenum, jejunum, terminal ileum, cecum and descending colon into 15 ml tubes by the endoscope suction apparatus and placed immediately liquid nitrogen. Brush cytology (US Endoscopy) was used to scrape the gut lining to obtain mucosal content from the gastric fundus, gastric antrum, duodenal bulb, jejunum, terminal ileum, cecum, ascending colon, transverse colon, descending colon, sigmoid colon and rectum. Brushes were placed in a screw cap micro tube and were immediately stored in liquid nitrogen. Biopsies from the gut epithelium were obtained from the stomach, duodenum, jejunum, terminal ileum, cecum and descending colon and were immediately stored in liquid nitrogen. By the end of each session, all samples were transferred to Weizmann Institute of Science and stored in −80° C. In the two endoscopic examinations arm the endoscopies were scheduled in sessions 3 weeks apart

Mouse study design: C57BL/6 male mice were purchased from Harlan Envigo and allowed to acclimatize to the animal facility environment for 2 weeks before used for experimentation. Germ-free Swiss-Webster mice were born in the Weizmann Institute germ-free facility, kept in gnotobiotic isolators and routinely monitored for sterility. In all experiments, age- and gender-matched mice were used. Every experimental group consisted of two cages per group (N=5 in each cage). Mice were 8-9 weeks of age and weighed 20 gr at average at the beginning of experiments. All mice were kept at a strict 24 hr light-dark cycle, with lights turned on from 6 am to 6 pm. Each experimental group consisted of two cages to control for cage effect. For antibiotic treatment, mice were given a combination of ciprofloxacin (0.2 g/l) and metronidazole (1 g/l) in their drinking water for two weeks as previously described⁷⁵. Both antibiotics were obtained from Sigma Aldrich. For probiotics consumption, a single pill (Supherb Bio-25) was dissolved in 10 mL of sterile PBS and immediately fed to mice by oral gavage during the dark phase. For auto-FMT, fecal pellets were collected prior to antibiotics administration and snap-frozen in liquid nitrogen; during the day of FMT, the pellets from each mouse were separately resuspended in sterile PBS under anaerobic conditions (Coy Laboratory Products, 75% N2, 20% CO2, 5% H2), vortexed for 3 minutes and allowed to settle by gravity for 2 min. Samples were immediately transferred to the animal facility in Hungate anaerobic culture tubes and the supernatant was administered to the mice by oral gavage. Stool was collected on pre-determined days at the beginning of the dark phase, and immediately snap-frozen and transferred for storage at −80° C. until further processing. Upon the termination of experiments, mice were sacrificed by CO2 asphyxiation, and laparotomy was performed by employing a vertical midline incision. After the exposure and removal of the digestive tract, it was dissected into eight parts: the stomach; beginning at the pylorus, the proximal 4 cm of the small intestine was collected as the duodenum; the following third of the small intestine was collected as the proximal and distal jejunum; the ileum was harvested as the distal third of the small intestine; the cecum; lastly, the colon was divided into its proximal and distal parts. For each section, the content within the cavity was extracted and collected for luminal microbiome isolation, and the remaining tissue was rinsed three times with sterile PBS and collected for mucosal microbiome isolation. During each time point, each group was handled by a different researcher in one biological hood to minimize cross-contamination. All animal studies were approved by the Weizmann Institute of Science Institutional Animal Care and Use committee (IACUC), application number 29530816-2.

Bacterial cultures: Bacterial strains used in this study are listed in Table 1 herein above. For culturing of bacteria from the probiotics pill, the following liquid media were used: De Man, Rogosa and Sharpe (MRS), modified reinforced clostridial (RC), M17, Brain-Heart Infusion (BHI), or chopped meat carbohydrate medium (CM). All growth media were purchased from BD. Cultures were grown under anaerobic conditions (Coy Laboratory Products, 75% N2, 20% CO2, 5% H2) in 37° C. without shaking. For fecal microbiome cultures, ˜200 mg of frozen human feces was vortexed in 5 ml of BHI under anaerobic conditions. 200 ul of the supernatant were transferred to fresh 5 mL of BHI for initiation of growth. Stationary phase probiotics cultures were filtered using a 0.22 uM filter and added to the fecal culture. For pure Lactobacillus cultures, each strain was grown in liquid MRS under anaerobic conditions.

Nucleic Acid Extraction

DNA purification: DNA was isolated from endoscopic samples, both luminal content and mucosal brushes, using PowerSoil DNA Isolation Kit (MOBIO Laboratories). DNA was isolated from stool swabs using PowerSoil DNA Isolation Kit (MOBIO Laboratories) optimized for an automated platform.

RNA Purification: Gastrointestinal biopsies obtained from the participants were purified using RNAeasy kit (Qiagen, 74104) according to the manufacturer's instructions. Most of the biopsies were kept in RNAlater solution (ThermoFisher, AM7020) and were immediately frozen at liquid nitrogen.

Nucleic Acid Processing and Library Preparation

16S qPCR Protocol for Quantification of Bacterial DNA: DNA templates were diluted to 1 ng/ul before amplifications with the primer sets (indicated in Table 3) using the Fast Sybr™ Green Master Mix (ThermoFisher) in duplicates. Amplification conditions were: Denaturation 95° C. for 3 minutes, followed by 40 cycles of Denaturation 95° C. for 3 seconds; annealing 64° C. for 30 seconds followed by meting curve. Duplicates with >2 cycle difference were excluded from analysis. The CT value for any sample not amplified after 40 cycles was defined as 40 (threshold of detection).

16S rDNA Sequencing—as in Example 1.

Whole genome shotgun sequencing: 100 ng of purified DNA was sheared with a Covaris E220X sonicator. Illumina compatible libraries were prepared as described⁷⁵, and sequenced on the Illumina NextSeq platform with a read length of 80 bp to a depth of XXX±XXX reads (mean±SD).

RNA-Seq

Ribosomal RNA was selectively depleted by RnaseH (New England Biolabs, M0297) according to a modified version of a published method⁷⁶. Specifically, a pool of 50 bp DNA oligos (25 nM, IDT, indicated in Table 4) that is complementary to murine rRNA18S and 28S, was resuspended in 75 μl of 10 mM Tris pH 8.0. Total RNA (100-1000 ng in 10 μl H₂O) were mixed with an equal amount of rRNA oligo pool, diluted to 2 μl and 3 μl 5×rRNA hybridization buffer (0.5 M Tris-HCl, 1 M NaCl, titrated with HCl to pH 7.4) was added. Samples were incubated at 95° C. for 2 minutes, then the temperature was slowly decreased (−0.1° C./s) to 37° C. RNAseH enzyme mix (2 μl of 10 U RNAseH, 2 μl 10×RNAseH buffer, 1 μl H₂O, total 5 μl mix) was prepared 5 minutes before the end of the hybridization and preheated to 37° C. The enzyme mix was added to the samples when they reached 37° C. and they were incubated at this temperature for 30 minutes. Samples were purified with 2.2×SPRI beads (Ampure XP, Beckmann Coulter) according to the manufacturers' instructions. Residual oligos were removed with DNAse treatment (ThermoFisher Scientific, AM2238) by incubation with 5 μl DNAse reaction mix (1 μl Trubo DNAse, 2.5 μl Turbo DNAse 10× buffer, 1.5 μl H₂O) that was incubated at 37° C. for 30 minutes. Samples were again purified with 2.2×SPRI beads and suspended in 3.6 μl priming mix (0.3 μl random primers of New England Biolab, E7420, 3.3 μl H₂O). Samples were subsequently primed at 65° C. for 5 minutes. Samples were then transferred to ice and 2 μl of the first strand mix was added (1 μl 5× first strand buffer, NEB E7420; 0.125 μl RNAse inhibitor, NEB E7420; 0.25 μl ProtoScript II reverse transcriptase, NEB E7420; and 0.625 μl of 0.2 μl /ml Actinomycin D, Sigma, A1410). The first strand synthesis and all subsequent library preparation steps were performed using NEBNext Ultra Directional RNA Library Prep Kit for Illumina (NEB, E7420) according to the manufacturers' instructions (all reaction volumes reduced to a quarter).

Analyses

16S rDNA analysis: The 2×250 bp reads were processed using the QIIMEapor⁶⁹ (Quantitative Insights Into Microbial Ecology) analysis pipeline. In brief, fasta quality files and a mapping file indicating the barcode sequence corresponding to each sample were used as inputs, paired reads were first assembled into longer reads based on sequence similarity, the assembled reads were then split to samples according to the barcodes, Sequences sharing 97% nucleotide sequence identity in the 16S rRNA region were binned into operational taxonomic units (97% ID OTUs). Each OTU was assigned a taxonomical classification by applying the Uclust algorithm against the Greengenes database, and an OTU table was created.

Metagenomic analysis: Data from the sequencer was converted to fastq files with bcl2fastq. Reads were then QC trimmed using Trimmomatic⁷⁰ with parameters PE -threads 10 -phred33 -validatePairs ILLUMINACLIP:TruSeq3-PE.fa:2:30:10 LEADING:3 TRAILING:3 MINLEN:50. We used MetaPhlAn2⁷¹ for taxonomic analysis with parameters: —ignore_viruses —ignore_archaea —ignore_eukaryotes.

Host sequences were removed by aligning the reads against human genome reference hg19 using bowtie2⁷² with parameters: -D 5 -R 1 -N 0 -L 22 -i 5,0,2.50. The resulting non-host reads were then mapped to the integrated gene catalogue⁷⁷ using bowtie2 with parameters: —local -D 25 -R 3 -N 1 -L 19 -i 5,1,0.25 -k 5 allowing to a single read to match up to five different entries.

Further filtering of the bacterial reads consisted of retaining only records with minimal base quality of 26. The bacterial quality filtered resulting bam files were then subsampled to 10⁵ bacterial hits. An entry's score was defined by its length, divided by the gene length. Entries scores were summarized according to KO annotations⁷⁸. Each sample was scaled to 1M. KEGG Pathway analysis was conducted using EMPANADA⁷³.

Probiotics strain identification by unique genomic sequences: Recovery of genomes for probiotic strains from pill metagenomics samples: Genomes for 9 of the 11 probiotic strains were recovered at >93% completeness and <4% contamination from metagenomics samples of the probiotics pill (Table 7). For one of the species (B. longum) only part of the genome was recovered due to strain heterogeneity. The samples were assembled in multiple cycles using IDBA-UD⁷⁹. Assemblies were manually improved using a mini-assembly approach⁵¹. Genomes were recovered based on similarity to reference genomes and connectivity between scaffolds as deduced from the mini-assembly analysis.

TABLE 7
statics for genomes recovered from metagenomics samples
of probiotics pill used in the study. Completeness and contamination
were evaluated using CheckM⁸⁰.

| | | # Scaf- | Complete- | Contam- |
|---|---|---|---|---|
| Species | Size | folds | ness | ination |
| Bifidobacterium breve | 2,051,417 | 128 | 93.66 | 0.69 |
| Bifidobacterium bifidum | 2,196,275 | 11 | 99.54 | 0.12 |
| Bifidobacterium longum | 1,200,324 | 180 | 46.03 | 0.96 |
| Lactococcus lactis | 2,472,057 | 36 | 100 | 0 |
| Lactobacillus acidophilus | 1,963,581 | 22 | 98.94 | 0 |
| Lactobacillus casei | 2,968,946 | 33 | 94.64 | 1.72 |
| Lactobacillus paracasei | 3,038,895 | 92 | 98.79 | 3.56 |
| Lactobacillus plantarum | 3,299,766 | 31 | 99.38 | 2.79 |
| Lactobacillus rhamnosus | 2,921,071 | 29 | 99.02 | 0 |
| Streptococcus thermophilus | 1,789,952 | 74 | 99.89 | 0.29 |

Strain-Level Analysis Probiotic Strains in Human Samples.

Identifying reads that belong to the probiotic strains in each sample: All human reads were first removed from all samples by mapping against the human genome (assembly GRCh38.p7) using bowtie2 with the -very_sensitive flag. Next, the non-human reads were mapped against all probiotics genomes recovered from the pill using bowtie2 to identify reads that potentially belong to these strains. Finally, the reads were mapped against a database of genomes for all species in the orders Lactobacillales and Bifidobacteriales to which the probiotic strains belong, including the probiotic genomes. Only reads that received their best hit from one of the probiotics strains were further analyzed.

Determining presence of probiotic species: we counted the number of genes in each probiotic genome whose coverage is greater than 0. A probiotic species was determined to be present in a sample if at least 400 of its genes were detected, with the threshold being set based on comparison to MetaPhlAn2 results and an analysis of gene number distribution across the different samples.

Determining strain-specific genes: we clustered each probiotic genome's proteins with other genomes available for the species using USEARCH⁸¹ with 90% identity threshold. All genes in clusters whose size was <10% of the number of genomes analyzed were determined to be strain specific. The analysis could be applied to the genomes of B. bifidum, B. breve, B. longum, L. acidophilus, L. casei, L. lactis, L. paracasei, L. plantarum and S. thermophilus. For B. longum, it is not possible to determine which of the probiotic strains is present.

Determining samples with probiotic strains: For each strain that passed the 400-genes threshold from step 3 we compared the fraction of strain-specific genes detected with the fraction of all genes on the genome that were detected. The probiotic strain was determined to be present if at least 65% of the total number of genes were detected and the difference between the fraction of the total and strain-specific genes that were found was 20% or less.

RNAseq Analysis

Data normalization: Initially, we normalized the sequenced data as previously described⁸². Briefly, genes with mean TPM<1 across all samples were filtered out from the analysis, and a value of 0.001 was added to remaining TPM values to avoid zero-values in downstream calculations. Then, sample median normalization was performed based on all constitutive gene reads with positive counts for all samples. Thus, all TPM values in each sample were scaled by the median TPM of constitutive reads in that sample, divided by the median TPM across all samples. We then performed a per-gene normalization by dividing each expression value by the median value of that gene across all samples. Finally, expression data was log-transformed (base 2). The above normalization steps were performed separately to data acquired from each of the different experimental batches, determined by the presence or absence of RNAlater solution for sample preservation.

Comparison of expression levels before and after treatment with probiotics: To account for inter-personal differences and reduce noise, we compared the effects of probiotics treatment on host expression patterns using a repeated-measures design. Thus, for each individual, in each biopsy region, the relative fold-changes (log, base 2) in expression levels of each gene were calculated between samples taken at baseline and after treatment with probiotics. Then, for each individual, genes were ranked from low to high, and sorted by their median rank across all available samples. These sorted lists were subsequently used for gene ontology (GO) enrichment analysis using GOrilla with a p-value threshold of 10⁻³ and a false-discovery rate (FDR) threshold of q<0.05.

Quantification and statistical analysis: The following statistical analyses were applied unless specifically stated otherwise: For 16S data, rare OTUs (<0.1% in relative abundance) were filtered out, and samples were then rarefied to a depth of 10,000 reads (5000 in mouse tissues). For metagenomic data, samples with <10⁵ assigned bacterial reads (after host removal) were excluded from further analysis. In the remaining samples, rare KEGG orthologous (KO) genes (<10-5) were removed. Beta diversity was calculated on OTUs (16S) or species (metagenomics) relative abundances using UniFrac distances or Bray-Curtis dissimilarity (R Vegan package, www(dot)CRAN.R-project(dot)org/package=vegan) respectively. PCA for KOs and functional bacterial pathways were calculated using Spearman's rank correlation coefficient. Alpha diversity was calculated on OTUs (16S) using the observed species index. For 16S data, measurements of alpha and beta diversity were calculated using QIIME tools v 1.9.1. In order to determine the effect of treatment on microbiota taxonomic composition and functional capacity repeated-measures Kruskal Wallis with Dunn's test was used. In order to compare the effect of treatment over time between two groups or more two-way ANOVA with Dunnet's test, or permutation tests performed by switching labels between participants, including all their assigned samples, were used. Mann-Whitney and Wilcoxon tests were used to conduct pairwise comparisons between two treatment arms or two groups of participants. Permutational multivariate ANOVA (Adonis PERMANOVA with 10,000 permutations) based on sample distances was used to test for changes in the community composition and function. To analyze qPCR data, Two way ANOVA with Sidak or Dunnett test was used. The threshold of significance was determined to be 0.05 both for p and q-values. Statistically significant findings were marked according to the following cutoffs: *, P<0.05; **, P<0.01; ***, P<0.001; ****, P<0.0001. Data were plotted with GraphPad Prism version 7.0c. Statistical details for all experiments, including sample size, the statistical test used, dispersion and precision measures and statistical significance, are specified in the result section and denoted in figure legends.

### Results

### Experimental Setup in Mice

Under homeostatic conditions (see Example 1), administration of a multi-strain probiotic preparation was associated with limited colonization in mice, and with person-specific gut mucosal colonization resistance in humans. To study gut mucosal colonization and resistance patterns to probiotics under microbiome-perturbing conditions, we chose the antibiotic treatment setting, in which probiotics are commonly recommended as means of preventing or ameliorating antibiotics-associated adverse effects³¹. In this setting, antibiotics are postulated to provide a ‘freed niche’ potentially enabling probiotics to serve as ‘place holders’ in counteracting antibiotics-induced adverse effects on the indigenous microbiome and mammalian host. However, neither the probiotic mucosal colonization capacity in this context, nor their impact on reconstitution of the indigenous gut mucosal microbiome, have been globally and directly explored to date.

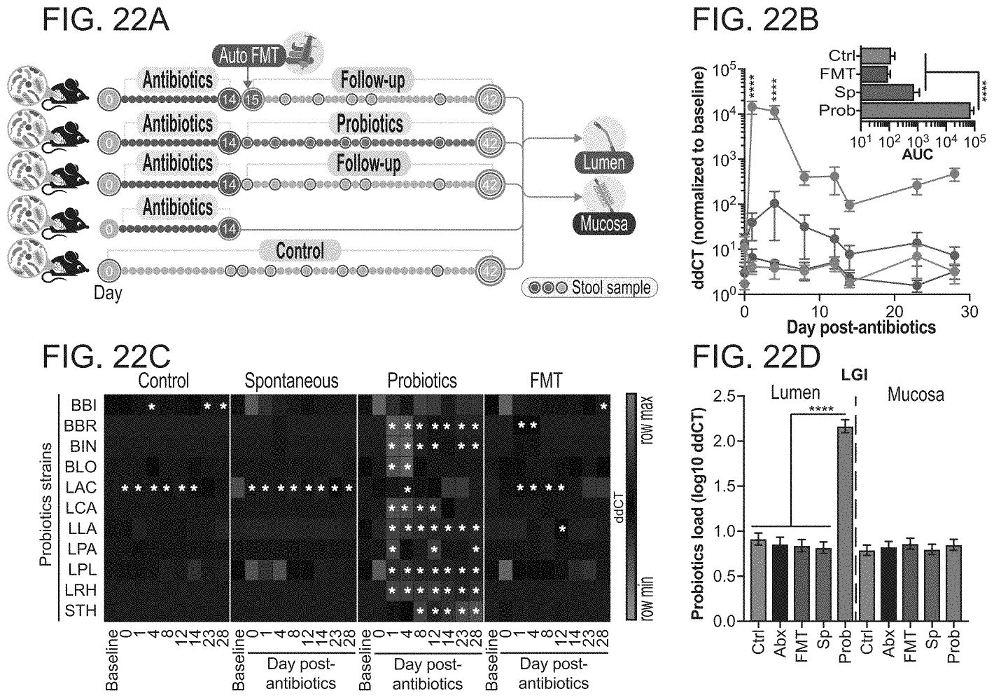

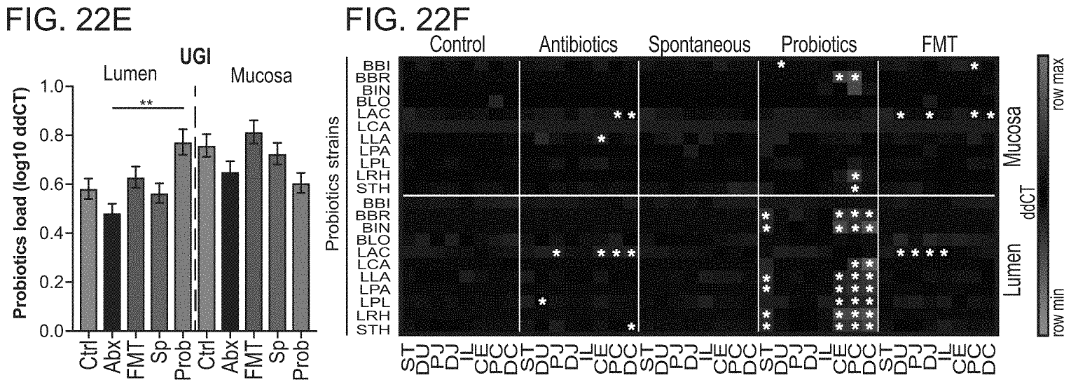

To study the mucosal colonization capacity of probiotics, and their impact on the indigenous mucosal microbiome as compared to aFMT or watchful waiting, we supplemented the drinking water of male adult WT C57Bl/6 mice (N=40) with a wide-spectrum antibiotics regimen of ciprofloxacin and metronidazole for two weeks. The immediate impact of antibiotic treatment on gut mucosal microbiome configuration was assessed in one group of mice, sacrificed after the two-week antibiotic exposure (“antibiotics”, N=10, FIG. 22A). The remaining animals (N=30) were divided into three post-antibiotic intervention groups. In the first group (“probiotics”, N=10), antibiotic treatment was followed by 4 weeks of daily administration by oral gavage of a commonly prescribed probiotics product sold for human use, including the following 11 strains: Lactobacillus acidophilus (abbreviated henceforth as LAC), Lactobacillus casei (LCA), Lactobacillus casei subsp. paracasei (LPA), Lactobacillus plantarum (LPL), Lactobacillus rhamnosus (LRH), Bifidobacterium longum (BLO), Bifidobacterium bifidum (BBI), Bifidobacterium breve (BBR), Bifidobacterium longum subsp. infantis (BIN), Lactococcus lactis (LLA) and Streptococcus thermophilus (STH). These probiotic strains were validated for composition and viability by multiple methods (see Example 1). Each mouse of the second group (“aFMT”, N=10) received, on the day following cessation of antibiotics, an oral gavage of its own pre-antibiotics stool microbiome. A third group (“watchful waiting”, N=10) remained untreated following antibiotic therapy to assess the spontaneous recovery of the indigenous gut microbiome in this setting. An additional group of mice (“control”, N=10) did not receive antibiotics or any other treatment and was followed throughout the study's duration. Stool samples were collected from all groups at the indicated time-points (FIG. 22A) before and during 4 weeks following antibiotics treatment, after which multiple lumen and mucosa samples were harvested from throughout the GI tract.

### Antibiotic Treatment Marginally Enhances Probiotic Gut Mucosal Colonization in Mice

We began our investigation by assessing the fecal and mucosal colonization of probiotics following wide-spectrum antibiotic treatment in mice. 16S rDNA rarefied to 10000 reads indicated three of the four genera comprising the probiotics mix to be present in stool samples even prior to antibiotic administration (Lactobacillus, Bifidobacterium and Streptococcus, FIGS. 29A-C). Following antibiotics treatment and even prior to probiotics administration, a significant increase in relative abundance was observed for the Lactobacillus (0.3-0.55 increase in relative abundance, Two-Way ANOVA & Dunnett P=0.0001, FIG. 29A), Bifidobacterium (P=0.0001, FIG. 29B), and Lactococcus genera (P=0.035, FIG. 29C). The Bifidobacterium and Lactococcus genera increased one day following probiotics administration (Two-Way ANOVA & Tukey P<0.001 vs. each group, FIGS. 29B, 29D), and Bifidobacterium remained elevated also on day 4, after which none of the genera were significantly higher in the treated group. Given the inability of 16s rDNA analysis to distinguish absolute abundance changes at the species level, we utilized a sensitive species-specific qPCR (see Example 1) targeting each of the 11-probiotic species. A pooled qPCR analysis for all species in stool indicated >10000-fold fecal enrichment of probiotic species on days one and four of probiotic supplementation (Two-Way ANOVA & Tukey P<0.0001 vs. each group, FIG. 22B), which rapidly declined in the following days losing statistical significance, though the trend persisted throughout the experiment (iAUC Kruskal-Wallis & Dunn's P<0.0001 vs. each group, FIG. 22B). A per-species analysis indicated nine of the 11 species (all but BBI and LAC) to be significantly enriched in stool during probiotics supplementation (average fold expansion 4846, range 0.03-230805, Two-Way ANOVA & Dunnett P<0.05, FIG. 22C).

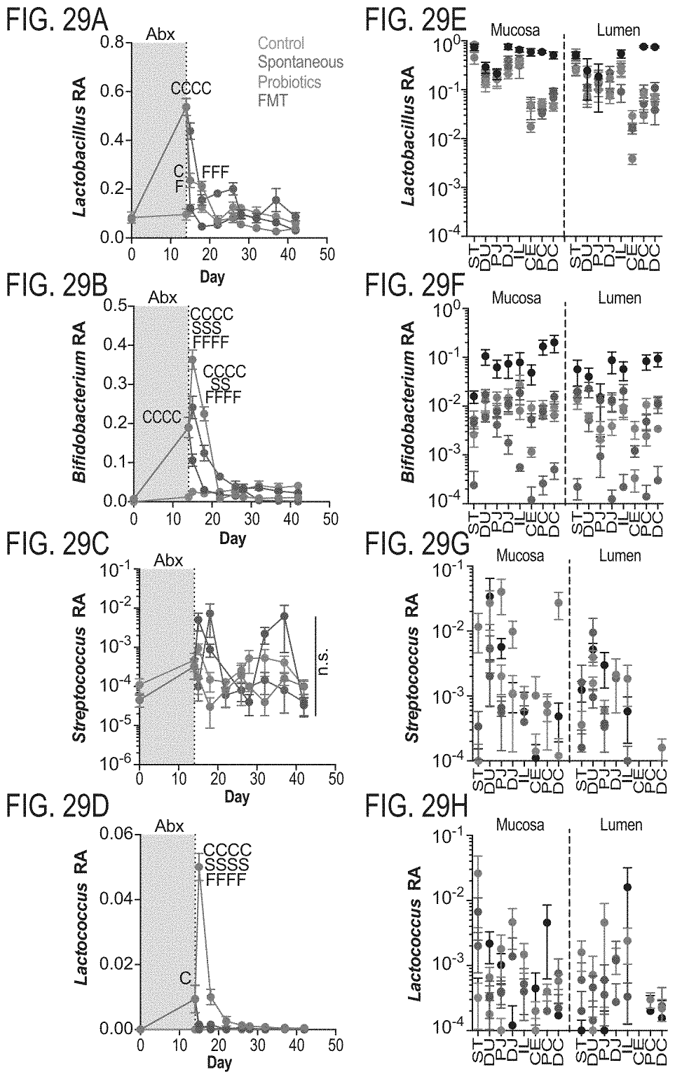

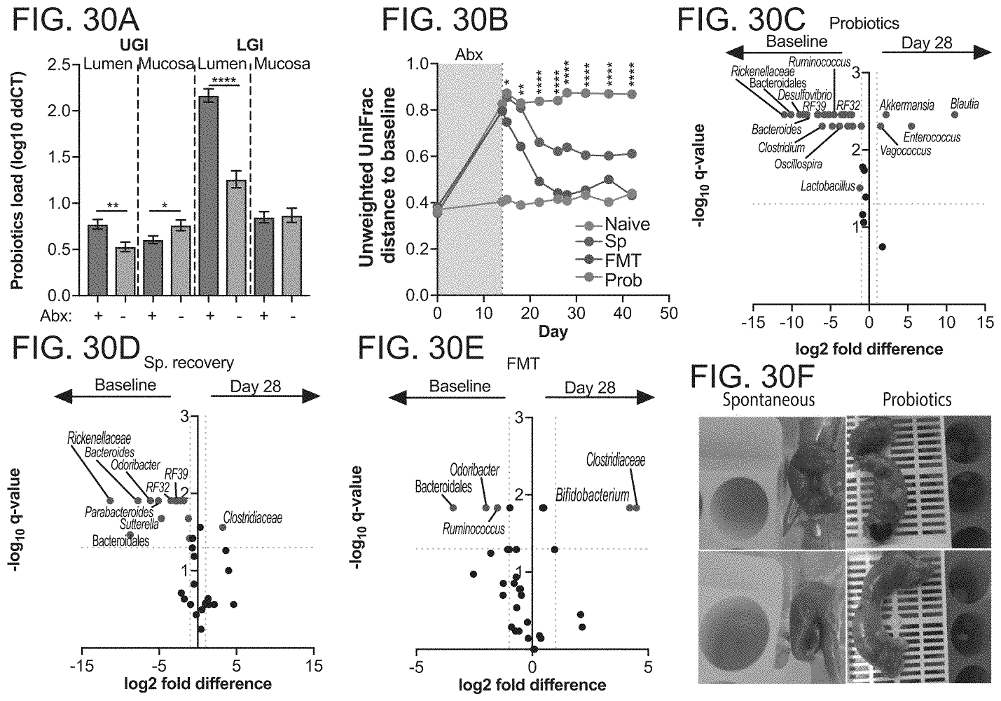

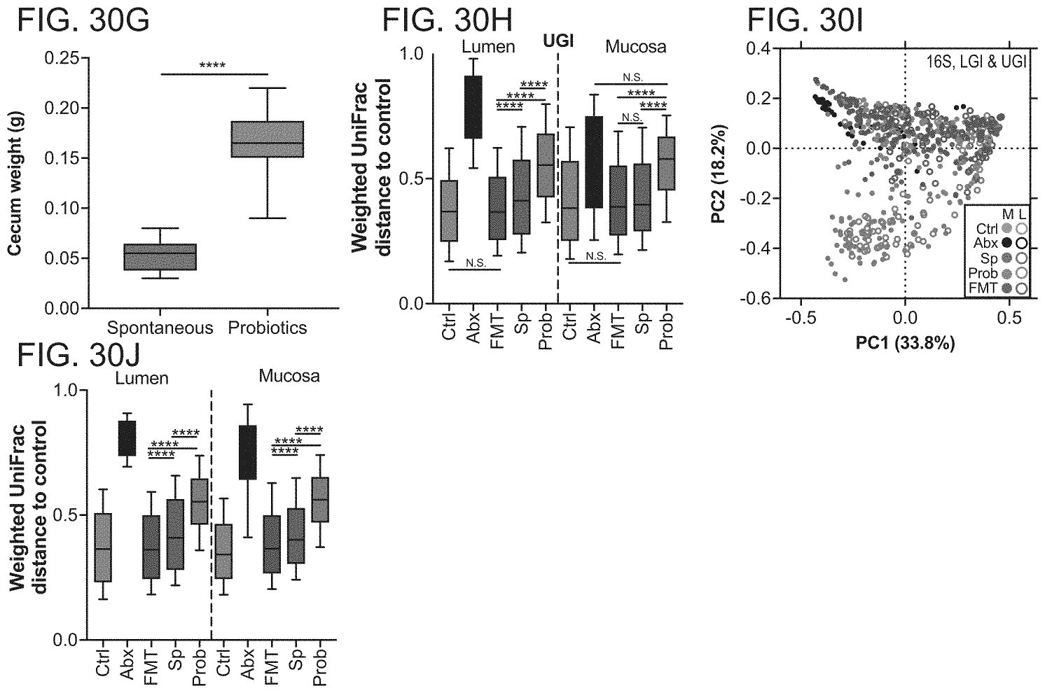

Like in stool, 16S-rDNA assessment of mucosal colonization did not detect significant elevation in the relative abundance of any of the probiotics genera in any of the regions (FIGS. 29E-H). A pooled qPCR analysis for all administered probiotic species indicated significantly higher abundance in the lumen of the LGI (Kruskal-Wallis & Dunn's P<0.0001 vs. each group, FIG. 22D), but not the LGI mucosa (P>0.999, FIG. 22D) or the UGI (P>0.09 for all except P=0.004 vs. antibiotics in the lumen, FIG. 22E). The species significantly elevated in the lumen of the LGI tissues and the stomach were consistent with those shed in stool, while only BBR, LRH and STH were significantly elevated in the LGI mucosa (FIG. 22F). In a separate cohort of mice that received probiotics using the same experimental design but without antibiotics pretreatment, the measured aggregated probiotics load from all targets in the GI lumen, but not the GI mucosa, was significantly lower (UGI Mann-Whitney P=0.0095, LGI P<0.0001, FIG. 30A). These results indicate resistance to the presence of probiotic species in the GI lumen conferred by the resident microbiome. This resistance is partially alleviated by antibiotics, although even after antibiotics pretreatment probiotics demonstrated mild and sporadic mucosal presence, potentially reflecting lower colonization capacity of ‘human compatible’ probiotics species in the murine gut mucosa.

### Probiotics Delay, and aFMT Improves the Post-Antibiotic Reconstitution of the Indigenous Murine Microbiome

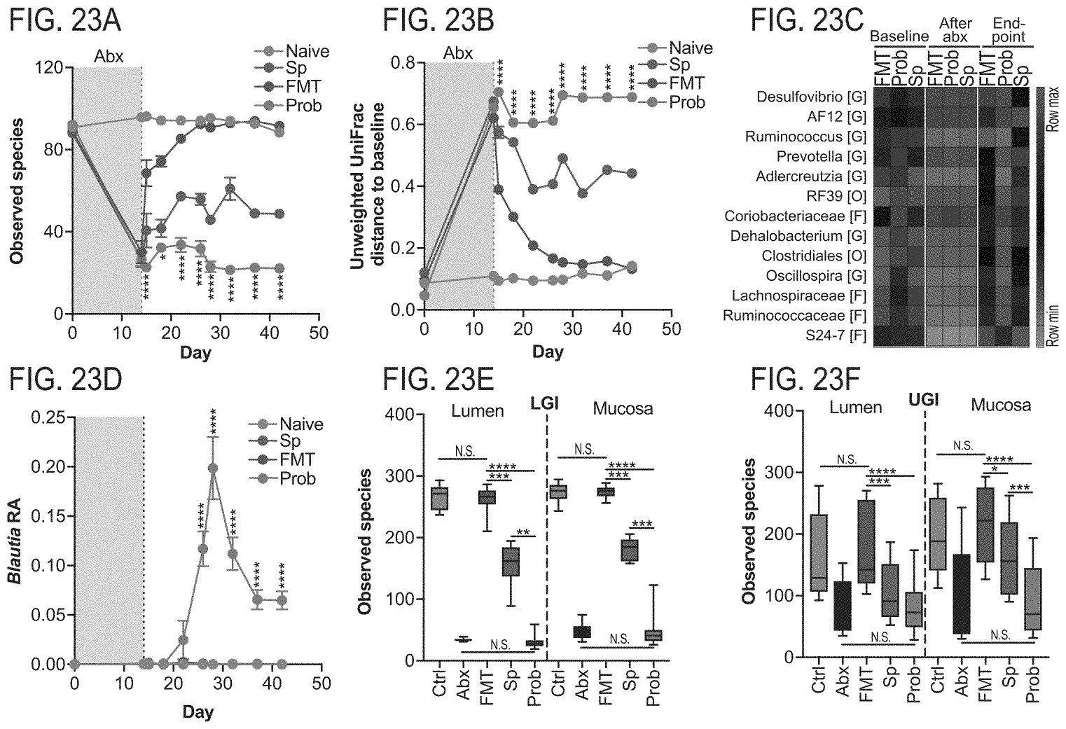

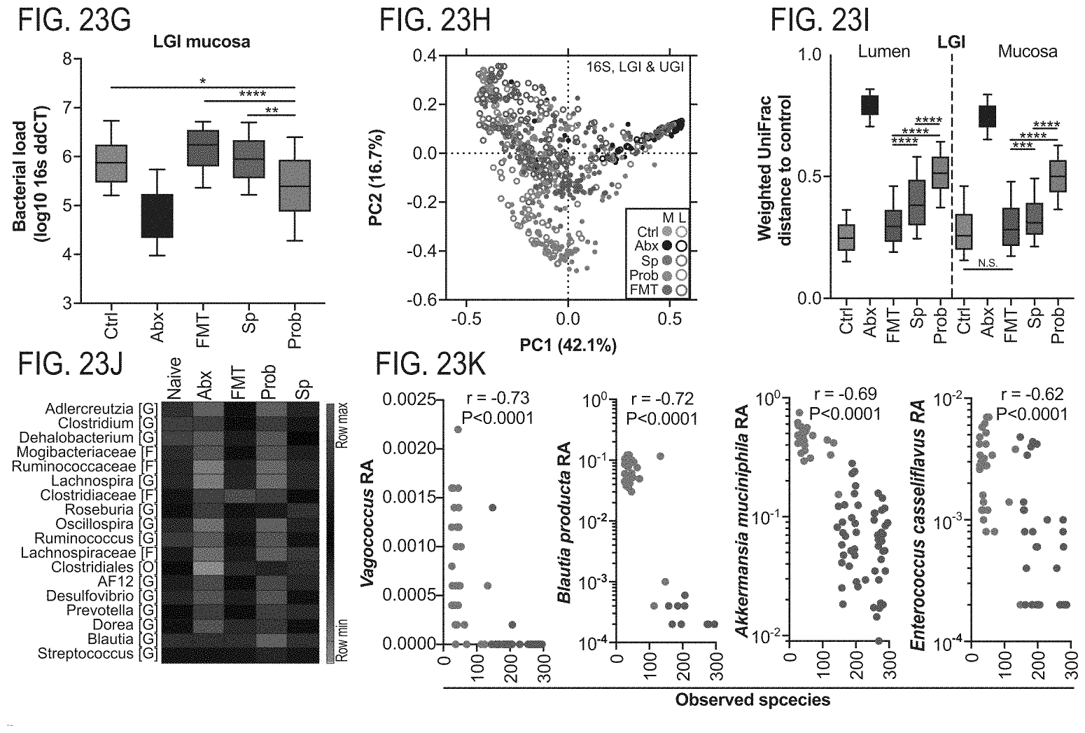

We next determined the impact of probiotics on reconstitution of the indigenous murine fecal and mucosal gut microbiome community following antibiotic treatment. Expectedly, antibiotic treatment resulted in a dramatic reduction in stool alpha-diversity (>66% reduction, Two-Way ANOVA & Dunnett P=0.0001 for all groups, FIG. 23A) and general disruption of the fecal bacterial community structure as evident by unweighted UniFrac distances to baseline (P=0.0001, FIG. 23B). Of the three post-antibiotic interventions, aFMT was most efficient in restoring fecal bacterial richness to that observed in the control, with alpha diversity becoming indistinguishable to control within eight days following aFMT (Two-Way ANOVA & Dunnett P=0.11). In contrast, both probiotics and spontaneous recovery did not restore fecal alpha-diversity to baseline levels 4 weeks following antibiotic cessation (FIG. 22A). Importantly, probiotics significantly delayed the return to baseline microbiome richness even compared to spontaneous recovery as evident in all tested time points (Two-Way ANOVA & Dunnett P=0.0001 in all but day 4 where P=0.04, FIG. 23A).

Delayed murine probiotics-induced microbiome reconstitution was also reflected in the kinetics of return to fecal baseline pre-antibiotics composition, as expressed by UniFrac distances. While all treatment groups were dramatically shifted from baseline stool composition upon antibiotic treatment, aFMT returned to baseline by day 28 (P=0.83, FIG. 23B), while both the probiotics and spontaneous recovery groups failed to fully return to baseline within 4 weeks of antibiotics cessation, with microbiome in the probiotics-administered group featuring the slowest recovery rate (Two-Way ANOVA and Dunnett P=0.0001 for each timepoint vs. each group). As a greater distance to baseline in the probiotics supplemented group may be merely a result of new exogenous bacteria introduced into the microbiome, we repeated the measurement after removing the four probiotics genera from the analysis and renormalizing relative abundances to 1, and corroborated the greater distance to baseline of the probiotics-supplemented group, reflecting an impaired indigenous mucosal microbiome reconstitution in this group (FIG. 30B). A pairwise comparison of fecal microbial composition between the last day of follow-up and baseline demonstrated multiple differentially represented taxa in the probiotics group (28 taxa, Mann-Whitney P<0.05, FIG. 30C), with a >10-fold increase in the abundance of Blautia, and no significant increase in any of the probiotics genera. Fewer significant differences were observed in the spontaneous recovery (16 taxa, FIG. 30D) and aFMT (6 taxa, FIG. 30E) groups. Of all taxa significantly reduced by antibiotics, 13 taxa belonging to 4 different phyla returned to baseline levels in both the aFMT and spontaneous recovery groups, but not in the probiotics group (FIG. 23C). In contrast, 5 taxa were over-represented in the stool samples of the probiotics and significantly inversely correlated with alpha diversity: Akkermansia (Spearman r=−0.62, P<0.0001), Vagococcus (r=−0.61, P<0.0001), Enterococcus (r=−0.49, P<0.0001), Blautia (r=−0.42, P<0.0001) and Lactococcus (r=−0.4, P<0.0001). Of these, only Blautia bloomed exclusively in the probiotics group after antibiotics cessation (Two-Way ANOVA & Dunnett P<0.0001 vs. each group in each time-point from day 12 post-antibiotics, FIG. 23D). Interestingly, macroscopic differences were noted between the ceca of probiotics-administered and spontaneously recovering mice, with the former being larger (representatives in FIG. 30F), and significantly heavier (Mann-Whitney P<0.0001, FIG. 30G), reminiscent of germ-free mice or mice treated with broad-spectrum antibiotics.

Consistent with the findings in stool, the number of observed species in the probiotics group was comparable to the group dissected immediately after two weeks of antibiotics, and significantly lower compared to the control, aFMT, and spontaneous recovery groups in both the lumen and the mucosa of the LGI (FIG. 23E) and UGI (FIG. 23F). There were no significant differences between the aFMT and control groups in any of the regions, whereas spontaneous recovery displayed a configuration in between that of aFMT and probiotics (FIGS. 23E-F). Reduced alpha diversity in the LGI of the probiotics group was at least partly due to a total reduction in LGI bacterial load (FIG. 23G). In agreement, the UniFrac distance to control of the mucosal and lumen aFMT microbiome configuration was lower than that of the spontaneously recovering group, with the largest distance to control featured by the probiotics-administered group (Kruskal-Wallis and Dunn's P<0.0001, FIGS. 23G-I, 30H). As in stool, these colonization differences could not be explained by the mere presence of probiotics genera in probiotics-administered mice, as the result remained unchanged even if probiotics genera were excluded from the analysis (FIGS. 30I-J). Interestingly, microbiome composition of aFMT-treated mice was indistinguishable from controls both in the LGI and the UGI, suggesting that fecal microbiome is sufficient to recapitulate the distinct UGI microbiome (FIG. 30H). Of the taxa significantly reduced in the LGI mucosa of the antibiotics group compared to control, 16 returned to control levels in both the aFMT and the spontaneous recovery groups, but not probiotics, of which 11 belonged to the Clostridiales order; two genera (Blautia and Streptococcus) significantly bloomed exclusively in probiotics (FIG. 23J). Four taxa predominant in the probiotics group had a high (Spearman r<−0.6) and significant (P<0.0001) inverse correlation with the alpha diversity in the LGI mucosa: Vagococcus, Akkermansia muciniphila, Blautia producta and Enterococcus casseliflavus (FIG. 23K). Compared to aFMT, spontaneous recovery failed to restore to control levels 10 OTUs in the LGI mucosa, of which four belonged to Bacteroidales and 3 to Clostridiales.

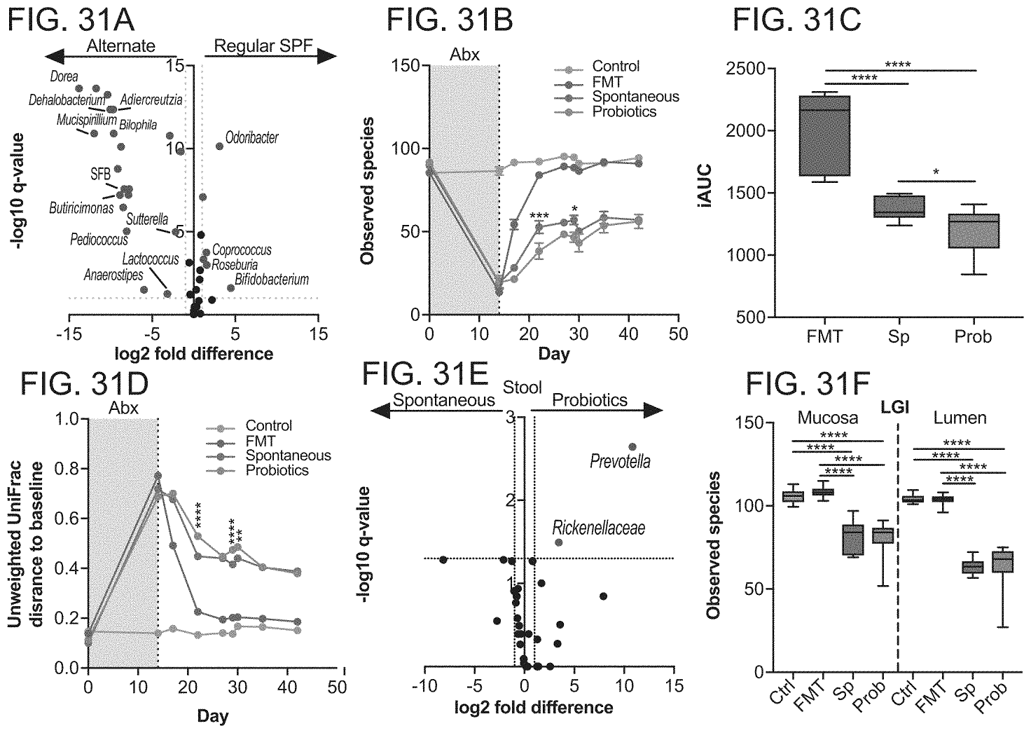

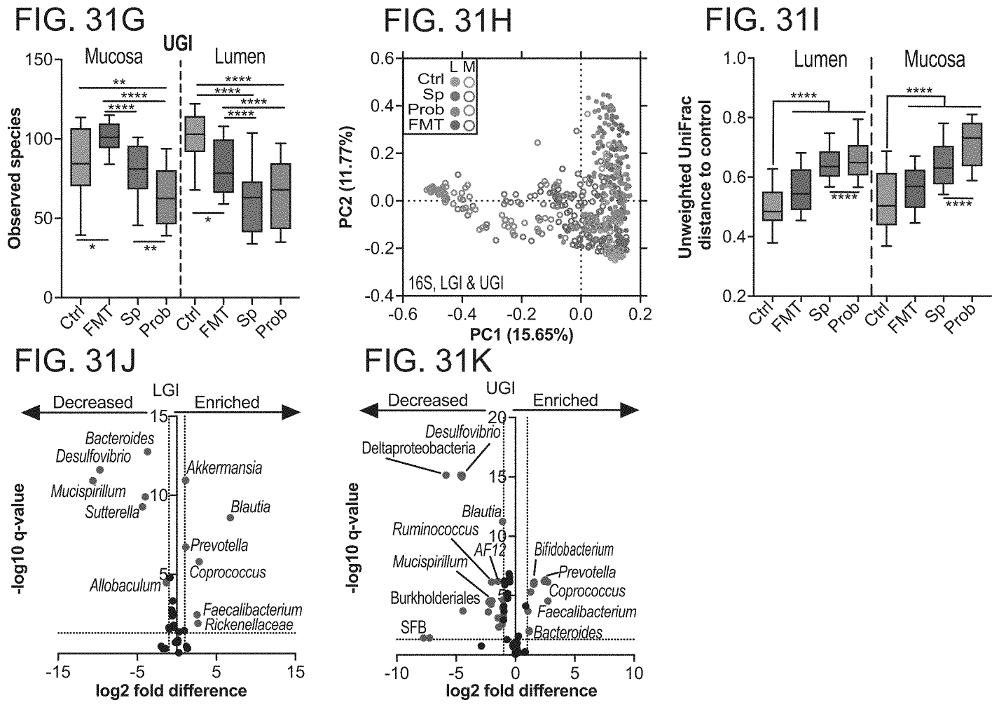

To ascertain that the delayed return to homeostatic indigenous microbiome configuration following probiotics treatment was not a unique feature of the studied vivarium, we performed the same set of interventions on mice housed in a different SPF animal facility with distinct baseline fecal microbiome (26 OTUs significantly differentially represented, FDR-corrected Mann-Whitney P<0.05, FIG. 31A). In this vivarium as well, aFMT induced a rapid indigenous microbiome post-antibiotic reconstitution as compared to watchful waiting, while probiotic treatment delayed the speed and magnitude of the recolonization process (FIGS. 31B-K).

Collectively, four weeks of spontaneous recovery following a wide-spectrum antibiotics treatment in mice partially restored baseline gut mucosal configuration and bacterial richness and load. Watchful waiting was superior, in its rate of induction of indigenous microbiome reconstitution, to consumption of probiotics, which demonstrated little improvement of the post-antibiotics microbiome configuration and delayed the restoration of homeostatic composition and richness of the pre-antibiotic gut mucosal microbiome (FIGS. 23A-K, FIGS. 30A-J, FIGS. 31A-K). In comparison to both watchful waiting and probiotics-administration, aFMT constituted the most efficient treatment modality enabling rapid restoration of both upper and lower homeostatic gut mucosal microbiome configuration post-antibiotic treatment in mice. As lower microbiome diversity is associated with multiple disease states, it will be important to determine the long-term physiological consequences of this persistent probiotics effect.

### Human Experimental Design

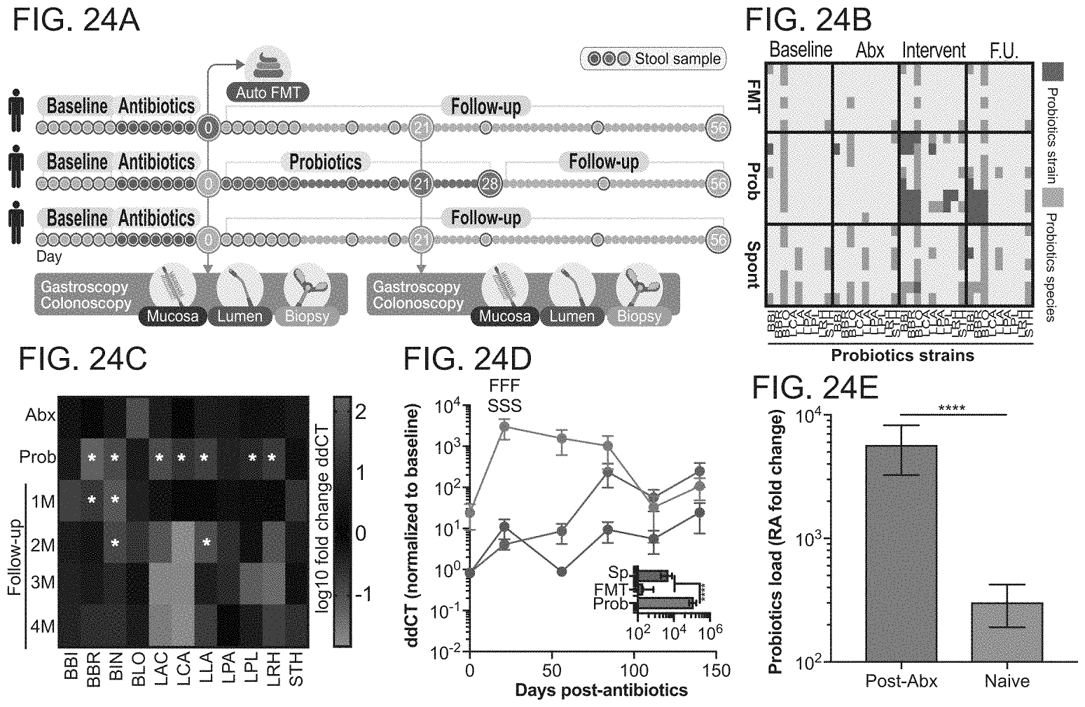

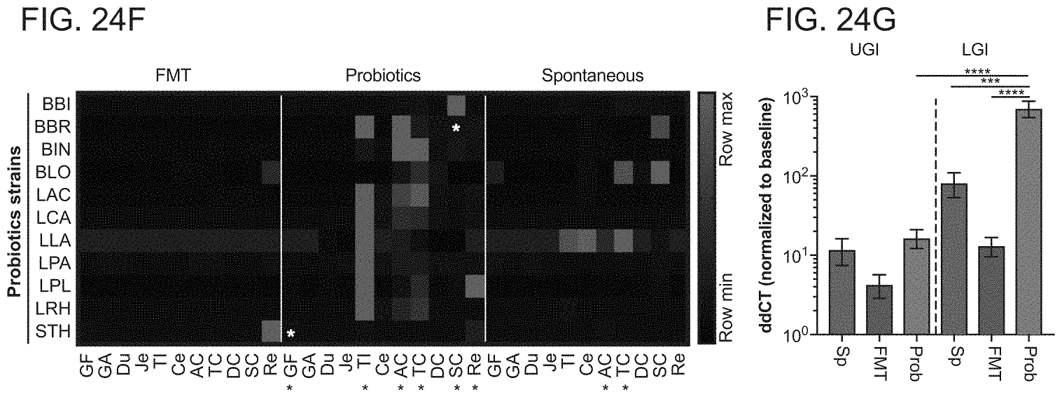

We next set out to determine how post-antibiotic probiotics or aFMT treatment would affect the human luminal and mucosa-associated microbiome reconstitution. To this aim, we conducted a prospective longitudinal interventional study in 21 healthy human volunteers not consuming probiotics (Table 6), who were given an oral broad-spectrum antibiotic treatment of ciprofloxacin and metronidazole at standard dosages for a period of 7 days (days −7 through −1, FIG. 24A). Following antibiotic treatment, 7 participants were followed by watchful waiting for spontaneous microbiome reconstitution; 6 participants were randomized to receive autologous fecal microbiome transplantation (aFMT) administered through a jejunal infusion of 150 ml of processed and liquefied stool (see methods); and 8 participants received a commonly consumed 11-strain probiotics preparation administered bi-daily for a period of 4 weeks (FIG. 24A). All participants collected stool samples at repetitive intervals prior to treatment with antibiotics (baseline period), during antibiotics administration (antibiotics period) and during reconstitution (follow-up period). Additional stool samples were obtained on a monthly basis after cessation of intervention, for a total period of 6 months.

Endoscopic examinations were performed twice in each of the 21 participants. A first colonoscopy and deep endoscopy were performed after completion of the weeklong antibiotic course, thereby characterizing the post-antibiotics dysbiosis throughout the gastrointestinal tract. A second colonoscopy and deep endoscopy were performed three weeks later (day 21), to assess the degree of mucosal and luminal reconstitution in each of the three treatment arms (FIG. 24A). Prior to the endoscopic procedure, all participants underwent bowel preparation using an identical protocol, and adherence was validated by a medical doctor to avoid differential effects of preparation on the gut microbiome (Example 1). All the endoscopic procedures were performed using an identical protocol (see methods) by one of three experienced board-certified gastroenterologists in a tertiary medical center setting. Collectively, 557 stool samples, 451 mucosal microbiome samples, 250 luminal microbiome samples, and 240 regional gastrointestinal biopsies were collected (FIG. 24A). All microbiome samples were processed and analyzed using both 16S rDNA and shotgun metagenomic sequencing; mucosal and selected stool samples were also analyzed by qPCR to quantify probiotics and total bacterial load.

### Probiotics in Antibiotics Perturbed Humans are Continuously Shed in Stool, and Colonize the LGI Mucosa

Expectedly, antibiotics treatment in humans triggered a profound fecal microbial depletion

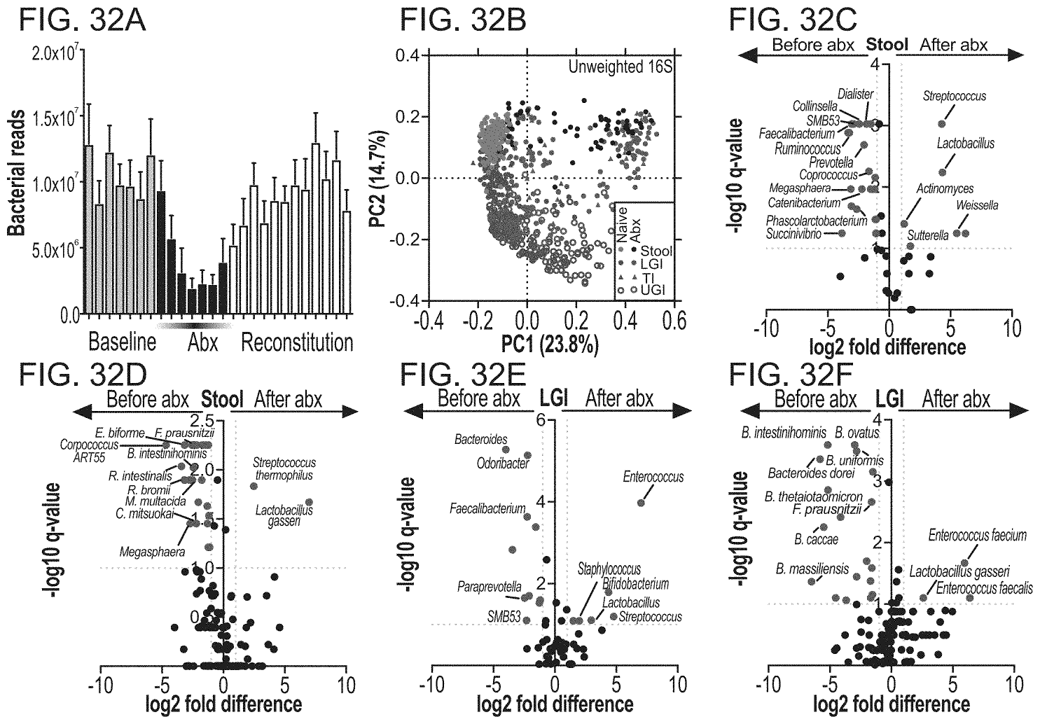

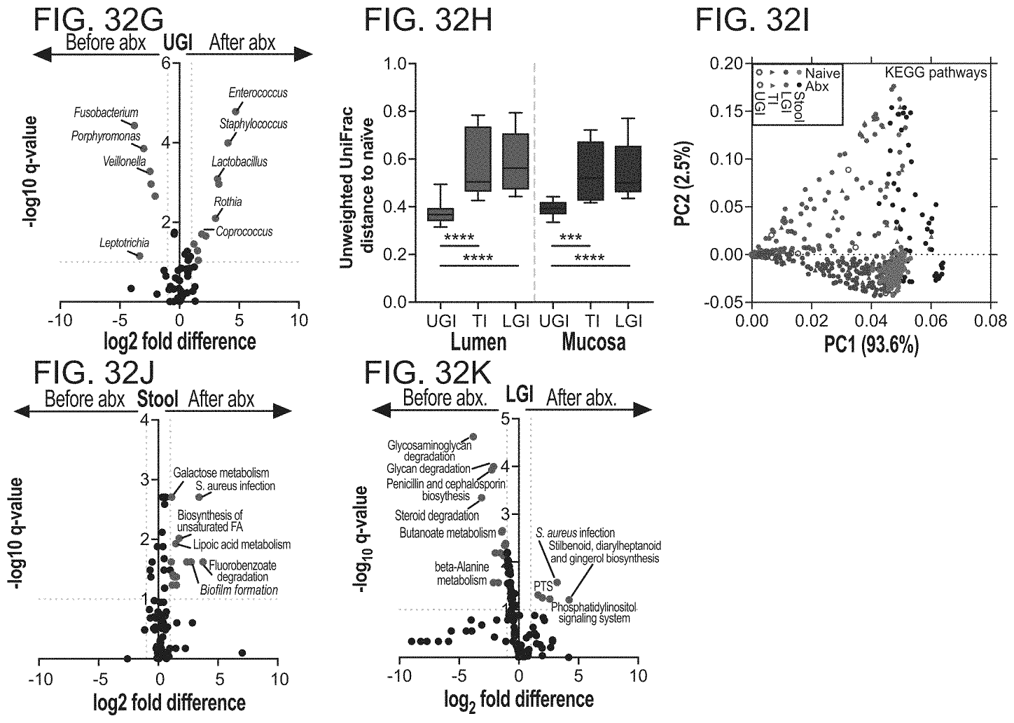

(FIG. 32A) and disruption of microbial community composition (FIG. 32B) as observed in stool (FIGS. 32C-D), LGI mucosa (FIGS. 32E-F) and UGI mucosa (FIG. 32G), with the latter region the least affected by antibiotics (FIG. 32H). Compositional changes were accompanied by alteration of microbiome function in the stool and LGI, as assessed by shotgun metagenomic sequencing (FIGS. 32I-K).

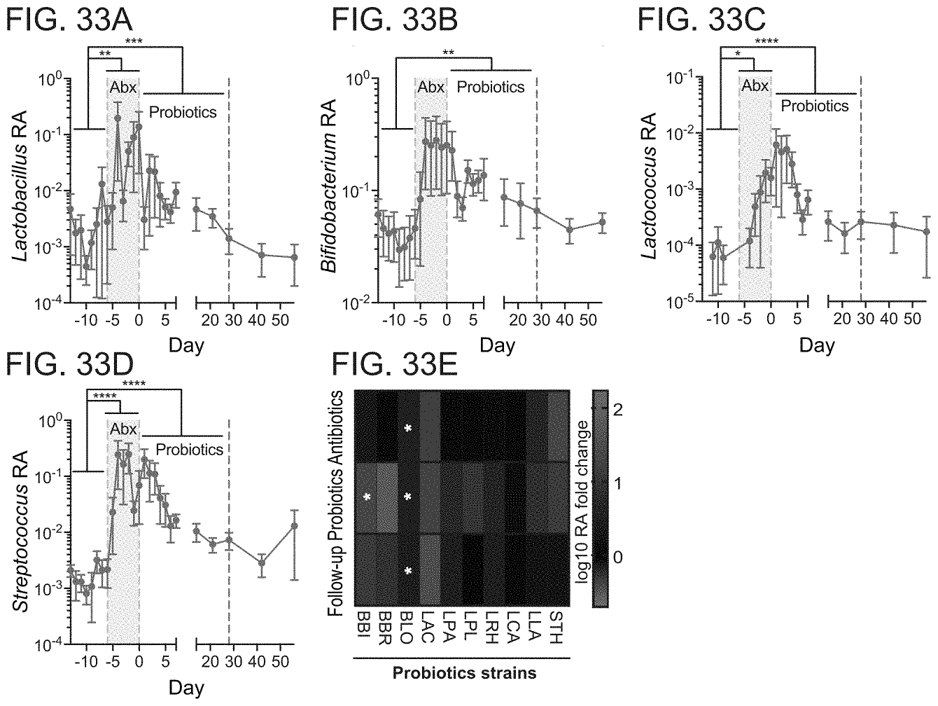

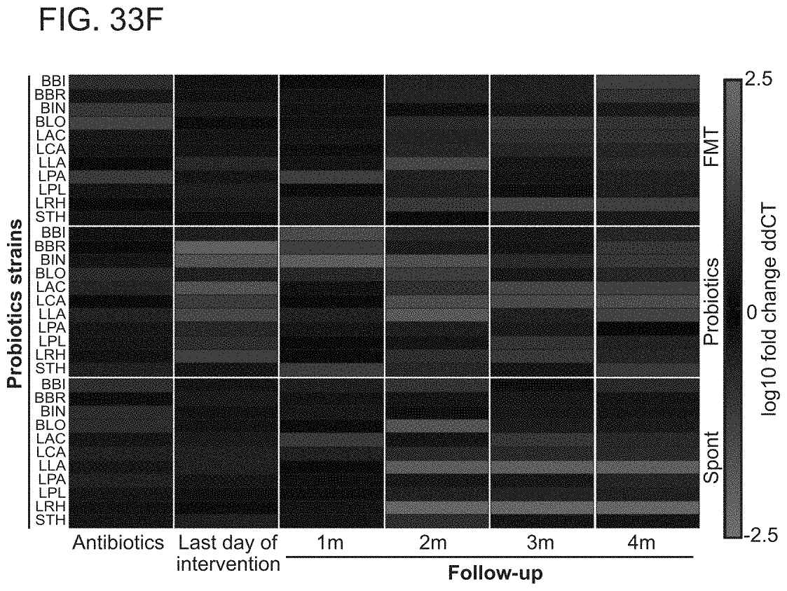

Fecal 16S rDNA analysis demonstrated that all probiotics-related genera were found in stools prior to probiotics supplementation (FIGS. 33A-D), and some expanded in RA following antibiotics treatment, including Lactobacillus (13.6-fold increase, Kruskal-Wallis & Dunn's P=0.002, FIG. 33A), Lactococcus (18-fold increase, P=0.04, FIG. 33C) and Streptococcus (64.7-fold increase, P<0.0001, FIG. 33D). During probiotics supplementation, significant expansion from baseline was noted in fecal Lactobacillus (5.3-fold increase, P=0.0009, FIG. 33A), Bifidobacterium (2.6-fold increase, P=0.004, FIG. 33B), Lactococcus (54.3-fold increase, P<0.0001, FIG. 33C) and Streptococcus (31.4-fold increase, P<0.0001, FIG. 33D), though none were further elevated compared to post-antibiotics levels. Following cessation of probiotic treatment, none of the genera remained significantly elevated compared to baseline (P>0.49, FIGS. 33A-D). A fecal species-level metagenomic (MetaPhlAn2) analysis also demonstrated antibiotics-induced expansion in RA of 6/11 species compared to baseline (BBI, BBR, BLO, LAC, LLA and STH, average 12.4-fold expansion, P=0.0002 for BLO, FIG. 33E), while during probiotic treatment, all species expanded compared to baseline (average 207-fold), but only BBI and BLO reached statistical significance (P=0.028 & P=0.0001, respectively, FIG. 33E). A shotgun metagenomic sequencing strain-specific method⁵¹ identified one of the probiotic strains in a single baseline day in stool, two of the probiotics strains (different than the one appearing at baseline) during antibiotic treatment, and 6 of the pill-specific strains (BBI, BBR, BLO, LLA, LPL and LRH) in multiple days during probiotics exposure. BBI, BLO and BBR were also shed after cessation by the same participants (FIG. 24B).

Fecal species-specific qPCR, the most sensitive method, revealed a significant fecal expansion during probiotics administration of the 11-probiotic species when considered together (Two-Way ANOVA & Dunnett P=0.0001), with 7/11 species being significantly elevated from baseline when separately analyzed (BBR, BIN, LAC, LCA, LLA, LPL and LRH, FIG. 24C). This probiotic species expansion was significant compared to both aFMT and spontaneous recovery (Two-Way ANOVA & Tukey P=0.001 & P=0.0008, respectively, FIGS. 24D, 33F). Even four months after probiotics cessation, several probiotics species remained elevated in stools of the probiotics supplemented group compared to baseline (FIGS. 24D, 33F, incremental area under the curve, calculated from the first day of probiotics treatment, Kruskal-Wallis and Dunn's P<0.0001). The strain-specific method validated that one month after cessation, these Bifidobacterium species were indeed the probiotics pill strains (FIG. 24B).

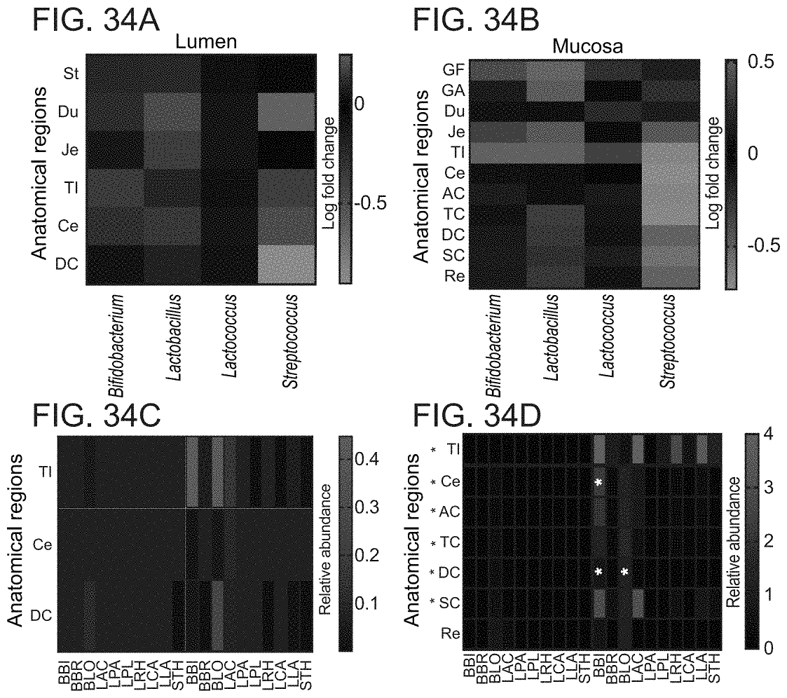

Given the above continuous shedding in stool, we assumed that the post-antibiotic gut mucosal colonization of probiotics is also enhanced as compared to that observed during homeostasis (Example 1). 16S rDNA analysis of luminal and mucosal GI samples collected before and after 3 weeks of probiotics, indicated no significant increases in the relative abundance of probiotic genera in the GI lumen (range 0.001-48, Two-Way ANOVA & Sidak P>0.05, FIG. 34A), or mucosa (range 0.001-229, FIG. 34B). MetaPhlAn2 analysis indicated that all probiotics species except LPA trended towards luminal expansion in RA from baseline, though none reached statistical significance (Two-Way ANOVA & Sidak P>0.5, FIG. 34C). In contrast, the mucosa of the TI and all LGI regions, except the rectum, featured significantly enhanced levels of probiotics species, stemming mostly from an elevation in BBI and BLO (P<0.05, FIG. 34D). Consequently, improved post-antibiotic probiotics colonization was noted as compared to the naïve probiotics-supplemented group (an 18.8-fold greater expansion in relative abundance in the post-antibiotics compared to naïve probiotics administration, Mann-Whitney P<0.0001, FIG. 24E).

In agreement, mucosal qPCR analysis indicated a significant probiotics colonization of the gastric fundus (Two-Way ANOVA P=0.03, FIG. 24F), terminal ileum (P=0.004), ascending (P<0.0001), transverse (P<0.0001), sigmoid (P=0.0002) colon, and the rectum (P=0.003). Probiotics species were also significantly elevated in the ascending and transverse colon of the post-antibiotics spontaneous recovery group (P=0.006 and P=0.02 respectively, FIG. 24F), while no significant elevation was observed in the aFMT group. On average, probiotics species expanded 8.7-fold more in the probiotics-supplemented group compared to spontaneous (P=0.0001) and 53.9-fold compared to aFMT (P<0.0001, FIG. 24G).

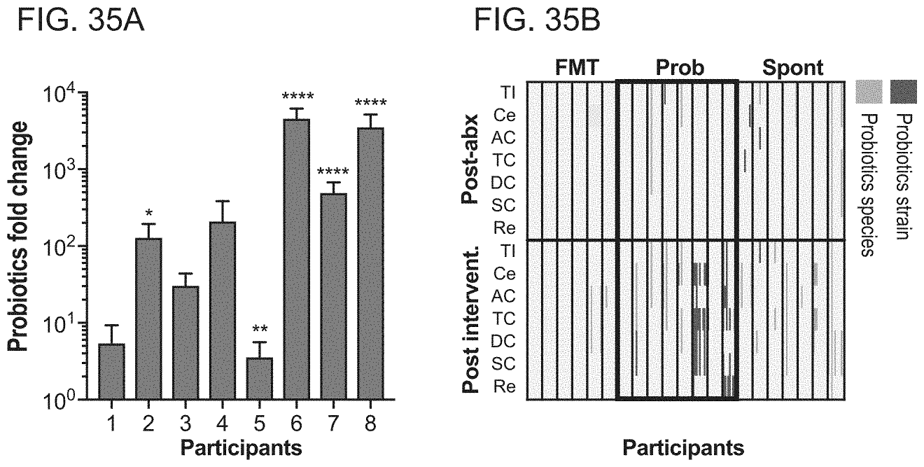

To determine whether antibiotics-treated individuals feature a person-specific, microbiome related colonization permissiveness/resistance to probiotics, similar to our observations under homeostatic conditions (Example 1), we calculated qPCR-based individual fold changes in the probiotic load between the first and last day of probiotics supplementation (FIG. 35A). In 4 participants, a significant >100-fold increase in mucosal probiotics load (aggregated for all targets) was observed (Paired Wilcoxon P<0.02). A fifth participant featured a milder but significant elevation (P=0.0096). Additional 3 participants experienced a non-significant trend towards probiotics mucosal expansion (5-211 fold). A probiotic-strain specific shotgun-based validation reflected this individualized pattern observed by qPCR and indicated that the colonizing strains originated from the supplemented pill (FIG. 35B). Due to the small subgroup size, we did not further pursue the etiology of these inter-individual differences, which merits further studies in larger cohorts.

Collectively in the antibiotics-perturbed gut, reversal of colonization resistance to probiotics enables incremental gut colonization by exogenously administered probiotic strains, mainly in the proximal large intestine, leading to long-term probiotic fecal shedding, indicative of stable colonization and active proliferation. Probiotic species belonging to Bifidobacterium were colonized at higher numbers compared to the other tested probiotics species.

### Probiotics Delay, while aFMT Improves the Post-Antibiotic Reconstitution of the Indigenous Human Fecal Microbiome

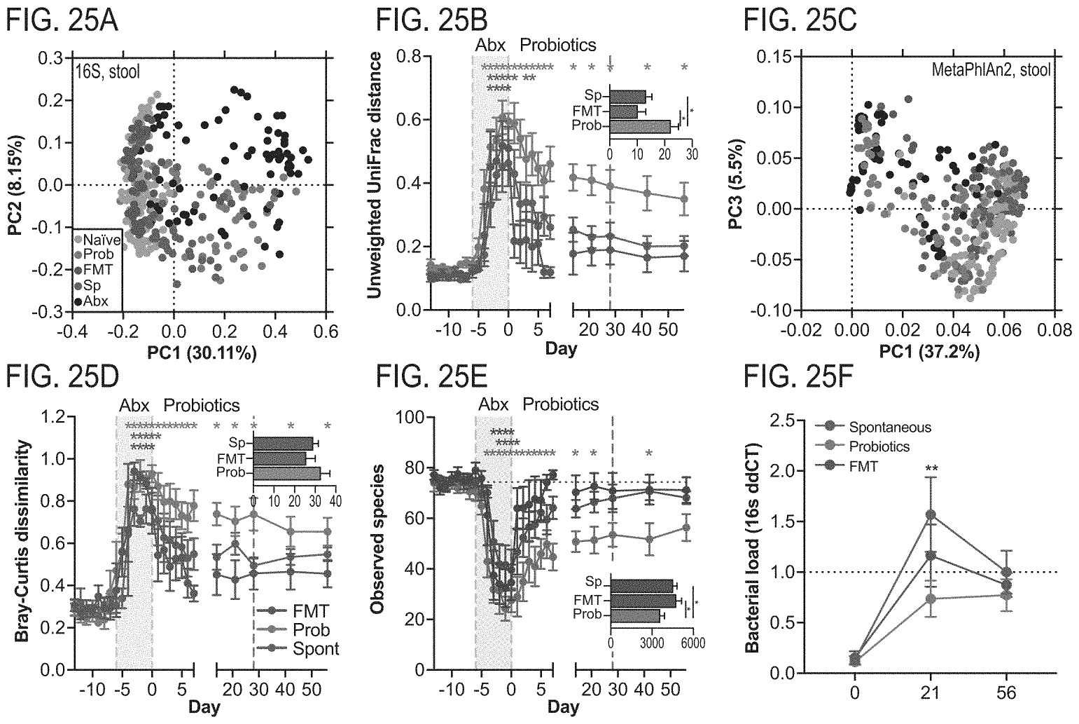

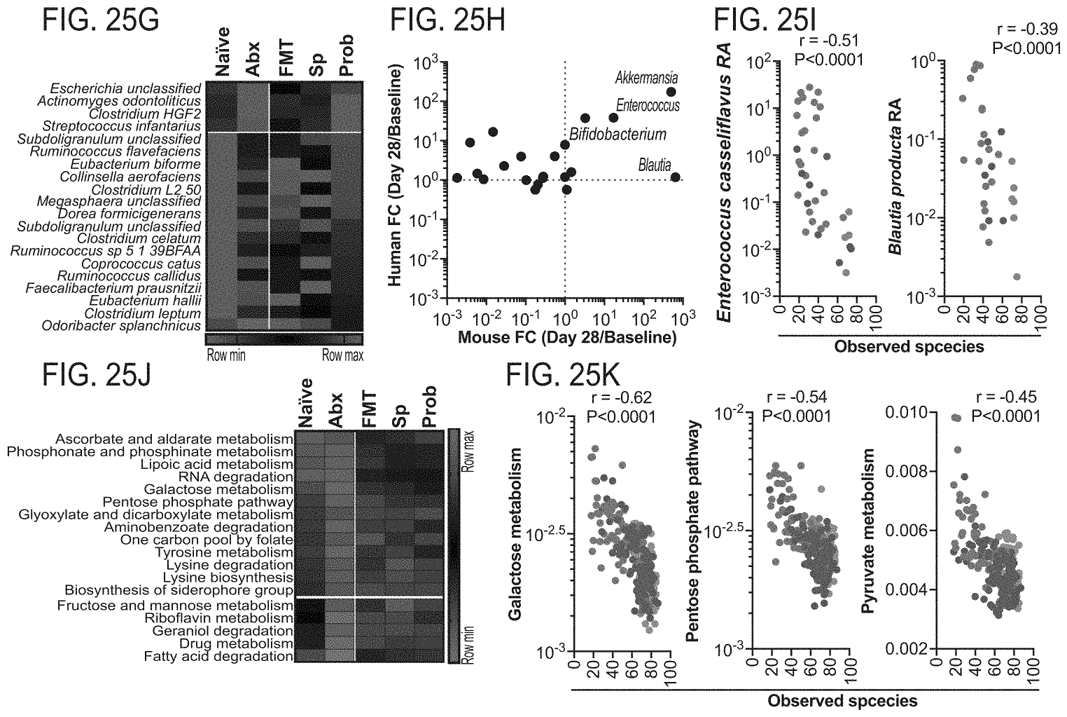

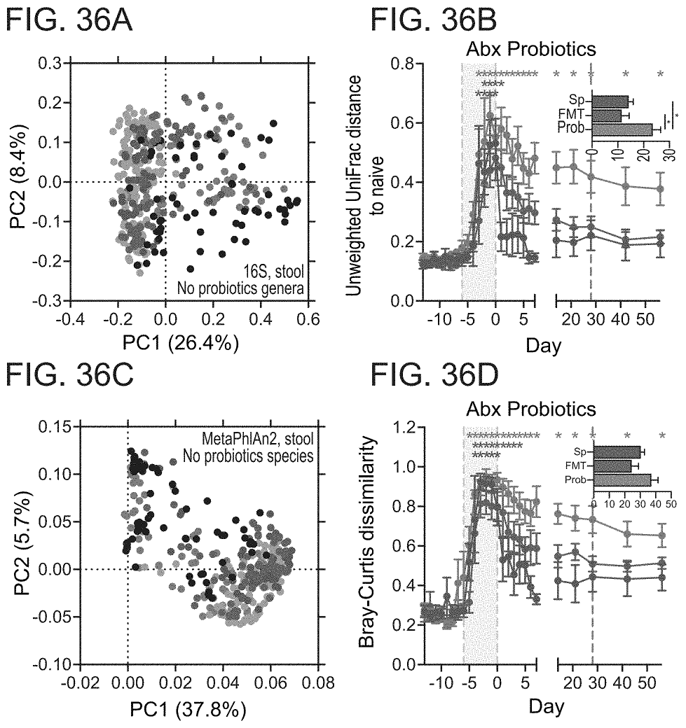

We next assessed the contribution of the three post-antibiotic treatment arms to reconstitution of the indigenous fecal microbiome in humans. We first utilized fecal 16s rDNA analysis, to calculate the unweighted UniFrac distances between stools collected during antibiotics treatment or during the reconstitution period to that of baseline stool microbiome configuration (FIGS. 25A-B). Of note, distance from baseline more than doubled during antibiotics treatment in all groups, reflecting the dramatic impact of antibiotics on stool microbiome configuration (Two-Way ANOVA & Dunnett P=0.0001). aFMT-treated individuals were quickest to return to baseline configuration, with differences in stool composition compared to baseline disappearing as early as 1 day following aFMT (FIG. 25B). In the spontaneous recovery group, significant differences in stool composition compared to baseline abated within 7 days of antibiotics cessation (FIG. 25B). In contrast, probiotics-consuming individuals did not return to their baseline stool microbiome configuration by the end of the intervention period, with all stool samples collected through day 56 (one month after probiotics cessation) remaining significantly different from baseline (Two-Way ANOVA & Dunnett P<0.01, FIGS. 25A-B). In addition to differences from baseline, probiotics-consuming individuals were also distinct from the spontaneous recovery group throughout the reconstitution (P=0.038, 10⁵ permutations). Consequently, the area under the probiotics-administered group reconstitution curve was significantly higher than aFMT (unpaired t-test P=0.01, FIG. 25B) and spontaneous recovery (P=0.02, FIG. 25B). As in mice, the distinct microbiome composition could not be explained by the mere presence of probiotics genera in probiotics-consuming individuals, as the result remained unchanged even if probiotics genera were excluded from the analysis and relative abundances renormalized (FIGS. 36A-B). Delayed reconstitution in probiotics consuming-individuals was also observed by a MetaPhlAn2 species-based analysis (FIGS. 25C-D), even when probiotics species were omitted from the analysis (FIGS. 36C-D).

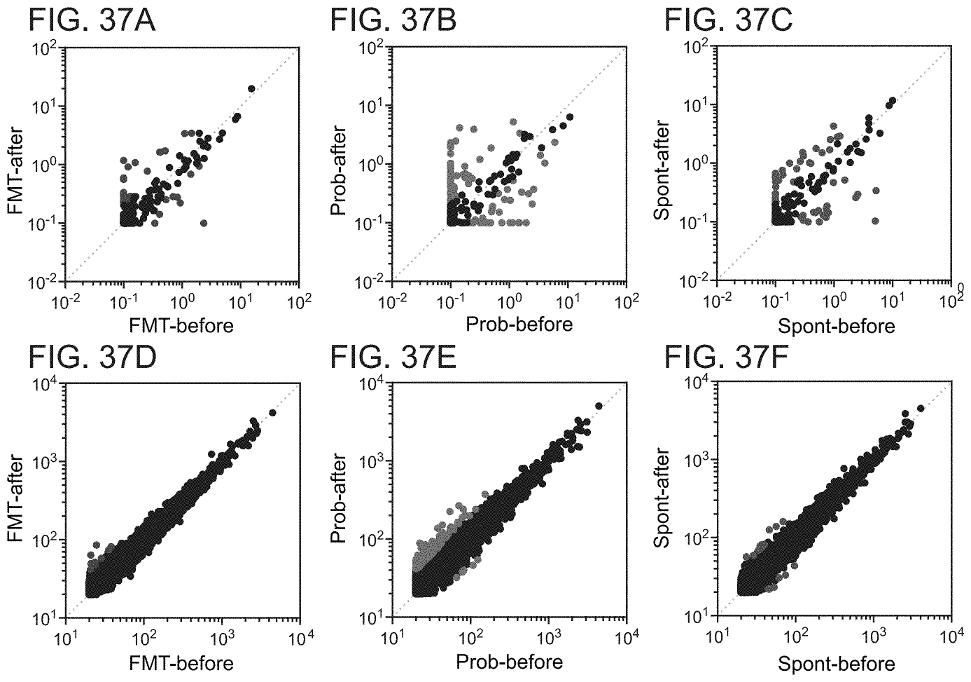

We then quantified species and functional KEGG orthologs (KOs) that were more than two-fold distinct in their fecal abundances between baseline (pre-antibiotics) and the end of reconstitution in the three arms; aFMT had the fewest number of fecal species distinct between baseline and endpoint (29 species, FIG. 37A), while probiotics had the most (96, FIG. 37B), almost double than spontaneous recovery (51, FIG. 37C). Of the three species significantly reverted to naive levels by aFMT but not by spontaneous recovery, one belonged to Bacteroidales (Alistipes shahii) and two to Clostridiales (Roseburia intestinalis and Coprococcus). Microbiome function, as determined by fecal KOs, displayed the same pattern (9 KOs in aFMT, 123 in probiotics, and 17 in spontaneous recovery, FIGS. 37D-F respectively). Importantly, probiotics not only shifted the microbiome composition and function from baseline, but also inhibited the post-antibiotics restoration of bacterial diversity (FIG. 25E) and load (FIG. 25F). Following antibiotics treatment, the number of observed species in feces was halved, but was restored in both the aFMT and the spontaneous recovery groups within one day (FIG. 25E). In contrast, the alpha diversity remained significantly low and did not return to baseline in the probiotics group throughout the intervention period (FIG. 25E). Likewise, fecal bacterial load failed to return to baseline after three weeks of probiotics supplementation, as compared to both aFMT and spontaneous recovery (Two-Way ANOVA & Tukey P=0.0079 vs. spontaneous, FIG. 25F), and remained lower than baseline one month after probiotics supplementation ceased.

Of the species altered in fecal RA by antibiotics, we identified 20 that returned to baseline comparable levels in the aFMT and spontaneous recovery groups, but not in the probiotics group (FIG. 25G). As in the mouse, the majority of the probiotics-inhibited species belonged to the Clostridiales order. As both humans and mice experienced probiotics-related inhibition of microbiome restoration, we compared fecal fold changes of taxa between the organisms. Four taxa, Enterococcus, Akkermansia, Bifidobacterium, and Blautia, bloomed after probiotics supplementation in both species (FIG. 25H). To assess which of the blooming taxa may be involved in microbiome inhibition we correlated 16S- and MetaPhlAn2-based abundances with alpha diversity. 14 genera and 107 species were significantly inversely correlated with alpha diversity, including the majority of probiotics species (excluding LPA and STH), and Enterococcus casseliflavus and Blautia producta that were also significantly inversely correlated with alpha diversity in the mouse LGI mucosa (Table 7, FIG. 25I, FIG. 23K). Likewise, we identified multiple pathways that returned to their pre-antibiotics state in aFMT and spontaneous recovery but not in probiotics (FIG. 25J). 37 KOs and 60 pathways were significantly inversely correlated with alpha diversity in stool, the majority of which relate to metabolism (Table 7). The highest anti-correlation was with galactose metabolism, which, along with additional pathways, may be related to lactate production by the probiotic species that bloom in the fecal samples (FIG. 25K).

Together, while probiotics species colonized the mucosa of the antibiotics-perturbed human gut, they delayed the stool microbiome compositional, functional and diversity-related reconstitution to a pre-antibiotic configuration. This delayed fecal reconstitution persisted even after probiotic cessation. In contrast, aFMT induced a rapid and nearly complete fecal microbiome reconstitution, as compared to either the watchful waiting or probiotics-administered groups.

### Probiotics Delay the Post-Antibiotic Reconstitution of the Indigenous Human Mucosal Microbiome

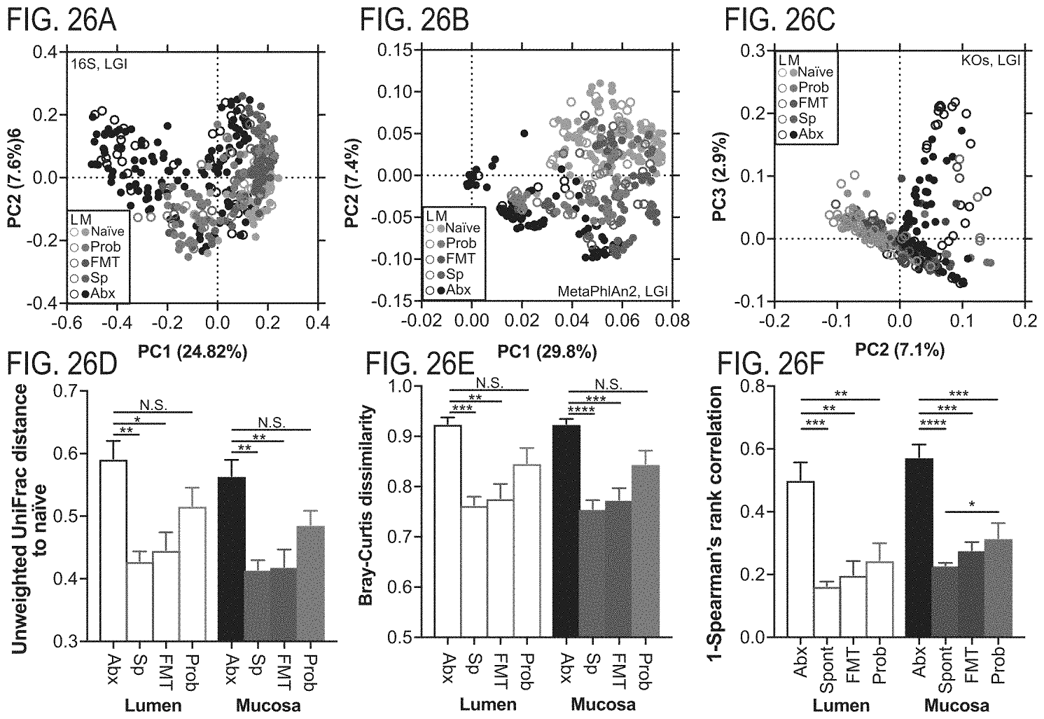

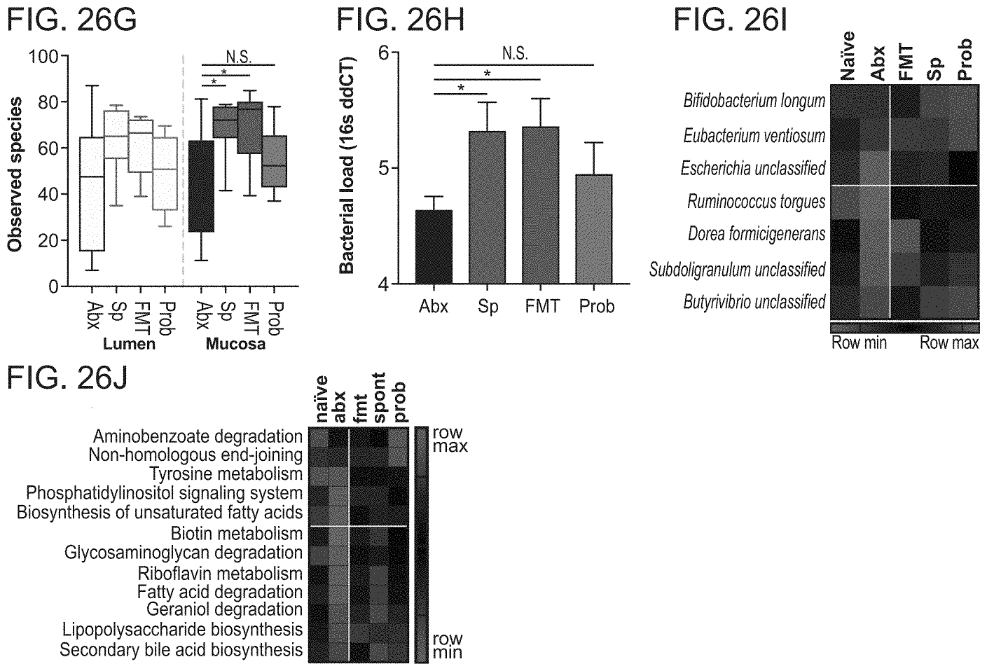

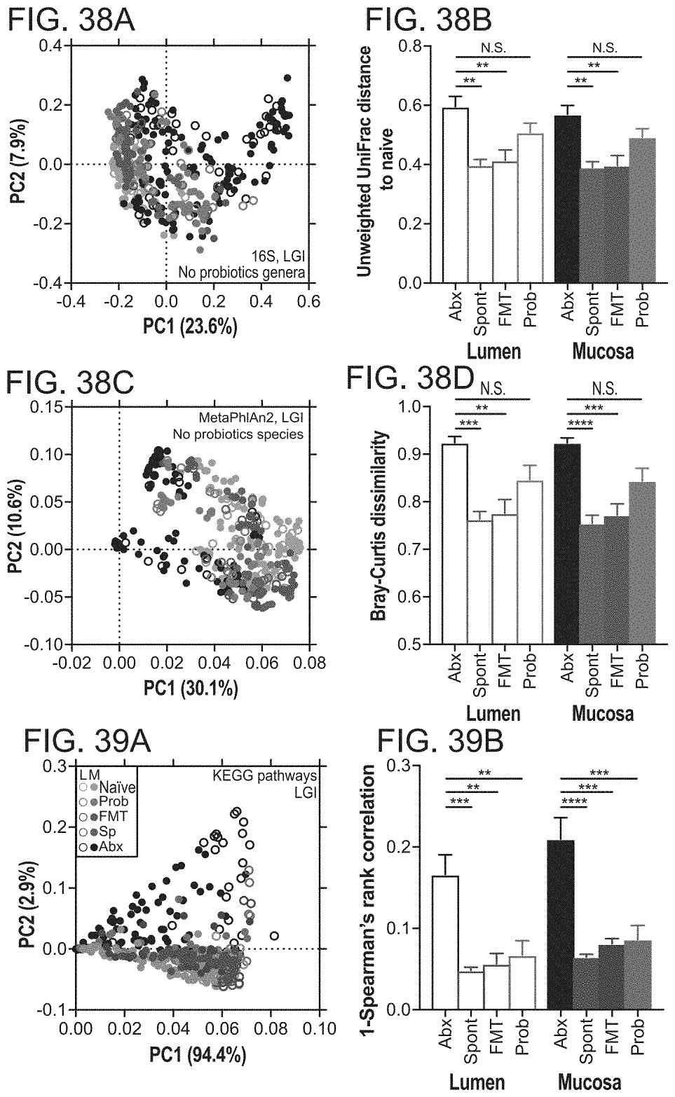

We next assessed whether the above probiotics- and aFMT-induced impacts on stool microbiome re-colonization could be documented in the gut mucosal level. We focused on the LGI, given the preferential probiotic post-antibiotic colonization at this region (FIG. 24G, 34C-D). Both 16S-rDNA and MetaPhlAn2-based analysis demonstrated that the aFMT and spontaneous recovery LGI luminal and mucosal configurations were significantly more similar to that of naïve non-antibiotics-treated controls than to the antibiotics-perturbed configuration (Lumen: 16S spontaneous Kruskal-Wallis & Dunn's P=0.0015, 16S aFMT P=0.015, MetaPhlAn2 spontaneous P=0.0003, MetaPhlAn2 aFMT P=0.002; Mucosa: 16S spontaneous 0.0027, 16S aFMT P=0.0066, MetaPhlAn2 spontaneous P<0.0001, MetaPhlAn2 aFMT P=0.0007, FIGS. 26A-D). In contrast, the probiotics LGI configuration remained similar to the antibiotics-perturbed configuration (Lumen: 16S P=0.76, MetaPhlAn2 P=0.092; Mucosa: 16S P=0.76, MetaPhlAn2 P=0.072, FIGS. 26A-D). The greater distance from the naive configuration of the probiotics group was not merely reflecting the presence of the probiotics species, as removal of the probiotics genera (FIGS. 38A-B) or species (FIGS. 38C-D) from the distance analysis maintained the aforementioned pattern. The function of the microbiome, in KOs (FIGS. 26E-F) and pathways (FIGS. 39A-B) also mirrored probiotics-associated delayed restoration of the indigenous mucosal LGI microbiome. As in stool, the LGI mucosa of the probiotics group displayed a lower alpha diversity, which was comparable to that of antibiotics (Probiotics P>0.999, aFMT & spontaneous P<0.05, FIG. 26G), reflected also in lower LGI mucosa bacterial load (Probiotics vs. abx P>0.999, aFMT & spontaneous vs. abx P<0.05, FIG. 26H). As in stool, multiple species (FIG. 26I) and pathways (FIG. 26J) were altered by antibiotics and reverted to homeostatic levels by aFMT and spontaneous recovery but not by probiotics, with all the inhibited species belonging to Clostridiales (FIG. 26I). 8 genera, 62 species, 80 KOs and 26 pathways were significantly anti-correlated with alpha diversity in the LGI mucosa, with high similarity in species (69%) and pathways (84%) between stool and mucosa (Table 7).

Collectively, enhanced post-antibiotic probiotics colonization in the LGI mucosa was associated with a compositional and functional persistence of post-antibiotic dysbiosis, reflected in both stool and LGI lumen and mucosa. This delayed return of the indigenous gut microbiome towards pre-antibiotic microbiome composition and function is in line with similar observations in mice (FIGS. 23A-K, 30A-J, 31A-K), suggestive of a global mechanism of interaction between the indigenous microbiome and exogenous probiotics across species.

### Reversion of Antibiotics-Associated GI Transcriptomic Landscape is Delayed by Probiotics

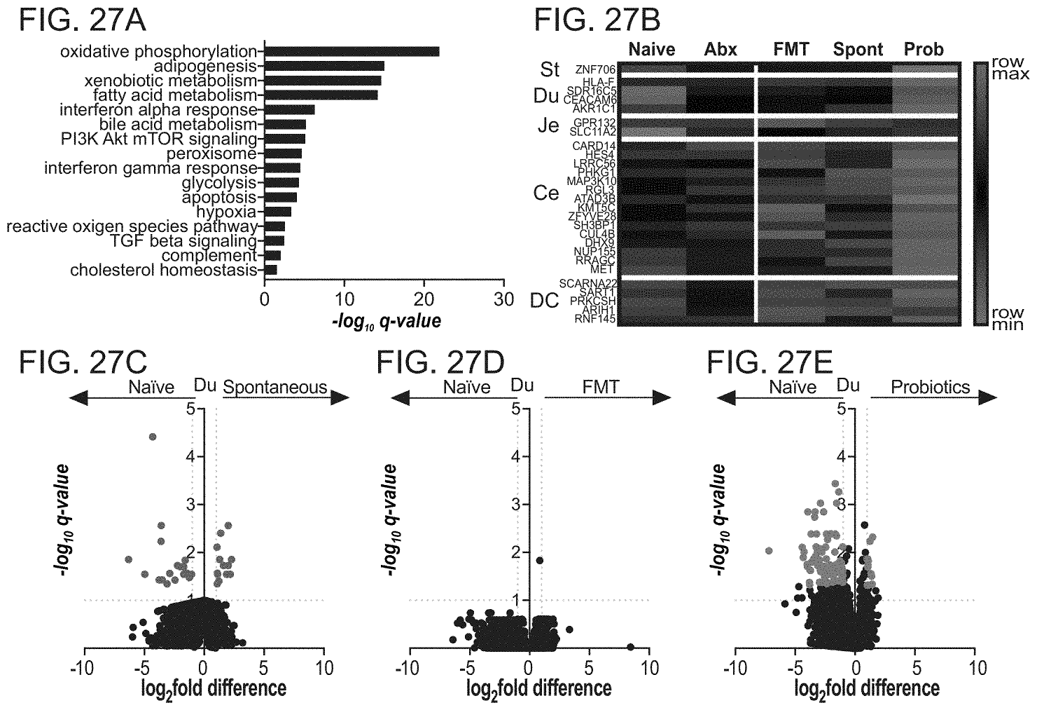

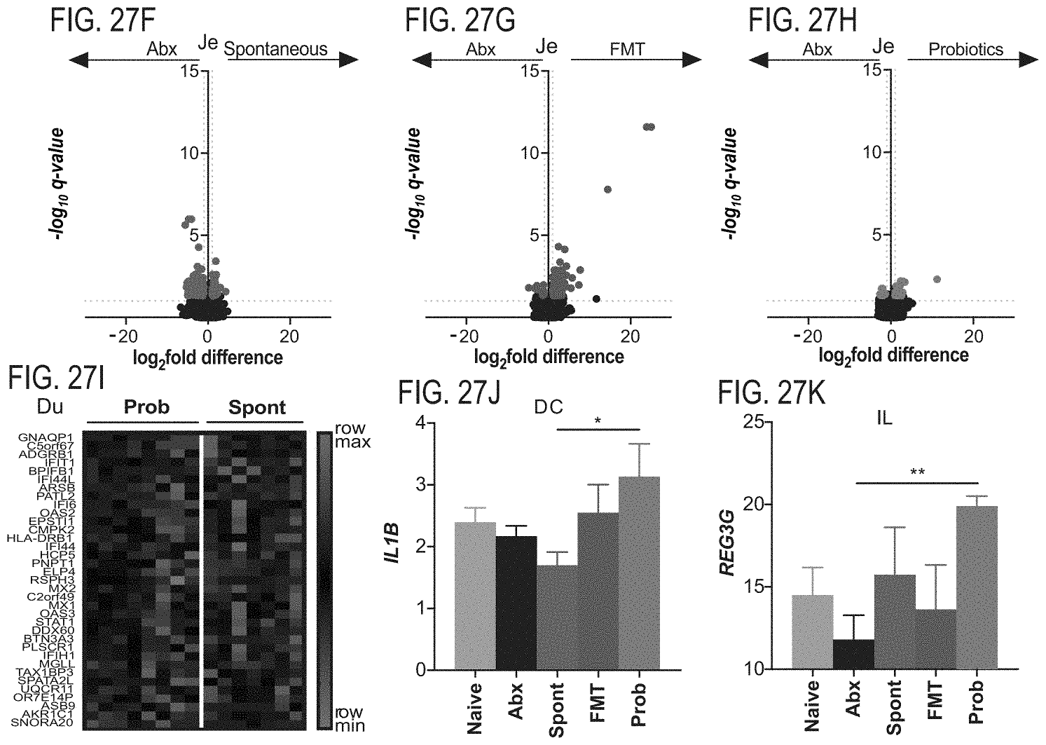

Given the differential impact of probiotics and aFMT, as compared to watchful waiting, or the recovery of mucosal gut microbiome composition and function, we next sought to characterize the effect of the three post-antibiotics interventions on the host. To this aim, we performed a global gene expression analysis through RNA sequencing of transcripts collected from stomach, duodenum, jejunum, terminal ileum, cecum and descending colon biopsies immediately after the antibiotics period and after three weeks of reconstitution (FIG. 24A). Of note, antibiotics affected the transcriptional landscape across the GI tract, though the majority of differences between naive and post-antibiotics state were observed in the descending colon (FIG. 27A). Importantly, restoration of the antibiotics-naive host transcriptional landscape by the three post-antibiotics intervention arms mirrored our findings in the microbiome, as multiple genes across the GI tract that were significantly affected by antibiotics were reverted towards homeostatic expression levels by spontaneous recovery and aFMT, but not by probiotics (FIG. 27B). When compared to the global naive (non-antibiotics exposed) transcriptional state, duodenal transcriptomes of the post-aFMT group featured the least amount of significantly differentially expressed genes (FIG. 27C), followed by the spontaneous recovery group (FIG. 27D), while the duodenal transcriptional landscape was most distinct from the naïve state in the probiotics group (FIG. 27E). Conversely, jejuna from the probiotic groups featured the greatest transcriptional similarity to the post-antibiotic transcriptional state, as compared to the transcriptome of the aFMT or watchful waiting groups (FIGS. 27F-H). The highest number of significant differences between the probiotics and spontaneous recovery groups was observed in the duodenum, including multiple genes belonging to the interferon-induced proteins (IFI) that were under-expressed in probiotic consumers (FIG. 271). Interestingly, probiotics led to a significant elevation in the transcript levels of inflammatory mediators & regulators of anti-microbial peptide secretion such as IL1B (FIG. 27J), and of some anti-microbial peptides such as REG3G (FIG. 27K), potentially contributing to the inhibition of indigenous commensal such as Clostridiales.

### Probiotics-Secreted Molecules Inhibit Human Microbiome In Vitro Growth

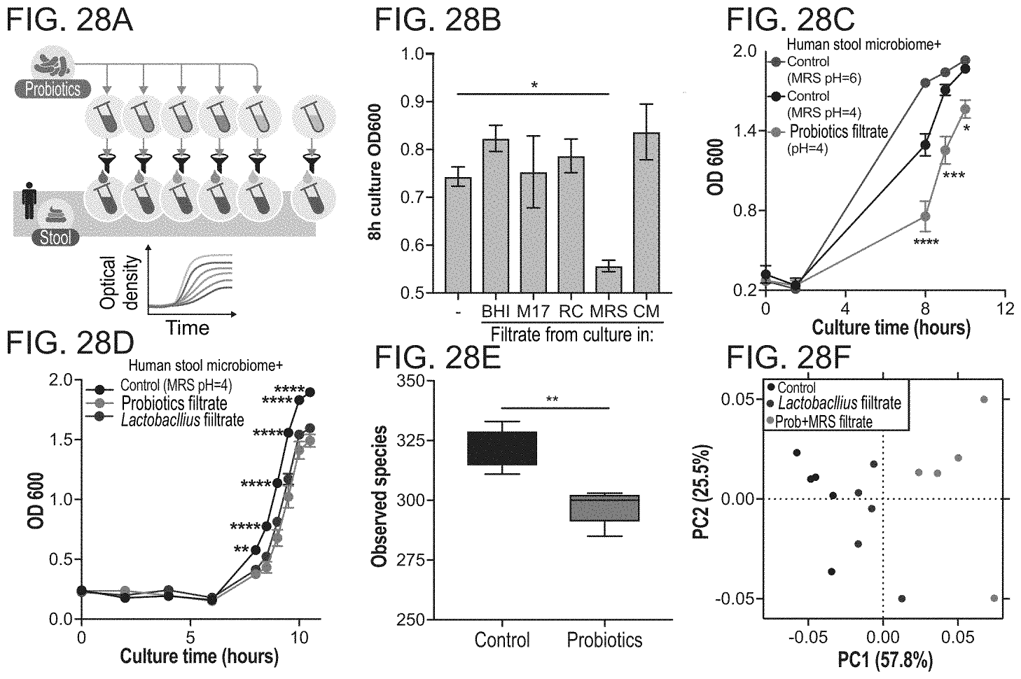

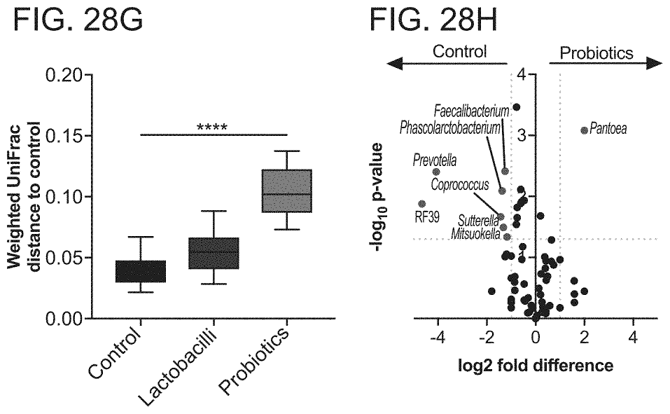

Finally, we explored potential direct probiotic-mediated mechanisms contributing to the inhibition of indigenous microbiome restoration. To this aim, we utilized a host-free, contact-independent system of probiotics-human microbiome culture. We began by culturing the probiotics pill content in five enriching growth media, differentially supporting the growth of distinct members of the probiotics consortium (FIG. 28A). Following 24 hours of anaerobic culture, supernatants from the five growth conditions were added to a lag-phase culture of fresh naive human fecal microbiome under anaerobic conditions. Optical density of the microbiome culture, measured after 8 hours, indicated that soluble factors in the MRS-probiotics culture supernatant (which mostly supports the growth of Lactobacillus) inhibited the growth of the naive human microbiome (One-Way ANOVA & Dunnett P=0.04, FIG. 28B). This inhibitory effect was not merely due to acid production by the probiotic bacteria, as the probiotics filtrate had an additive inhibitory effect to that of a comparably acidified, non-bacterial exposed medium (pH=4, FIG. 28C). We next sought to corroborate that Lactobacillus was indeed the microbiome inhibitory probiotic. To this aim, we collected supernatants from I. a MRS anaerobic culture of a probiotic pill content; II. A MRS x anaerobic culture of a mix of the 5 Lactobacillus species present in the pill; and III. A non-cultured MRS medium acidified to the levels measured with the other two cultures (PH=4, FIG. 28D). The three supernatants were then cultured with a naïve human microbiome under anaerobic conditions. Importantly, a significant growth inhibition was induced by both probiotics- and Lactobacillus-supernatants as compared to acidified MRS, suggestive of secreted Lactobacillus factors promoting the inhibitory effects (Two-Way ANOVA & Tukey P<0.05 for each time point of each group starting from 8 hours, FIG. 28B). 16S rDNA analysis of the filtrate-supplemented human microbiome cultures following 11 hours of culturing indicated that, indeed, these soluble factors significantly reduced the number of observed species (Unpaired t-test P=0.001, FIG. 28E) and modulated community structure (One-Way ANOVA & Dunnett P=0.0001, FIGS. 28F-G). This resulted in reduced levels of Prevotella and several taxa belonging to Clostridiales (Coprococcus, Faecalibacterium, Mitsuokella), in line with our observations with in vivo probiotic administration (FIG. 28H).

### REFERENCES FOR EXAMPLE 2

1 Van Boeckel, T. P. et al. Global antibiotic consumption 2000 to 2010: an analysis of national pharmaceutical sales data. Lancet Infect Dis 14, 742-750, doi:10.1016/S1473-3099(14)70780-7 (2014).
2 Wistrom, J. et al. Frequency of antibiotic-associated diarrhoea in 2462 antibiotic-treated hospitalized patients: a prospective study. J Antimicrob Chemother 47, 43-50 (2001).
3 McFarland, L. V. Epidemiology, risk factors and treatments for antibiotic-associated diarrhea. Dig Dis 16, 292-307, doi:10.1159/000016879 (1998).
4 Bartlett, J. G., Chang, T. W., Gurwith, M., Gorbach, S. L. & Onderdonk, A. B. Antibiotic-associated pseudomembranous colitis due to toxin-producing clostridia. N Engl J Med 298, 531-534, doi:10.1056/NEJM197803092981003 (1978).
5 Bartlett, J. G. Clinical practice. Antibiotic-associated diarrhea. N Engl J Med 346, 334-339, doi:10.1056/NEJMcp011603 (2002).
6 Ferrer, M., Martins dos Santos, V. A., Ott, S. J. & Moya, A. Gut microbiota disturbance during antibiotic therapy: a multi-omic approach. Gut Microbes 5, 64-70, doi:10.4161/gmic.27128 (2014).
7 Lichtman, J. S. et al. Host-Microbiota Interactions in the Pathogenesis of Antibiotic-Associated Diseases. Cell Rep 14, 1049-1061, doi:10.1016/j.celrep.2016.01.009 (2016).
8 Dethlefsen, L., Huse, S., Sogin, M. L. & Relman, D. A. The pervasive effects of an antibiotic on the human gut microbiota, as revealed by deep 16S rRNA sequencing. PLoS Biol 6, e280, doi:10.1371/journal.pbio.0060280 (2008).
9 Dethlefsen, L. & Relman, D. A. Incomplete recovery and individualized responses of the human distal gut microbiota to repeated antibiotic perturbation. Proc Natl Acad Sci USA 108 Suppl 1, 4554-4561, doi:10.1073/pnas.1000087107 (2011).
10 Jernberg, C., Lofmark, S., Edlund, C. & Jansson, J. K. Long-term ecological impacts of antibiotic administration on the human intestinal microbiota. ISME J 1, 56-66, doi:10.1038/ismej.2007.3 (2007).
11 Lankelma, J. M. et al. Antibiotic-induced gut microbiota disruption during human endotoxemia: a randomised controlled study. Gut 66, 1623-1630, doi:10.1136/gutjnl-2016-312132 (2017).
12 Trasande, L. et al. Infant antibiotic exposures and early-life body mass. Int J Obes (Lond) 37, 16-23, doi:10.1038/ijo.2012.132 (2013).
13 Azad, M. B., Bridgman, S. L., Becker, A. B. & Kozyrskyj, A. L. Infant antibiotic exposure and the development of childhood overweight and central adiposity. Int J Obes (Lond) 38, 1290-1298, doi:10.1038/ijo.2014.119 (2014).
14 Murphy, R. et al. Antibiotic treatment during infancy and increased body mass index in boys: an international cross-sectional study. Int J Obes (Lond) 38, 1115-1119, doi:10.1038/ijo.2013.218 (2014).
15 Ajslev, T. A., Andersen, C. S., Gamborg, M., Sorensen, T. I. & Jess, T. Childhood overweight after establishment of the gut microbiota: the role of delivery mode, pre-pregnancy weight and early administration of antibiotics. Int J Obes (Lond) 35, 522-529, doi:10.1038/ijo.2011.27 (2011).
16 Saari, A., Virta, L. J., Sankilampi, U., Dunkel, L. & Saxen, H. Antibiotic exposure in infancy and risk of being overweight in the first 24 months of life. Pediatrics 135, 617-626, doi:10.1542/peds.2014-3407 (2015).
17 Bailey, L. C. et al. Association of antibiotics in infancy with early childhood obesity. JAMA Pediatr 168, 1063-1069, doi:10.1001/jamapediatrics.2014.1539 (2014).
18 Ong, M. S., Umetsu, D. T. & Mandl, K. D. Consequences of antibiotics and infections in infancy: bugs, drugs, and wheezing. Ann Allergy Asthma Immunol 112, 441-445 e441, doi:10.1016/j.anai.2014.01.022 (2014).
19 Droste, J. H. et al. Does the use of antibiotics in early childhood increase the risk of asthma and allergic disease? Clin Exp Allergy 30, 1547-1553 (2000).
20 Johnson, C. C. et al. Antibiotic exposure in early infancy and risk for childhood atopy. J Allergy Clin Immunol 115, 1218-1224, doi:10.1016/j.jaci.2005.04.020 (2005).
21 McKeever, T. M. et al. Early exposure to infections and antibiotics and the incidence of allergic disease: a birth cohort study with the West Midlands General Practice Research Database. J Allergy Clin Immunol 109, 43-50 (2002).
22 Risnes, K. R., Belanger, K., Murk, W. & Bracken, M. B. Antibiotic exposure by 6 months and asthma and allergy at 6 years: Findings in a cohort of 1,401 US children. Am J Epidemiol 173, 310-318, doi:10.1093/aje/kwq400 (2011).
23 Hoskin-Parr, L., Teyhan, A., Blocker, A. & Henderson, A. J. Antibiotic exposure in the first two years of life and development of asthma and other allergic diseases by 7.5 yr: a dose-dependent relationship. Pediatr Allergy Immunol 24, 762-771, doi:10.1111/pai.12153 (2013).
24 Horton, D. B. et al. Antibiotic Exposure, Infection, and the Development of Pediatric Psoriasis: A Nested Case-Control Study. JAMA Dermatol 152, 191-199, doi:10.1001/jamadermatol.2015.3650 (2016).
25 Arvonen, M., Virta, L. J., Pokka, T., Kroger, L. & Vahasalo, P. Repeated exposure to antibiotics in infancy: a predisposing factor for juvenile idiopathic arthritis or a sign of this group's greater susceptibility to infections? J Rheumatol 42, 521-526, doi:10.3899/jrheum.140348 (2015).
26 Virta, L., Auvinen, A., Helenius, H., Huovinen, P. & Kolho, K. L. Association of repeated exposure to antibiotics with the development of pediatric Crohn's disease—a nationwide, register-based finish case-control study. Am J Epidemiol 175, 775-784, doi:10.1093/aje/kwr400 (2012).
27 Kronman, M. P., Zaoutis, T. E., Haynes, K., Feng, R. & Coffin, S. E. Antibiotic exposure and IBD development among children: a population-based cohort study. Pediatrics 130, e794-803, doi:10.1542/peds.2011-3886 (2012).
28 Ekmekciu, I. et al. The Probiotic Compound VSL #3 Modulates Mucosal, Peripheral, and Systemic Immunity Following Murine Broad-Spectrum Antibiotic Treatment. Front Cell Infect Microbiol 7, 167, doi:10.3389/fcimb.2017.00167 (2017).
29 Hill, C. et al. Expert consensus document. The International Scientific Association for Probiotics and Prebiotics consensus statement on the scope and appropriate use of the term probiotic. Nat Rev Gastroenterol Hepatol 11, 506-514, doi:10.1038/nrgastro.2014.66 (2014).
30 Goldenberg, J. Z. et al. Probiotics for the prevention of pediatric antibiotic-associated diarrhea. Cochrane Database Syst Rev, CD004827, doi:10.1002/14651858.CD004827.pub4 (2015).
31 Blaabjerg, S., Artzi, D. M. & Aabenhus, R. Probiotics for the Prevention of Antibiotic-Associated Diarrhea in Outpatients-A Systematic Review and Meta-Analysis. Antibiotics (Basel) 6, doi:10.3390/antibiotics6040021 (2017).
32 Cremonini, F. et al. Effect of different probiotic preparations on anti-Helicobacter pylori therapy-related side effects: a parallel group, triple blind, placebo-controlled study. Am J Gastroenterol 97, 2744-2749, doi:10.1111/j.1572-0241.2002.07063.x (2002).
33 Hickson, M. et al. Use of probiotic Lactobacillus preparation to prevent diarrhoea associated with antibiotics: randomised double blind placebo controlled trial. BMJ 335, 80, doi:10.1136/bmj.39231.599815.55 (2007).
34 McFarland, L. V., Huang, Y., Wang, L. & Malfertheiner, P. Systematic review and meta-analysis: Multi-strain probiotics as adjunct therapy for Helicobacter pylori eradication and prevention of adverse events. United European Gastroenterol J 4, 546-561, doi:10.1177/2050640615617358 (2016).
35 Olek, A. et al. Efficacy and Safety of Lactobacillus plantarum DSM 9843 (LP299V) in the Prevention of Antibiotic-Associated Gastrointestinal Symptoms in Children-Randomized, Double-Blind, Placebo-Controlled Study. J Pediatr 186, 82-86, doi:10.1016/j.jpeds.2017.03.047 (2017).
36 Klarin, B., Johansson, M. L., Molin, G., Larsson, A. & Jeppsson, B. Adhesion of the probiotic bacterium Lactobacillus plantarum 299v onto the gut mucosa in critically ill patients: a randomised open trial. Crit Care 9, R285-293, doi:10.1186/cc3522 (2005).
37 Grazul, H., Kanda, L. L. & Gondek, D. Impact of probiotic supplements on microbiome diversity following antibiotic treatment of mice. Gut microbes 7, 101-114 (2016).
38 Fujiwara, S., Seto, Y., Kimura, A. & Hashiba, H. Establishment of orally-administered Lactobacillus gasseri SBT2055SR in the gastrointestinal tract of humans and its influence on intestinal microflora and metabolism. J Appl Microbiol 90, 343-352 (2001).
39 Hutt, P. et al. Safety and persistence of orally administered human Lactobacillus sp. strains in healthy adults. Benef Microbes 2, 79-90, doi:10.3920/BM2010.0023 (2011).
40 McFarland, L. V. Use of probiotics to correct dysbiosis of normal microbiota following disease or disruptive events: a systematic review. BMJ Open 4, e005047, doi:10.1136/bmjopen-2014-005047 (2014).
41 Lidbeck, A., Edlund, C., Gustafsson, J. A., Kager, L. & Nord, C. E. Impact of Lactobacillus acidophilus on the normal intestinal microflora after administration of two antimicrobial agents. Infection 16, 329-336 (1988).
42 Allen, S. J. et al. Lactobacilli and bifidobacteria in the prevention of antibiotic-associated diarrhoea and Clostridium difficile diarrhoea in older inpatients (PLACIDE): a randomised, double-blind, placebo-controlled, multicentre trial. Lancet 382, 1249-1257, doi:10.1016/50140-6736(13)61218-0 (2013).
43 Black, F., Einarsson, K., Lidbeck, A., Orrhage, K. & Nord, C. E. Effect of lactic acid producing bacteria on the human intestinal microflora during ampicillin treatment. Scand J Infect Dis 23, 247-254 (1991).
44 Plummer, S. F. et al. Effects of probiotics on the composition of the intestinal microbiota following antibiotic therapy. Int J Antimicrob Agents 26, 69-74, doi:10.1016/j.ijantimicag.2005.04.004 (2005).
45 Koning, C. J. et al. The effect of a multispecies probiotic on the intestinal microbiota and bowel movements in healthy volunteers taking the antibiotic amoxycillin. Am J Gastroenterol 103, 178-189, doi:10.1111/j.1572-0241.2007.01547.x (2008).
46 Ferrario, C. et al. Modulation of fecal Clostridiales bacteria and butyrate by probiotic intervention with Lactobacillus paracasei DG varies among healthy adults. J Nutr 144, 1787-1796, doi:10.3945/jn.114.197723 (2014).
47 Maldonado-Gomez, M. X. et al. Stable Engraftment of Bifidobacterium longum AH1206 in the Human Gut Depends on Individualized Features of the Resident Microbiome. Cell Host Microbe 20, 515-526, doi:10.1016/j.chom.2016.09.001 (2016).
48 Nord, C. E., Lidbeck, A., Orrhage, K. & Sjostedt, S. Oral supplementation with lactic acid-producing bacteria during intake of clindamycin. Clin Microbiol Infect 3, 124-132 (1997).
49 Jernberg, C., Sullivan, A., Edlund, C. & Jansson, J. K. Monitoring of antibiotic-induced alterations in the human intestinal microflora and detection of probiotic strains by use of terminal restriction fragment length polymorphism. Appl Environ Microbiol 71, 501-506, doi:10.1128/AEM.71.1.501-506.2005 (2005).
50 Madden, J. A. et al. Effect of probiotics on preventing disruption of the intestinal microflora following antibiotic therapy: a double-blind, placebo-controlled pilot study. Int Immunopharmacol 5, 1091-1097, doi:10.1016/j.intimp.2005.02.006 (2005).
51 Sharon, I. et al. Time series community genomics analysis reveals rapid shifts in bacterial species, strains, and phage during infant gut colonization. Genome research 23, 111-120 (2013).
52 Yatsunenko, T. et al. Human gut microbiome viewed across age and geography. Nature 486, 222-227, doi:10.1038/nature11053 (2012).
53 Zeevi, D. et al. Personalized Nutrition by Prediction of Glycemic Responses. Cell 163, 1079-1094, doi:10.1016/j.cell.2015.11.001 (2015).
54 Goossens, D. A., Jonkers, D. M., Russel, M. G., Stobberingh, E. E. & Stockbrugger, R. W. The effect of a probiotic drink with Lactobacillus plantarum 299v on the bacterial composition in faeces and mucosal biopsies of rectum and ascending colon. Aliment Pharmacol Ther 23, 255-263, doi:10.1111/j.1365-2036.2006.02749.x (2006).
55 Vangay, P., Ward, T., Gerber, J. S. & Knights, D. Antibiotics, pediatric dysbiosis, and disease. Cell host & microbe 17, 553-564 (2015).
56 Caballero, S. et al. Cooperating commensals restore colonization resistance to vancomycin-resistant Enterococcus faecium. Cell host & microbe 21, 592-602. e594 (2017).
57 Franz, C. M., Van Belkum, M. J., Holzapfel, W. H., Abriouel, H. & Gálvez, A. Diversity of enterococcal bacteriocins and their grouping in a new classification scheme. FEMS microbiology reviews 31, 293-310 (2007).
58 Cotter, P. D., Ross, R. P. & Hill, C. Bacteriocins—a viable alternative to antibiotics? Nature Reviews Microbiology 11, 95 (2013).
59 Franker, C. K., Herbert, C. A. & Ueda, S. Bacteriocin from Actinomyces odontolyticus with temperature-dependent killing properties. Antimicrobial agents and chemotherapy 12, 410-417 (1977).
60 Kootte, R. S. et al. Improvement of Insulin Sensitivity after Lean Donor Feces in Metabolic Syndrome Is Driven by Baseline Intestinal Microbiota Composition. Cell Metab 26, 611-619 e616, doi:10.1016/j.cmet.2017.09.008 (2017).
61 Smith, M., Kassam, Z., Edelstein, C., Burgess, J. & Alm, E. OpenBiome remains open to serve the medical community. Nat Biotechnol 132, 867, doi:10.1038/nbt.3006 (2014).
62 Terveer, E. M. et al. How to: Establish and run a stool bank. Clin Microbiol Infect, doi:10.1016/j.cmi.2017.05.015 (2017).
63 Herbel, S. et al. Species-specific quantification of probiotic lactobacilli in yoghurt by quantitative real-time PCR. Journal of applied microbiology 115, 1402-1410 (2013).
64 Furet, J.-P., Quénée, P. & Tailliez, P. Molecular quantification of lactic acid bacteria in fermented milk products using real-time quantitative PCR. International journal of food microbiology 97, 197-207 (2004).
65 Schwendimann, L., Kauf, P., Fieseler, L., Gantenbein-Demarchi, C. & Schwenninger, S. M. Development of a quantitative PCR assay for rapid detection of Lactobacillus plantarum and Lactobacillus fermentum in cocoa bean fermentation. Journal of microbiological methods 115, 94-99 (2015).
66 Haarman, M. & Knol, J. Quantitative real-time PCR assays to identify and quantify fecal Bifidobacterium species in infants receiving a prebiotic infant formula. Applied and environmental microbiology 71, 2318-2324 (2005).
67 Ruggirello, M., Dolci, P. & Cocolin, L. Detection and viability of Lactococcus lactis throughout cheese ripening. PloS one 9, e114280 (2014).
68 Huber, J. A. et al. Microbial population structures in the deep marine biosphere. science 318, 97-100 (2007).
69 Caporaso, J. G. et al. QIIME allows analysis of high-throughput community sequencing data. Nature methods 7, 335 (2010).
70 Bolger, A. M., Lohse, M. & Usadel, B. Trimmomatic: a flexible trimmer for Illumina sequence data. Bioinformatics 30, 2114-2120 (2014).
71 Truong, D. T. et al. MetaPhlAn2 for enhanced metagenomic taxonomic profiling. Nature methods 12, 902 (2015).
72 Langmead, B. & Salzberg, S. L. Fast gapped-read alignment with Bowtie 2. Nature methods 9, 357 (2012).
73 Manor, O. & Borenstein, E. Revised computational metagenomic processing uncovers hidden and biologically meaningful functional variation in the human microbiome. Microbiome 5, 19 (2017).
74 Eden, E., Navon, R., Steinfeld, I., Lipson, D. & Yakhini, Z. GOrilla: a tool for discovery and visualization of enriched GO terms in ranked gene lists. BMC bioinformatics 10, 48 (2009).
75 Suez, J. et al. Artificial sweeteners induce glucose intolerance by altering the gut microbiota. Nature 514, 181 (2014).
76 Adiconis, X. et al. Comparative analysis of RNA sequencing methods for degraded or low-input samples. Nature methods 10, 623-629 (2013).
77 Qin, J. et al. A human gut microbial gene catalogue established by metagenomic sequencing. nature 464, 59 (2010).
78 Kanehisa, M. & Goto, S. KEGG: kyoto encyclopedia of genes and genomes. Nucleic acids research 28, 27-30 (2000).
79 Peng, Y., Leung, H. C., Yiu, S.-M. & Chin, F. Y. IDBA-UD: a de novo assembler for single-cell and metagenomic sequencing data with highly uneven depth. Bioinformatics 28, 1420-1428 (2012).
80 Parks, D. H., Imelfort, M., Skennerton, C. T., Hugenholtz, P. & Tyson, G. W. CheckM: assessing the quality of microbial genomes recovered from isolates, single cells, and metagenomes. Genome research 25, 1043-1055 (2015).
81 Edgar, R. C. Search and clustering orders of magnitude faster than BLAST. Bioinformatics 26, 2460-2461 (2010).
82 Li, X. et al. A comparison of per sample global scaling and per gene normalization methods for differential expression analysis of RNA-seq data. PloS one 12, e0176185 (2017).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, the priority document of this application is hereby incorporated herein by reference in its entirety.

1. A method of assessing whether a candidate subject is suitable for probiotic treatment comprising determining a signature of the gut microbiome of the candidate subject, wherein when said signature of the microbiome of the candidate subject is statistically significantly similar to a signature of a gut microbiome of a control subject known to be responsive to probiotic treatment, it is indicative that the subject is suitable for probiotic treatment.

2. The method of claim 1, wherein said determining said signature is effected by analyzing feces of the subject.

3. The method of claim 1, wherein said gut microbiome comprises a mucosal gut microbiome or a lumen gut microbiome.

4. The method of claim 1, wherein said probiotic comprises at least one of the bacterial species selected from the group consisting of B. bifidum, L. rhamnosus, L. lactis, L. casei, B. breve, S. thermophilus, B. longum, L. paracasei, L. plantarum and B. infantis.

5. The method of claim 1, wherein the candidate subject does not have a chronic disease.

6. The method of claim 1, wherein said signature of said gut microbiome is a presence or level of microbes of said microbiome.

7. The method of claim 1, wherein said signature of said gut microbiome is a presence or level of genes of microbes of said microbiome.

8. (canceled)

9. The method of claim 1, wherein said signature of said gut microbiome is an alpha diversity.

10. (canceled)

11. The method of claim 6, wherein said microbes of said microbiome are of an identical species to said microbes of the probiotic.

12. The method of claim 6, wherein said determining said signature is effected by analyzing feces of the subject.

13. The method of claim 12, wherein said microbes of said microbiome are of the species selected from the group consisting of those set forth in Table A and/or are of the genus Bifidobacterium or Dialister.

14. The method of claim 12, wherein said microbes of said microbiome utilize at least one pathway set forth in Table B.

15-43. (canceled)

44. A method of treating a disease of a subject for which an antibiotic is therapeutic comprising:
(a) administering to the subject an antibiotic which is suitable for treating the disease; and subsequently
(b)administering to the subject a an autologous fecal transplant, thereby treating the disease.

45-54. (canceled)

55. The method of claim 44, wherein the autologous fecal transplant is derived from the subject when he is healthy.

56. The method of claim 44, wherein the disease is a chronic disease.

57. The method of claim 44, wherein the disease is not a bacterial disease.

58. The method of claim 44, wherein the subject is deemed unsuitable for probiotic treatment.

59. The method of claim 58, wherein the subject is deemed unsuitable for probiotic treatment by determining a signature of the gut microbiome of the subject, wherein when said signature of the microbiome of the subject is statistically significantly similar to a signature of a gut microbiome of a control subject known to be non-responsive to probiotic treatment, it is indicative that the subject is not suitable for probiotic treatment.

60. A method of treating a subject having a disease associated with an antibiotics-perturbed gut comprising administering to the subject a therapeutically effective amount of an autologous fecal transplant thereby treating the subject having the disease associated with the antibiotics-perturbed gut.

61. The method of claim 60, wherein the autologous fecal transplant is derived from the subject when he is healthy.

62. The method of claim 60, wherein the disease is a chronic disease.

63. The method of claim 60, wherein the disease is not a bacterial disease.


SEQUENCE LISTING (154 sequences)

SEQ ID NO: 1 - DNA, 31 bp, Artificial sequence
  Single strand DNA oligonucleotide
tatggtaattgtgtgccagcmgccgcggtaa

SEQ ID NO: 2 - DNA, 32 bp, Artificial sequence
  Single strand DNA oligonucleotide
agtcagtcagccggactachvgggtwtctaat

SEQ ID NO: 3 - DNA, 32 bp, Artificial sequence
  Single strand DNA oligonucleotide
attagawacccbdgtagtccggctgactgact

SEQ ID NO: 4 - DNA, 32 bp, Artificial sequence
  Single strand DNA oligonucleotide
ctttgactcaggcaattgctcgtgaaggtatg

SEQ ID NO: 5 - DNA, 35 bp, Artificial sequence
  Single strand DNA oligonucleotide
caacttctttagatgctgaagaaacagcagctacg

SEQ ID NO: 6 - DNA, 24 bp, Artificial sequence
  Single strand DNA oligonucleotide
gtgcttgcatcttgatttaatttt

SEQ ID NO: 7 - DNA, 21 bp, Artificial sequence
  Single strand DNA oligonucleotide
tgcggttcttggatctatgcg

SEQ ID NO: 8 - DNA, 24 bp, Artificial sequence
  Single strand DNA oligonucleotide
gtgcttgcactgagattcgactta

SEQ ID NO: 9 - DNA, 21 bp, Artificial sequence
  Single strand DNA oligonucleotide
tgcggttcttggatctatgcg

SEQ ID NO: 10 - DNA, 24 bp, Artificial sequence
  Single strand DNA oligonucleotide
gtgcttgcaccgagattcaacatg

SEQ ID NO: 11 - DNA, 21 bp, Artificial sequence
  Single strand DNA oligonucleotide
tgcggttcttggatctatgcg

SEQ ID NO: 12 - DNA, 24 bp, Artificial sequence
  Single strand DNA oligonucleotide
ttacatttgagtgagtggcgaact

SEQ ID NO: 13 - DNA, 20 bp, Artificial sequence
  Single strand DNA oligonucleotide
aggtgttatcccccgcttct

SEQ ID NO: 14 - DNA, 19 bp, Artificial sequence
  Single strand DNA oligonucleotide
cgcgagcaaaacaatggtt

SEQ ID NO: 15 - DNA, 25 bp, Artificial sequence
  Single strand DNA oligonucleotide
aacgatcgaaacgaacaatagagtt

SEQ ID NO: 16 - DNA, 21 bp, Artificial sequence
  Single strand DNA oligonucleotide
gttgatttcgccggactcttc

SEQ ID NO: 17 - DNA, 18 bp, Artificial sequence
  Single strand DNA oligonucleotide
gcaagcctatcgcgcaaa

SEQ ID NO: 18 - DNA, 23 bp, Artificial sequence
  Single strand DNA oligonucleotide
gtggtggcttgagaactggatag

SEQ ID NO: 19 - DNA, 25 bp, Artificial sequence
  Single strand DNA oligonucleotide
caaaacgatcgaaacaaacactaaa

SEQ ID NO: 20 - DNA, 20 bp, Artificial sequence
  Single strand DNA oligonucleotide
tggaagacgtcgttggcttt

SEQ ID NO: 21 - DNA, 16 bp, Artificial sequence
  Single strand DNA oligonucleotide
atcgcgccaggcaaaa

SEQ ID NO: 22 - DNA, 20 bp, Artificial sequence
  Single strand DNA oligonucleotide
tgaaccacaatgggttgcta

SEQ ID NO: 23 - DNA, 20 bp, Artificial sequence
  Single strand DNA oligonucleotide
tcgactggaagaaggagtgg

SEQ ID NO: 24 - DNA, 22 bp, Artificial sequence
  Single strand DNA oligonucleotide
ttatttgaaaggggcaattgct

SEQ ID NO: 25 - DNA, 21 bp, Artificial sequence
  Single strand DNA oligonucleotide
gtgaactttccactctcacac

SEQ ID NO: 26 - DNA, 19 bp, Artificial sequence
  Single strand DNA oligonucleotide
  misc_feature (2)..(2): n is a, c, g, or t
  misc_feature (18)..(18): n is a, c, g, or t
cnacgcgaagaaccttanc

SEQ ID NO: 27 - DNA, 19 bp, Artificial sequence
  Single strand DNA oligonucleotide
atacgcgargaaccttacc

SEQ ID NO: 28 - DNA, 19 bp, Artificial sequence
  Single strand DNA oligonucleotide
  misc_feature (9)..(9): n is a, c, g, or t
ctaaccgangaacctyacc

SEQ ID NO: 29 - DNA, 19 bp, Artificial sequence
  Single strand DNA oligonucleotide
caacgcgmaraaccttacc

SEQ ID NO: 30 - DNA, 19 bp, Artificial sequence
  Single strand DNA oligonucleotide
  misc_feature (14)..(14): n is a, c, g, or t
cgacrrccatgcancacct

SEQ ID NO: 31 - DNA, 32 bp, Artificial sequence; identical to SEQ ID NO: 4
  Single strand DNA oligonucleotide

SEQ ID NO: 32 - DNA, 35 bp, Artificial sequence; identical to SEQ ID NO: 5
  Single strand DNA oligonucleotide

SEQ ID NO: 33 - DNA, 24 bp, Artificial sequence
  Single strand DNA oligonucleotide
gtgcttgcatcttgatttaatttt

SEQ ID NO: 34 - DNA, 21 bp, Artificial sequence
  Single strand DNA oligonucleotide
tgcggttcttggatctatgcg

SEQ ID NO: 35 - DNA, 24 bp, Artificial sequence
  Single strand DNA oligonucleotide
gtgcttgcactgagattcgactta

SEQ ID NO: 36 - DNA, 21 bp, Artificial sequence
  Single strand DNA oligonucleotide
tgcggttcttggatctatgcg

SEQ ID NO: 37 - DNA, 24 bp, Artificial sequence
  Single strand DNA oligonucleotide
gtgcttgcaccgagattcaacatg

SEQ ID NO: 38 - DNA, 21 bp, Artificial sequence
  Single strand DNA oligonucleotide
tgcggttcttggatctatgcg

SEQ ID NO: 39 - DNA, 24 bp, Artificial sequence
  Single strand DNA oligonucleotide
ttacatttgagtgagtggcgaact

SEQ ID NO: 40 - DNA, 20 bp, Artificial sequence
  Single strand DNA oligonucleotide
aggtgttatcccccgcttct

SEQ ID NO: 41 - DNA, 19 bp, Artificial sequence
  Single strand DNA oligonucleotide
cgcgagcaaaacaatggtt

SEQ ID NO: 42 - DNA, 25 bp, Artificial sequence
  Single strand DNA oligonucleotide
aacgatcgaaacgaacaatagagtt

SEQ ID NO: 43 - DNA, 21 bp, Artificial sequence
  Single strand DNA oligonucleotide
gttgatttcgccggactcttc

SEQ ID NO: 44 - DNA, 18 bp, Artificial sequence
  Single strand DNA oligonucleotide
gcaagcctatcgcgcaaa

SEQ ID NO: 45 - DNA, 23 bp, Artificial sequence
  Single strand DNA oligonucleotide
gtggtggcttgagaactggatag

SEQ ID NO: 46 - DNA, 25 bp, Artificial sequence
  Single strand DNA oligonucleotide
caaaacgatcgaaacaaacactaaa

SEQ ID NO: 47 - DNA, 20 bp, Artificial sequence
  Single strand DNA oligonucleotide
tggaagacgtcgttggcttt

SEQ ID NO: 48 - DNA, 16 bp, Artificial sequence
  Single strand DNA oligonucleotide
atcgcgccaggcaaaa

SEQ ID NO: 49 - DNA, 20 bp, Artificial sequence
  Single strand DNA oligonucleotide
tgaaccacaatgggttgcta

SEQ ID NO: 50 - DNA, 20 bp, Artificial sequence
  Single strand DNA oligonucleotide
tcgactggaagaaggagtgg

SEQ ID NO: 51 - DNA, 22 bp, Artificial sequence
  Single strand DNA oligonucleotide
ttatttgaaaggggcaattgct

SEQ ID NO: 52 - DNA, 21 bp, Artificial sequence
  Single strand DNA oligonucleotide
gtgaactttccactctcacac

SEQ ID NO: 53 - DNA, 19 bp, Artificial sequence
  Single strand DNA oligonucleotide
  misc_feature (2)..(2): n is a, c, g, or t
  misc_feature (18)..(18): n is a, c, g, or t
cnacgcgaagaaccttanc

SEQ ID NO: 54 - DNA, 19 bp, Artificial sequence
  Single strand DNA oligonucleotide
atacgcgargaaccttacc

SEQ ID NO: 55 - DNA, 19 bp, Artificial sequence
  Single strand DNA oligonucleotide
  misc_feature (9)..(9): n is a, c, g, or t
ctaaccgangaacctyacc

SEQ ID NO: 56 - DNA, 19 bp, Artificial sequence
  Single strand DNA oligonucleotide
caacgcgmaraaccttacc

SEQ ID NO: 57 - DNA, 19 bp, Artificial sequence
  Single strand DNA oligonucleotide
  misc_feature (14)..(14): n is a, c, g, or t
cgacrrccatgcancacct

SEQ ID NO: 58 - DNA, 31 bp, Artificial sequence; identical to SEQ ID NO: 1
  Single strand DNA oligonucleotide

SEQ ID NO: 59 - DNA, 32 bp, Artificial sequence; identical to SEQ ID NO: 2
  Single strand DNA oligonucleotide

SEQ ID NO: 60 - DNA, 39 bp, Artificial sequence
  Single strand DNA oligonucleotide
attagawacccbdgtagtccggctgactgactattagaa

SEQ ID NO: 61 - DNA, 50 bp, Artificial sequence
  Single strand DNA oligonucleotide
taatgatccttccgcaggttcacctacggaaaccttgttacgacttttac

SEQ ID NO: 62 - DNA, 50 bp, Artificial sequence
  Single strand DNA oligonucleotide
ttcctctagatagtcaagttcgaccgtcttctcagcgctccgccagggcc

SEQ ID NO: 63 - DNA, 50 bp, Artificial sequence
  Single strand DNA oligonucleotide
gtgggccgaccccggcggggccgatccgagggcctcactaaaccatccaa

SEQ ID NO: 64 - DNA, 50 bp, Artificial sequence
  Single strand DNA oligonucleotide
tcggtagtagcgacgggcggtgtgtacaaagggcagggacttaatcaacg

SEQ ID NO: 65 - DNA, 50 bp, Artificial sequence
  Single strand DNA oligonucleotide
caagcttatgacccgcacttactcgggaattccctcgttcatggggaata

SEQ ID NO: 66 - DNA, 50 bp, Artificial sequence
  Single strand DNA oligonucleotide
attgcaatccccgatccccatcacgaatggggttcaacgggttacccgcg

SEQ ID NO: 67 - DNA, 50 bp, Artificial sequence
  Single strand DNA oligonucleotide
cctgccggcgtagggtaggcacacgctgagccagtcagtgtagcgcgcgt

SEQ ID NO: 68 - DNA, 50 bp, Artificial sequence
  Single strand DNA oligonucleotide
gcagccccggacatctaagggcatcacagacctgttattgctcaatctcg

SEQ ID NO: 69 - DNA, 50 bp, Artificial sequence
  Single strand DNA oligonucleotide
ggtggctgaacgccacttgtccctctaagaagttgggggacgccgaccgc

SEQ ID NO: 70 - DNA, 50 bp, Artificial sequence
  Single strand DNA oligonucleotide
tcgggggtcgcgtaactagttagcatgccagagtctcgttcgttatcgga

SEQ ID NO: 71 - DNA, 50 bp, Artificial sequence
  Single strand DNA oligonucleotide
attaaccagacaaatcgctccaccaactaagaacggccatgcaccaccac

SEQ ID NO: 72 - DNA, 50 bp, Artificial sequence
  Single strand DNA oligonucleotide
ccacggaatcgagaaagagctatcaatctgtcaatcctgtccgtgtccgg

SEQ ID NO: 73 - DNA, 50 bp, Artificial sequence
  Single strand DNA oligonucleotide
gccgggtgaggtttcccgtgttgagtcaaattaagccgcaggctccactc

SEQ ID NO: 74 - DNA, 50 bp, Artificial sequence
  Single strand DNA oligonucleotide
ctggtggtgcccttccgtcaattcctttaagtttcagctttgcaaccata

SEQ ID NO: 75 - DNA, 50 bp, Artificial sequence
  Single strand DNA oligonucleotide
ctccccccggaacccaaagactttggtttcccggaagctgcccggcgggt

SEQ ID NO: 76 - DNA, 50 bp, Artificial sequence
  Single strand DNA oligonucleotide
catgggaataacgccgccgcatcgccggtcggcatcgtttatggtcggaa

SEQ ID NO: 77 - DNA, 50 bp, Artificial sequence
  Single strand DNA oligonucleotide
ctacgacggtatctgatcgtcttcgaacctccgactttcgttcttgatta

SEQ ID NO: 78 - DNA, 50 bp, Artificial sequence
  Single strand DNA oligonucleotide
atgaaaacattcttggcaaatgctttcgctctggtccgtcttgcgccggt

SEQ ID NO: 79 - DNA, 50 bp, Artificial sequence
  Single strand DNA oligonucleotide
ccaagaatttcacctctagcggcgcaatacgaatgcccccggccgtccct

SEQ ID NO: 80 - DNA, 50 bp, Artificial sequence
  Single strand DNA oligonucleotide
cttaatcatggcctcagttccgaaaaccaacaaaatagaaccgcggtcct

SEQ ID NO: 81 - DNA, 50 bp, Artificial sequence
  Single strand DNA oligonucleotide
attccattattcctagctgcggtatccaggcggctcgggcctgctttgaa

SEQ ID NO: 82 - DNA, 50 bp, Artificial sequence
  Single strand DNA oligonucleotide
cactctaattttttcaaagtaaacgcttcgggccccgcgggacactcagc

SEQ ID NO: 83 - DNA, 50 bp, Artificial sequence
  Single strand DNA oligonucleotide
taagagcatcgagggggcgccgagaggcaaggggcggggacgggcggtgg

SEQ ID NO: 84 - DNA, 50 bp, Artificial sequence
  Single strand DNA oligonucleotide
ctcgcctcgcggcggaccgcccgcccgctcccaagatccaactacgagct

SEQ ID NO: 85 - DNA, 50 bp, Artificial sequence
  Single strand DNA oligonucleotide
ttttaactgcagcaactttaatatacgctattggagctggaattaccgcg

SEQ ID NO: 86 - DNA, 50 bp, Artificial sequence
  Single strand DNA oligonucleotide
gctgctggcaccagacttgccctccaatggatcctcgttaaaggatttaa

SEQ ID NO: 87 - DNA, 50 bp, Artificial sequence
  Single strand DNA oligonucleotide
agtggactcattccaattacagggcctcgaaagagtcctgtattgttatt

SEQ ID NO: 88 - DNA, 50 bp, Artificial sequence
  Single strand DNA oligonucleotide
tttcgtcactacctccccgggtcgggagtgggtaatttgcgcgcctgctg

SEQ ID NO: 89 - DNA, 50 bp, Artificial sequence
  Single strand DNA oligonucleotide
ccttccttggatgtggtagccgtttctcaggctccctctccggaatcgaa

SEQ ID NO: 90 - DNA, 50 bp, Artificial sequence
  Single strand DNA oligonucleotide
ccctgattccccgtcacccgtggtcaccatggtaggcacggcgactacca

SEQ ID NO: 91 - DNA, 48 bp, Artificial sequence
  Single strand DNA oligonucleotide
tcgaaagttgatagggcagacgttcgaatgggtcgtcgccgccacggg

SEQ ID NO: 92 - DNA, 50 bp, Artificial sequence
  Single strand DNA oligonucleotide
gcgtgcgatcggcccgaggttatctagagtcaccaaagccgccggcgccc

SEQ ID NO: 93 - DNA, 50 bp, Artificial sequence
  Single strand DNA oligonucleotide
gccccccggccggggccggagaggggctgaccgggttggttttgatctga

SEQ ID NO: 94 - DNA, 50 bp, Artificial sequence
  Single strand DNA oligonucleotide
taaatgcacgcatcccccccgcgaagggggtcagcgcccgtcggcatgta

SEQ ID NO: 95 - DNA, 50 bp, Artificial sequence
  Single strand DNA oligonucleotide
ttagctctagaattaccacagttatccaagtaggagaggagcgagcgacc

SEQ ID NO: 96 - DNA, 50 bp, Artificial sequence
  Single strand DNA oligonucleotide
aaaggaaccataactgatttaatgagccattcgcagtttcactgtaccgg

SEQ ID NO: 97 - DNA, 50 bp, Artificial sequence
  Single strand DNA oligonucleotide
ccgtgcgtacttagacatgcatggcttaatctttgagacaagcatatgct

SEQ ID NO: 98 - DNA, 50 bp, Artificial sequence
  Single strand DNA oligonucleotide
tggcttaatctttgagacaagcatatgctactggcaggatcaaccaggta

SEQ ID NO: 99 - DNA, 50 bp, Artificial sequence
  Single strand DNA oligonucleotide
aagcgacgctcagacaggcgtagccccgggaggaacccggggccgcaagt

SEQ ID NO: 100 - DNA, 50 bp, Artificial sequence
  Single strand DNA oligonucleotide
gcgttcgaagtgtcgatgatcaatgtgtcctgcaattcacattaattctc

SEQ ID NO: 101 - DNA, 50 bp, Artificial sequence
  Single strand DNA oligonucleotide
gcagctagctgcgttcttcatcgacgcacgagccgagtgatccaccgcta

SEQ ID NO: 102 - DNA, 50 bp, Artificial sequence
  Single strand DNA oligonucleotide
aaaccctgttcttgggtgggtgtgggtataatactaagttgagatgatat

SEQ ID NO: 103 - DNA, 50 bp, Artificial sequence
  Single strand DNA oligonucleotide
catttacgggggaaggcgctttgtgaagtaggccttatttctcttgtcct

SEQ ID NO: 104 - DNA, 50 bp, Artificial sequence
  Single strand DNA oligonucleotide
  misc_feature (23)..(23): n is a, c, g, or t
ttcgtacagggaggaatttgaangtagatagaaaccgacctggattactc

SEQ ID NO: 105 - DNA, 50 bp, Artificial sequence
  Single strand DNA oligonucleotide
cggtctgaactcagatcacgtaggactttaatcgttgaacaaacgaacct

SEQ ID NO: 106 - DNA, 50 bp, Artificial sequence
  Single strand DNA oligonucleotide
ttaatagcggctgcaccatcgggatgtcctgatccaacatcgaggtcgta

SEQ ID NO: 107 - DNA, 50 bp, Artificial sequence
  Single strand DNA oligonucleotide
aaccctattgttgatatggactctagaataggattgcgctgttatcccta

SEQ ID NO: 108 - DNA, 50 bp, Artificial sequence
  Single strand DNA oligonucleotide
gggtaacttgttccgttggtcaagttattggatcaattgagtatagtagt

SEQ ID NO: 109 - DNA, 50 bp, Artificial sequence
  Single strand DNA oligonucleotide
tcgctttgactggtgaagtcttagcatgtactgctcggaggttgggttct

SEQ ID NO: 110 - DNA, 50 bp, Artificial sequence
  Single strand DNA oligonucleotide
gctccgaggtcgccccaaccgaaatttttaatgcaggtttggtagtttag

SEQ ID NO: 111 - DNA, 50 bp, Artificial sequence
  Single strand DNA oligonucleotide
gacctgtgggtttgttaggtactgtttgcattaataaattaaagctccat

SEQ ID NO: 112 - DNA, 50 bp, Artificial sequence
  Single strand DNA oligonucleotide
agggtcttctcgtcttgctgtgttatgcccgcctcttcacgggcaggtca

SEQ ID NO: 113 - DNA, 50 bp, Artificial sequence
  Single strand DNA oligonucleotide
atttcactggttaaaagtaagagacagctgaaccctcgtggagccattca

SEQ ID NO: 114 - DNA, 50 bp, Artificial sequence
  Single strand DNA oligonucleotide
tacaggtccctatttaaggaacaagtgattatgctacctttgcacggtta

SEQ ID NO: 115 - DNA, 50 bp, Artificial sequence
  Single strand DNA oligonucleotide
gggtaccgcggccgttaaacatgtgtcactgggcaggcggtgcctctaat

SEQ ID NO: 116 - DNA, 50 bp, Artificial sequence
  Single strand DNA oligonucleotide
actggtgatgctagaggtgatgtttttggtaaacaggcggggtaagattt

SEQ ID NO: 117 - DNA, 50 bp, Artificial sequence
  Single strand DNA oligonucleotide
gccgagttccttttactttttttaacctttccttatgagcatgcctgtgt

SEQ ID NO: 118 - DNA, 50 bp, Artificial sequence
  Single strand DNA oligonucleotide
tgggttgacagtgagggtaataatgacttgttggttgattgtagatattg

SEQ ID NO: 119 - DNA, 50 bp, Artificial sequence
  Single strand DNA oligonucleotide
ggctgttaattgtcagttcagtgttttaatctgacgcaggcttatgcgga

SEQ ID NO: 120 - DNA, 50 bp, Artificial sequence
  Single strand DNA oligonucleotide
ggagaatgttttcatgttacttatactaacattagttcttctatagggtg

SEQ ID NO: 121 - DNA, 50 bp, Artificial sequence
  Single strand DNA oligonucleotide
atagattggtccaattgggtgtgaggagttcagttatatgtttgggattt

SEQ ID NO: 122 - DNA, 50 bp, Artificial sequence
  Single strand DNA oligonucleotide
tttaggtagtgggtgttgagcttgaacgctttcttaattggtggctgctt

SEQ ID NO: 123 - DNA, 50 bp, Artificial sequence
  Single strand DNA oligonucleotide
ttaggcctactatgggtgttaaattttttactctctctacaaggtttttt

SEQ ID NO: 124 - DNA, 50 bp, Artificial sequence
  Single strand DNA oligonucleotide
cctagtgtccaaagagctgttcctctttggactaacagttaaatttacaa

SEQ ID NO: 125 - DNA, 49 bp, Artificial sequence
  Single strand DNA oligonucleotide
gggatttagagggttctgtgggcaaatttaaagttgaactaagattcta

SEQ ID NO: 126 - DNA, 50 bp, Artificial sequence
  Single strand DNA oligonucleotide
tcttggacaaccagctatcaccaggctcggtaggtttgtcgcctctacct

SEQ ID NO: 127 - DNA, 50 bp, Artificial sequence
  Single strand DNA oligonucleotide
ataaatcttcccactattttgctacatagacgggtgtgctcttttagctg

SEQ ID NO: 128 - DNA, 50 bp, Artificial sequence
  Single strand DNA oligonucleotide
ttcttaggtagctcgtctggtttcgggggtcttagctttggctctccttg

SEQ ID NO: 129 - DNA, 50 bp, Artificial sequence
  Single strand DNA oligonucleotide
caaagttatttctagttaattcattatgcagaaggtataggggttagtcc

SEQ ID NO: 130 - DNA, 50 bp, Artificial sequence
  Single strand DNA oligonucleotide
ttgctatattatgcttggttataatttttcatctttcccttgcggtacta

SEQ ID NO: 131 - DNA, 50 bp, Artificial sequence
  Single strand DNA oligonucleotide
tatctattgcgccaggtttcaatttctatcgcctatactttatttgggta

SEQ ID NO: 132 - DNA, 50 bp, Artificial sequence
  Single strand DNA oligonucleotide
aatggtttggctaaggttgtctggtagtaaggtggagtgggtttggggct

SEQ ID NO: 133 - DNA, 50 bp, Artificial sequence
  Single strand DNA oligonucleotide
gttcgtccaagtgcactttccagtacacttaccatgttacgacttgtctc

SEQ ID NO: 134 - DNA, 50 bp, Artificial sequence
  Single strand DNA oligonucleotide
ctctatataaatgcgtaggggttttagttaaatgtcctttgaagtatact

SEQ ID NO: 135 - DNA, 50 bp, Artificial sequence
  Single strand DNA oligonucleotide
tgaggagggtgacgggcggtgtgtacgcgcttcagggccctgttcaacta

SEQ ID NO: 136 - DNA, 50 bp, Artificial sequence
  Single strand DNA oligonucleotide
agcactctactcttagtttactgctaaatccaccttcgacccttaagttt

SEQ ID NO: 137 - DNA, 50 bp, Artificial sequence
  Single strand DNA oligonucleotide
cataagggctatcgtagttttctggggtagaaaatgtagcccatttcttg

SEQ ID NO: 138 - DNA, 50 bp, Artificial sequence
  Single strand DNA oligonucleotide
ccacctcatgggctacaccttgacctaacgtctttacgtgggtacttgcg

SEQ ID NO: 139 - DNA, 50 bp, Artificial sequence
  Single strand DNA oligonucleotide
cttactttgtagccttcatcagggtttgctgaagatggcggtatataggc

SEQ ID NO: 140 - DNA, 50 bp, Artificial sequence
  Single strand DNA oligonucleotide
tgagcaagaggtggtgaggttgatcggggtttatcgattacagaacaggc

SEQ ID NO: 141 - DNA, 50 bp, Artificial sequence
  Single strand DNA oligonucleotide
tcctctagagggatatgaagcaccgccaggtcctttgagttttaagctgt

SEQ ID NO: 142 - DNA, 50 bp, Artificial sequence
  Single strand DNA oligonucleotide
ggctcgtagtgttctggcgagcagttttgttgatttaactgttgaggttt

SEQ ID NO: 143 - DNA, 50 bp, Artificial sequence
  Single strand DNA oligonucleotide
agggctaagcatagtggggtatctaatcccagtttgggtcttagctattg

SEQ ID NO: 144 - DNA, 50 bp, Artificial sequence
  Single strand DNA oligonucleotide
tgtgttcagatatgttaaagccactttcgtagtctattttgtgtcaactg

SEQ ID NO: 145 - DNA, 50 bp, Artificial sequence
  Single strand DNA oligonucleotide
gagttttttacaactcaggtgagttttagctttattggggagggggtgat

SEQ ID NO: 146 - DNA, 50 bp, Artificial sequence
  Single strand DNA oligonucleotide
ctaaaacactctttacgccggcttctattgacttgggttaatcgtgtgac

SEQ ID NO: 147 - DNA, 50 bp, Artificial sequence
  Single strand DNA oligonucleotide
cgcggtggctggcacgaaattgaccaaccctggggttagtatagcttagt

SEQ ID NO: 148 - DNA, 48 bp, Artificial sequence
  Single strand DNA oligonucleotide
taaactttcgtttattgctaaaggttaatcactgctgtttcccgtggg

SEQ ID NO: 149 - DNA, 50 bp, Artificial sequence
  Single strand DNA oligonucleotide
tgtggctaggctaagcgttttgagctgcattgctgcgtgcttgatgcttg

SEQ ID NO: 150 - DNA, 50 bp, Artificial sequence
  Single strand DNA oligonucleotide
ttccttttgatcgtggtgatttagagggtgaactcactggaacggggatg

SEQ ID NO: 151 - DNA, 50 bp, Artificial sequence
  Single strand DNA oligonucleotide
cttgcatgtgtaatcttactaagagctaatagaaaggctaggaccaaacc

SEQ ID NO: 152 - DNA, 50 bp, Artificial sequence
  Single strand DNA oligonucleotide
aaagcctacagcacccggtattcccaggcggtctcccatccaagtactaa

SEQ ID NO: 153 - DNA, 50 bp, Artificial sequence
  Single strand DNA oligonucleotide
ccaggcccgaccctgcttagcttccgagatcagacgagatcgggcgcgtt

SEQ ID NO: 154 - DNA, 50 bp, Artificial sequence
  Single strand DNA oligonucleotide
ttccgagatcagacgagatcgggcgcgttcagggtggtatggccgtagac